(19) United States
(12) Patent Application Publication
(10) Pub. No.: US 2024/0237516 A1
(43) Pub. Date: Jul. 11, 2024

(54) HETEROCYCLIC COMPOUND AND ORGANIC LIGHT EMITTING DEVICE COMPRISING THE SAME

(71) Applicant: LT MATERIALS CO., LTD., Yongin-si (KR)

(72) Inventors: Dong Jin LEE, Yongin-si (KR); Won Jang JEONG, Yongin-si (KR); Dong Jun KIM, Yongin-si (KR); Dae Hyuk CHOI, Yongin-si (KR)

(73) Assignee: LT MATERIALS CO., LTD., Yongin-si (KR)

(21) Appl. No.: 18/512,180

(22) Filed: Nov. 17, 2023

(30) Foreign Application Priority Data
Dec. 12, 2022 (KR) 10-2022-0172858

(51) Int. Cl. H10K 85/60; C07D 313/06; C07D 407/12; C07D 409/12; C09K 11/06

(52) U.S. Cl. CPC H10K 85/636; C07D 313/06; C07D 407/12; C07D 409/12; C09K 11/06; H10K 85/633; H10K 50/15

(57) ABSTRACT

The present specification relates to a heterocyclic compound represented by the following Chemical Formula 1 and an organic light emitting device including the same:

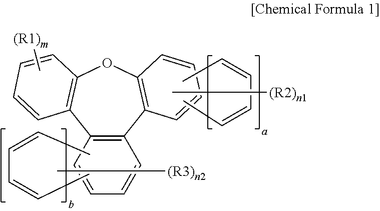

in Chemical Formula 1,
R1 to R3 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; and a group represented by the following Chemical Formula 2,
any one of R1 to R3 is the group represented by the following Chemical Formula 2, however, the remaining two are not the group represented by the following Chemical Formula 2,
a and b are each independently an integer of 0 or 1, and satisfy a+b=1,
m is one of integers of 1 to 4,
when a or b is 0, n1 or n2 is each independently one of integers of 1 to 4, and
when a or b is 1, n1 or n2 is each independently one of integers of 1 to 6,

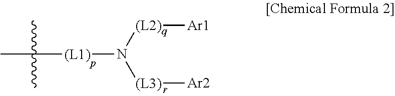

in Chemical Formula 2,
L1, L2 and L3 are the same as or different from each other, and each independently a single bond; a substituted or unsubstituted C6 to C60 arylene group; or a substituted or unsubstituted C2 to C60 heteroarylene group,
Ar1 and Ar2 are the same as or different from each other, and each independently a substituted or unsubstituted C6 to C60 aryl group; or a substituted or unsubstituted C2 to C60 heteroaryl group, and
p, q and r are the same as or different from each other, and each independently an integer of 0 to 3.

### CROSS-REFERENCE TO RELATED APPLICATION (S)

This application claims priority to Korean Patent Application No. 10-2022-0172858, filed on Dec. 12, 2022, the disclosure of which is incorporated herein by reference in its entirety.

### TECHNICAL FIELD

The present disclosure relates to a heterocyclic compound and an organic light emitting device including the same.

### DESCRIPTION OF THE RELATED ART

An organic light emitting device is one type of self-emissive display devices, and has advantages of having a wide viewing angle and a high response speed as well as having an excellent contrast.

The organic light emitting device has a structure of disposing an organic thin film between two electrodes. When a voltage is applied to the organic light emitting device having such a structure, electrons and holes injected from the two electrodes bind and pair in the organic thin film, and then light is emitted as these annihilate. The organic thin film may be formed in a single layer or a multilayer as necessary.

A material of the organic thin film may have a light emitting function as necessary. For example, as a material of the organic thin film, compounds each capable of forming a light emitting layer themselves alone may be used, or compounds each capable of serving as a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

(Patent Document 1) U.S. Pat. No. 4,356,429

### SUMMARY

The present disclosure is directed to providing a heterocyclic compound and an organic light emitting device including the same.

One embodiment of the present disclosure provides a heterocyclic compound represented by the following Chemical Formula 1:

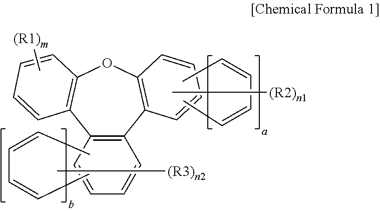

in Chemical Formula 1,
R1 to R3 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; and a group represented by the following Chemical Formula 2,
any one of R1 to R3 is the group represented by the following Chemical Formula 2, however, the remaining two are not the group represented by the following Chemical Formula 2,
a and b are each independently an integer of 0 or 1, and satisfy a+b=1,
m is one of integers of 1 to 4,
when a or b is 0, n1 or n2 is each independently one of integers of 1 to 4, and
when a or b is 1, n1 or n2 is each independently one of integers of 1 to 6,

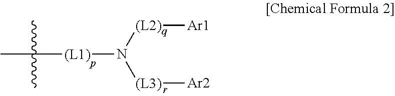

in Chemical Formula 2,
L1, L2 and L3 are the same as or different from each other, and each independently a single bond; a substituted or unsubstituted C6 to C60 arylene group; or a substituted or unsubstituted C2 to C60 heteroarylene group,
Ar1 and Ar2 are the same as or different from each other, and each independently a substituted or unsubstituted C6 to C60 aryl group; or a substituted or unsubstituted C2 to C60 heteroaryl group, and
p, q and r are the same as or different from each other, and each independently an integer of 0 to 3.

In addition, one embodiment of the present disclosure provides an organic light emitting device including: a first electrode; a second electrode provided opposite to the first electrode; and one or more organic material layers provided between the first electrode and the second electrode, wherein one or more layers of the organic material layers include the heterocyclic compound according to the present disclosure.

A heterocyclic compound according to one embodiment can be used as an organic material layer material of an organic light emitting device. The compound is capable of performing roles of a hole injection layer material, an electron blocking layer material, a hole transport layer material, a light emitting layer material, an electron transport layer material, a hole blocking layer material, an electron injection layer material and the like in an organic light emitting device. Particularly, the compound can be used as a material of a hole transport layer or an electron blocking layer of an organic light emitting device.

Specifically, the heterocyclic compound can be used as a hole transport layer material or an electron blocking layer material either alone or as a combination with other compounds.

In addition, using the heterocyclic compound according to one embodiment as a hole transport layer or an electron blocking layer strengthens hole properties, enhances a hole transfer ability through adjusting a band gap and a triplet energy level (T1 level) value, and increases molecular stability, thereby lowering a driving voltage of an organic light emitting device, enhancing light efficiency, and enhancing lifetime properties of an organic light emitting device by enhanced thermal stability of the compound.

### BRIEF DESCRIPTION OF THE DRAWINGS

FIGS. 1 to 4 are diagrams each schematically illustrating a lamination structure of an organic light emitting device according to one embodiment of the present disclosure.

### DESCRIPTION OF SPECIFIC EMBODIMENTS

Hereinafter, the present disclosure will be described in more detail.

In the present specification, a term “substitution” means that a hydrogen atom bonding to a carbon atom of a compound is changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of deuterium; halogen; a cyano group; a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; —P(═O)RR′; a C1 to C20 alkylamine group; a C6 to C60 monocyclic or polycyclic arylamine group; and a C2 to C60 monocyclic or polycyclic heteroarylamine group or being unsubstituted, or being substituted with a substituent in which two or more substituents selected from among the substituents exemplified above are linked or being unsubstituted.

In the present specification, R, R′ and R″ are the same as or different from each other, and may be each independently a substituted or unsubstituted C1 to C60 alkyl group; a substituted or unsubstituted C6 to C60 aryl group; or a substituted or unsubstituted C2 to C60 heteroaryl group.

In the present specification, “the number of protons” means the number of substituents that a specific compound may have, and specifically, the number of protons may mean the number of hydrogens. For example, unsubstituted benzene may be expressed to have the number of protons of 5, an unsubstituted naphthyl group may be expressed to have the number of protons of 7, a naphthyl group substituted with a phenyl group may be expressed to have the number of protons of 6, and an unsubstituted biphenyl group may be expressed to have the number of protons of 9.

In the present specification, the halogen may be fluorine, chlorine, bromine or iodine.

In the present specification, the alkyl group includes a linear or branched form having 1 to 60 carbon atoms, and may be further substituted with other substituents. The number of carbon atoms of the alkyl group may be from 1 to 60, specifically from 1 to 40 and more specifically from 1 to 20. Specific examples of the alkyl group may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an group, n-heptyl a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 4-methylhexyl group, a 5-methylhexyl group and the like, but are not limited thereto.

In the present specification, the alkenyl group includes a linear or branched form having 2 to 60 carbon atoms, and may be further substituted with other substituents. The number of carbon atoms of the alkenyl group may be from 2 to 60, specifically from 2 to 40 and more specifically from 2 to 20. Specific examples of the alkenyl group may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group and the like, but are not limited thereto.

In the present specification, the alkynyl group includes a linear or branched form having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples of the alkoxy group may include a methoxy group, an ethoxy group, an n-propoxy group, an isopropoxy group, an n-butoxy group, an isobutoxy group, a tert-butoxy group, a sec-butoxy group, an n-pentyloxy group, a neopentyloxy group, an isopentyloxy group, an n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group and the like, but are not limited thereto.

In the present specification, the cycloalkyl group includes a monocyclic or polycyclic group having 3 to 60 carbon atoms, and may be further substituted with other substituents. Herein, the polycyclic group means a group in which the cycloalkyl group is directly linked to or fused with another cyclic group. Herein, the another cyclic group may be a cycloalkyl group, but may also be different types of cyclic groups such as a heterocycloalkyl group, an aryl group and a heteroaryl group. The number of carbon atoms of the cycloalkyl group may be from 3 to 60, specifically from 3 to 40 and more specifically from 5 to 20. Specific examples of the cycloalkyl group may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes a monocyclic or polycyclic group having 2 to 60 carbon atoms, and may be further substituted with other substituents. Herein, the polycyclic group means a group in which the heterocycloalkyl group is directly linked to or fused with another cyclic group. Herein, the another cyclic group may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes a monocyclic or polycyclic group having 6 to 60 carbon atoms, and may be further substituted with other substituents. Herein, the polycyclic group means a group in which the aryl group is directly linked to or fused with another cyclic group. Herein, the another cyclic group may be an aryl group, but may also be different types of cyclic groups such as a cycloalkyl group, a heterocycloalkyl group and a heteroaryl group. The aryl group may include a spiro group. The number of carbon atoms of the aryl group may be from 6 to 60, specifically from 6 to 40 and more specifically from 6 to 25. Specific examples of the aryl group may include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the phosphine oxide group is represented by —P(═O) R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, the examples described above may be applied. Examples of the phosphine oxide group may include a diphenylphosphine oxide group, a dinaphthylphosphine oxide group and the like, but are not limited thereto.

In the present specification, the silyl group is a substituent including Si and having the Si atom directly linked as a radical, and is represented by —SiR101R102R103. R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

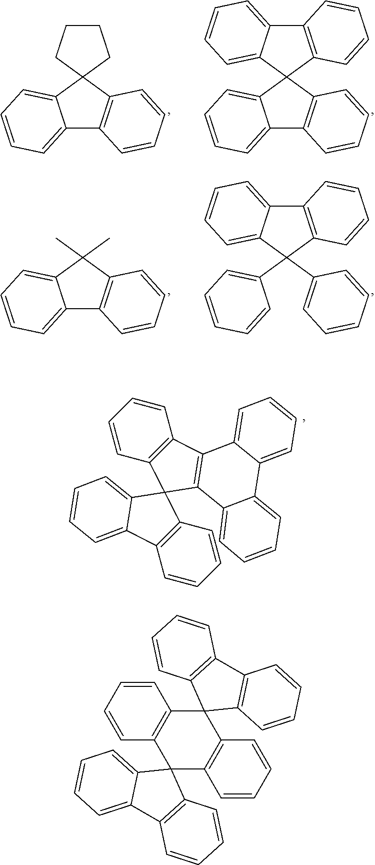

and the like may be included, however, the structure is not limited thereto.

In the present specification, the spiro group is a group including a spiro structure, and may have 15 to 60 carbon atoms. For example, the spiro group may include a structure in which a 2,3-dihydro-1H-indene group or a cyclohexane group spiro bonds to a fluorenyl group. Specifically, the spiro group may include any one of groups of the following structural formulae.

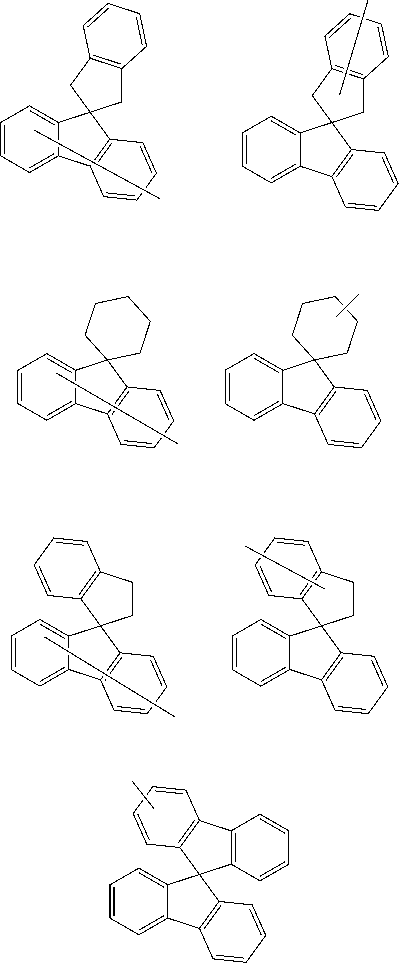

In the present specification, the heteroaryl group includes S, O, Se, N or Si as a heteroatom, includes a monocyclic or polycyclic group having 2 to 60 carbon atoms, and may be further substituted with other substituents. Herein, the polycyclic group means a group in which the heteroaryl group is directly linked to or fused with another cyclic group. Herein, the another cyclic group may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolinyl group, a naphthyridyl group, an acridinyl group, a phenanthridinyl group, an imidazopyridinyl group, a diazanaphthalenyl group, a triazaindenyl group, 2-indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilole group, a spirobi (dibenzosilole) group, a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydro-dibenzo[b,f]azepinyl group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms is not particularly limited, but preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, another substituent substituting an atom substituted by the corresponding substituent. For example, two substituents substituting at ortho positions in a benzene ring, and two substituents substituting at the same carbon in an aliphatic ring may be interpreted as groups “adjacent” to each other.

In the present disclosure, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present disclosure, a “case of a substituent being not indicated in a chemical formula or compound structure” may mean that positions to which substituents may come are all hydrogen or deuterium. In other words, since deuterium is an isotope of hydrogen, some hydrogen atoms may be deuterium that is an isotope, and herein, a content of the deuterium may be from 0% to 100%.

In one embodiment of the present disclosure, in a “case of a substituent being not indicated in a chemical formula or compound structure”, hydrogen and deuterium may be used interchangeably in compounds when deuterium is not explicitly excluded such as “a deuterium content being 0%”, “a hydrogen content being 100%” or “substituents being all hydrogen”.

In one embodiment of the present disclosure, deuterium is one of isotopes of hydrogen, is an element having deuteron formed with one proton and one neutron as a nucleus, and may be expressed as hydrogen-2, and the elemental symbol thereof may also be written as D or ²H.

In one embodiment of the present disclosure, an isotope means an atom with the same atomic number (Z) but with a different mass number (A), and may also be interpreted as an element with the same number of protons but with a different number of neutrons.

In one embodiment of the present disclosure, a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

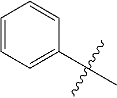 [SMILES: *c1ccccc1]

may mean that the total number of substituents that the phenyl group may have is 5 (T1 in the formula), and the number of deuterium atoms among these is 1 (T2 in the formula). In other words, having a deuterium content of 20% in a phenyl group may be represented by the following structural formulae:

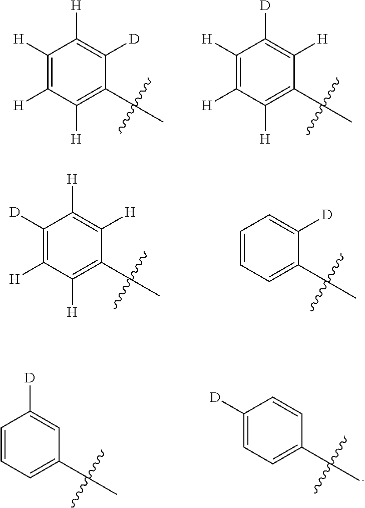

In addition, in one embodiment of the present disclosure, “a phenyl group having a deuterium content of 0%” may mean a phenyl group that does not include a deuterium atom, that is, a phenyl group that has 5 hydrogen atoms.

In the present disclosure, the C6 to C60 aromatic hydrocarbon ring means a compound including an aromatic ring formed with C6 to C60 carbons and hydrogens. Examples thereof may include benzene, biphenyl, terphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art and satisfying the above-mentioned number of carbon atoms.

One embodiment of the present disclosure provides a heterocyclic compound represented by the following Chemical Formula 1:

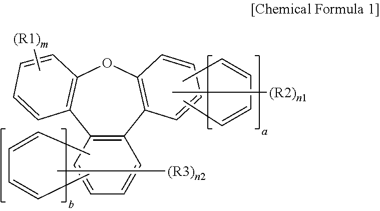

in Chemical Formula 1,
R1 to R3 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; and a group represented by the following Chemical Formula 2,
any one of R1 to R3 is the group represented by the following Chemical Formula 2, however, the remaining two are not the group represented by the following Chemical Formula 2,
a and b are each independently an integer of 0 or 1, and satisfy a+b=1,
m is one of integers of 1 to 4,
when a or b is 0, n1 or n2 is each independently one of integers of 1 to 4, and
when a or b is 1, n1 or n2 is each independently one of integers of 1 to 6,

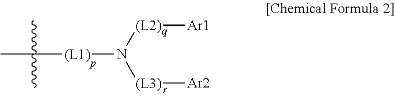

in Chemical Formula 2,
L1, L2 and L3 are the same as or different from each other, and each independently a single bond; a substituted or unsubstituted C6 to C60 arylene group; or a substituted or unsubstituted C2 to C60 heteroarylene group,
Ar1 and Ar2 are the same as or different from each other, and each independently a substituted or unsubstituted C6 to C60 aryl group; or a substituted or unsubstituted C2 to C60 heteroaryl group, and
p, q and r are the same as or different from each other, and each independently an integer of 0 to 3.

In one embodiment of the present disclosure, a and b are each independently an integer of 0 or 1, but may satisfy a+b=1.

When a is 0, b may be 1.

When b is 0, a may be 1.

In one embodiment of the present disclosure, the compound of Chemical Formula 1 may be represented by the following Chemical Formula 1-a, Chemical Formula 1-b or Chemical Formula 1-c:

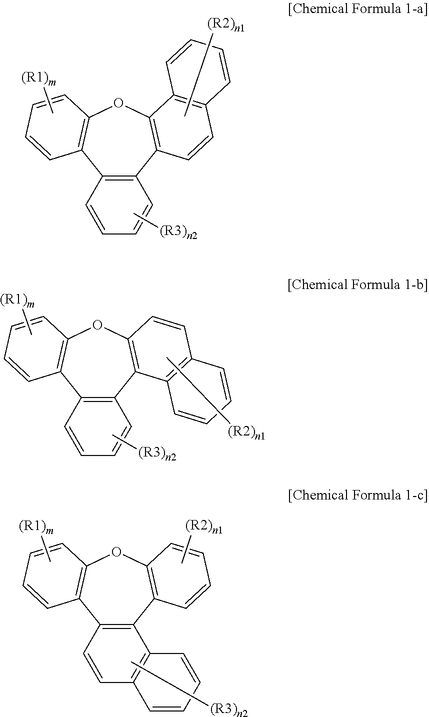

in Chemical Formulae 1-a to 1-c,
R1, R2, R3, m, n1 and n2 have the same definitions as in Chemical Formula 1.

In one embodiment of the present disclosure, R1 to R3 are the same as or different from each other and each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; and the group represented by Chemical Formula 2, and any one of R1 to R3 is the group represented by Chemical Formula 2, however, the remaining two may not be the group represented by Chemical Formula 2.

In one embodiment of the present disclosure, R1 to R3 are the same as or different from each other and each independently selected from the group consisting of hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; and the group represented by Chemical Formula 2, and any one of R1 to R3 is the group represented by Chemical Formula 2, however, the remaining two may not be the group represented by Chemical Formula 2.

In one embodiment of the present disclosure, R1 to R3 are the same as or different from each other and each independently selected from the group consisting of hydrogen; deuterium; halogen; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C1 to C10 alkoxy group; a substituted or unsubstituted C3 to C10 cycloalkyl group; a substituted or unsubstituted C2 to C10 heterocycloalkyl group; a substituted or unsubstituted C6 to C10 aryl group; a substituted or unsubstituted C2 to C10 heteroaryl group; and the group represented by Chemical Formula 2, and any one of R1 to R3 is the group represented by Chemical Formula 2, however, the remaining two may not be the group represented by Chemical Formula 2.

In one embodiment of the present disclosure, R1 to R3 are the same as or different from each other and each independently selected from the group consisting of hydrogen; deuterium; a substituted or unsubstituted C6 to C10 aryl group; a substituted or unsubstituted C2 to C10 heteroaryl group; and the group represented by Chemical Formula 2, and any one of R1 to R3 is the group represented by Chemical Formula 2, however, the remaining two may not be the group represented by Chemical Formula 2.

In one embodiment of the present disclosure, m may be an integer of 1, 2, 3 or 4.

In one embodiment of the present disclosure, when a is 0, n1 may be an integer of 1, 2, 3 or 4.

In one embodiment of the present disclosure, when b is 0, n2 may be an integer of 1, 2, 3 or 4.

In one embodiment of the present disclosure, when a is 1, n1 may be an integer of 1, 2, 3,4,5 or 6.

In one embodiment of the present disclosure, when b is 1, n2 may be an integer of 1, 2, 3,4,5 or 6.

In one embodiment of the present disclosure, the compound of Chemical Formula 1 may be represented by the following Chemical Formula 1-a-1, Chemical Formula 1-a-2, Chemical Formula 1-a-3, Chemical Formula 1-b-1, Chemical Formula 1-b-2, Chemical Formula 1-b-3, Chemical Formula 1-c-1, Chemical Formula 1-c-2 or Chemical Formula 1-c-3:

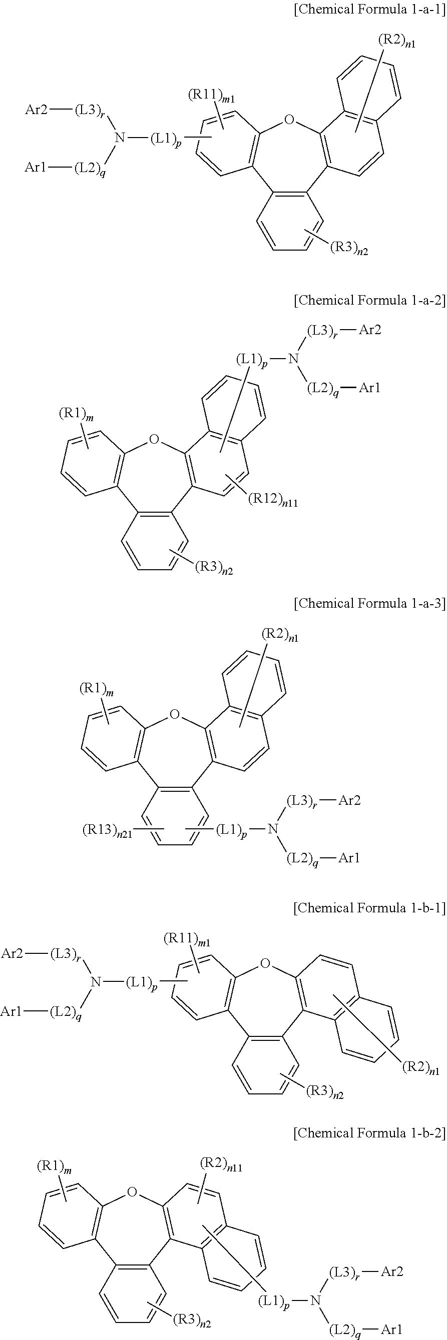

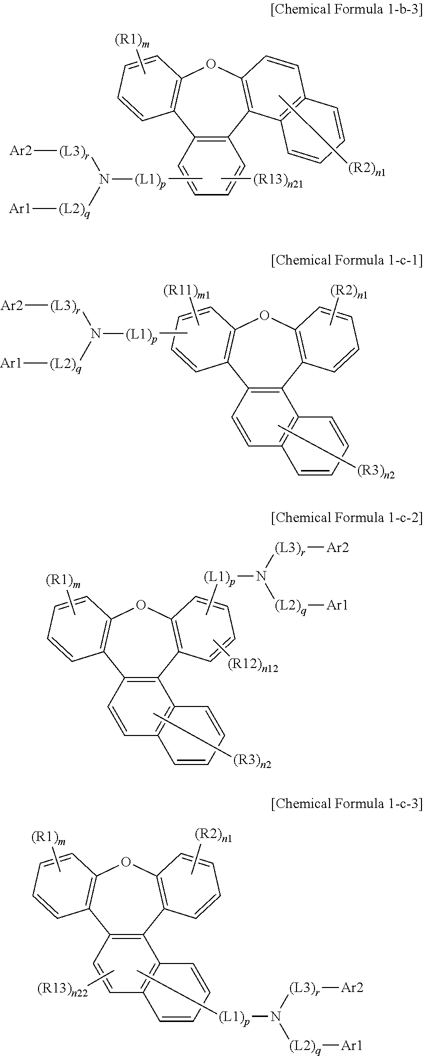

in Chemical Formulae 1-a-1 to 1-c-3,
R1, R2, R3, m, n1, n2, L1, L2, L3, Ar1, Ar2, p, q and r have the same definitions as in Chemical Formula 1,
R11, R12 and R13 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; and a substituted or unsubstituted C2 to C60 heterocycloalkyl group,
m1 is one of integers of 1 to 3,
n11 is one of integers of 1 to 5,
n12 is one of integers of 1 to 3,
n21 is one of integers of 1 to 3, and
n22 is one of integers of 1 to 5.

In one embodiment of the present disclosure, R11, R12 and R13 are the same as or different from each other, and may be each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; and a substituted or unsubstituted C2 to C30 heterocycloalkyl group.

In one embodiment of the present disclosure, R11, R12 and R13 are the same as or different from each other, and may be each independently selected from the group consisting of hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; and a substituted or unsubstituted C2 to C20 heterocycloalkyl group.

In one embodiment of the present disclosure, R11, R12 and R13 are the same as or different from each other, and may be each independently selected from the group consisting of hydrogen; deuterium; halogen; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C1 to C10 alkoxy group; a substituted or unsubstituted C3 to C10 cycloalkyl group; and a substituted or unsubstituted C2 to C10 heterocycloalkyl group.

In one embodiment of the present disclosure, R11, R12 and R13 are the same as or different from each other, and may be each independently hydrogen or deuterium.

In one embodiment of the present disclosure, m1 may be an integer of 1, 2 or 3.

In one embodiment of the present disclosure, n11 may be an integer of 1, 2, 3, 4 or 5.

In one embodiment of the present disclosure, n12 may be an integer of 1, 2 or 3.

In one embodiment of the present disclosure, n21 may be an integer of 1, 2 or 3.

In one embodiment of the present disclosure, n22 may be an integer of 1, 2, 3 or 5.

In one embodiment of the present disclosure, when the compound of Chemical Formula 1 is represented by Chemical Formula 1-a-1, Chemical Formula 1-a-2, Chemical Formula 1-a-3, Chemical Formula 1-b-1, Chemical Formula 1-b-2, Chemical Formula 1-b-3, Chemical Formula 1-c-1, Chemical Formula 1-c-2 or Chemical Formula 1-c-3, at least one of R1 to R3 may be hydrogen; deuterium; or a substituted or unsubstituted C6 to C60 aryl group.

In one embodiment of the present disclosure, when the compound of Chemical Formula 1 is represented by Chemical Formula 1-a-1, Chemical Formula 1-a-2, Chemical Formula 1-a-3, Chemical Formula 1-b-1, Chemical Formula 1-b-2, Chemical Formula 1-b-3, Chemical Formula 1-c-1, Chemical Formula 1-c-2 or Chemical Formula 1-c-3 and at least one of R1 to R3 is a substituted or unsubstituted C6 to C60 aryl group, the aryl group may be selected from the group consisting of the following structural formulae:

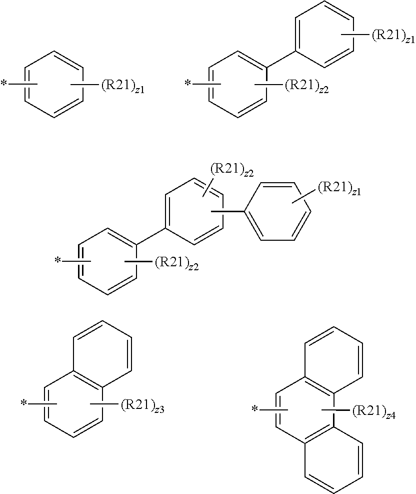

wherein,
R21 is selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; and a substituted or unsubstituted C2 to C60 heterocycloalkyl group,
z1 is one of integers of 1 to 5,
z2 is one of integers of 1 to 4,
z3 is one of integers of 1 to 7,
z4 is one of integers of 1 to 9, and
* is a position bonding to the atom of Chemical Formula 1.

In one embodiment of the present disclosure, R21 may be selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; and a substituted or unsubstituted C2 to C30 heterocycloalkyl group.

In one embodiment of the present disclosure, R21 may be selected from the group consisting of hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; and a substituted or unsubstituted C2 to C20 heterocycloalkyl group.

In one embodiment of the present disclosure, R21 may be selected from the group consisting of hydrogen; deuterium; halogen; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C1 to C10 alkoxy group; a substituted or unsubstituted C3 to C10 cycloalkyl group; and a substituted or unsubstituted C2 to C10 heterocycloalkyl group.

In one embodiment of the present disclosure, R21 may be selected from the group consisting of hydrogen; deuterium; halogen; and a cyano group.

In one embodiment of the present disclosure, R21 may be hydrogen or deuterium.

In one embodiment of the present disclosure, z1 may be an integer of 1, 2, 3, 4 or 5.

In one embodiment of the present disclosure, z2 may be an integer of 1, 2, 3 or 4.

In one embodiment of the present disclosure, z3 may be an integer of 1, 2, 3,4,5, 6 or 7.

In one embodiment of the present disclosure, z4 may be an integer of 1, 2, 3,4,5, 6, 7, 8 or 9.

In one embodiment of the present disclosure, in Chemical Formula 2, L1, L2 and L3 are the same as or different from each other, and may be each independently a single bond; a substituted or unsubstituted C6 to C30 arylene group; or a substituted or unsubstituted C2 to C30 heteroarylene group.

In one embodiment of the present disclosure, in Chemical Formula 2, L1, L2 and L3 are the same as or different from each other, and may be each independently a single bond; a substituted or unsubstituted C6 to C20 arylene group; or a substituted or unsubstituted C2 to C20 heteroarylene group.

In one embodiment of the present disclosure, in Chemical Formula 2, L1, L2 and L3 are the same as or different from each other, and may be each independently a single bond; or a substituted or unsubstituted C6 to C20 arylene group.

In one embodiment of the present disclosure, in Chemical Formula 2, L1, L2 and L3 are the same as or different from each other, and may be each independently a single bond; a substituted or unsubstituted group; or a substituted or unsubstituted biphenylene group.

In one embodiment of the present disclosure, in Chemical Formula 2, Ar1 and Ar2 are the same as or different from each other, and may be each independently a substituted or unsubstituted C6 to C40 aryl group; or a substituted or unsubstituted C2 to C40 heteroaryl group.

In one embodiment of the present disclosure, in Chemical Formula 2, Ar1 and Ar2 are the same as or different from each other, and may be each independently a substituted or unsubstituted C6 to C30 aryl group; or a substituted or unsubstituted C2 to C30 heteroaryl group.

In one embodiment of the present disclosure, in Chemical Formula 2, Ar1 and Ar2 are the same as or different from each other, and may be each independently selected from the group consisting of a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group; a substituted or unsubstituted fluorenyl group; a substituted or unsubstituted benzofluorenyl group; a substituted or unsubstituted dibenzofluorenyl group; a substituted or spiro-bifluorenyl group; unsubstituted a substituted or unsubstituted benzofuranyl group; a substituted or unsubstituted dibenzofuranyl group; a substituted or unsubstituted benzothiophenyl group; and a substituted or unsubstituted dibenzothiophenyl group.

In one embodiment of the present disclosure, in Chemical Formula 2, Ar1 and Ar2 may be each independently selected from the group consisting of the following structural formulae:

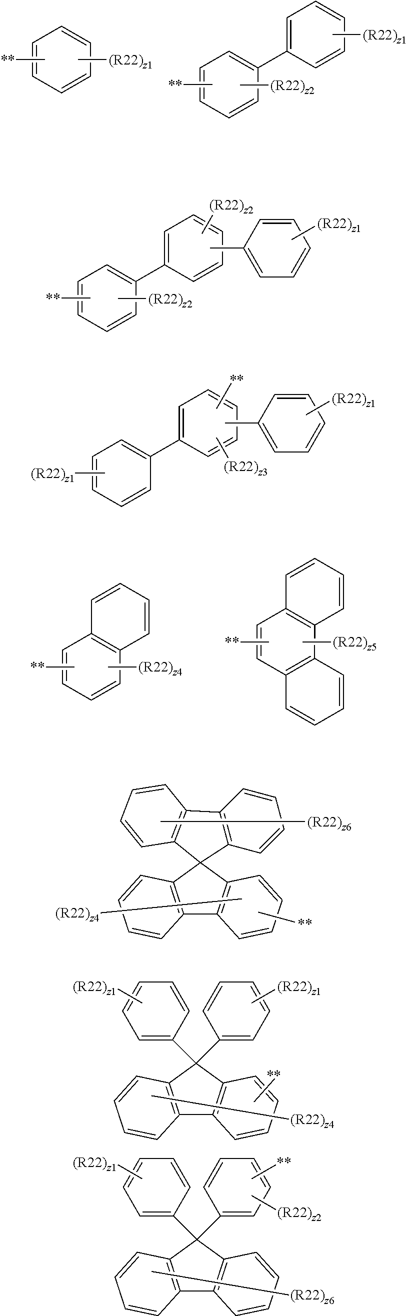

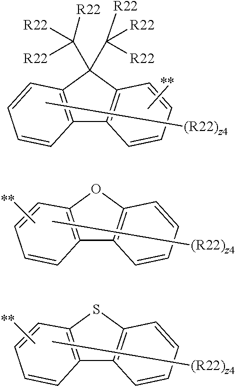

wherein,
R22 is selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; and a substituted or unsubstituted C2 to C60 heterocycloalkyl group,
z1 is one of integers of 1 to 5,
z2 is one of integers of 1 to 4,
z3 is one of integers of 1 to 3,
z4 is one of integers of 1 to 7,
z5 is one of integers of 1 to 9,
z6 is one of integers of 1 to 8, and
** is a position bonding to the N atom, L2 or L3 of Chemical Formula 2.

In one embodiment of the present disclosure, R22 may be selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; and a substituted or unsubstituted C2 to C30 heterocycloalkyl group.

In one embodiment of the present disclosure, R22 may be selected from the group consisting of hydrogen; deuterium; halogen; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C1 to C10 alkoxy group; a substituted or unsubstituted C3 to C10 cycloalkyl group; and a substituted or unsubstituted C2 to C10 heterocycloalkyl group.

In one embodiment of the present disclosure, R22 may be hydrogen or deuterium.

In one embodiment of the present disclosure, z1 may be an integer of 1, 2, 3, 4 or 5.

In one embodiment of the present disclosure, z2 may be an integer of 1, 2, 3 or 4.

In one embodiment of the present disclosure, z3 may be an integer of 1, 2 or 3.

In one embodiment of the present disclosure, z4 may be an integer of 1, 2, 3,4,5, 6 or 7.

In one embodiment of the present disclosure, z5 may be an integer of 1, 2, 3,4,5, 6, 7, 8 or 9.

In one embodiment of the present disclosure, z6 may be an integer of 1, 2, 3,4,5, 6, 7 or 8.

In one embodiment of the present disclosure, the heterocyclic compound represented by Chemical Formula 1 may not include deuterium as a substituent, or may have a deuterium content of, for example, greater than 0%, 18 or greater, 10% or greater, 20% or greater, 30% or greater, 40% or greater or 50% or greater, and 100% or less, 90% or less, 80% or less, 70% or less or 60% or less, with respect to the total number of hydrogen atoms and deuterium atoms.

In one embodiment of the present disclosure, the heterocyclic compound represented by Chemical Formula 1 may not include deuterium, or may have a deuterium content of 1% to 100% with respect to the total number of hydrogen atoms and deuterium atoms.

In one embodiment of the present disclosure, the heterocyclic compound represented by Chemical Formula 1 may not include deuterium, or may have a deuterium content of 20% to 90% with respect to the total number of hydrogen atoms and deuterium atoms.

In one embodiment of the present disclosure, the heterocyclic compound represented by Chemical Formula 1 may not include deuterium, or may have a deuterium content of 30% to 80% with respect to the total number of hydrogen atoms and deuterium atoms.

In one embodiment of the present disclosure, the heterocyclic compound represented by Chemical Formula 1 may not include deuterium, or may have a deuterium content of 40% to 70% with respect to the total number of hydrogen atoms and deuterium atoms.

For example, the heterocyclic compound represented by Chemical Formula 1 may have a deuterium content of 0% or greater, 1% or greater, 5% or greater, 10% or greater, 15% or greater, 20% or greater, 25% or greater, 30% or greater, 35% or greater, 40% or greater, 45% or greater or 50% or greater, and 100% or less, 95% or less, 90% or less, 85% or less, 80% or less, 75% or less, 70% or less, 65% or less or 60% or less based on the total number of hydrogen atoms and deuterium atoms.

In one embodiment of the present disclosure, the heterocyclic compound represented by Chemical Formula 1 may be any one selected from the group consisting of the following compounds:

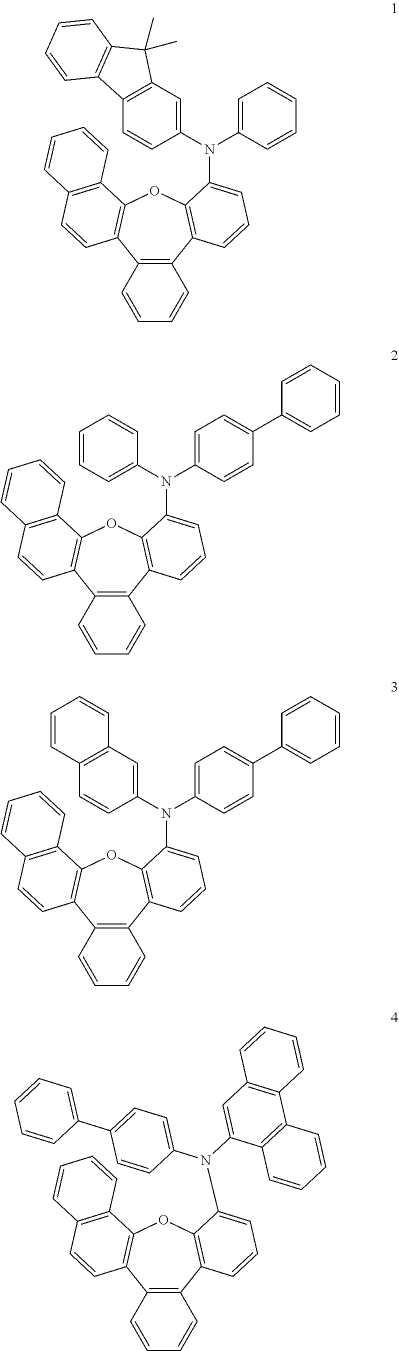

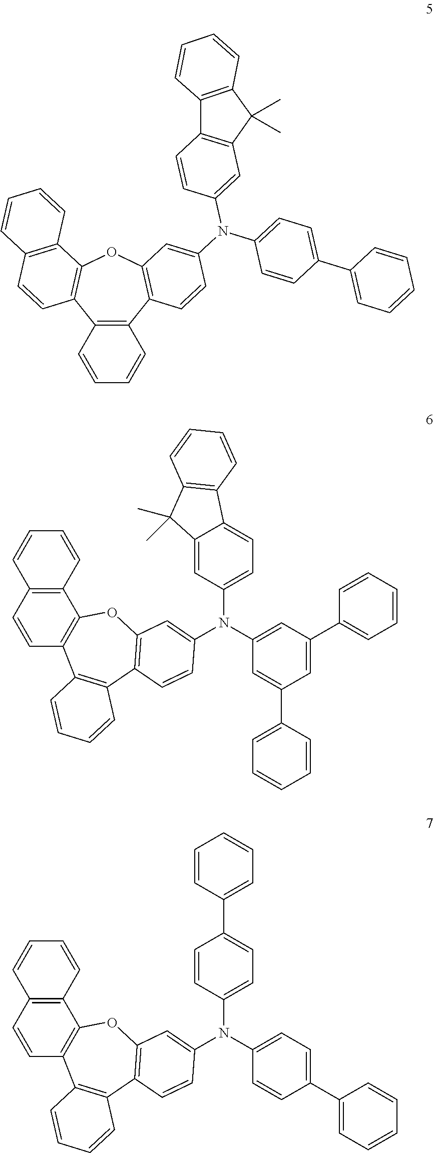 [SMILES: CC1(C)c2ccccc2-c2ccc(N(c3ccc(-c4ccccc4)cc3)c3ccc4c(c3)Oc3c(ccc5ccccc35)-c3ccccc3-4)cc21 ; CC1(C)c2ccccc2-c2ccc(N(c3cc(-c4ccccc4)cc(-c4ccccc4)c3)c3ccc4c(c3)Oc3c(ccc5ccccc35)-c3ccccc3-4)cc21 ; c1ccc(-c2ccc(N(c3ccc(-c4ccccc4)cc3)c3ccc4c(c3)Oc3c(ccc5ccccc35)-c3ccccc3-4)cc2)cc1]

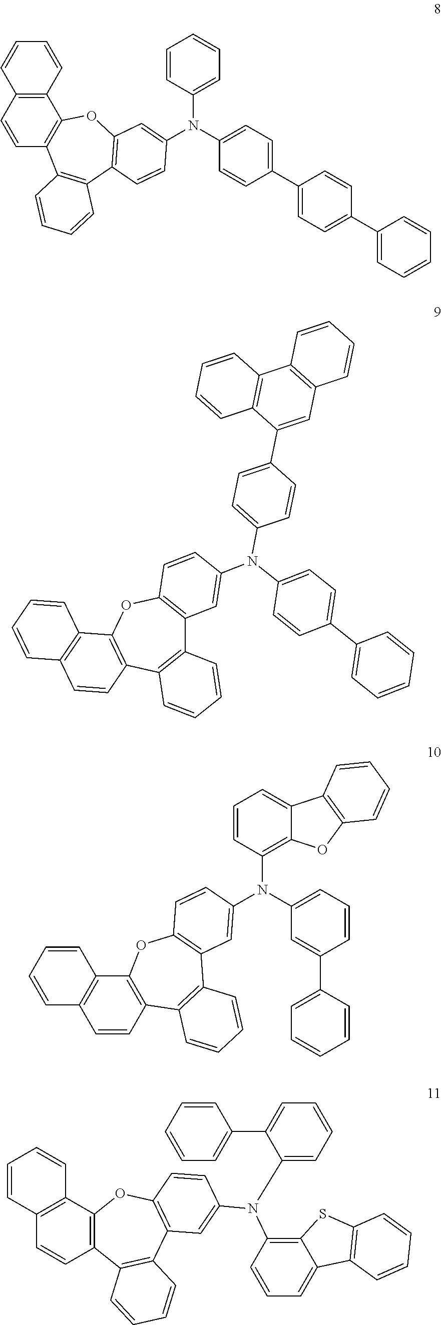 [SMILES: c1ccc(-c2ccc(-c3ccc(N(c4ccccc4)c4ccc5c(c4)Oc4c(ccc6ccccc46)-c4ccccc4-5)cc3)cc2)cc1 ; c1ccc(-c2ccc(N(c3ccc(-c4cc5ccccc5c5ccccc45)cc3)c3ccc4c(c3)-c3ccccc3-c3ccc5ccccc5c3O4)cc2)cc1 ; c1ccc(-c2cccc(N(c3ccc4c(c3)-c3ccccc3-c3ccc5ccccc5c3O4)c3cccc4c3oc3ccccc34)c2)cc1 ; c1ccc(-c2ccccc2N(c2ccc3c(c2)-c2ccccc2-c2ccc4ccccc4c2O3)c2cccc3c2sc2ccccc23)cc1]

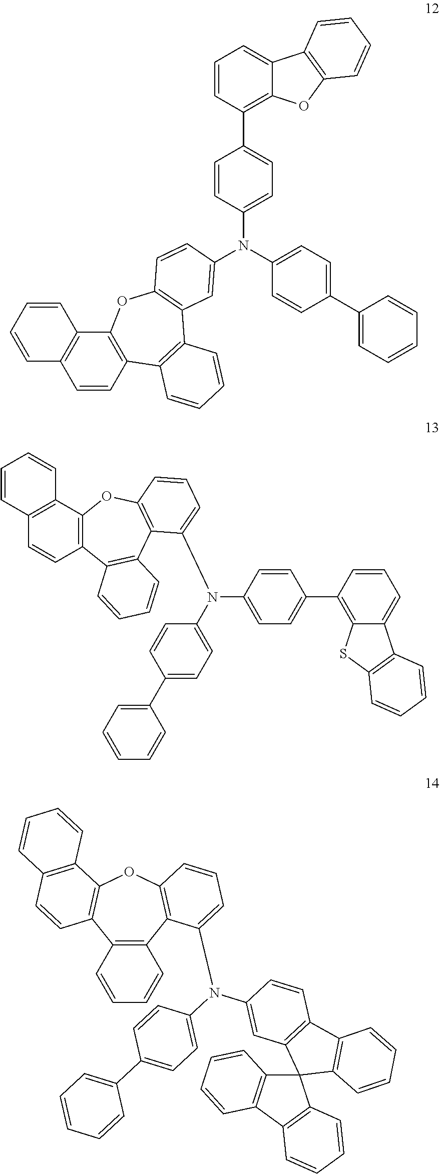 [SMILES: c1ccc(-c2ccc(N(c3ccc(-c4cccc5c4oc4ccccc45)cc3)c3ccc4c(c3)-c3ccccc3-c3ccc5ccccc5c3O4)cc2)cc1 ; c1ccc(-c2ccc(N(c3ccc(-c4cccc5c4sc4ccccc45)cc3)c3cccc4c3-c3ccccc3-c3ccc5ccccc5c3O4)cc2)cc1 ; c1ccc(-c2ccc(N(c3ccc4c(c3)C3(c5ccccc5-c5ccccc53)c3ccccc3-4)c3cccc4c3-c3ccccc3-c3ccc5ccccc5c3O4)cc2)cc1]

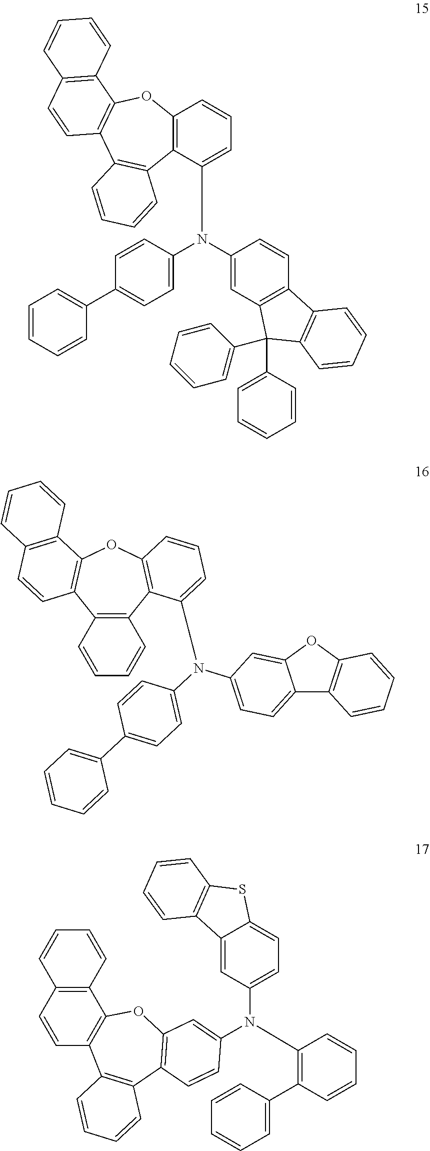 [SMILES: c1ccc(-c2ccc(N(c3ccc4c(c3)C(c3ccccc3)(c3ccccc3)c3ccccc3-4)c3cccc4c3-c3ccccc3-c3ccc5ccccc5c3O4)cc2)cc1 ; c1ccc(-c2ccc(N(c3ccc4c(c3)oc3ccccc34)c3cccc4c3-c3ccccc3-c3ccc5ccccc5c3O4)cc2)cc1 ; c1ccc(-c2ccccc2N(c2ccc3c(c2)Oc2c(ccc4ccccc24)-c2ccccc2-3)c2ccc3sc4ccccc4c3c2)cc1]

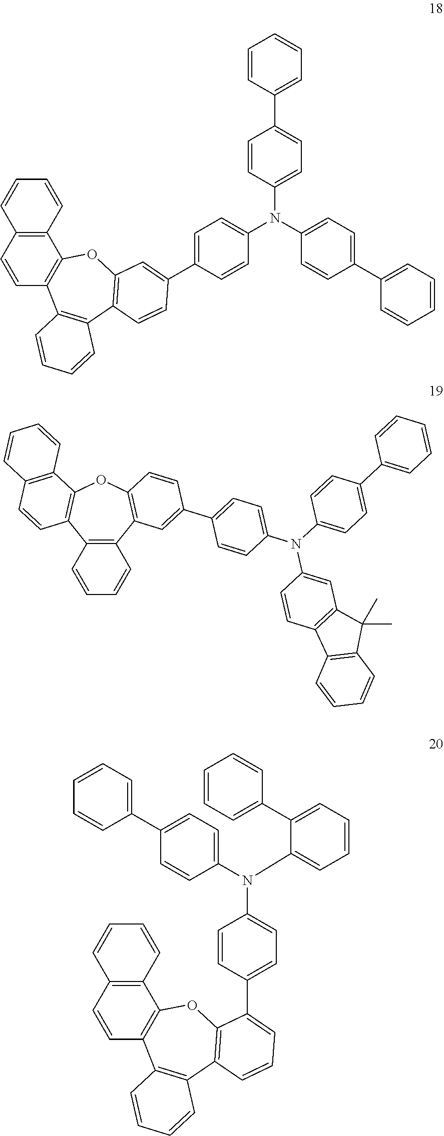 [SMILES: c1ccc(-c2ccc(N(c3ccc(-c4ccccc4)cc3)c3ccc(-c4ccc5c(c4)Oc4c(ccc6ccccc46)-c4ccccc4-5)cc3)cc2)cc1 ; CC1(C)c2ccccc2-c2ccc(N(c3ccc(-c4ccccc4)cc3)c3ccc(-c4ccc5c(c4)-c4ccccc4-c4ccc6ccccc6c4O5)cc3)cc21 ; c1ccc(-c2ccc(N(c3ccc(-c4cccc5c4Oc4c(ccc6ccccc46)-c4ccccc4-5)cc3)c3ccccc3-c3ccccc3)cc2)cc1]

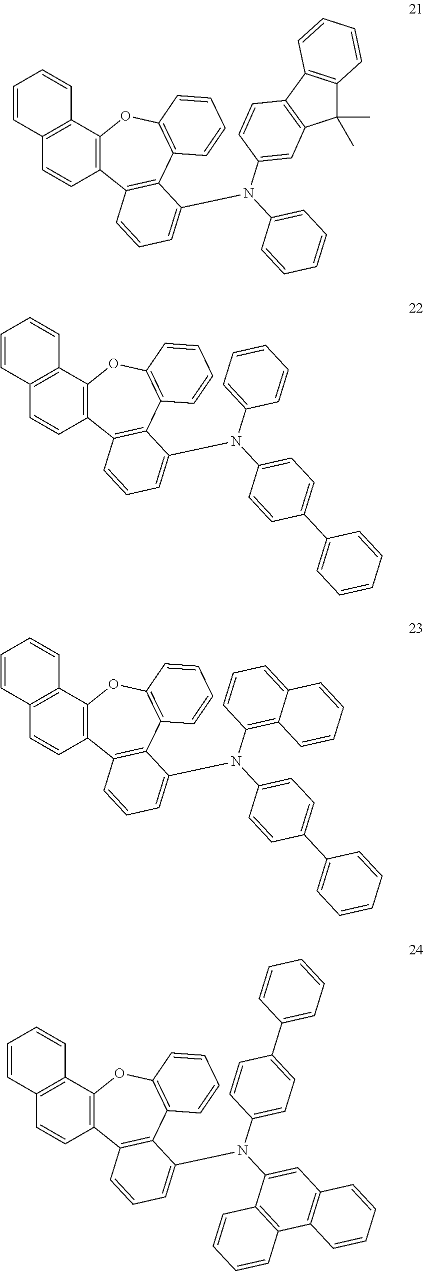 [SMILES: CC1(C)c2ccccc2-c2ccc(N(c3ccccc3)c3cccc4c3-c3ccccc3Oc3c-4ccc4ccccc34)cc21 ; c1ccc(-c2ccc(N(c3ccccc3)c3cccc4c3-c3ccccc3Oc3c-4ccc4ccccc34)cc2)cc1 ; c1ccc(-c2ccc(N(c3cccc4c3-c3ccccc3Oc3c-4ccc4ccccc34)c3cccc4ccccc34)cc2)cc1 ; c1ccc(-c2ccc(N(c3cccc4c3-c3ccccc3Oc3c-4ccc4ccccc34)c3cc4ccccc4c4ccccc34)cc2)cc1]

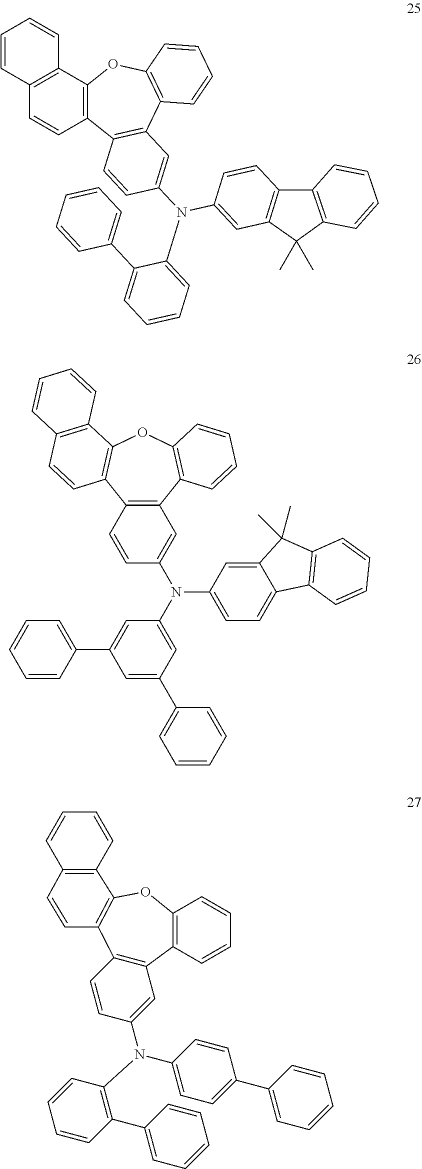 [SMILES: CC1(C)c2ccccc2-c2ccc(N(c3ccc4c(c3)-c3ccccc3Oc3c-4ccc4ccccc34)c3ccccc3-c3ccccc3)cc21 ; CC1(C)c2ccccc2-c2ccc(N(c3cc(-c4ccccc4)cc(-c4ccccc4)c3)c3ccc4c(c3)-c3ccccc3Oc3c-4ccc4ccccc34)cc21 ; c1ccc(-c2ccc(N(c3ccc4c(c3)-c3ccccc3Oc3c-4ccc4ccccc34)c3ccccc3-c3ccccc3)cc2)cc1]

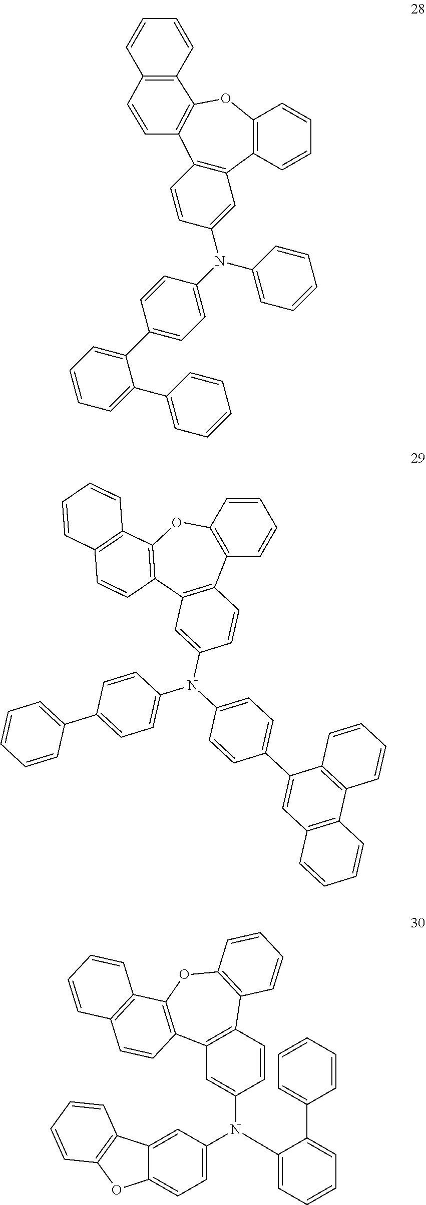 [SMILES: c1ccc(-c2ccccc2-c2ccc(N(c3ccccc3)c3ccc4c(c3)-c3ccccc3Oc3c-4ccc4ccccc34)cc2)cc1 ; c1ccc(-c2ccc(N(c3ccc(-c4cc5ccccc5c5ccccc45)cc3)c3ccc4c(c3)-c3ccc5ccccc5c3Oc3ccccc3-4)cc2)cc1 ; c1ccc(-c2ccccc2N(c2ccc3c(c2)-c2ccc4ccccc4c2Oc2ccccc2-3)c2ccc3oc4ccccc4c3c2)cc1]

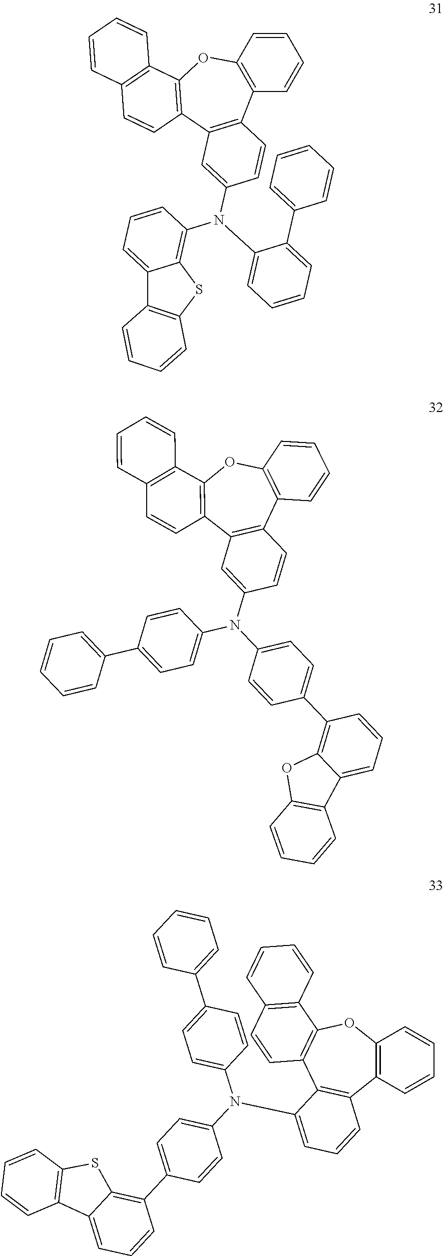 [SMILES: c1ccc(-c2ccccc2N(c2ccc3c(c2)-c2ccc4ccccc4c2Oc2ccccc2-3)c2cccc3c2sc2ccccc23)cc1 ; c1ccc(-c2ccc(N(c3ccc(-c4cccc5c4oc4ccccc45)cc3)c3ccc4c(c3)-c3ccc5ccccc5c3Oc3ccccc3-4)cc2)cc1 ; c1ccc(-c2ccc(N(c3ccc(-c4cccc5c4sc4ccccc45)cc3)c3cccc4c3-c3ccc5ccccc5c3Oc3ccccc3-4)cc2)cc1]

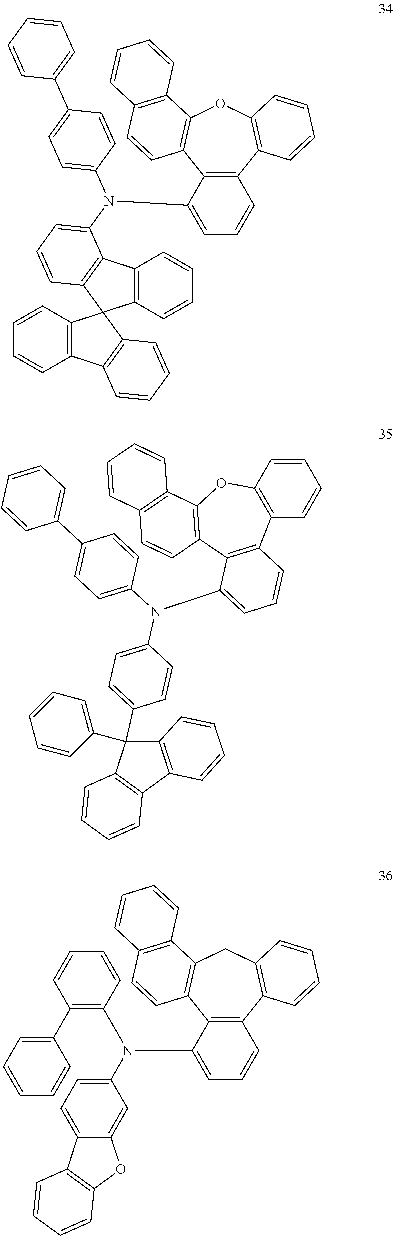

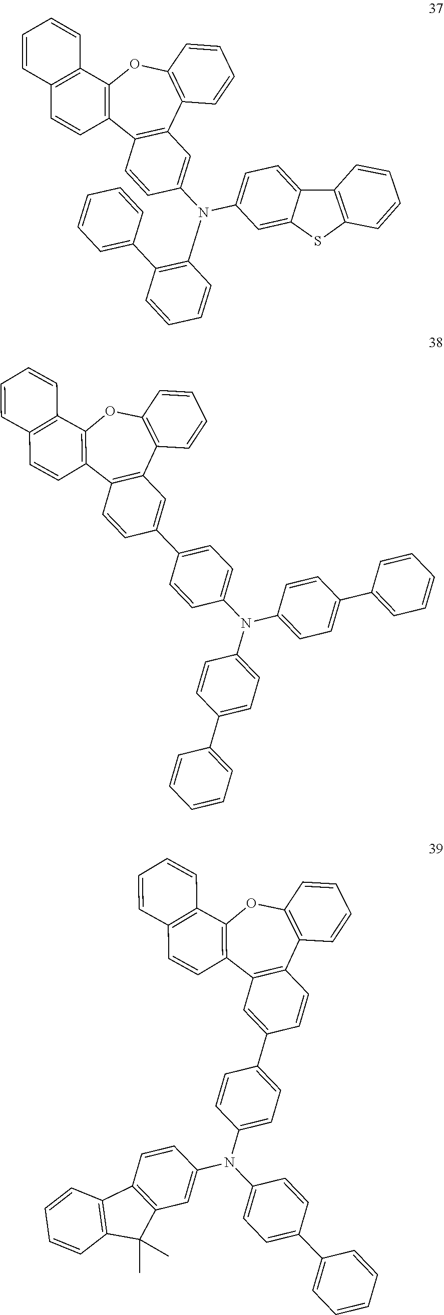 [SMILES: c1ccc(-c2ccccc2N(c2ccc3c(c2)-c2ccccc2Oc2c-3ccc3ccccc23)c2ccc3c(c2)sc2ccccc23)cc1 ; c1ccc(-c2ccc(N(c3ccc(-c4ccccc4)cc3)c3ccc(-c4ccc5c(c4)-c4ccccc4Oc4c-5ccc5ccccc45)cc3)cc2)cc1 ; CC1(C)c2ccccc2-c2ccc(N(c3ccc(-c4ccccc4)cc3)c3ccc(-c4ccc5c(c4)-c4ccc6ccccc6c4Oc4ccccc4-5)cc3)cc21]

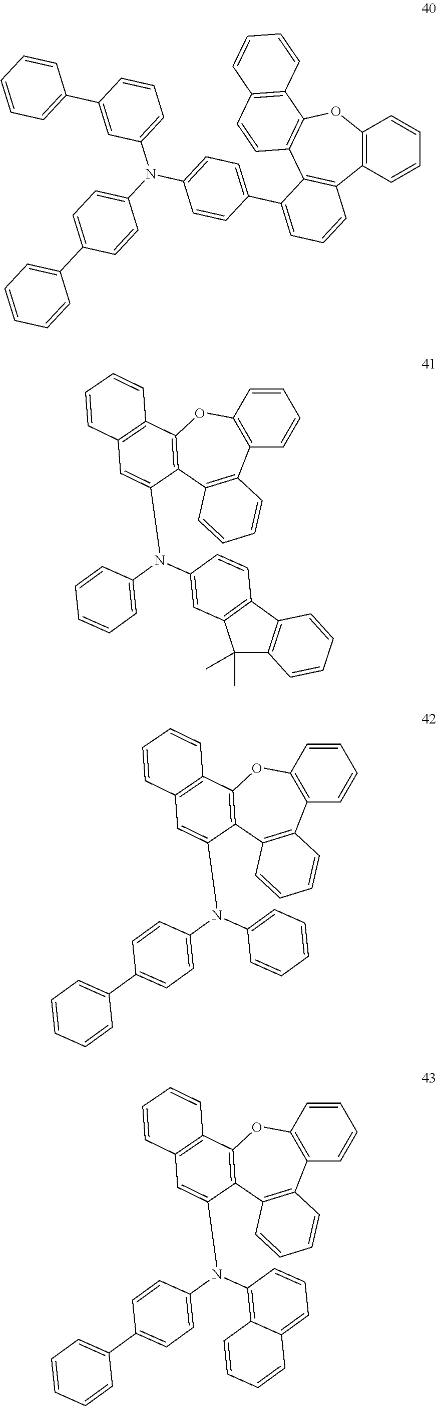 [SMILES: c1ccc(-c2ccc(N(c3ccc(-c4cccc5c4-c4ccc6ccccc6c4Oc4ccccc4-5)cc3)c3cccc(-c4ccccc4)c3)cc2)cc1 ; CC1(C)c2ccccc2-c2ccc(N(c3ccccc3)c3cc4ccccc4c4c3-c3ccccc3-c3ccccc3O4)cc21 ; c1ccc(-c2ccc(N(c3ccccc3)c3cc4ccccc4c4c3-c3ccccc3-c3ccccc3O4)cc2)cc1 ; c1ccc(-c2ccc(N(c3cc4ccccc4c4c3-c3ccccc3-c3ccccc3O4)c3cccc4ccccc34)cc2)cc1]

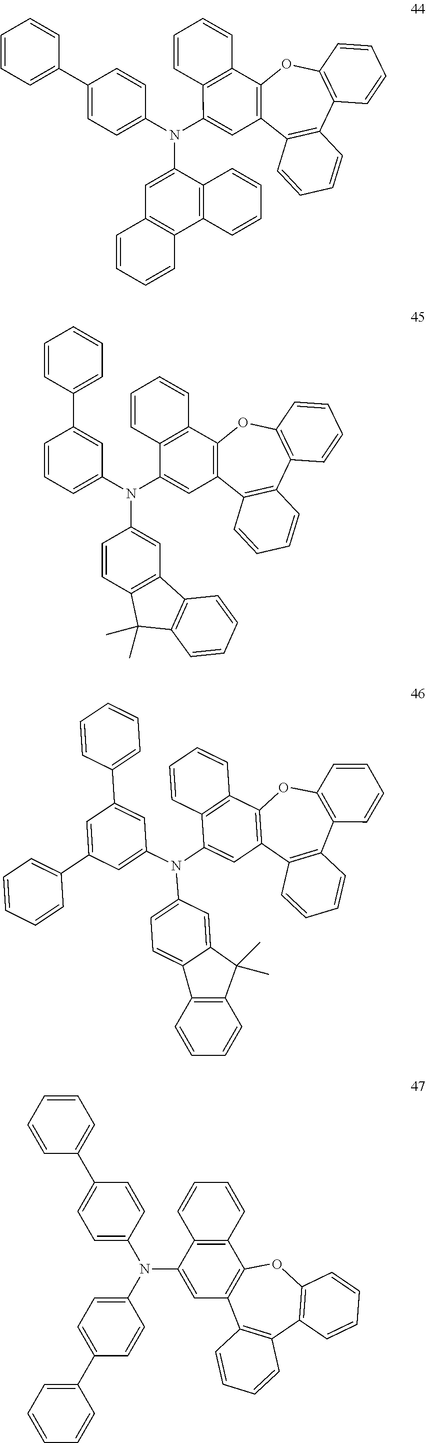 [SMILES: c1ccc(-c2ccc(N(c3cc4c(c5ccccc35)Oc3ccccc3-c3ccccc3-4)c3cc4ccccc4c4ccccc34)cc2)cc1 ; CC1(C)c2ccccc2-c2cc(N(c3cccc(-c4ccccc4)c3)c3cc4c(c5ccccc35)Oc3ccccc3-c3ccccc3-4)ccc21 ; CC1(C)c2ccccc2-c2ccc(N(c3cc(-c4ccccc4)cc(-c4ccccc4)c3)c3cc4c(c5ccccc35)Oc3ccccc3-c3ccccc3-4)cc21 ; c1ccc(-c2ccc(N(c3ccc(-c4ccccc4)cc3)c3cc4c(c5ccccc35)Oc3ccccc3-c3ccccc3-4)cc2)cc1]

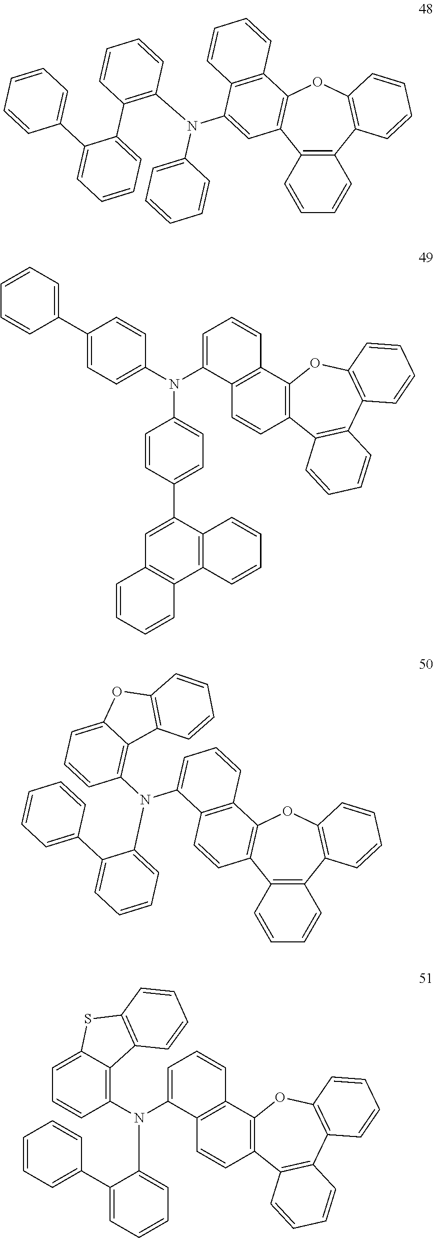 [SMILES: c1ccc(-c2ccccc2-c2ccccc2N(c2ccccc2)c2cc3c(c4ccccc24)Oc2ccccc2-c2ccccc2-3)cc1 ; c1ccc(-c2ccc(N(c3ccc(-c4cc5ccccc5c5ccccc45)cc3)c3cccc4c5c(ccc34)-c3ccccc3-c3ccccc3O5)cc2)cc1 ; c1ccc(-c2ccccc2N(c2cccc3c4c(ccc23)-c2ccccc2-c2ccccc2O4)c2cccc3oc4ccccc4c23)cc1 ; c1ccc(-c2ccccc2N(c2cccc3c4c(ccc23)-c2ccccc2-c2ccccc2O4)c2cccc3sc4ccccc4c23)cc1]

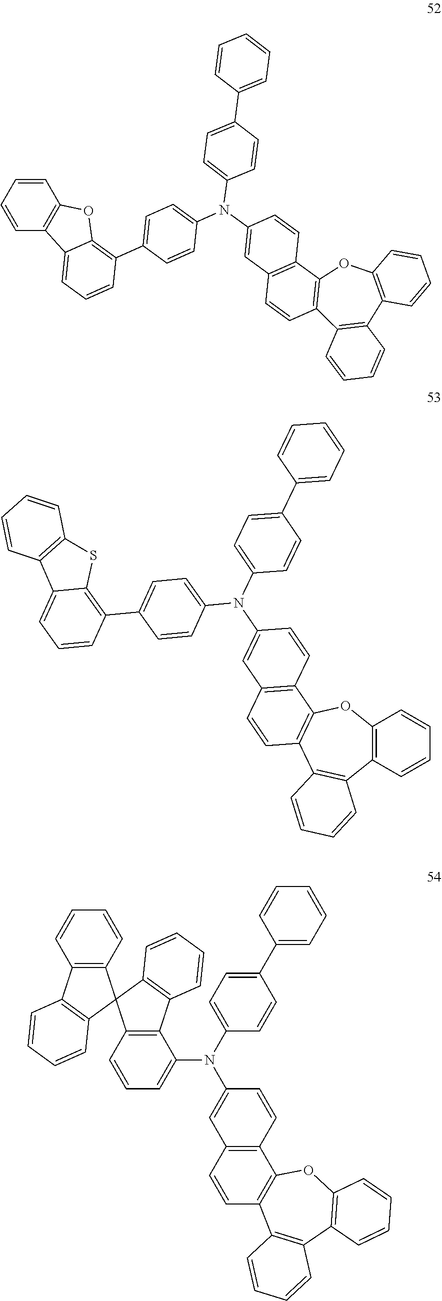

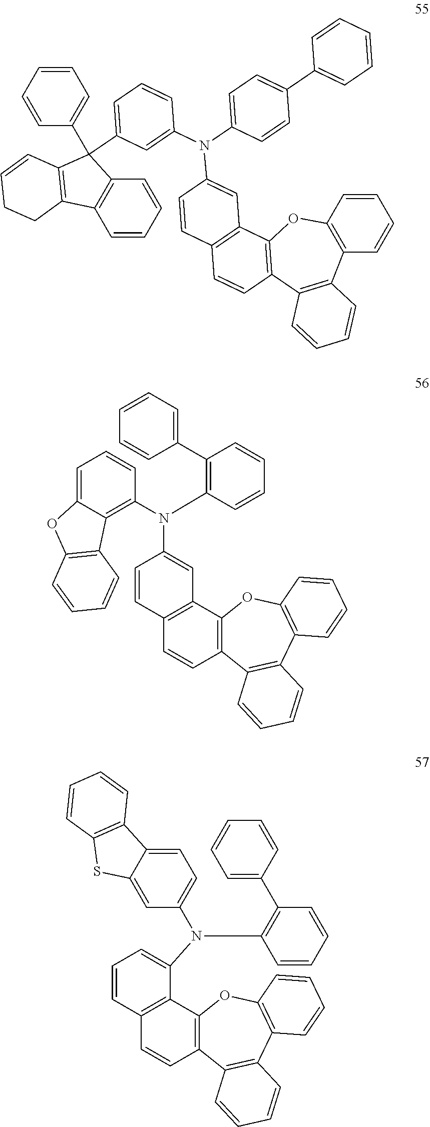 [SMILES: C1=CC2=C(CC1)c1ccccc1C2(c1ccccc1)c1cccc(N(c2ccc(-c3ccccc3)cc2)c2ccc3ccc4c(c3c2)Oc2ccccc2-c2ccccc2-4)c1 ; c1ccc(-c2ccccc2N(c2ccc3ccc4c(c3c2)Oc2ccccc2-c2ccccc2-4)c2cccc3oc4ccccc4c23)cc1 ; c1ccc(-c2ccccc2N(c2ccc3c(c2)sc2ccccc23)c2cccc3ccc4c(c23)Oc2ccccc2-c2ccccc2-4)cc1]

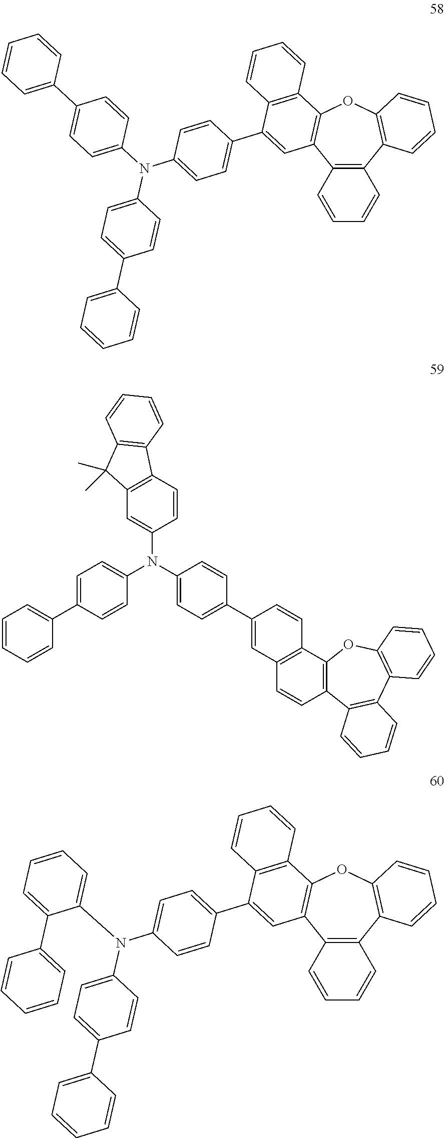 [SMILES: c1ccc(-c2ccc(N(c3ccc(-c4ccccc4)cc3)c3ccc(-c4cc5c(c6ccccc46)Oc4ccccc4-c4ccccc4-5)cc3)cc2)cc1 ; CC1(C)c2ccccc2-c2ccc(N(c3ccc(-c4ccccc4)cc3)c3ccc(-c4ccc5c6c(ccc5c4)-c4ccccc4-c4ccccc4O6)cc3)cc21 ; c1ccc(-c2ccc(N(c3ccc(-c4cc5c(c6ccccc46)Oc4ccccc4-c4ccccc4-5)cc3)c3ccccc3-c3ccccc3)cc2)cc1]

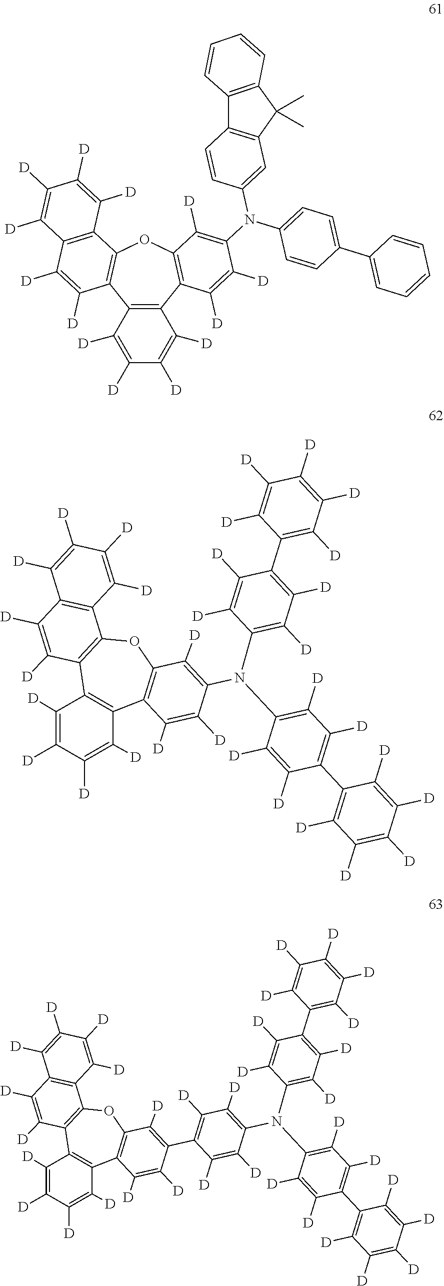

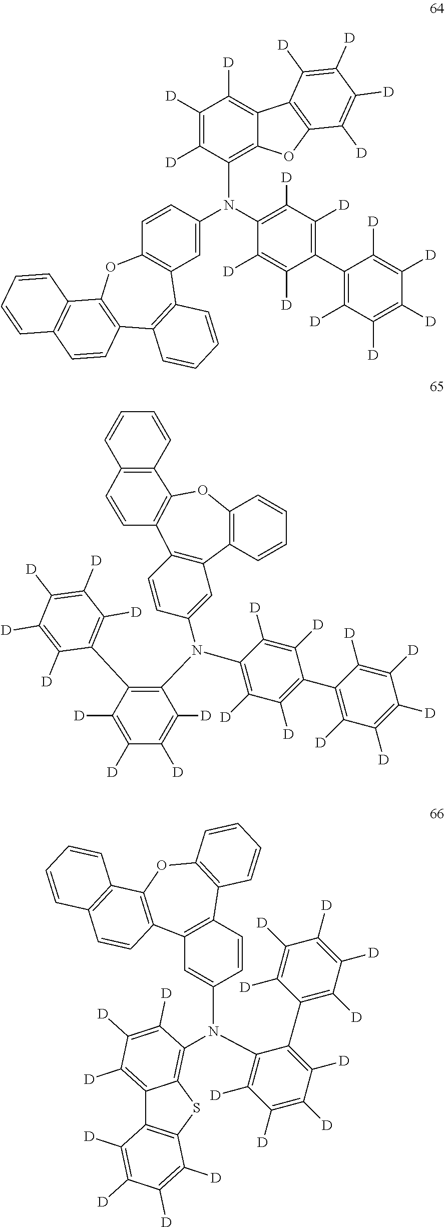

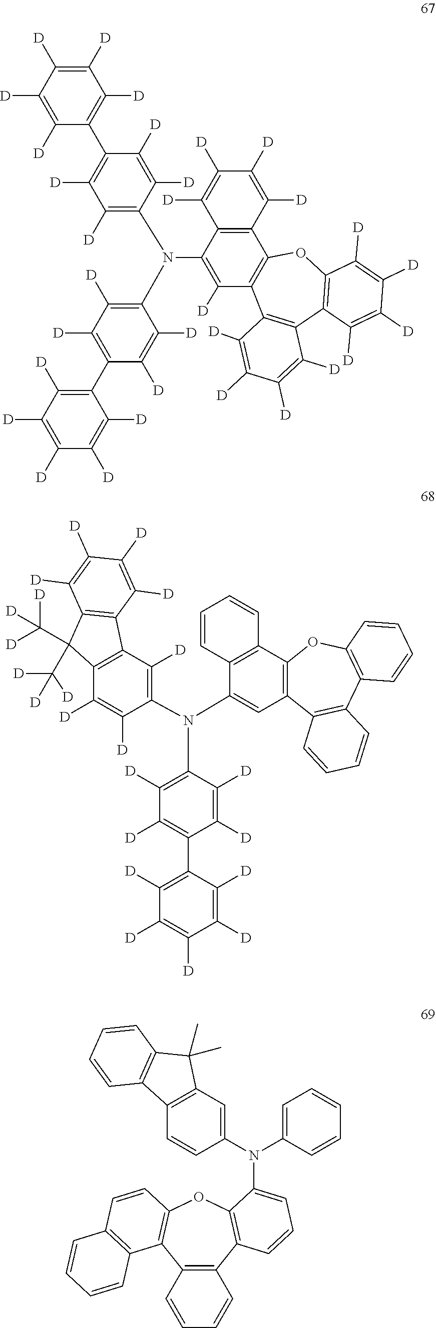

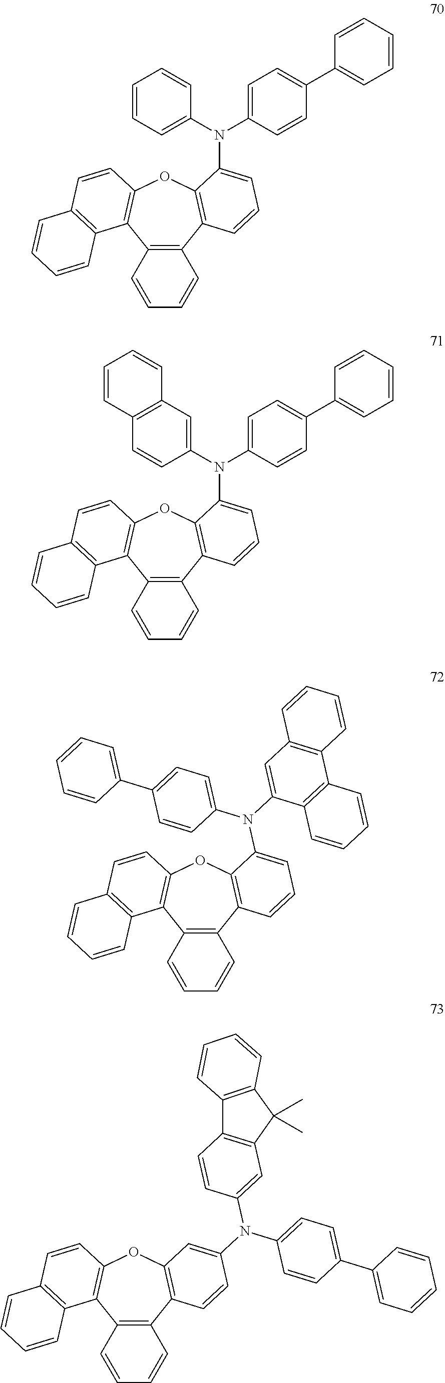

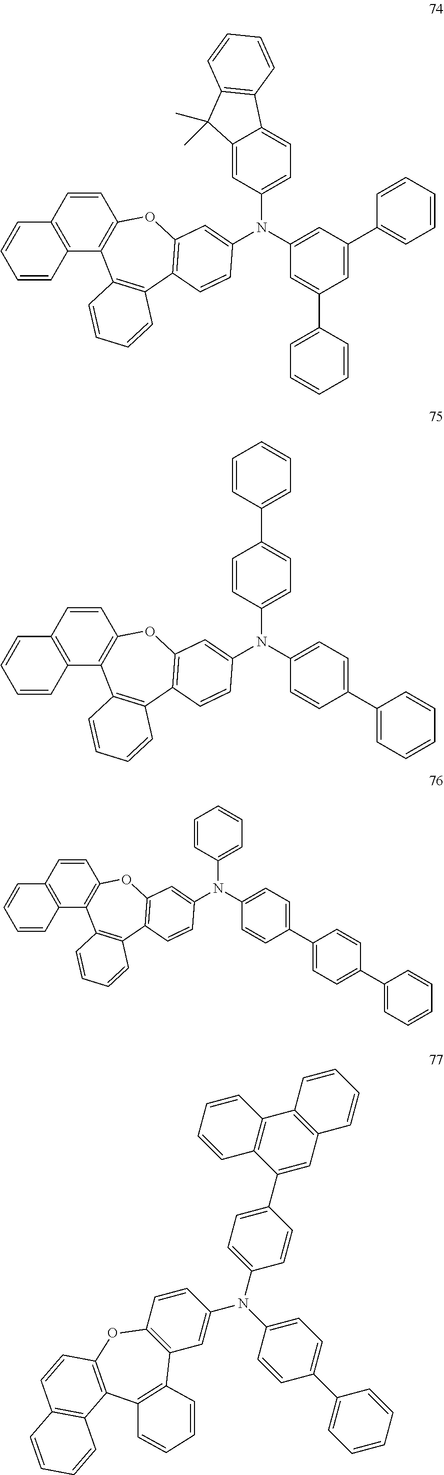 [SMILES: CC1(C)c2ccccc2-c2ccc(N(c3cc(-c4ccccc4)cc(-c4ccccc4)c3)c3ccc4c(c3)Oc3ccc5ccccc5c3-c3ccccc3-4)cc21 ; c1ccc(-c2ccc(N(c3ccc(-c4ccccc4)cc3)c3ccc4c(c3)Oc3ccc5ccccc5c3-c3ccccc3-4)cc2)cc1 ; c1ccc(-c2ccc(-c3ccc(N(c4ccccc4)c4ccc5c(c4)Oc4ccc6ccccc6c4-c4ccccc4-5)cc3)cc2)cc1 ; c1ccc(-c2ccc(N(c3ccc(-c4cc5ccccc5c5ccccc45)cc3)c3ccc4c(c3)-c3ccccc3-c3c(ccc5ccccc35)O4)cc2)cc1]

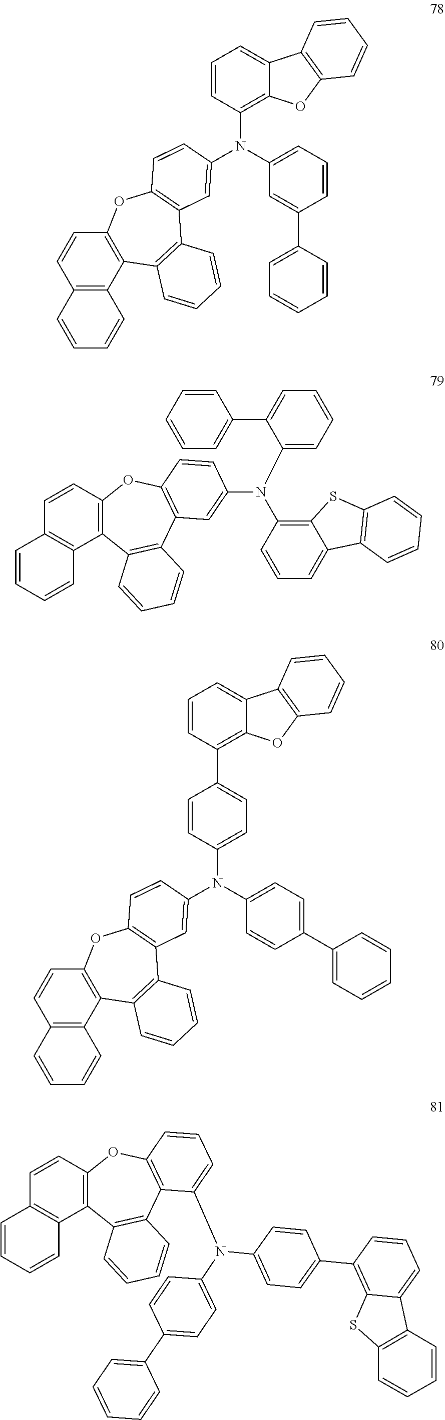 [SMILES: c1ccc(-c2cccc(N(c3ccc4c(c3)-c3ccccc3-c3c(ccc5ccccc35)O4)c3cccc4c3oc3ccccc34)c2)cc1 ; c1ccc(-c2ccccc2N(c2ccc3c(c2)-c2ccccc2-c2c(ccc4ccccc24)O3)c2cccc3c2sc2ccccc23)cc1 ; c1ccc(-c2ccc(N(c3ccc(-c4cccc5c4oc4ccccc45)cc3)c3ccc4c(c3)-c3ccccc3-c3c(ccc5ccccc35)O4)cc2)cc1 ; c1ccc(-c2ccc(N(c3ccc(-c4cccc5c4sc4ccccc45)cc3)c3cccc4c3-c3ccccc3-c3c(ccc5ccccc35)O4)cc2)cc1]

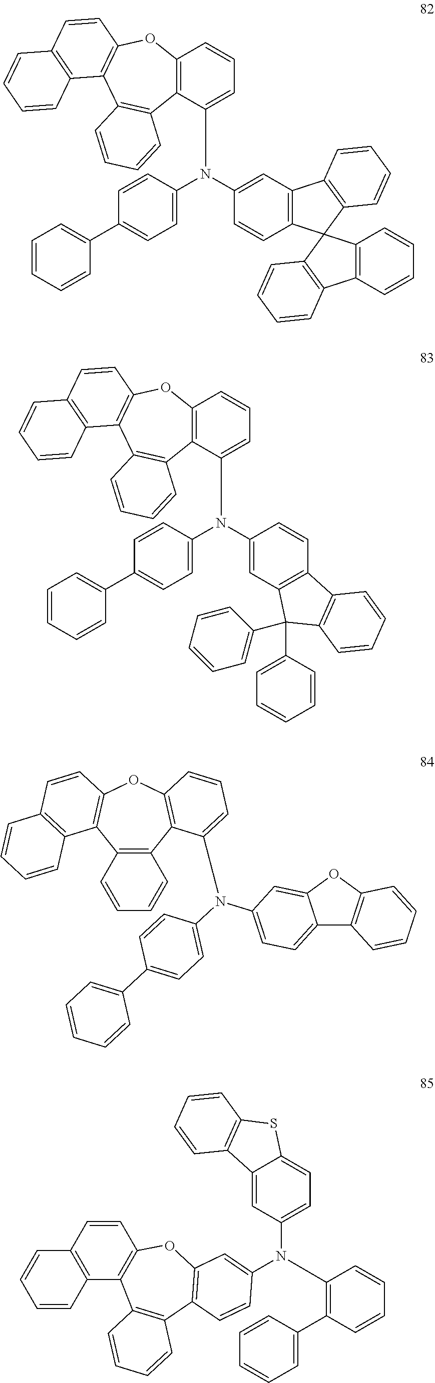 [SMILES: c1ccc(-c2ccc(N(c3ccc4c(c3)-c3ccccc3C43c4ccccc4-c4ccccc43)c3cccc4c3-c3ccccc3-c3c(ccc5ccccc35)O4)cc2)cc1 ; c1ccc(-c2ccc(N(c3ccc4c(c3)C(c3ccccc3)(c3ccccc3)c3ccccc3-4)c3cccc4c3-c3ccccc3-c3c(ccc5ccccc35)O4)cc2)cc1 ; c1ccc(-c2ccc(N(c3ccc4c(c3)oc3ccccc34)c3cccc4c3-c3ccccc3-c3c(ccc5ccccc35)O4)cc2)cc1 ; c1ccc(-c2ccccc2N(c2ccc3c(c2)Oc2ccc4ccccc4c2-c2ccccc2-3)c2ccc3sc4ccccc4c3c2)cc1]

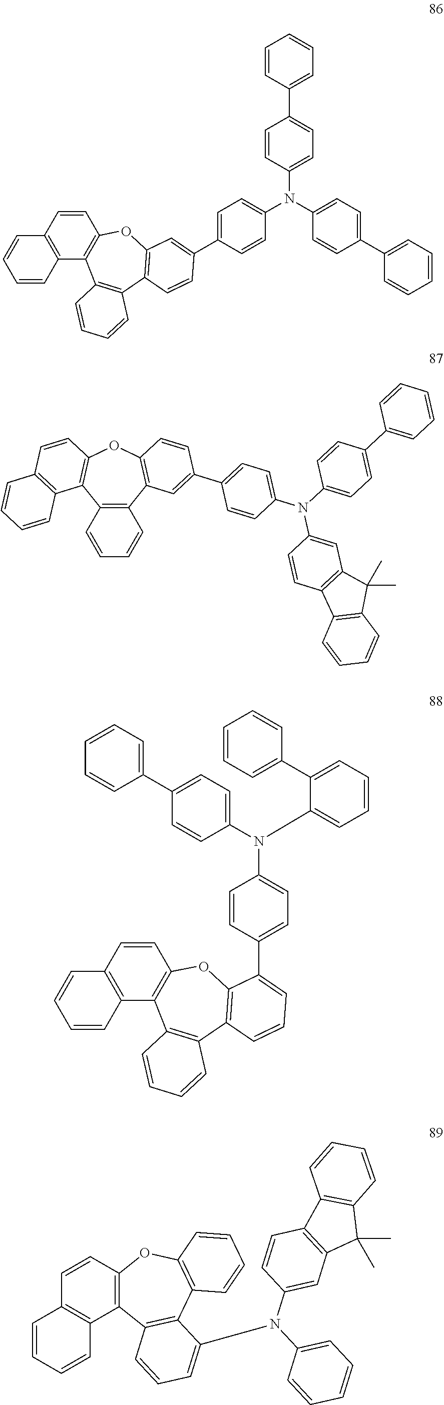 [SMILES: c1ccc(-c2ccc(N(c3ccc(-c4ccccc4)cc3)c3ccc(-c4ccc5c(c4)Oc4ccc6ccccc6c4-c4ccccc4-5)cc3)cc2)cc1 ; CC1(C)c2ccccc2-c2ccc(N(c3ccc(-c4ccccc4)cc3)c3ccc(-c4ccc5c(c4)-c4ccccc4-c4c(ccc6ccccc46)O5)cc3)cc21 ; c1ccc(-c2ccc(N(c3ccc(-c4cccc5c4Oc4ccc6ccccc6c4-c4ccccc4-5)cc3)c3ccccc3-c3ccccc3)cc2)cc1 ; CC1(C)c2ccccc2-c2ccc(N(c3ccccc3)c3cccc4c3-c3ccccc3Oc3ccc5ccccc5c3-4)cc21]

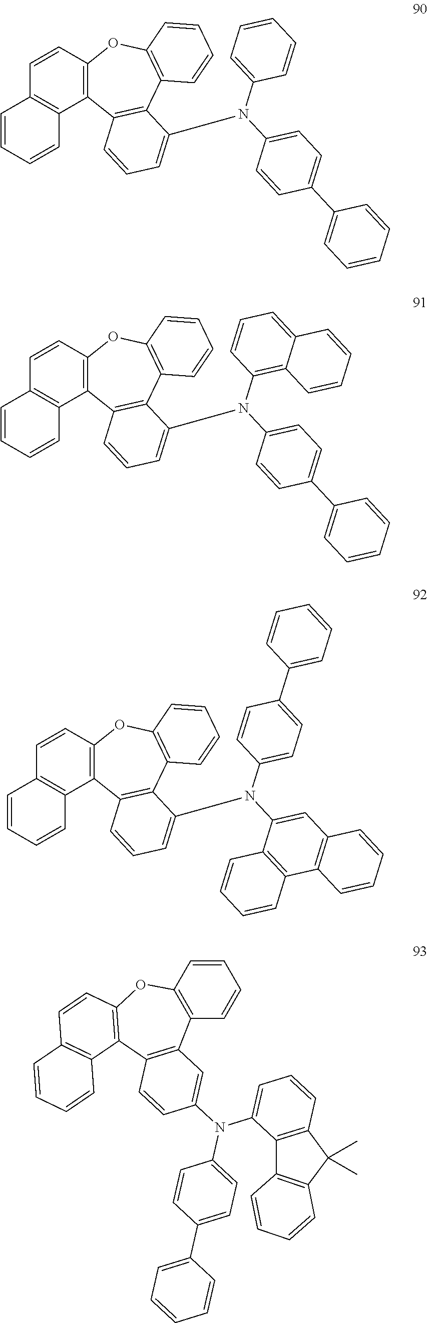 [SMILES: c1ccc(-c2ccc(N(c3ccccc3)c3cccc4c3-c3ccccc3Oc3ccc5ccccc5c3-4)cc2)cc1 ; c1ccc(-c2ccc(N(c3cccc4c3-c3ccccc3Oc3ccc5ccccc5c3-4)c3cccc4ccccc34)cc2)cc1 ; c1ccc(-c2ccc(N(c3cccc4c3-c3ccccc3Oc3ccc5ccccc5c3-4)c3cc4ccccc4c4ccccc34)cc2)cc1 ; CC1(C)c2ccccc2-c2c(N(c3ccc(-c4ccccc4)cc3)c3ccc4c(c3)-c3ccccc3Oc3ccc5ccccc5c3-4)cccc21]

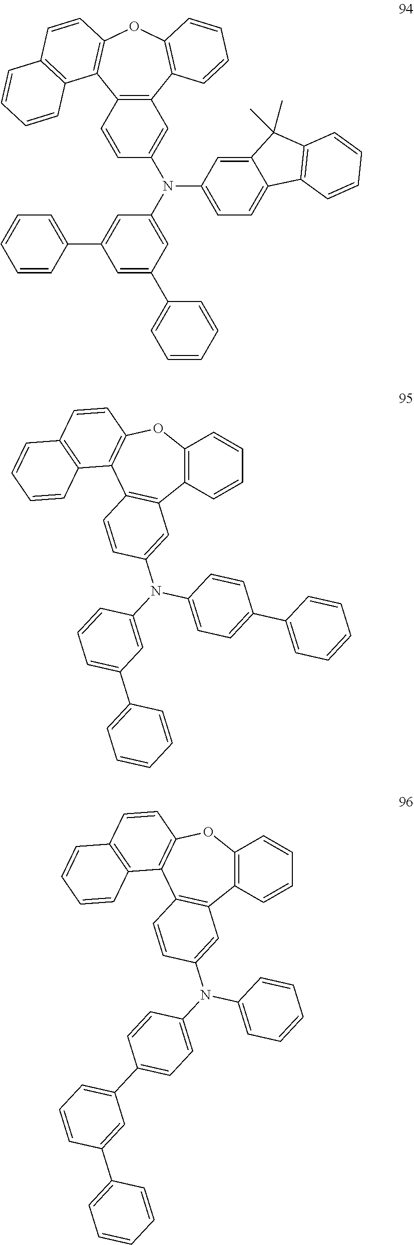

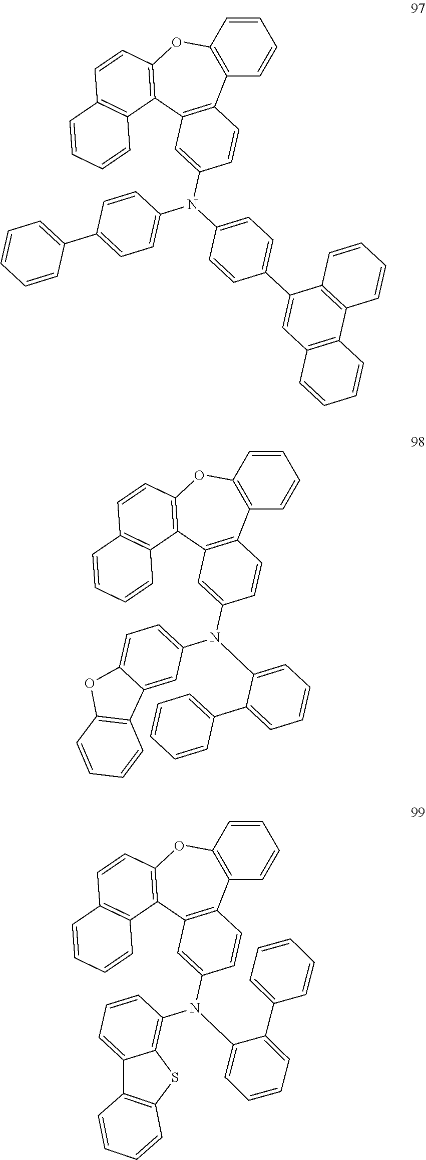 [SMILES: c1ccc(-c2ccc(N(c3ccc(-c4cc5ccccc5c5ccccc45)cc3)c3ccc4c(c3)-c3c(ccc5ccccc35)Oc3ccccc3-4)cc2)cc1 ; c1ccc(-c2ccccc2N(c2ccc3c(c2)-c2c(ccc4ccccc24)Oc2ccccc2-3)c2ccc3oc4ccccc4c3c2)cc1 ; c1ccc(-c2ccccc2N(c2ccc3c(c2)-c2c(ccc4ccccc24)Oc2ccccc2-3)c2cccc3c2sc2ccccc23)cc1]

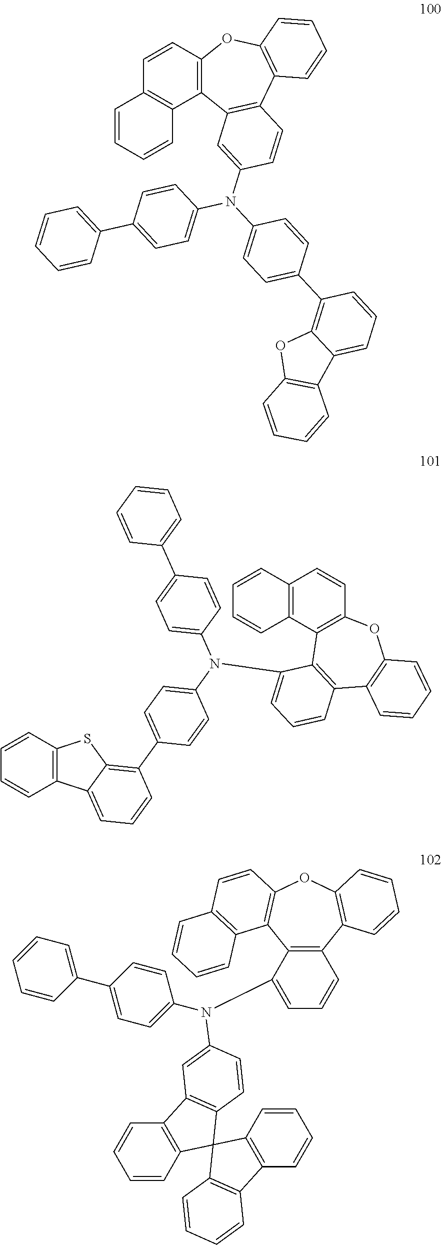 [SMILES: c1ccc(-c2ccc(N(c3ccc(-c4cccc5c4oc4ccccc45)cc3)c3ccc4c(c3)-c3c(ccc5ccccc35)Oc3ccccc3-4)cc2)cc1 ; c1ccc(-c2ccc(N(c3ccc(-c4cccc5c4sc4ccccc45)cc3)c3cccc4c3-c3c(ccc5ccccc35)Oc3ccccc3-4)cc2)cc1 ; c1ccc(-c2ccc(N(c3ccc4c(c3)-c3ccccc3C43c4ccccc4-c4ccccc43)c3cccc4c3-c3c(ccc5ccccc35)Oc3ccccc3-4)cc2)cc1]

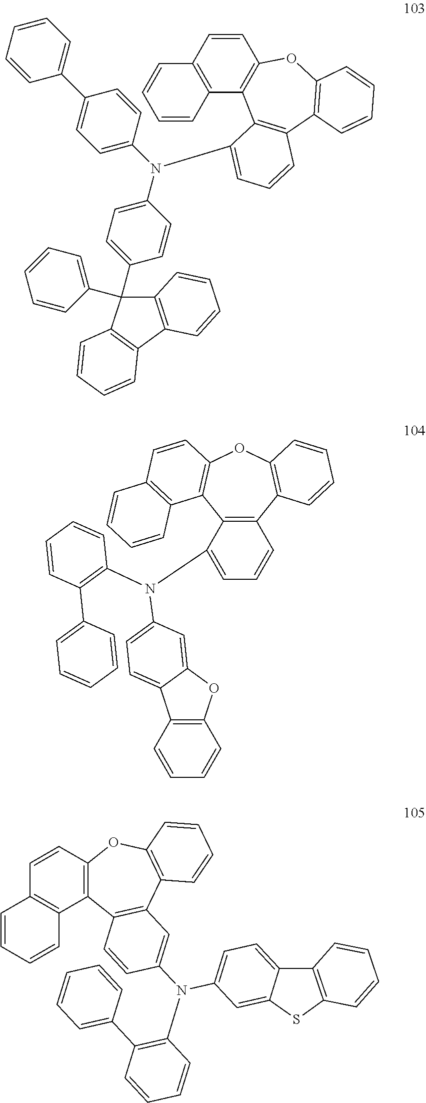

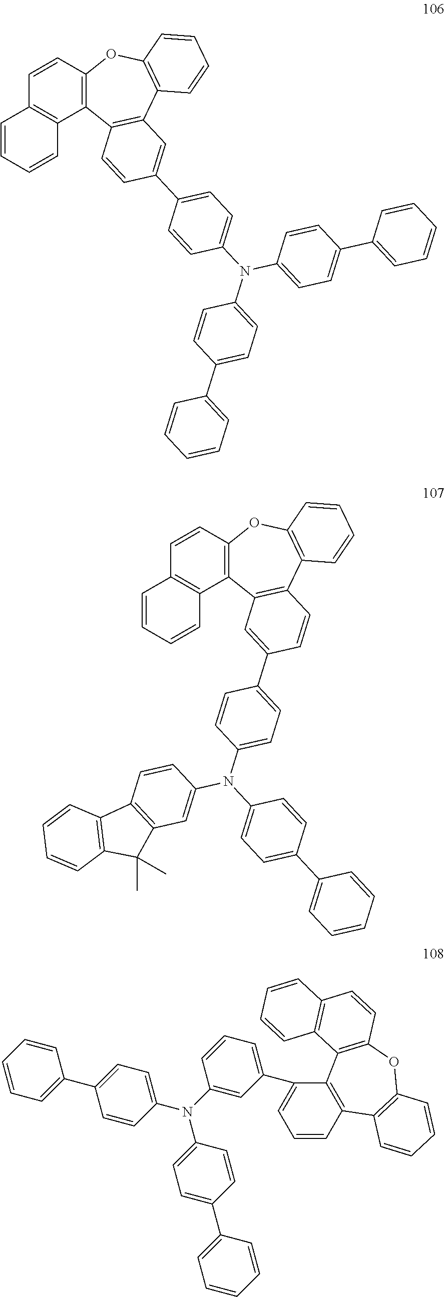 [SMILES: c1ccc(-c2ccc(N(c3ccc(-c4ccccc4)cc3)c3ccc(-c4ccc5c(c4)-c4ccccc4Oc4ccc6ccccc6c4-5)cc3)cc2)cc1 ; CC1(C)c2ccccc2-c2ccc(N(c3ccc(-c4ccccc4)cc3)c3ccc(-c4ccc5c(c4)-c4c(ccc6ccccc46)Oc4ccccc4-5)cc3)cc21 ; c1ccc(-c2ccc(N(c3ccc(-c4ccccc4)cc3)c3cccc(-c4cccc5c4-c4c(ccc6ccccc46)Oc4ccccc4-5)c3)cc2)cc1]

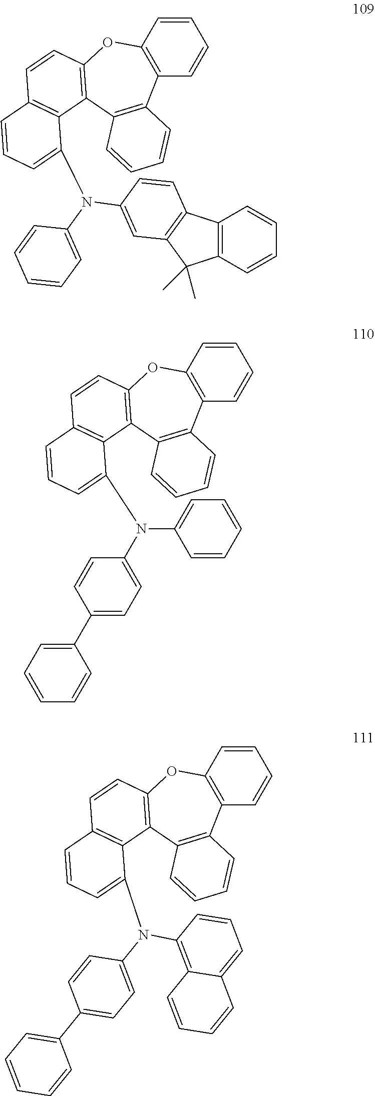

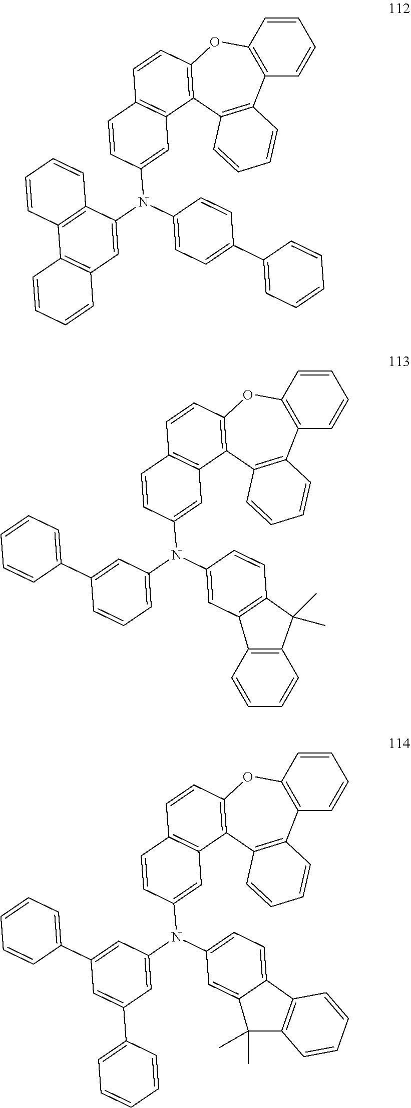 [SMILES: c1ccc(-c2ccc(N(c3ccc4ccc5c(c4c3)-c3ccccc3-c3ccccc3O5)c3cc4ccccc4c4ccccc34)cc2)cc1 ; CC1(C)c2ccccc2-c2cc(N(c3cccc(-c4ccccc4)c3)c3ccc4ccc5c(c4c3)-c3ccccc3-c3ccccc3O5)ccc21 ; CC1(C)c2ccccc2-c2ccc(N(c3cc(-c4ccccc4)cc(-c4ccccc4)c3)c3ccc4ccc5c(c4c3)-c3ccccc3-c3ccccc3O5)cc21]

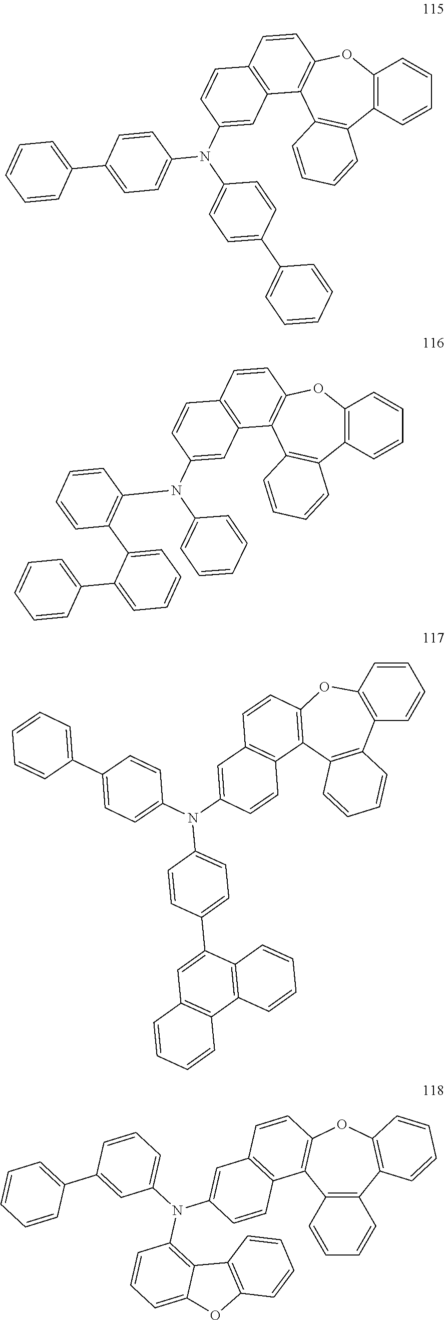 [SMILES: c1ccc(-c2ccc(N(c3ccc(-c4ccccc4)cc3)c3ccc4ccc5c(c4c3)-c3ccccc3-c3ccccc3O5)cc2)cc1 ; c1ccc(-c2ccccc2-c2ccccc2N(c2ccccc2)c2ccc3ccc4c(c3c2)-c2ccccc2-c2ccccc2O4)cc1 ; c1ccc(-c2ccc(N(c3ccc(-c4cc5ccccc5c5ccccc45)cc3)c3ccc4c5c(ccc4c3)Oc3ccccc3-c3ccccc3-5)cc2)cc1 ; c1ccc(-c2cccc(N(c3ccc4c5c(ccc4c3)Oc3ccccc3-c3ccccc3-5)c3cccc4oc5ccccc5c34)c2)cc1]

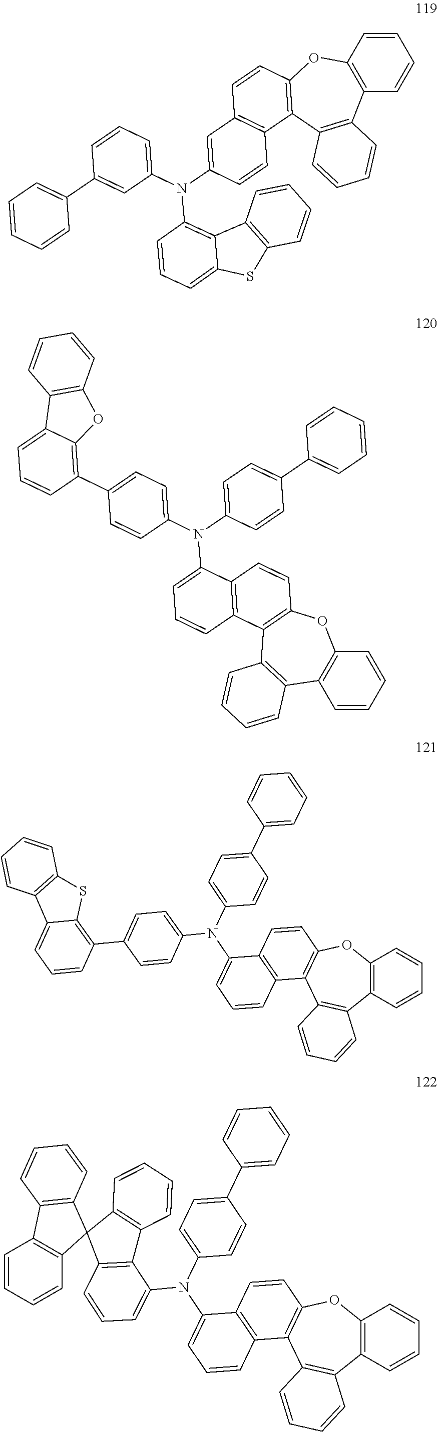 [SMILES: c1ccc(-c2cccc(N(c3ccc4c5c(ccc4c3)Oc3ccccc3-c3ccccc3-5)c3cccc4sc5ccccc5c34)c2)cc1 ; c1ccc(-c2ccc(N(c3ccc(-c4cccc5c4oc4ccccc45)cc3)c3cccc4c5c(ccc34)Oc3ccccc3-c3ccccc3-5)cc2)cc1 ; c1ccc(-c2ccc(N(c3ccc(-c4cccc5c4sc4ccccc45)cc3)c3cccc4c5c(ccc34)Oc3ccccc3-c3ccccc3-5)cc2)cc1 ; c1ccc(-c2ccc(N(c3cccc4c3-c3ccccc3C43c4ccccc4-c4ccccc43)c3cccc4c5c(ccc34)Oc3ccccc3-c3ccccc3-5)cc2)cc1]

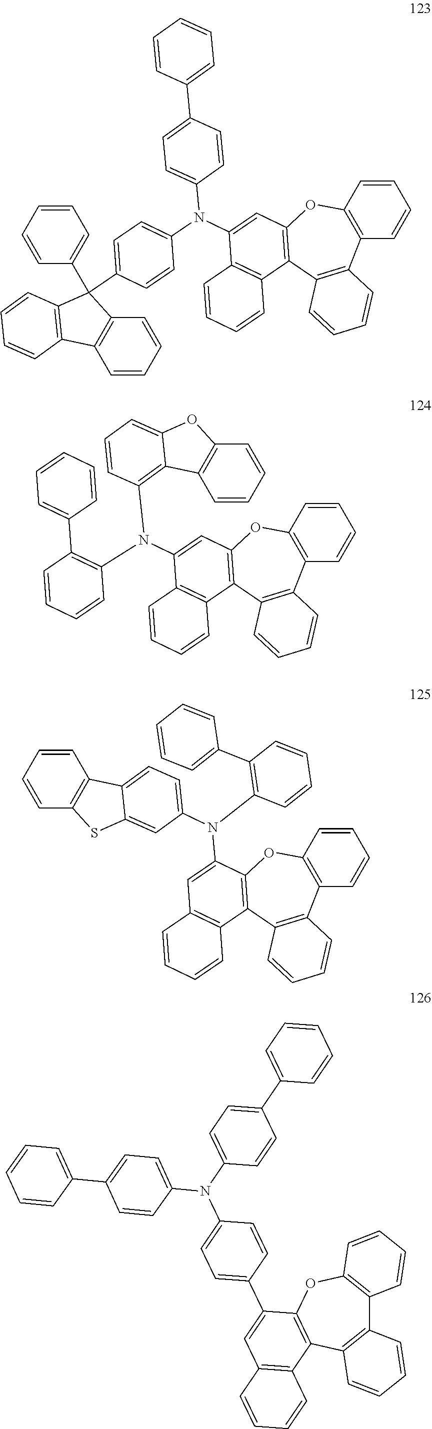

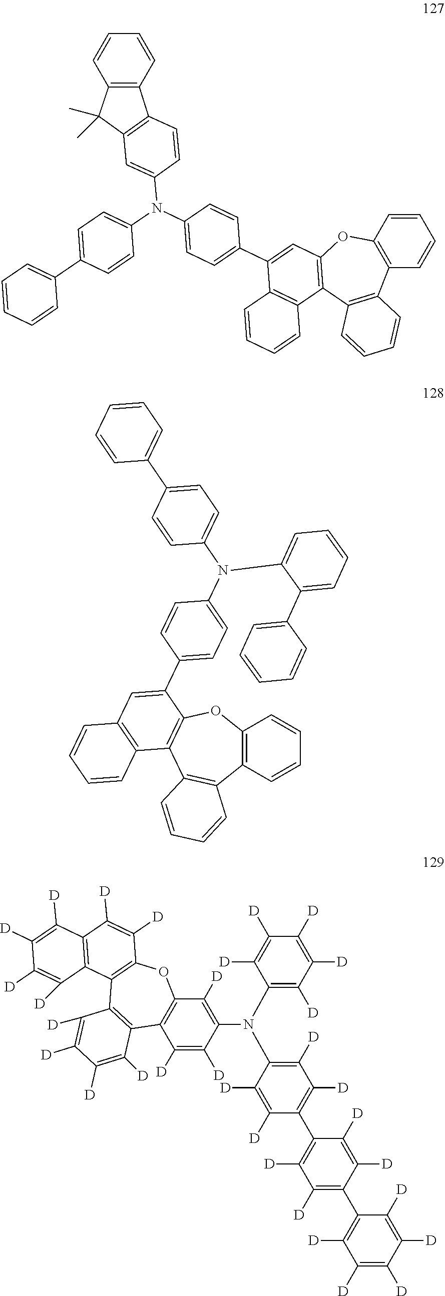 [SMILES: CC1(C)c2ccccc2-c2ccc(N(c3ccc(-c4ccccc4)cc3)c3ccc(-c4cc5c(c6ccccc46)-c4ccccc4-c4ccccc4O5)cc3)cc21 ; c1ccc(-c2ccc(N(c3ccc(-c4cc5ccccc5c5c4Oc4ccccc4-c4ccccc4-5)cc3)c3ccccc3-c3ccccc3)cc2)cc1 ; [2H]c1c([2H])c([2H])c(-c2c([2H])c([2H])c(-c3c([2H])c([2H])c(N(c4c([2H])c([2H])c([2H])c([2H])c4[2H])c4c([2H])c([2H])c5c(c4[2H])Oc4c([2H])c([2H])c6c([2H])c([2H])c([2H])c([2H])c6c4-c4c([2H])c([2H])c([2H])c([2H])c4-5)c([2H])c3[2H])c([2H])c2[2H])c([2H])c1[2H]]

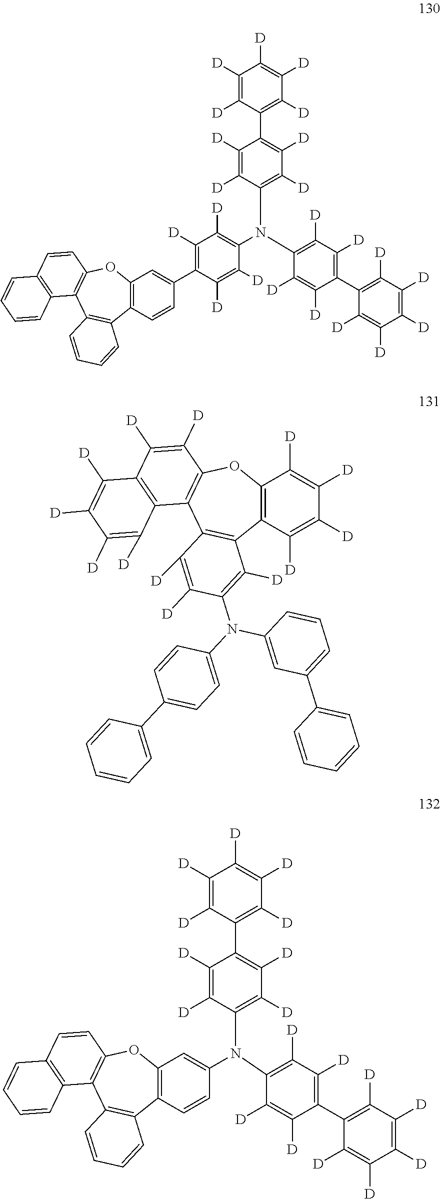

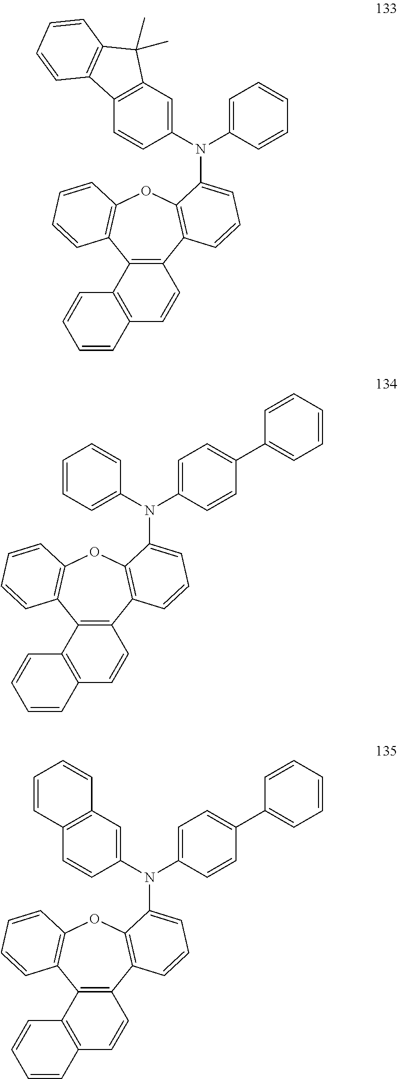 [SMILES: CC1(C)c2ccccc2-c2ccc(N(c3ccccc3)c3cccc4c3Oc3ccccc3-c3c-4ccc4ccccc34)cc21 ; c1ccc(-c2ccc(N(c3ccccc3)c3cccc4c3Oc3ccccc3-c3c-4ccc4ccccc34)cc2)cc1 ; c1ccc(-c2ccc(N(c3ccc4ccccc4c3)c3cccc4c3Oc3ccccc3-c3c-4ccc4ccccc34)cc2)cc1]

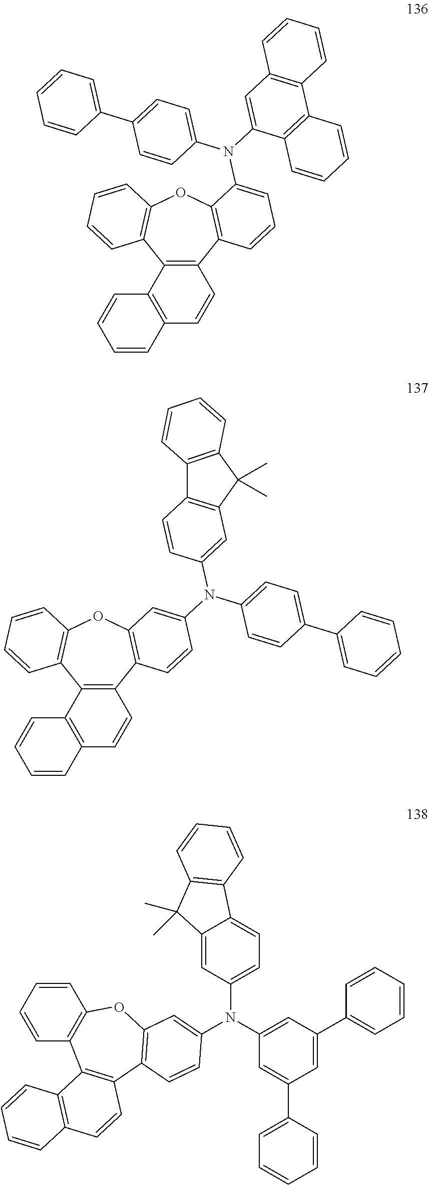 [SMILES: c1ccc(-c2ccc(N(c3cccc4c3Oc3ccccc3-c3c-4ccc4ccccc34)c3cc4ccccc4c4ccccc34)cc2)cc1 ; CC1(C)c2ccccc2-c2ccc(N(c3ccc(-c4ccccc4)cc3)c3ccc4c(c3)Oc3ccccc3-c3c-4ccc4ccccc34)cc21 ; CC1(C)c2ccccc2-c2ccc(N(c3cc(-c4ccccc4)cc(-c4ccccc4)c3)c3ccc4c(c3)Oc3ccccc3-c3c-4ccc4ccccc34)cc21]

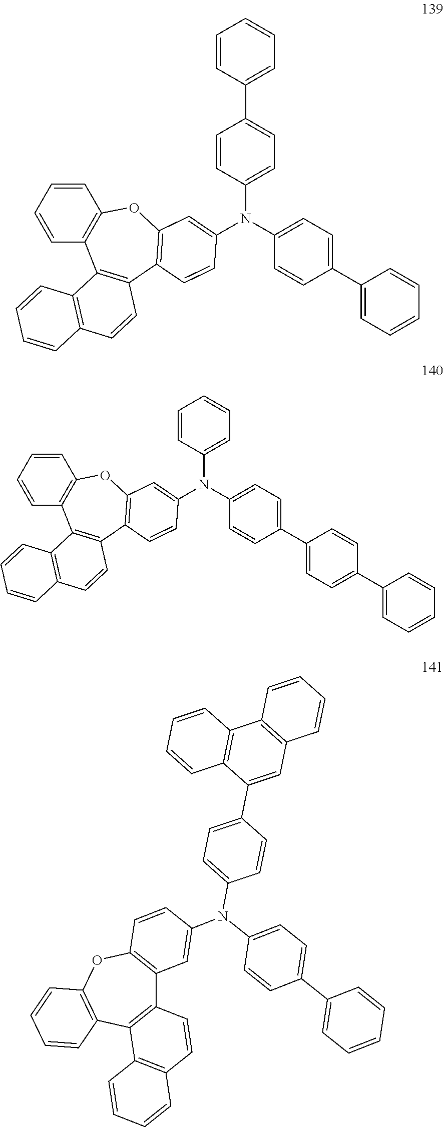 [SMILES: c1ccc(-c2ccc(N(c3ccc(-c4ccccc4)cc3)c3ccc4c(c3)Oc3ccccc3-c3c-4ccc4ccccc34)cc2)cc1 ; c1ccc(-c2ccc(-c3ccc(N(c4ccccc4)c4ccc5c(c4)Oc4ccccc4-c4c-5ccc5ccccc45)cc3)cc2)cc1 ; c1ccc(-c2ccc(N(c3ccc(-c4cc5ccccc5c5ccccc45)cc3)c3ccc4c(c3)-c3ccc5ccccc5c3-c3ccccc3O4)cc2)cc1]

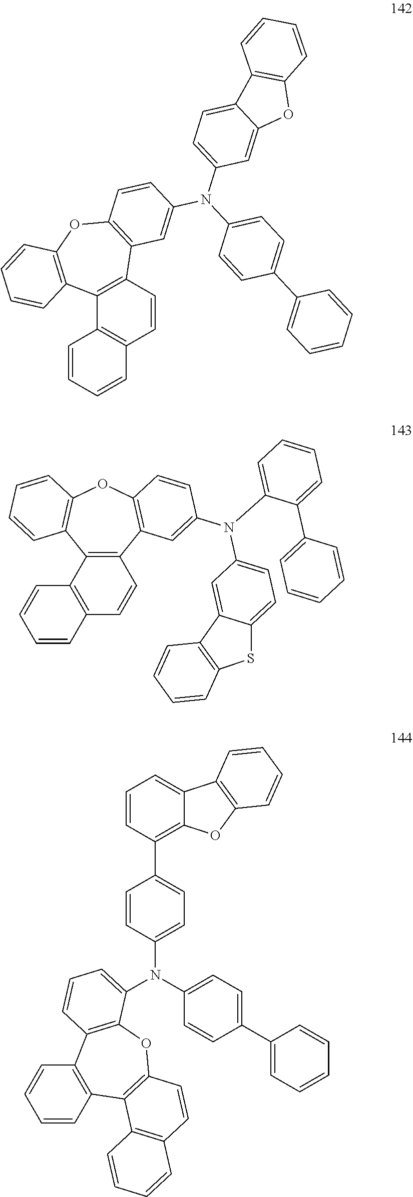 [SMILES: c1ccc(-c2ccc(N(c3ccc4c(c3)-c3ccc5ccccc5c3-c3ccccc3O4)c3ccc4c(c3)oc3ccccc34)cc2)cc1 ; c1ccc(-c2ccccc2N(c2ccc3c(c2)-c2ccc4ccccc4c2-c2ccccc2O3)c2ccc3sc4ccccc4c3c2)cc1 ; c1ccc(-c2ccc(N(c3ccc(-c4cccc5c4oc4ccccc45)cc3)c3cccc4c3Oc3ccc5ccccc5c3-c3ccccc3-4)cc2)cc1]

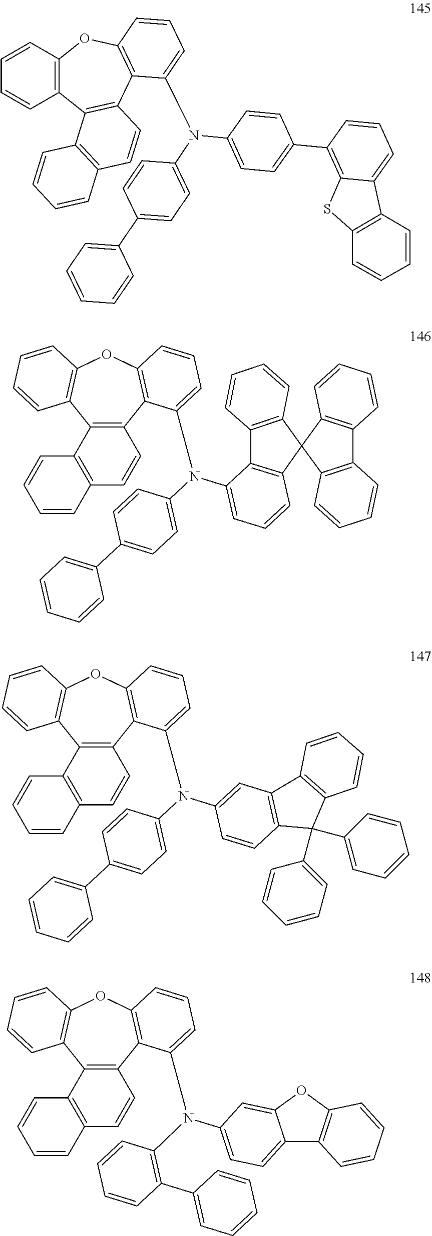 [SMILES: c1ccc(-c2ccc(N(c3ccc(-c4cccc5c4sc4ccccc45)cc3)c3cccc4c3-c3ccc5ccccc5c3-c3ccccc3O4)cc2)cc1 ; c1ccc(-c2ccc(N(c3cccc4c3-c3ccc5ccccc5c3-c3ccccc3O4)c3cccc4c3-c3ccccc3C43c4ccccc4-c4ccccc43)cc2)cc1 ; c1ccc(-c2ccc(N(c3ccc4c(c3)-c3ccccc3C4(c3ccccc3)c3ccccc3)c3cccc4c3-c3ccc5ccccc5c3-c3ccccc3O4)cc2)cc1 ; c1ccc(-c2ccccc2N(c2ccc3c(c2)oc2ccccc23)c2cccc3c2-c2ccc4ccccc4c2-c2ccccc2O3)cc1]

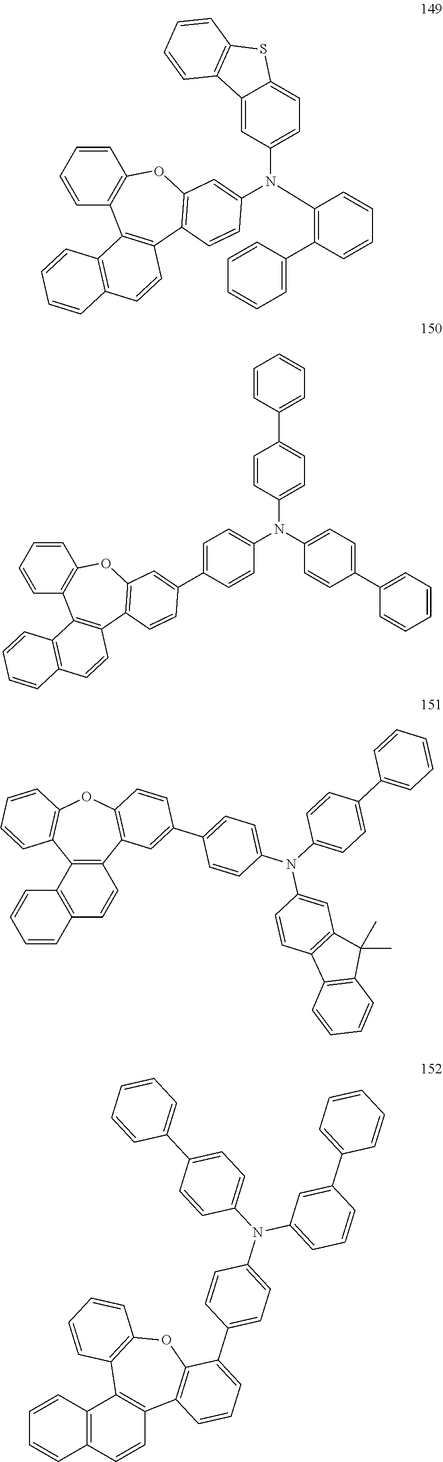 [SMILES: c1ccc(-c2ccccc2N(c2ccc3c(c2)Oc2ccccc2-c2c-3ccc3ccccc23)c2ccc3sc4ccccc4c3c2)cc1 ; c1ccc(-c2ccc(N(c3ccc(-c4ccccc4)cc3)c3ccc(-c4ccc5c(c4)Oc4ccccc4-c4c-5ccc5ccccc45)cc3)cc2)cc1 ; CC1(C)c2ccccc2-c2ccc(N(c3ccc(-c4ccccc4)cc3)c3ccc(-c4ccc5c(c4)-c4ccc6ccccc6c4-c4ccccc4O5)cc3)cc21 ; c1ccc(-c2ccc(N(c3ccc(-c4cccc5c4Oc4ccccc4-c4c-5ccc5ccccc45)cc3)c3cccc(-c4ccccc4)c3)cc2)cc1]

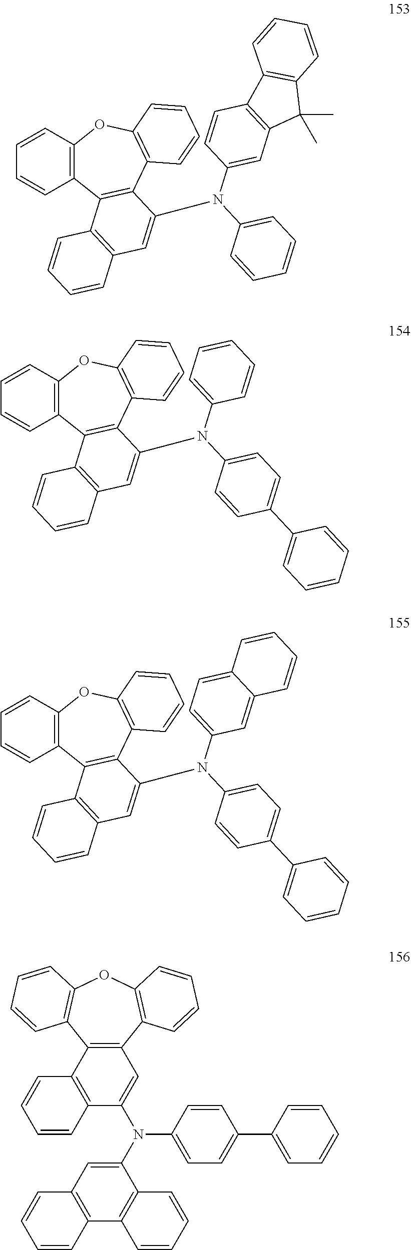 [SMILES: CC1(C)c2ccccc2-c2ccc(N(c3ccccc3)c3cc4ccccc4c4c3-c3ccccc3Oc3ccccc3-4)cc21 ; c1ccc(-c2ccc(N(c3ccccc3)c3cc4ccccc4c4c3-c3ccccc3Oc3ccccc3-4)cc2)cc1 ; c1ccc(-c2ccc(N(c3ccc4ccccc4c3)c3cc4ccccc4c4c3-c3ccccc3Oc3ccccc3-4)cc2)cc1 ; c1ccc(-c2ccc(N(c3cc4c(c5ccccc35)-c3ccccc3Oc3ccccc3-4)c3cc4ccccc4c4ccccc34)cc2)cc1]

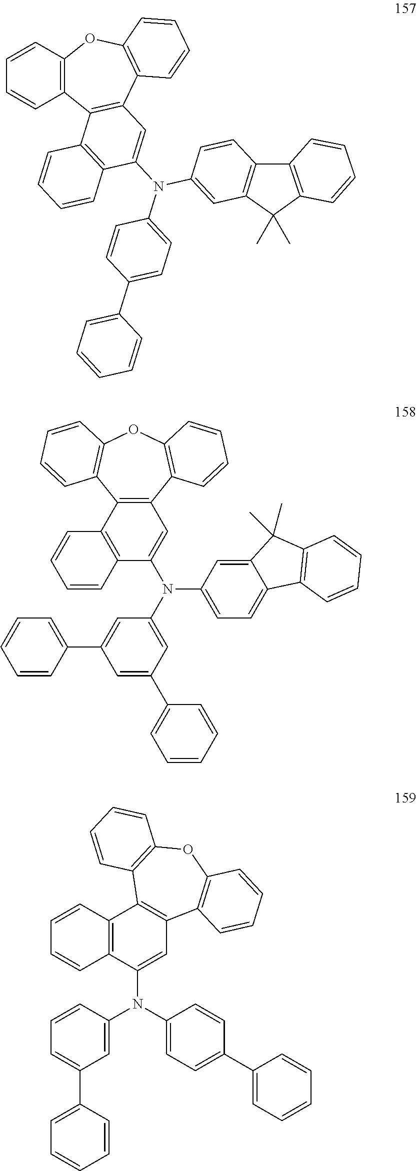 [SMILES: CC1(C)c2ccccc2-c2ccc(N(c3ccc(-c4ccccc4)cc3)c3cc4c(c5ccccc35)-c3ccccc3Oc3ccccc3-4)cc21 ; CC1(C)c2ccccc2-c2ccc(N(c3cc(-c4ccccc4)cc(-c4ccccc4)c3)c3cc4c(c5ccccc35)-c3ccccc3Oc3ccccc3-4)cc21 ; c1ccc(-c2ccc(N(c3cccc(-c4ccccc4)c3)c3cc4c(c5ccccc35)-c3ccccc3Oc3ccccc3-4)cc2)cc1]

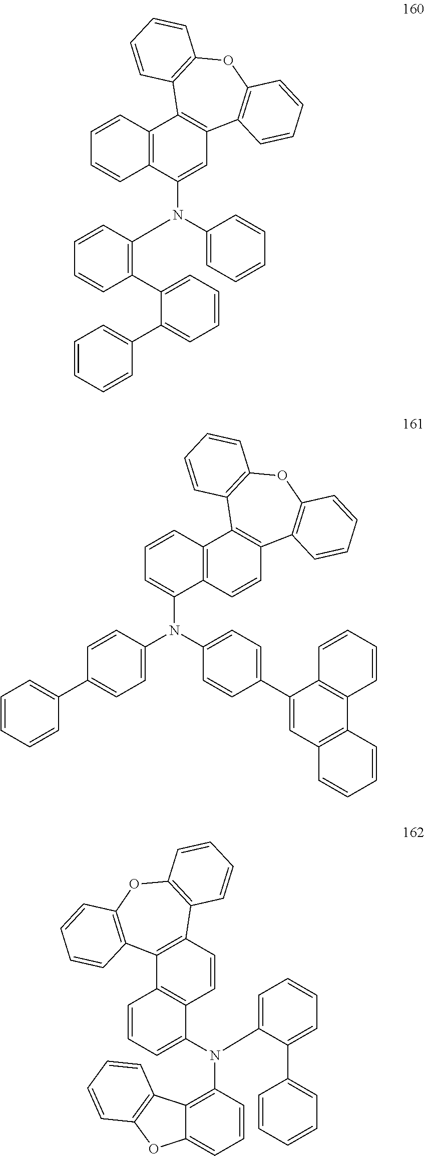 [SMILES: c1ccc(-c2ccccc2-c2ccccc2N(c2ccccc2)c2cc3c(c4ccccc24)-c2ccccc2Oc2ccccc2-3)cc1 ; c1ccc(-c2ccc(N(c3ccc(-c4cc5ccccc5c5ccccc45)cc3)c3cccc4c5c(ccc34)-c3ccccc3Oc3ccccc3-5)cc2)cc1 ; c1ccc(-c2ccccc2N(c2cccc3c4c(ccc23)-c2ccccc2Oc2ccccc2-4)c2cccc3oc4ccccc4c23)cc1]

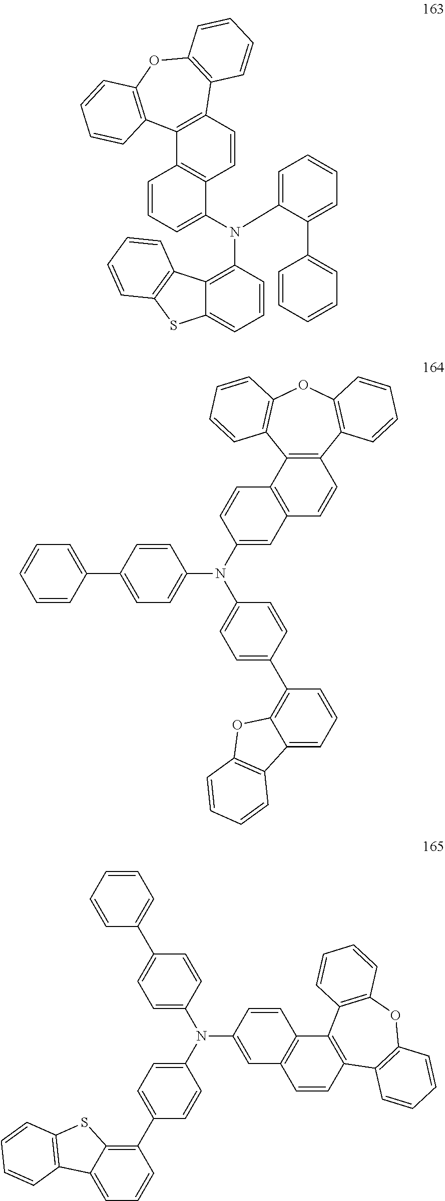 [SMILES: c1ccc(-c2ccccc2N(c2cccc3c4c(ccc23)-c2ccccc2Oc2ccccc2-4)c2cccc3sc4ccccc4c23)cc1 ; c1ccc(-c2ccc(N(c3ccc(-c4cccc5c4oc4ccccc45)cc3)c3ccc4c5c(ccc4c3)-c3ccccc3Oc3ccccc3-5)cc2)cc1 ; c1ccc(-c2ccc(N(c3ccc(-c4cccc5c4sc4ccccc45)cc3)c3ccc4c5c(ccc4c3)-c3ccccc3Oc3ccccc3-5)cc2)cc1]

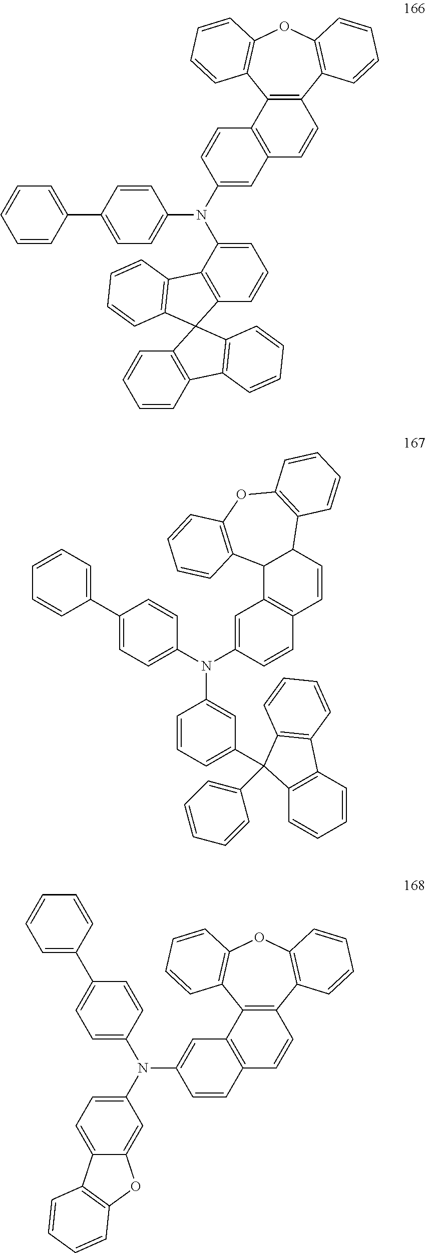 [SMILES: c1ccc(-c2ccc(N(c3ccc4c5c(ccc4c3)-c3ccccc3Oc3ccccc3-5)c3cccc4c3-c3ccccc3C43c4ccccc4-c4ccccc43)cc2)cc1 ; C1=CC2c3ccccc3Oc3ccccc3C2c2cc(N(c3ccc(-c4ccccc4)cc3)c3cccc(C4(c5ccccc5)c5ccccc5-c5ccccc54)c3)ccc21 ; c1ccc(-c2ccc(N(c3ccc4c(c3)oc3ccccc34)c3ccc4ccc5c(c4c3)-c3ccccc3Oc3ccccc3-5)cc2)cc1]

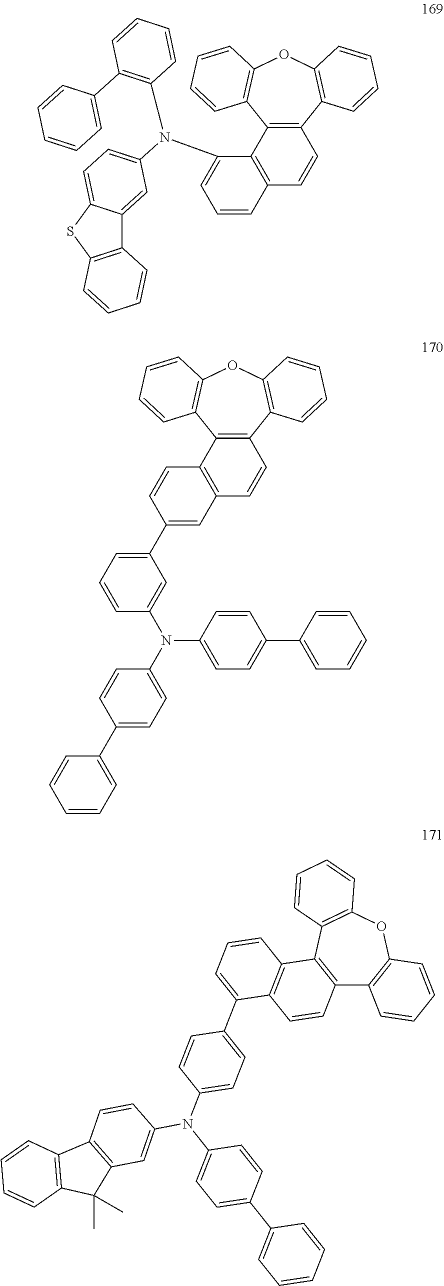 [SMILES: c1ccc(-c2ccccc2N(c2ccc3sc4ccccc4c3c2)c2cccc3ccc4c(c23)-c2ccccc2Oc2ccccc2-4)cc1 ; c1ccc(-c2ccc(N(c3ccc(-c4ccccc4)cc3)c3cccc(-c4ccc5c6c(ccc5c4)-c4ccccc4Oc4ccccc4-6)c3)cc2)cc1 ; CC1(C)c2ccccc2-c2ccc(N(c3ccc(-c4ccccc4)cc3)c3ccc(-c4cccc5c6c(ccc45)-c4ccccc4Oc4ccccc4-6)cc3)cc21]

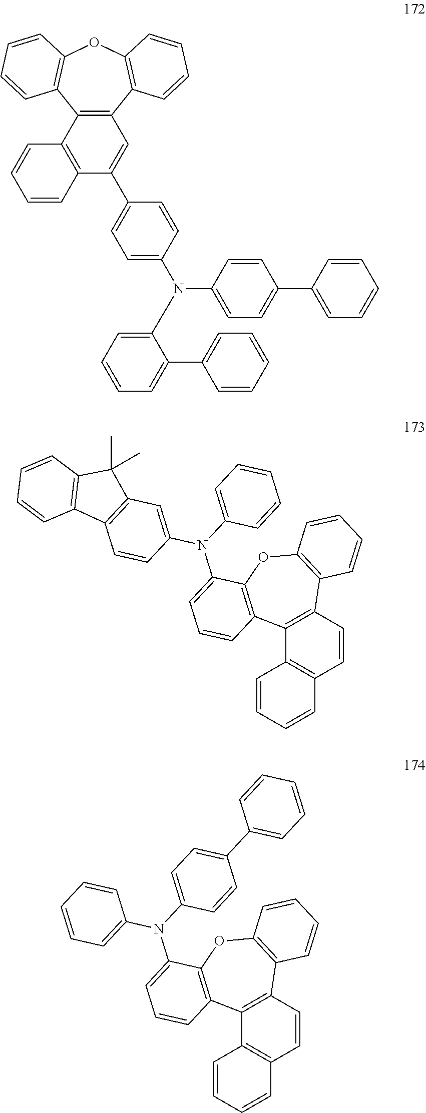

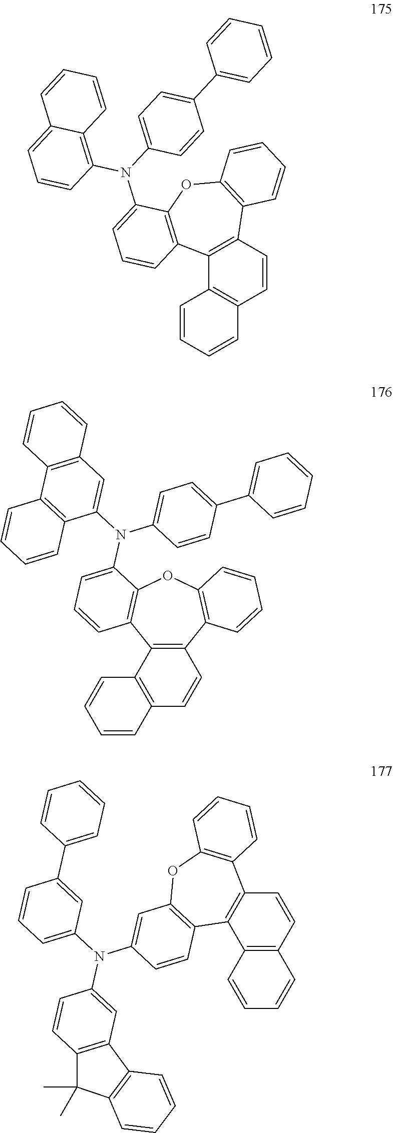 [SMILES: c1ccc(-c2ccc(N(c3cccc4c3Oc3ccccc3-c3ccc5ccccc5c3-4)c3cccc4ccccc34)cc2)cc1 ; c1ccc(-c2ccc(N(c3cccc4c3Oc3ccccc3-c3ccc5ccccc5c3-4)c3cc4ccccc4c4ccccc34)cc2)cc1 ; CC1(C)c2ccccc2-c2cc(N(c3cccc(-c4ccccc4)c3)c3ccc4c(c3)Oc3ccccc3-c3ccc5ccccc5c3-4)ccc21]

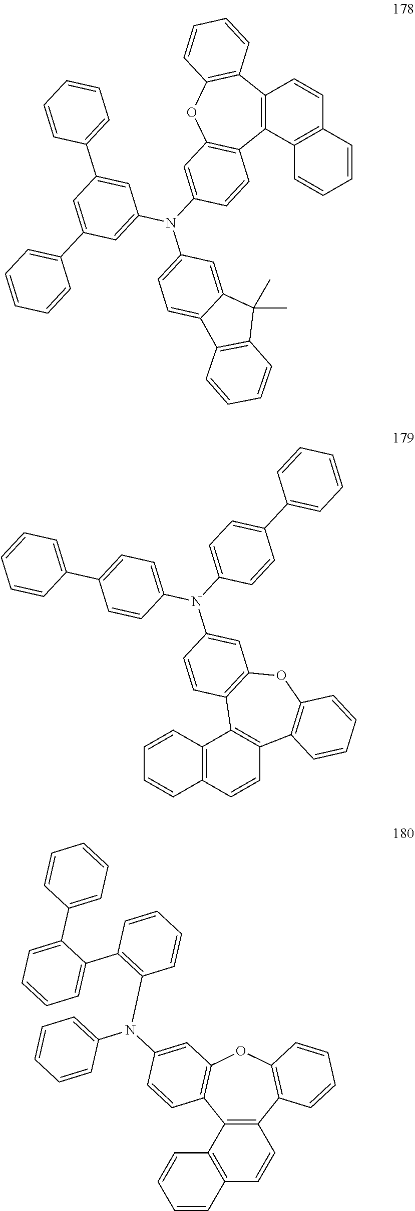 [SMILES: CC1(C)c2ccccc2-c2ccc(N(c3cc(-c4ccccc4)cc(-c4ccccc4)c3)c3ccc4c(c3)Oc3ccccc3-c3ccc5ccccc5c3-4)cc21 ; c1ccc(-c2ccc(N(c3ccc(-c4ccccc4)cc3)c3ccc4c(c3)Oc3ccccc3-c3ccc5ccccc5c3-4)cc2)cc1 ; c1ccc(-c2ccccc2-c2ccccc2N(c2ccccc2)c2ccc3c(c2)Oc2ccccc2-c2ccc4ccccc4c2-3)cc1]

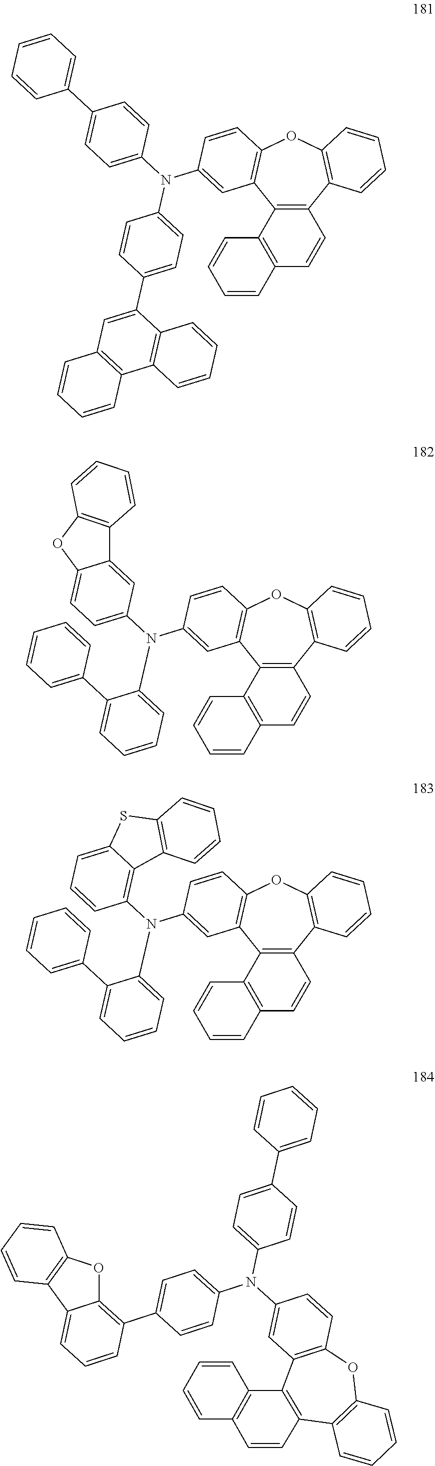

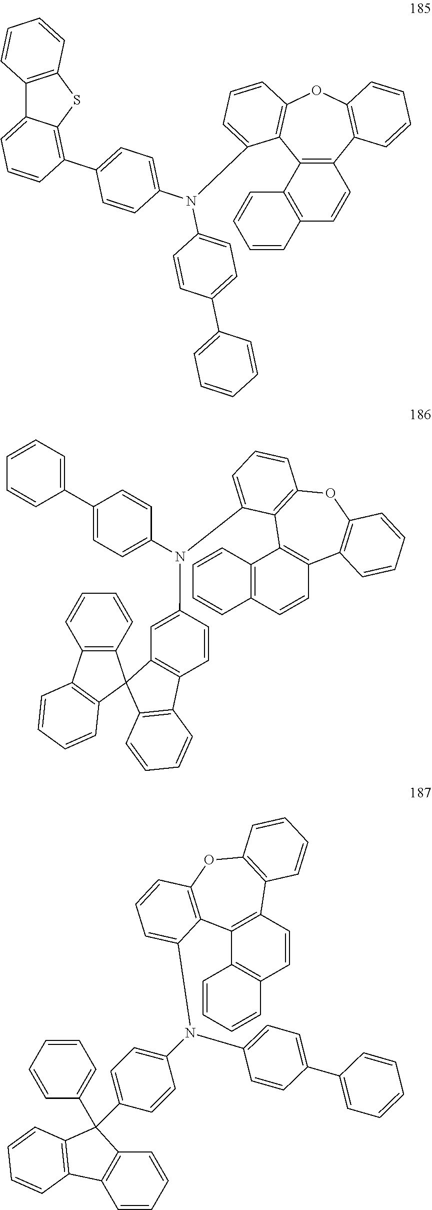 [SMILES: c1ccc(-c2ccc(N(c3ccc(-c4cccc5c4sc4ccccc45)cc3)c3cccc4c3-c3c(ccc5ccccc35)-c3ccccc3O4)cc2)cc1 ; c1ccc(-c2ccc(N(c3ccc4c(c3)C3(c5ccccc5-c5ccccc53)c3ccccc3-4)c3cccc4c3-c3c(ccc5ccccc35)-c3ccccc3O4)cc2)cc1 ; c1ccc(-c2ccc(N(c3ccc(C4(c5ccccc5)c5ccccc5-c5ccccc54)cc3)c3cccc4c3-c3c(ccc5ccccc35)-c3ccccc3O4)cc2)cc1]

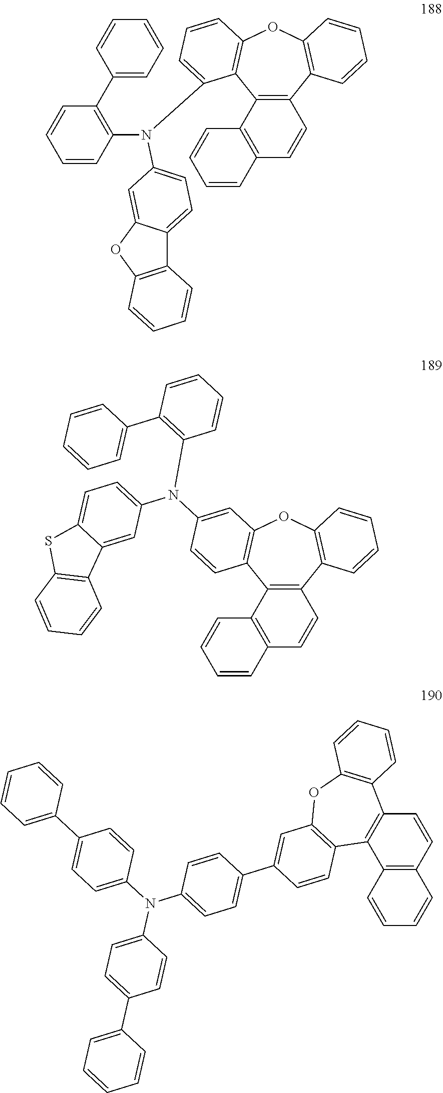 [SMILES: c1ccc(-c2ccccc2N(c2ccc3c(c2)oc2ccccc23)c2cccc3c2-c2c(ccc4ccccc24)-c2ccccc2O3)cc1 ; c1ccc(-c2ccccc2N(c2ccc3c(c2)Oc2ccccc2-c2ccc4ccccc4c2-3)c2ccc3sc4ccccc4c3c2)cc1 ; c1ccc(-c2ccc(N(c3ccc(-c4ccccc4)cc3)c3ccc(-c4ccc5c(c4)Oc4ccccc4-c4ccc6ccccc6c4-5)cc3)cc2)cc1]

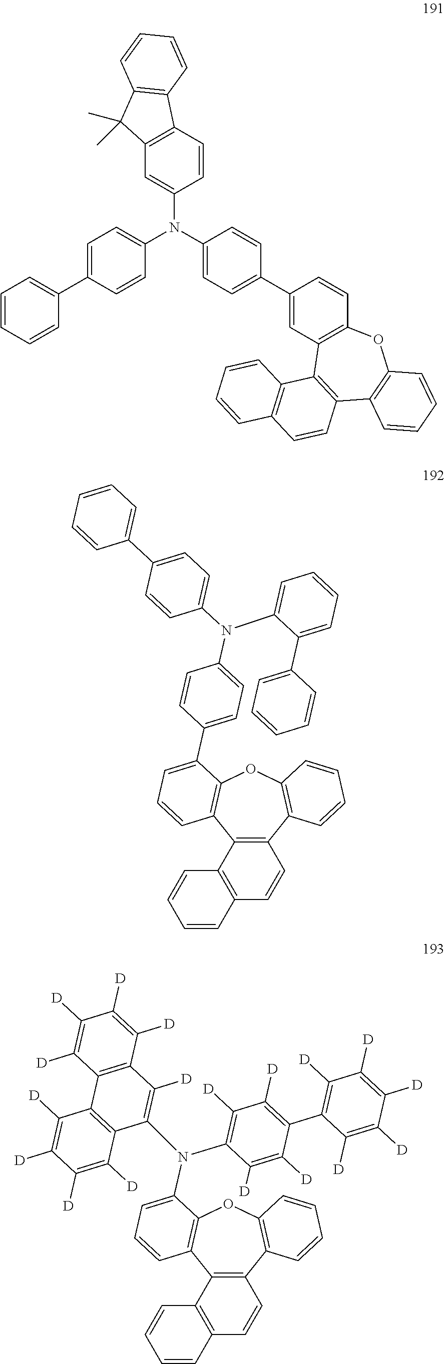 [SMILES: CC1(C)c2ccccc2-c2ccc(N(c3ccc(-c4ccccc4)cc3)c3ccc(-c4ccc5c(c4)-c4c(ccc6ccccc46)-c4ccccc4O5)cc3)cc21 ; c1ccc(-c2ccc(N(c3ccc(-c4cccc5c4Oc4ccccc4-c4ccc6ccccc6c4-5)cc3)c3ccccc3-c3ccccc3)cc2)cc1 ; [2H]c1c([2H])c([2H])c(-c2c([2H])c([2H])c(N(c3cccc4c3Oc3ccccc3-c3ccc5ccccc5c3-4)c3c([2H])c4c([2H])c([2H])c([2H])c([2H])c4c4c([2H])c([2H])c([2H])c([2H])c34)c([2H])c2[2H])c([2H])c1[2H]]

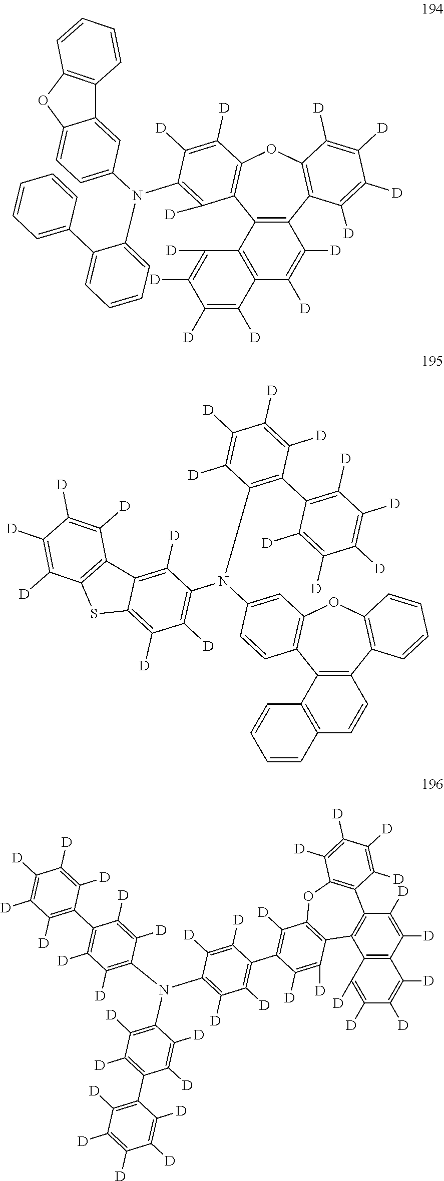

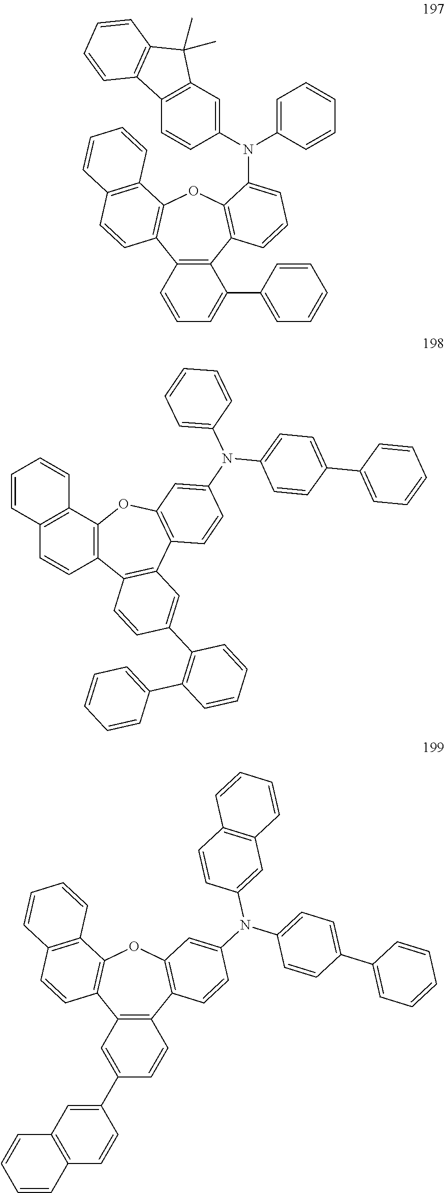 [SMILES: CC1(C)c2ccccc2-c2ccc(N(c3ccccc3)c3cccc4c3Oc3c(ccc5ccccc35)-c3cccc(-c5ccccc5)c3-4)cc21 ; c1ccc(-c2ccc(N(c3ccccc3)c3ccc4c(c3)Oc3c(ccc5ccccc35)-c3ccc(-c5ccccc5-c5ccccc5)cc3-4)cc2)cc1 ; c1ccc(-c2ccc(N(c3ccc4c(c3)Oc3c(ccc5ccccc35)-c3cc(-c5ccc6ccccc6c5)ccc3-4)c3ccc4ccccc4c3)cc2)cc1]

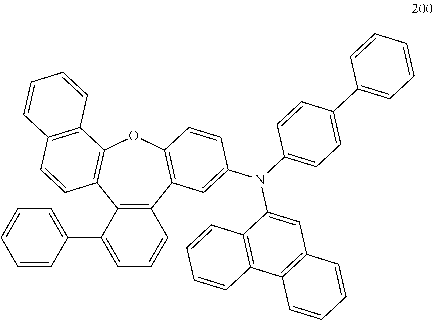 [SMILES: c1ccc(-c2ccc(N(c3ccc4c(c3)-c3cccc(-c5ccccc5)c3-c3ccc5ccccc5c3O4)c3cc4ccccc4c4ccccc34)cc2)cc1]

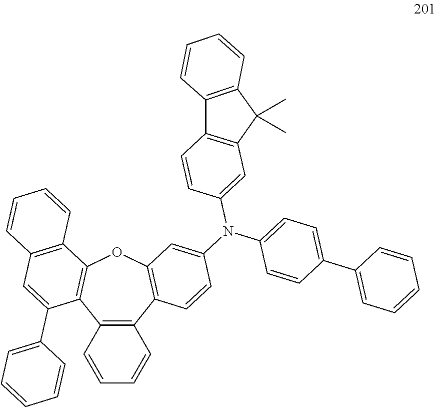 [SMILES: CC1(C)c2ccccc2-c2ccc(N(c3ccc(-c4ccccc4)cc3)c3ccc4c(c3)Oc3c(c(-c5ccccc5)cc5ccccc35)-c3ccccc3-4)cc21]

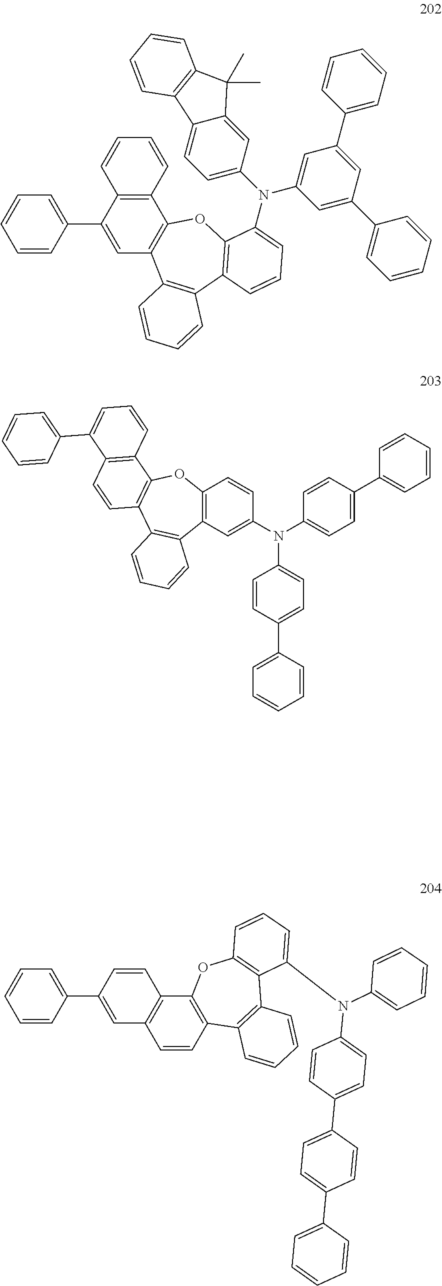 [SMILES: CC1(C)c2ccccc2-c2ccc(N(c3cc(-c4ccccc4)cc(-c4ccccc4)c3)c3cccc4c3Oc3c(cc(-c5ccccc5)c5ccccc35)-c3ccccc3-4)cc21 ; c1ccc(-c2ccc(N(c3ccc(-c4ccccc4)cc3)c3ccc4c(c3)-c3ccccc3-c3ccc5c(-c6ccccc6)cccc5c3O4)cc2)cc1 ; c1ccc(-c2ccc(-c3ccc(N(c4ccccc4)c4cccc5c4-c4ccccc4-c4ccc6cc(-c7ccccc7)ccc6c4O5)cc3)cc2)cc1]

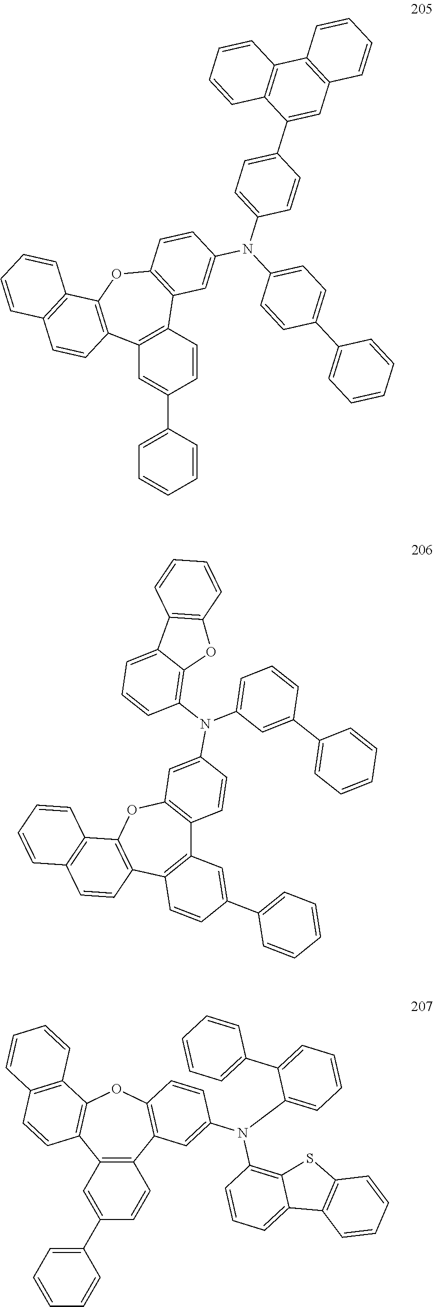 [SMILES: c1ccc(-c2ccc(N(c3ccc(-c4cc5ccccc5c5ccccc45)cc3)c3ccc4c(c3)-c3ccc(-c5ccccc5)cc3-c3ccc5ccccc5c3O4)cc2)cc1 ; c1ccc(-c2cccc(N(c3ccc4c(c3)Oc3c(ccc5ccccc35)-c3ccc(-c5ccccc5)cc3-4)c3cccc4c3oc3ccccc34)c2)cc1 ; c1ccc(-c2ccc3c(c2)-c2ccc4ccccc4c2Oc2ccc(N(c4ccccc4-c4ccccc4)c4cccc5c4sc4ccccc45)cc2-3)cc1]

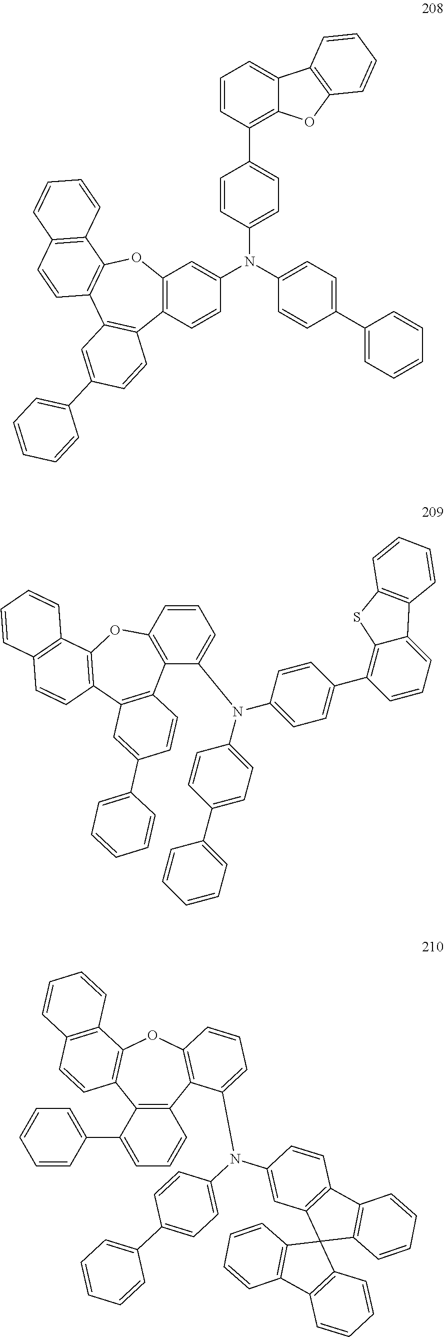 [SMILES: c1ccc(-c2ccc(N(c3ccc(-c4cccc5c4oc4ccccc45)cc3)c3ccc4c(c3)Oc3c(ccc5ccccc35)-c3cc(-c5ccccc5)ccc3-4)cc2)cc1 ; c1ccc(-c2ccc(N(c3ccc(-c4cccc5c4sc4ccccc45)cc3)c3cccc4c3-c3ccc(-c5ccccc5)cc3-c3ccc5ccccc5c3O4)cc2)cc1 ; c1ccc(-c2ccc(N(c3ccc4c(c3)C3(c5ccccc5-c5ccccc53)c3ccccc3-4)c3cccc4c3-c3cccc(-c5ccccc5)c3-c3ccc5ccccc5c3O4)cc2)cc1]

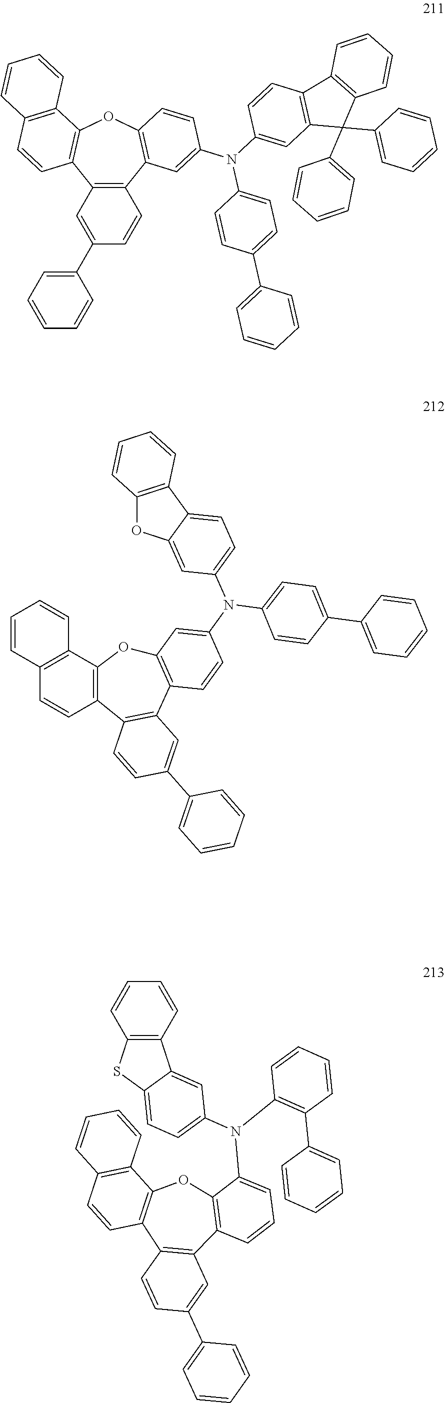 [SMILES: c1ccc(-c2ccc(N(c3ccc4c(c3)-c3ccc(-c5ccccc5)cc3-c3ccc5ccccc5c3O4)c3ccc4c(c3)C(c3ccccc3)(c3ccccc3)c3ccccc3-4)cc2)cc1 ; c1ccc(-c2ccc(N(c3ccc4c(c3)Oc3c(ccc5ccccc35)-c3ccc(-c5ccccc5)cc3-4)c3ccc4c(c3)oc3ccccc34)cc2)cc1 ; c1ccc(-c2ccc3c(c2)-c2cccc(N(c4ccc5sc6ccccc6c5c4)c4ccccc4-c4ccccc4)c2Oc2c-3ccc3ccccc23)cc1]

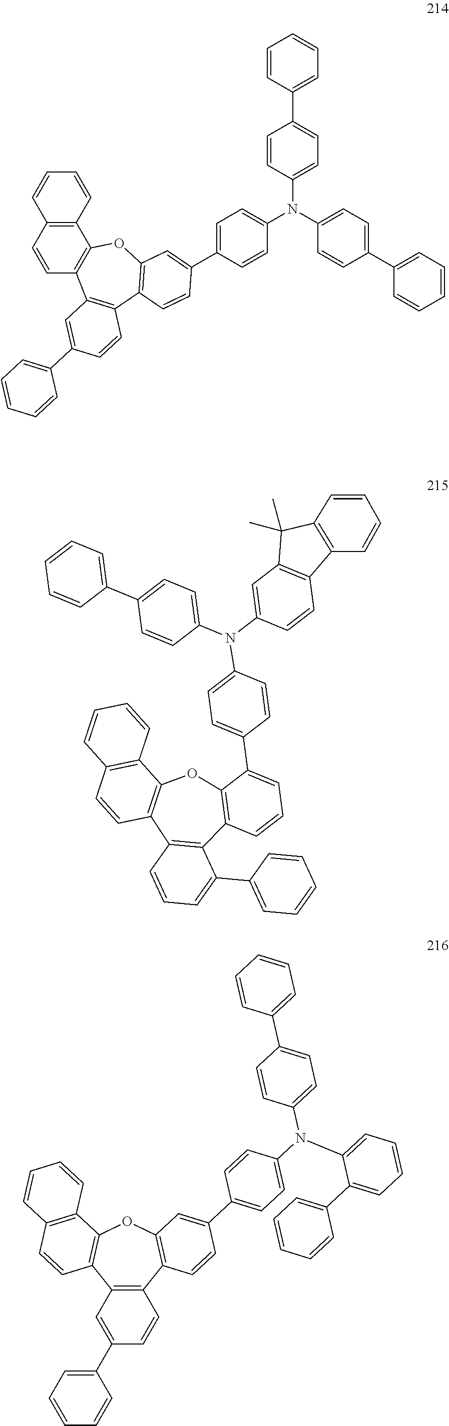 [SMILES: c1ccc(-c2ccc(N(c3ccc(-c4ccccc4)cc3)c3ccc(-c4ccc5c(c4)Oc4c(ccc6ccccc46)-c4cc(-c6ccccc6)ccc4-5)cc3)cc2)cc1 ; CC1(C)c2ccccc2-c2ccc(N(c3ccc(-c4ccccc4)cc3)c3ccc(-c4cccc5c4Oc4c(ccc6ccccc46)-c4cccc(-c6ccccc6)c4-5)cc3)cc21 ; c1ccc(-c2ccc(N(c3ccc(-c4ccc5c(c4)Oc4c(ccc6ccccc46)-c4cc(-c6ccccc6)ccc4-5)cc3)c3ccccc3-c3ccccc3)cc2)cc1]

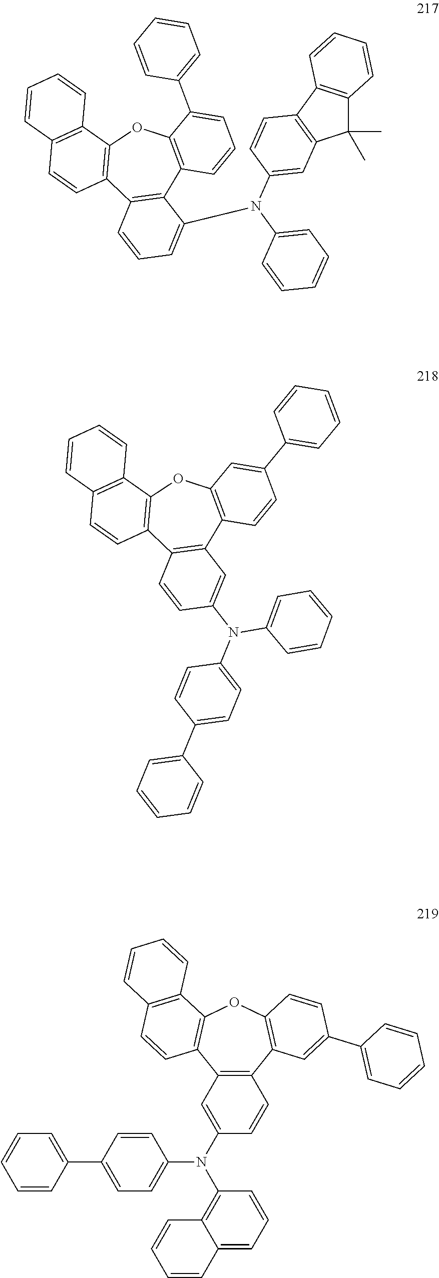 [SMILES: CC1(C)c2ccccc2-c2ccc(N(c3ccccc3)c3cccc4c3-c3cccc(-c5ccccc5)c3Oc3c-4ccc4ccccc34)cc21 ; c1ccc(-c2ccc(N(c3ccccc3)c3ccc4c(c3)-c3ccc(-c5ccccc5)cc3Oc3c-4ccc4ccccc34)cc2)cc1 ; c1ccc(-c2ccc(N(c3ccc4c(c3)-c3ccc5ccccc5c3Oc3ccc(-c5ccccc5)cc3-4)c3cccc4ccccc34)cc2)cc1]

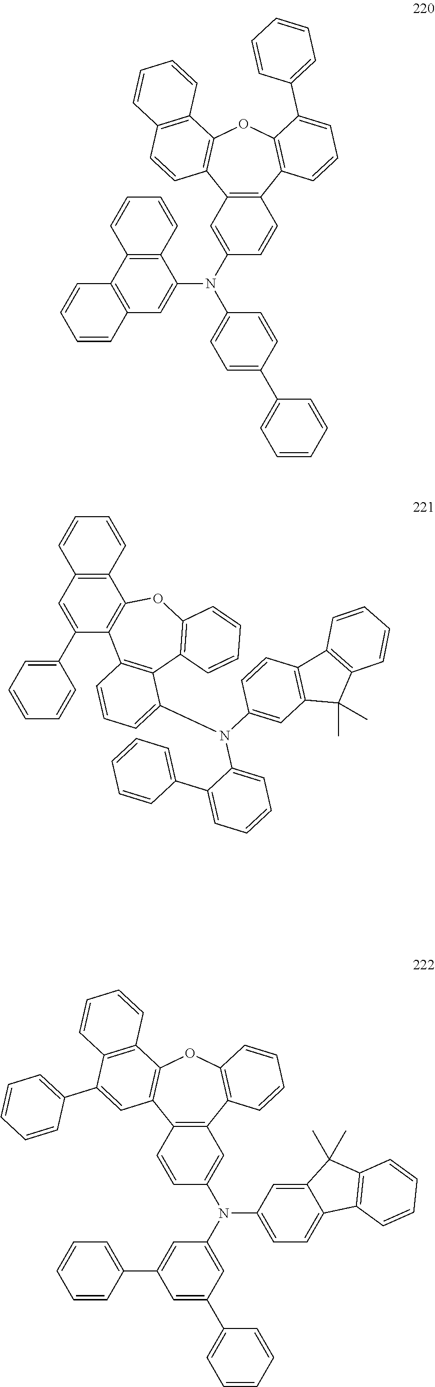 [SMILES: c1ccc(-c2ccc(N(c3ccc4c(c3)-c3ccc5ccccc5c3Oc3c(-c5ccccc5)cccc3-4)c3cc4ccccc4c4ccccc34)cc2)cc1 ; CC1(C)c2ccccc2-c2ccc(N(c3ccccc3-c3ccccc3)c3cccc4c3-c3ccccc3Oc3c-4c(-c4ccccc4)cc4ccccc34)cc21 ; CC1(C)c2ccccc2-c2ccc(N(c3cc(-c4ccccc4)cc(-c4ccccc4)c3)c3ccc4c(c3)-c3ccccc3Oc3c-4cc(-c4ccccc4)c4ccccc34)cc21]

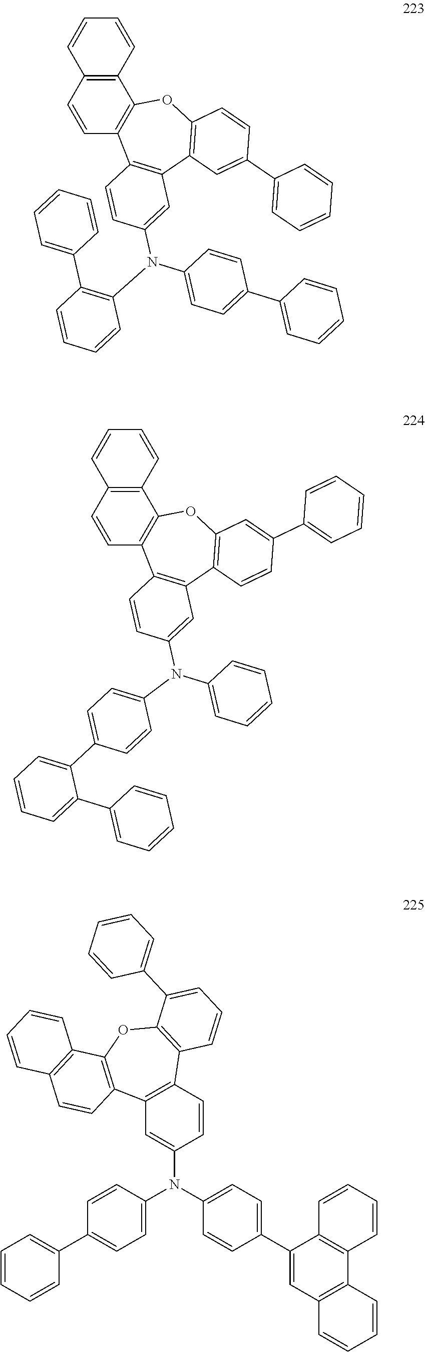 [SMILES: c1ccc(-c2ccc(N(c3ccc4c(c3)-c3cc(-c5ccccc5)ccc3Oc3c-4ccc4ccccc34)c3ccccc3-c3ccccc3)cc2)cc1 ; c1ccc(-c2ccc3c(c2)Oc2c(ccc4ccccc24)-c2ccc(N(c4ccccc4)c4ccc(-c5ccccc5-c5ccccc5)cc4)cc2-3)cc1 ; c1ccc(-c2ccc(N(c3ccc(-c4cc5ccccc5c5ccccc45)cc3)c3ccc4c(c3)-c3ccc5ccccc5c3Oc3c(-c5ccccc5)cccc3-4)cc2)cc1]

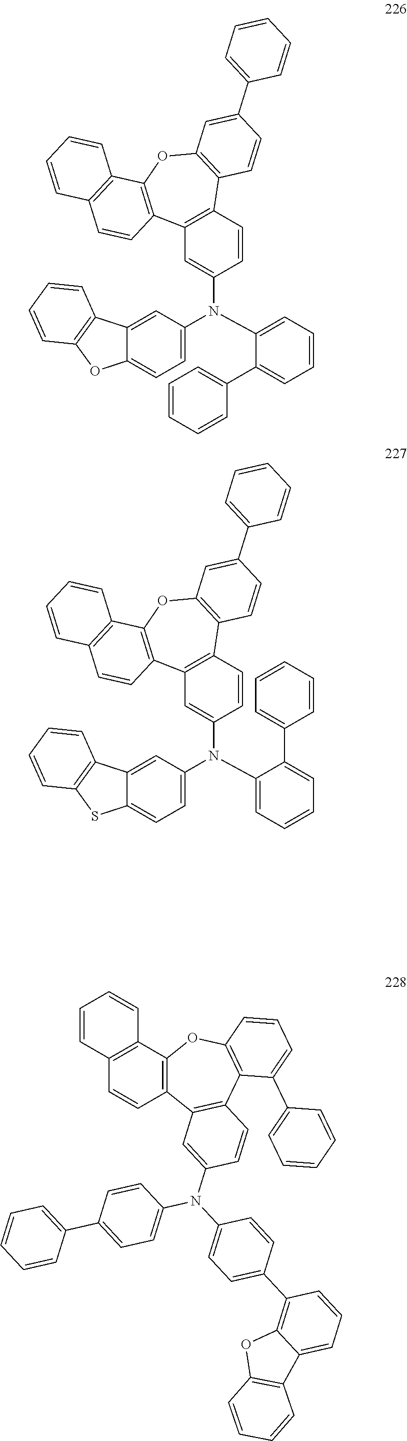 [SMILES: c1ccc(-c2ccc3c(c2)Oc2c(ccc4ccccc24)-c2cc(N(c4ccc5oc6ccccc6c5c4)c4ccccc4-c4ccccc4)ccc2-3)cc1 ; c1ccc(-c2ccc3c(c2)Oc2c(ccc4ccccc24)-c2cc(N(c4ccc5sc6ccccc6c5c4)c4ccccc4-c4ccccc4)ccc2-3)cc1 ; c1ccc(-c2ccc(N(c3ccc(-c4cccc5c4oc4ccccc45)cc3)c3ccc4c(c3)-c3ccc5ccccc5c3Oc3cccc(-c5ccccc5)c3-4)cc2)cc1]

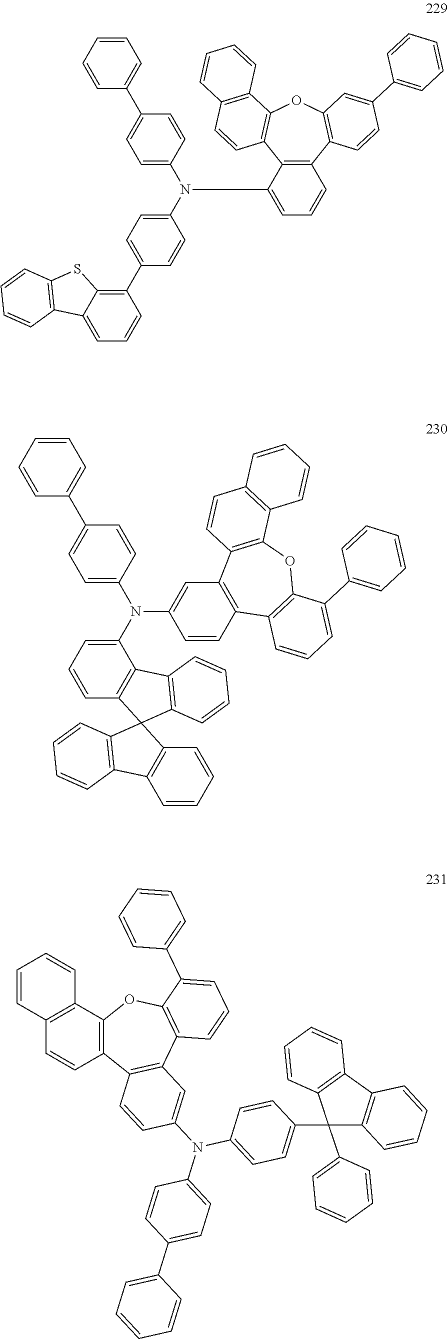 [SMILES: c1ccc(-c2ccc(N(c3ccc(-c4cccc5c4sc4ccccc45)cc3)c3cccc4c3-c3ccc5ccccc5c3Oc3cc(-c5ccccc5)ccc3-4)cc2)cc1 ; c1ccc(-c2ccc(N(c3ccc4c(c3)-c3ccc5ccccc5c3Oc3c(-c5ccccc5)cccc3-4)c3cccc4c3-c3ccccc3C43c4ccccc4-c4ccccc43)cc2)cc1 ; c1ccc(-c2ccc(N(c3ccc(C4(c5ccccc5)c5ccccc5-c5ccccc54)cc3)c3ccc4c(c3)-c3cccc(-c5ccccc5)c3Oc3c-4ccc4ccccc34)cc2)cc1]

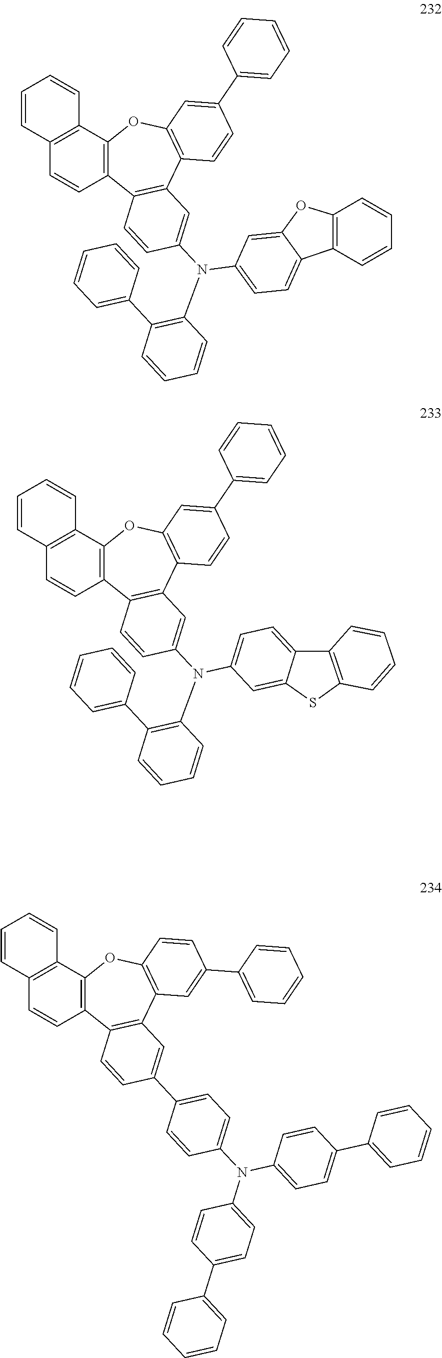 [SMILES: c1ccc(-c2ccc3c(c2)Oc2c(ccc4ccccc24)-c2ccc(N(c4ccc5c(c4)oc4ccccc45)c4ccccc4-c4ccccc4)cc2-3)cc1 ; c1ccc(-c2ccc3c(c2)Oc2c(ccc4ccccc24)-c2ccc(N(c4ccc5c(c4)sc4ccccc45)c4ccccc4-c4ccccc4)cc2-3)cc1 ; c1ccc(-c2ccc(N(c3ccc(-c4ccccc4)cc3)c3ccc(-c4ccc5c(c4)-c4cc(-c6ccccc6)ccc4Oc4c-5ccc5ccccc45)cc3)cc2)cc1]

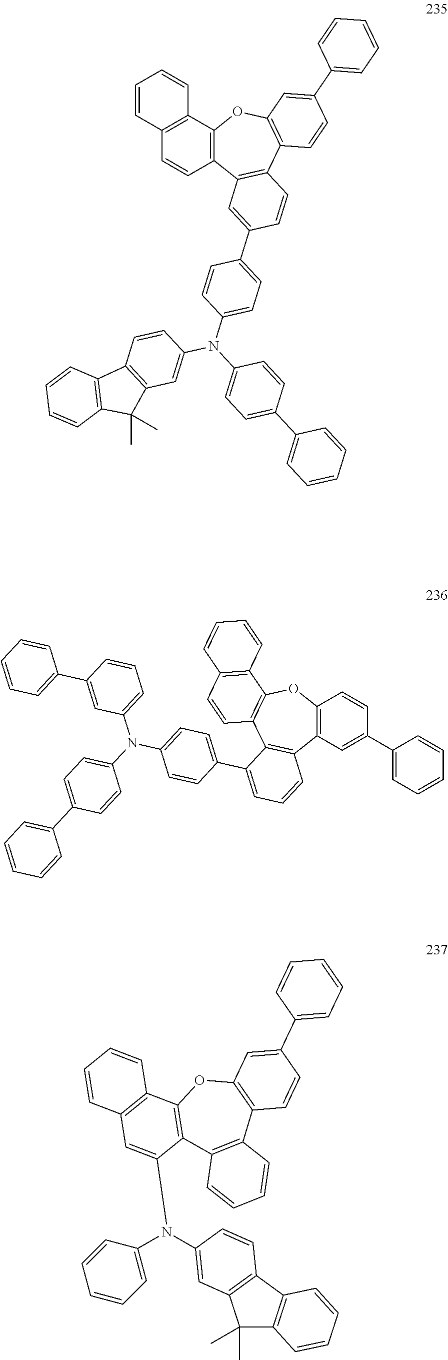 [SMILES: CC1(C)c2ccccc2-c2ccc(N(c3ccc(-c4ccccc4)cc3)c3ccc(-c4ccc5c(c4)-c4ccc6ccccc6c4Oc4cc(-c6ccccc6)ccc4-5)cc3)cc21 ; c1ccc(-c2ccc(N(c3ccc(-c4cccc5c4-c4ccc6ccccc6c4Oc4ccc(-c6ccccc6)cc4-5)cc3)c3cccc(-c4ccccc4)c3)cc2)cc1 ; CC1(C)c2ccccc2-c2ccc(N(c3ccccc3)c3cc4ccccc4c4c3-c3ccccc3-c3ccc(-c5ccccc5)cc3O4)cc21]

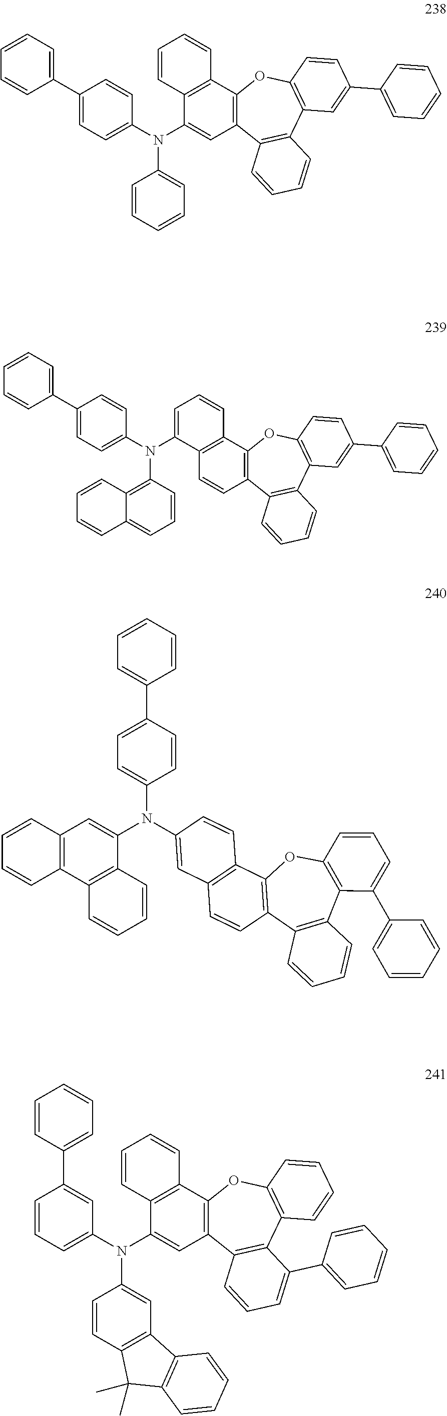 [SMILES: c1ccc(-c2ccc(N(c3ccccc3)c3cc4c(c5ccccc35)Oc3ccc(-c5ccccc5)cc3-c3ccccc3-4)cc2)cc1 ; c1ccc(-c2ccc(N(c3cccc4ccccc34)c3cccc4c5c(ccc34)-c3ccccc3-c3cc(-c4ccccc4)ccc3O5)cc2)cc1 ; c1ccc(-c2ccc(N(c3ccc4c5c(ccc4c3)-c3ccccc3-c3c(cccc3-c3ccccc3)O5)c3cc4ccccc4c4ccccc34)cc2)cc1 ; CC1(C)c2ccccc2-c2cc(N(c3cccc(-c4ccccc4)c3)c3cc4c(c5ccccc35)Oc3ccccc3-c3c(-c5ccccc5)cccc3-4)ccc21]

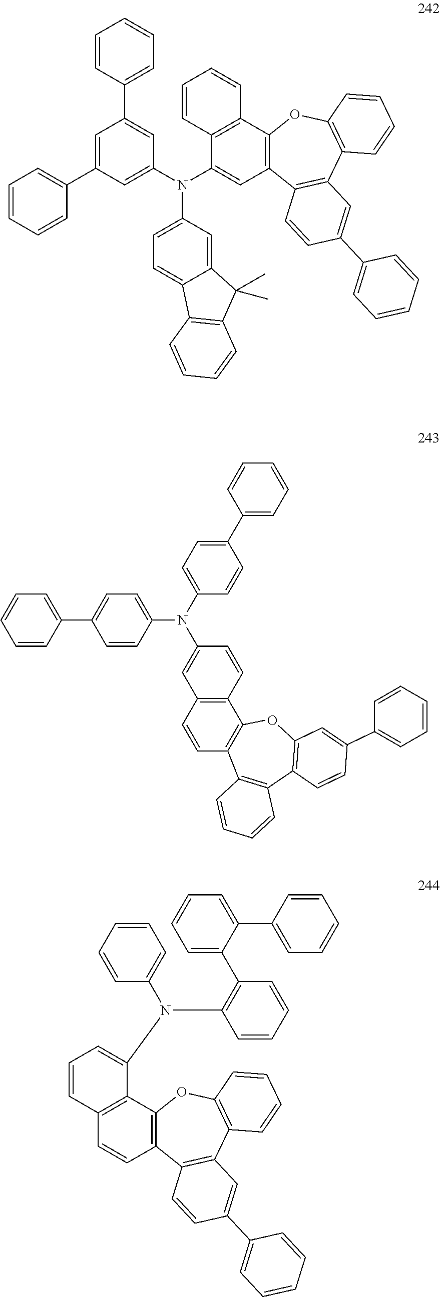 [SMILES: CC1(C)c2ccccc2-c2ccc(N(c3cc(-c4ccccc4)cc(-c4ccccc4)c3)c3cc4c(c5ccccc35)Oc3ccccc3-c3cc(-c5ccccc5)ccc3-4)cc21 ; c1ccc(-c2ccc(N(c3ccc(-c4ccccc4)cc3)c3ccc4c5c(ccc4c3)-c3ccccc3-c3ccc(-c4ccccc4)cc3O5)cc2)cc1 ; c1ccc(-c2ccc3c(c2)-c2ccccc2Oc2c-3ccc3cccc(N(c4ccccc4)c4ccccc4-c4ccccc4-c4ccccc4)c23)cc1]

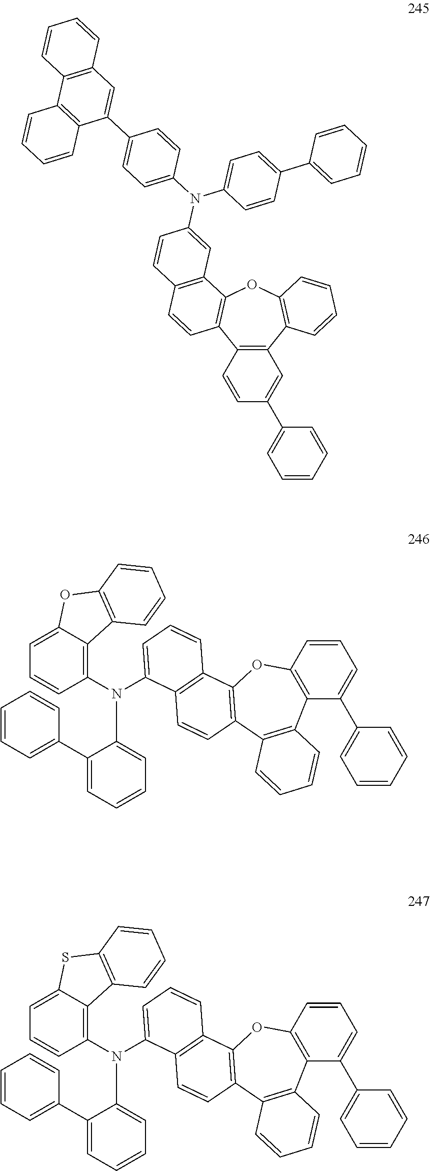 [SMILES: c1ccc(-c2ccc(N(c3ccc(-c4cc5ccccc5c5ccccc45)cc3)c3ccc4ccc5c(c4c3)Oc3ccccc3-c3cc(-c4ccccc4)ccc3-5)cc2)cc1 ; c1ccc(-c2ccccc2N(c2cccc3c4c(ccc23)-c2ccccc2-c2c(cccc2-c2ccccc2)O4)c2cccc3oc4ccccc4c23)cc1 ; c1ccc(-c2ccccc2N(c2cccc3c4c(ccc23)-c2ccccc2-c2c(cccc2-c2ccccc2)O4)c2cccc3sc4ccccc4c23)cc1]

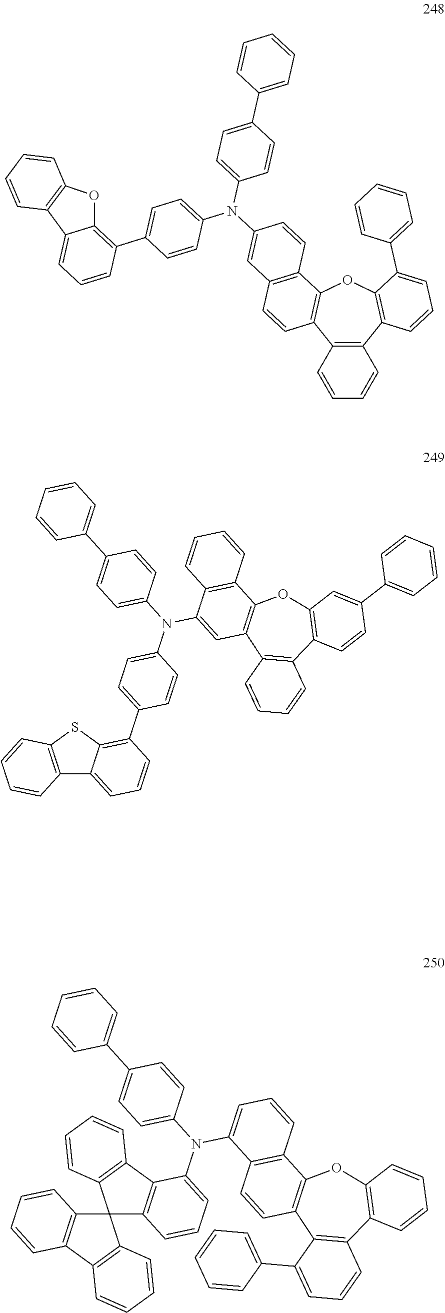 [SMILES: c1ccc(-c2ccc(N(c3ccc(-c4cccc5c4oc4ccccc45)cc3)c3ccc4c5c(ccc4c3)-c3ccccc3-c3cccc(-c4ccccc4)c3O5)cc2)cc1 ; c1ccc(-c2ccc(N(c3ccc(-c4cccc5c4sc4ccccc45)cc3)c3cc4c(c5ccccc35)Oc3cc(-c5ccccc5)ccc3-c3ccccc3-4)cc2)cc1 ; c1ccc(-c2ccc(N(c3cccc4c3-c3ccccc3C43c4ccccc4-c4ccccc43)c3cccc4c5c(ccc34)-c3c(-c4ccccc4)cccc3-c3ccccc3O5)cc2)cc1]

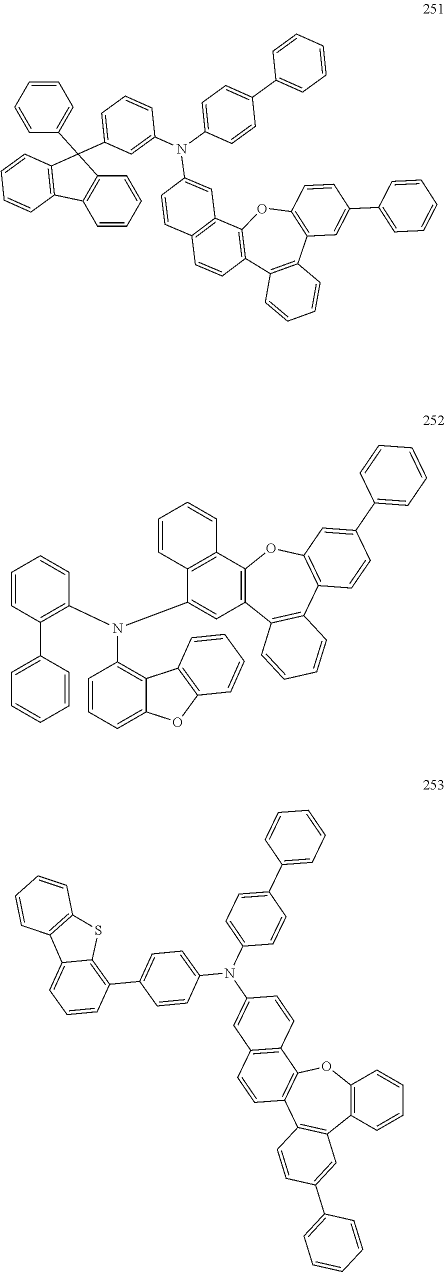 [SMILES: c1ccc(-c2ccc(N(c3cccc(C4(c5ccccc5)c5ccccc5-c5ccccc54)c3)c3ccc4ccc5c(c4c3)Oc3ccc(-c4ccccc4)cc3-c3ccccc3-5)cc2)cc1 ; c1ccc(-c2ccc3c(c2)Oc2c(cc(N(c4ccccc4-c4ccccc4)c4cccc5oc6ccccc6c45)c4ccccc24)-c2ccccc2-3)cc1 ; c1ccc(-c2ccc(N(c3ccc(-c4cccc5c4sc4ccccc45)cc3)c3ccc4c5c(ccc4c3)-c3ccc(-c4ccccc4)cc3-c3ccccc3O5)cc2)cc1]

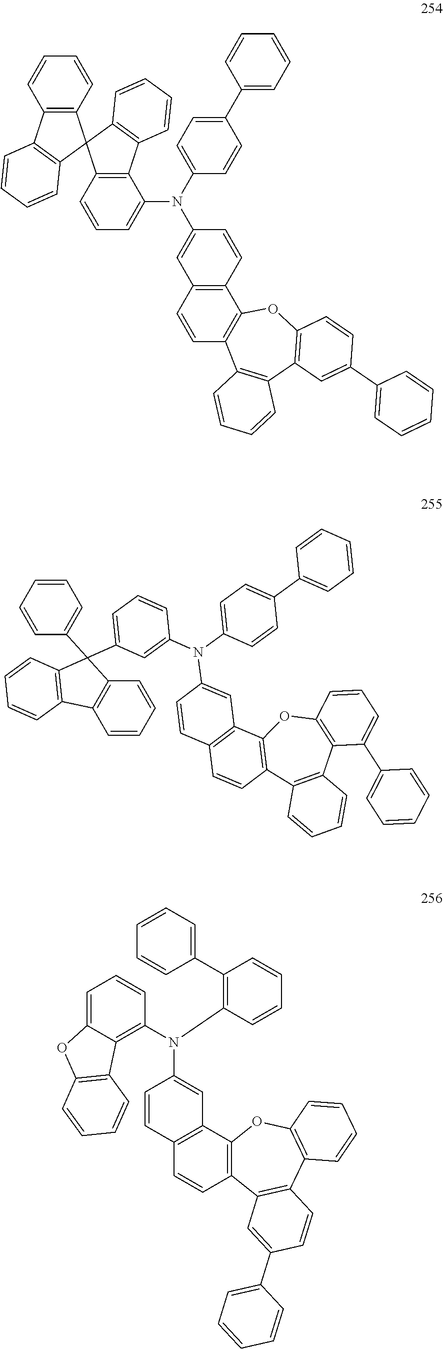 [SMILES: c1ccc(-c2ccc(N(c3ccc4c5c(ccc4c3)-c3ccccc3-c3cc(-c4ccccc4)ccc3O5)c3cccc4c3-c3ccccc3C43c4ccccc4-c4ccccc43)cc2)cc1 ; c1ccc(-c2ccc(N(c3cccc(C4(c5ccccc5)c5ccccc5-c5ccccc54)c3)c3ccc4ccc5c(c4c3)Oc3cccc(-c4ccccc4)c3-c3ccccc3-5)cc2)cc1 ; c1ccc(-c2ccc3c(c2)-c2ccc4ccc(N(c5ccccc5-c5ccccc5)c5cccc6oc7ccccc7c56)cc4c2Oc2ccccc2-3)cc1]

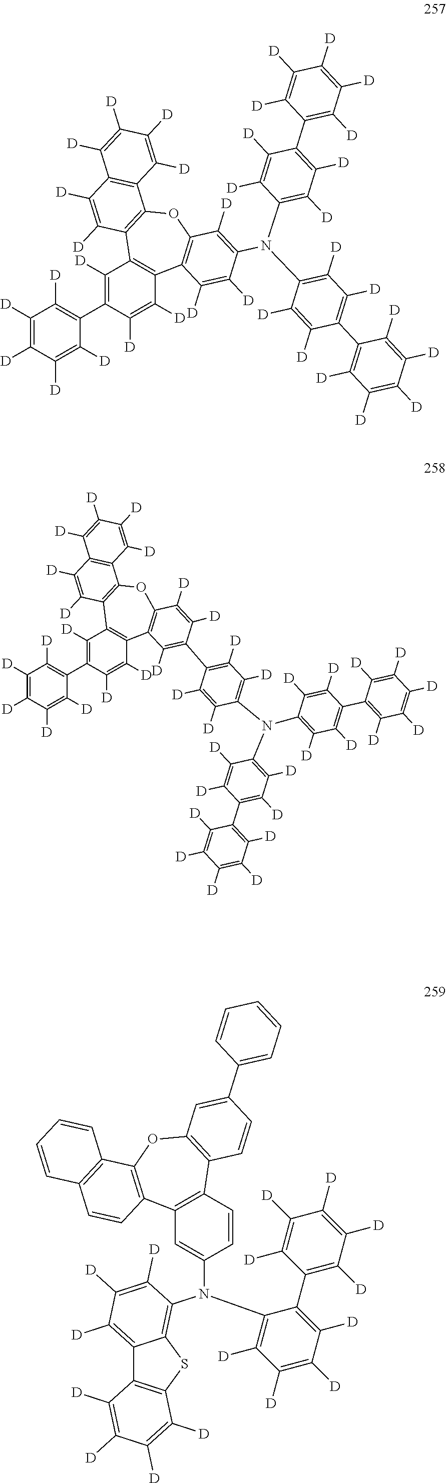

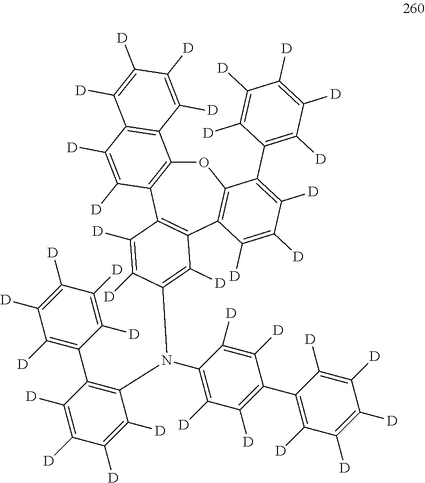 [SMILES: [2H]c1c([2H])c([2H])c(-c2c([2H])c([2H])c(N(c3c([2H])c([2H])c4c(c3[2H])-c3c([2H])c([2H])c([2H])c(-c5c([2H])c([2H])c([2H])c([2H])c5[2H])c3Oc3c-4c([2H])c([2H])c4c([2H])c([2H])c([2H])c([2H])c34)c3c([2H])c([2H])c([2H])c([2H])c3-c3c([2H])c([2H])c([2H])c([2H])c3[2H])c([2H])c2[2H])c([2H])c1[2H]]

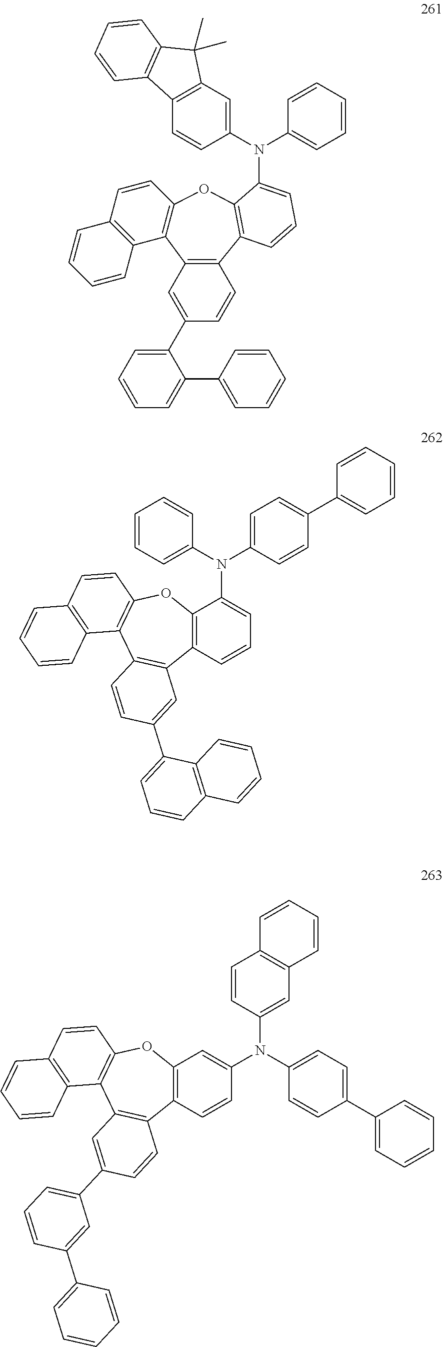

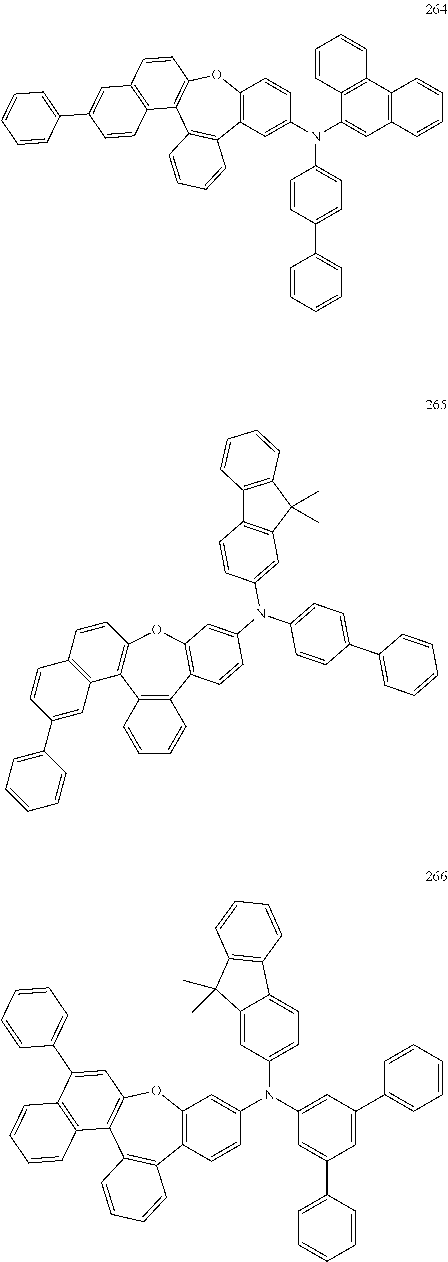

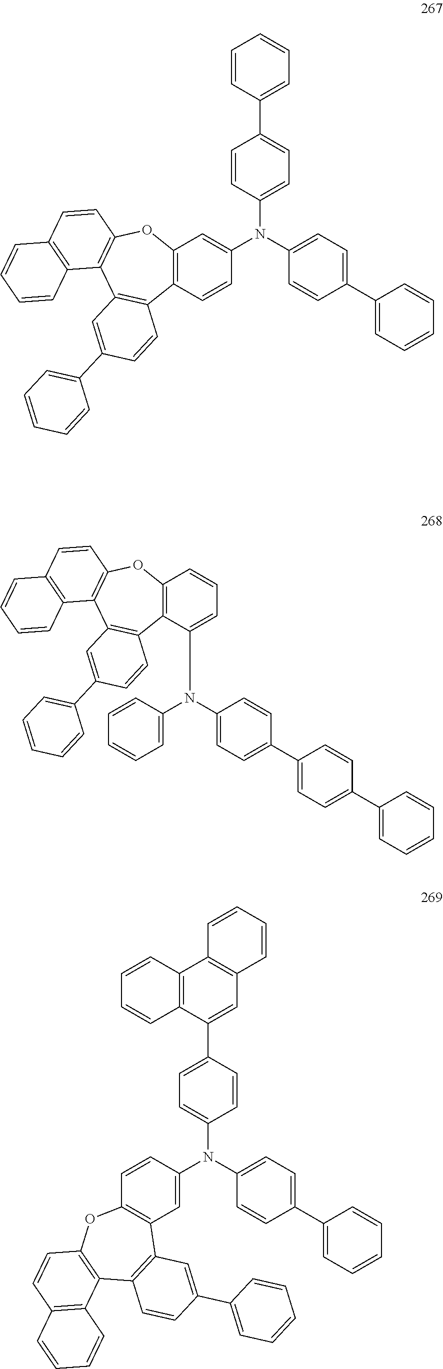 [SMILES: c1ccc(-c2ccc(N(c3ccc(-c4ccccc4)cc3)c3ccc4c(c3)Oc3ccc5ccccc5c3-c3cc(-c5ccccc5)ccc3-4)cc2)cc1 ; c1ccc(-c2ccc(-c3ccc(N(c4ccccc4)c4cccc5c4-c4ccc(-c6ccccc6)cc4-c4c(ccc6ccccc46)O5)cc3)cc2)cc1 ; c1ccc(-c2ccc(N(c3ccc(-c4cc5ccccc5c5ccccc45)cc3)c3ccc4c(c3)-c3cc(-c5ccccc5)ccc3-c3c(ccc5ccccc35)O4)cc2)cc1]

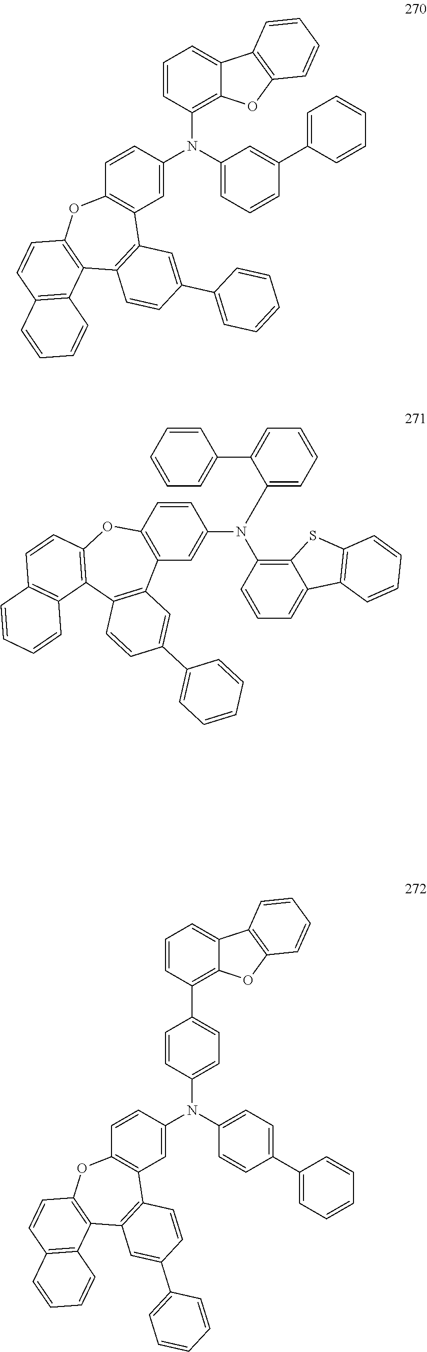 [SMILES: c1ccc(-c2cccc(N(c3ccc4c(c3)-c3cc(-c5ccccc5)ccc3-c3c(ccc5ccccc35)O4)c3cccc4c3oc3ccccc34)c2)cc1 ; c1ccc(-c2ccc3c(c2)-c2cc(N(c4ccccc4-c4ccccc4)c4cccc5c4sc4ccccc45)ccc2Oc2ccc4ccccc4c2-3)cc1 ; c1ccc(-c2ccc(N(c3ccc(-c4cccc5c4oc4ccccc45)cc3)c3ccc4c(c3)-c3ccc(-c5ccccc5)cc3-c3c(ccc5ccccc35)O4)cc2)cc1]

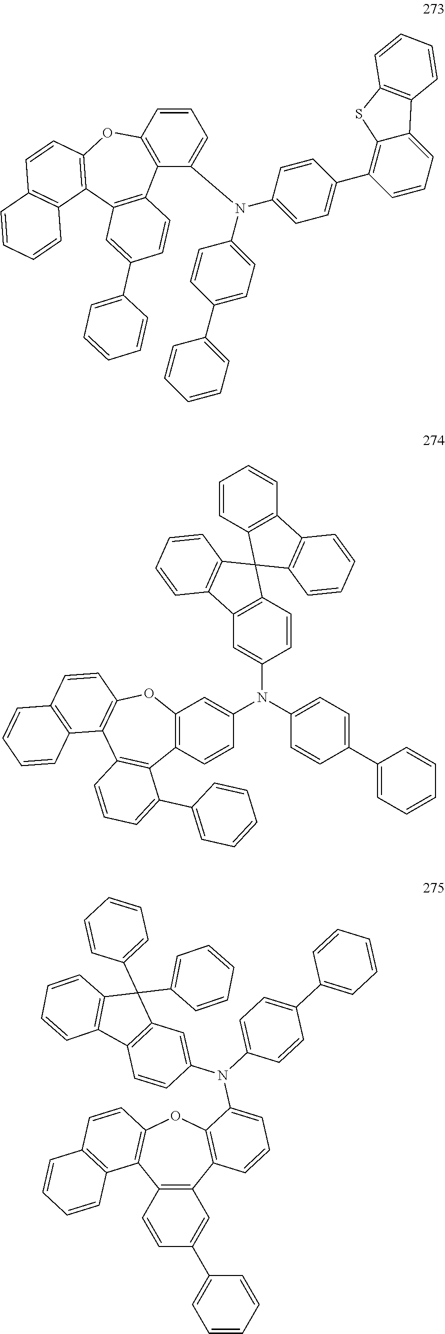 [SMILES: c1ccc(-c2ccc(N(c3ccc(-c4cccc5c4sc4ccccc45)cc3)c3cccc4c3-c3ccc(-c5ccccc5)cc3-c3c(ccc5ccccc35)O4)cc2)cc1 ; c1ccc(-c2ccc(N(c3ccc4c(c3)Oc3ccc5ccccc5c3-c3cccc(-c5ccccc5)c3-4)c3ccc4c(c3)-c3ccccc3C43c4ccccc4-c4ccccc43)cc2)cc1 ; c1ccc(-c2ccc(N(c3ccc4c(c3)C(c3ccccc3)(c3ccccc3)c3ccccc3-4)c3cccc4c3Oc3ccc5ccccc5c3-c3ccc(-c5ccccc5)cc3-4)cc2)cc1]

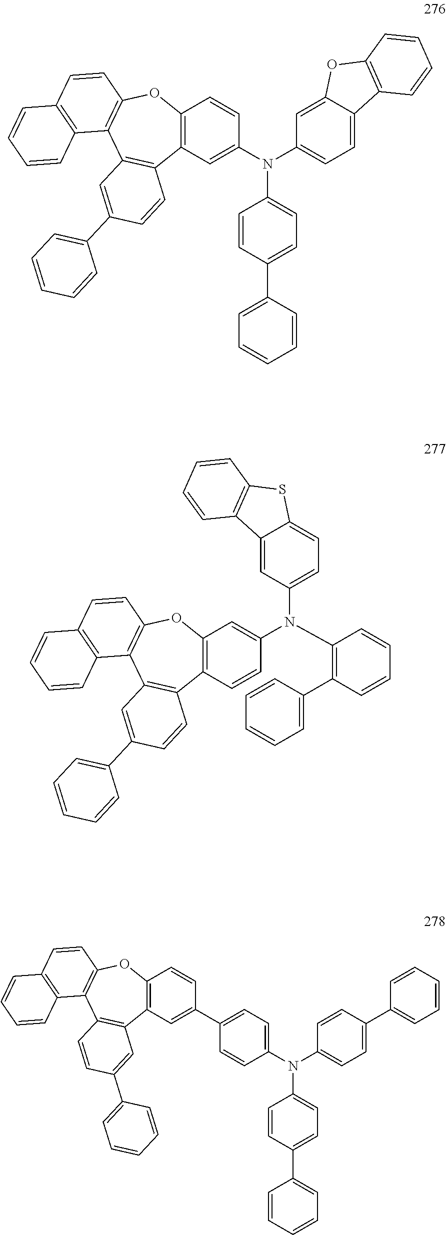

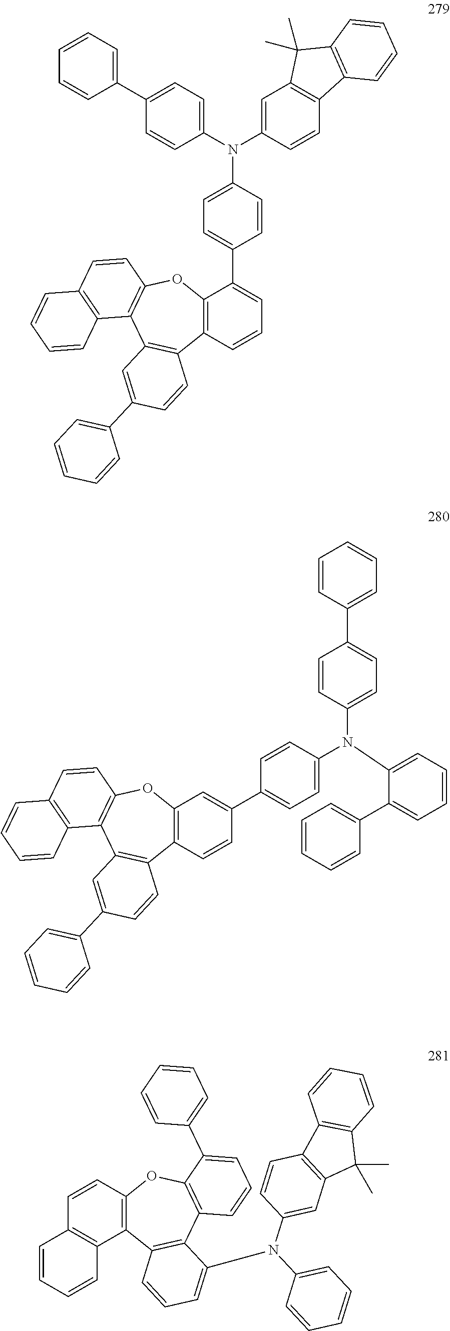 [SMILES: CC1(C)c2ccccc2-c2ccc(N(c3ccc(-c4ccccc4)cc3)c3ccc(-c4cccc5c4Oc4ccc6ccccc6c4-c4cc(-c6ccccc6)ccc4-5)cc3)cc21 ; c1ccc(-c2ccc(N(c3ccc(-c4ccc5c(c4)Oc4ccc6ccccc6c4-c4cc(-c6ccccc6)ccc4-5)cc3)c3ccccc3-c3ccccc3)cc2)cc1 ; CC1(C)c2ccccc2-c2ccc(N(c3ccccc3)c3cccc4c3-c3cccc(-c5ccccc5)c3Oc3ccc5ccccc5c3-4)cc21]

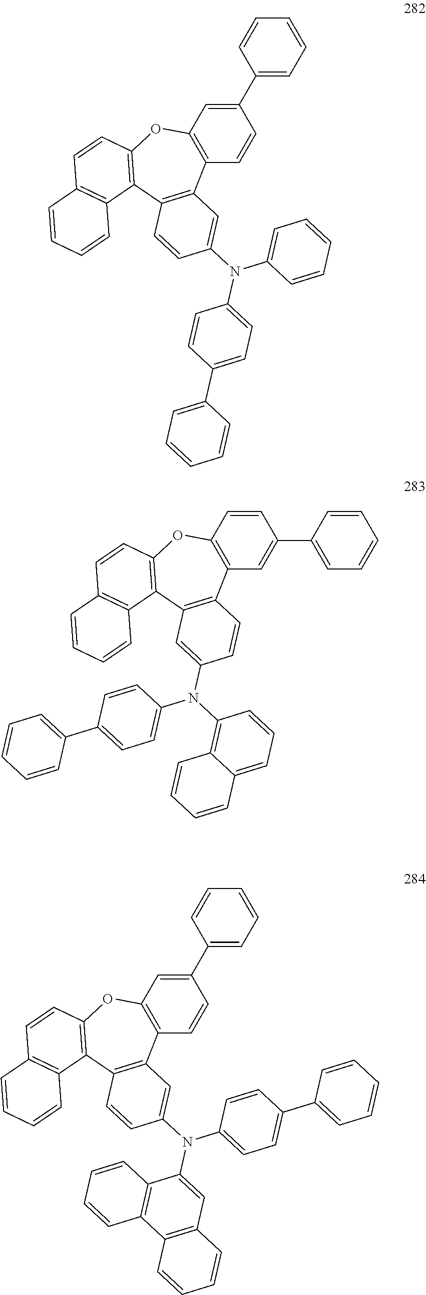 [SMILES: c1ccc(-c2ccc(N(c3ccccc3)c3ccc4c(c3)-c3ccc(-c5ccccc5)cc3Oc3ccc5ccccc5c3-4)cc2)cc1 ; c1ccc(-c2ccc(N(c3ccc4c(c3)-c3c(ccc5ccccc35)Oc3ccc(-c5ccccc5)cc3-4)c3cccc4ccccc34)cc2)cc1 ; c1ccc(-c2ccc(N(c3ccc4c(c3)-c3ccc(-c5ccccc5)cc3Oc3ccc5ccccc5c3-4)c3cc4ccccc4c4ccccc34)cc2)cc1]

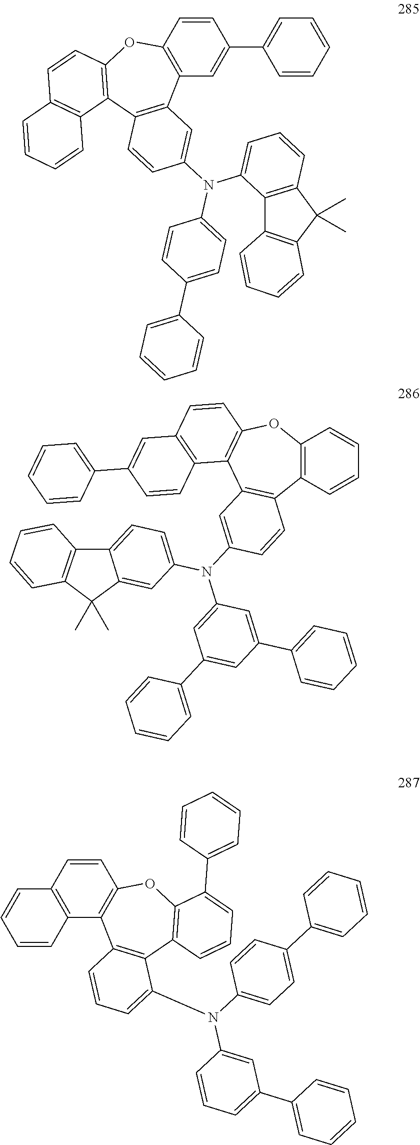

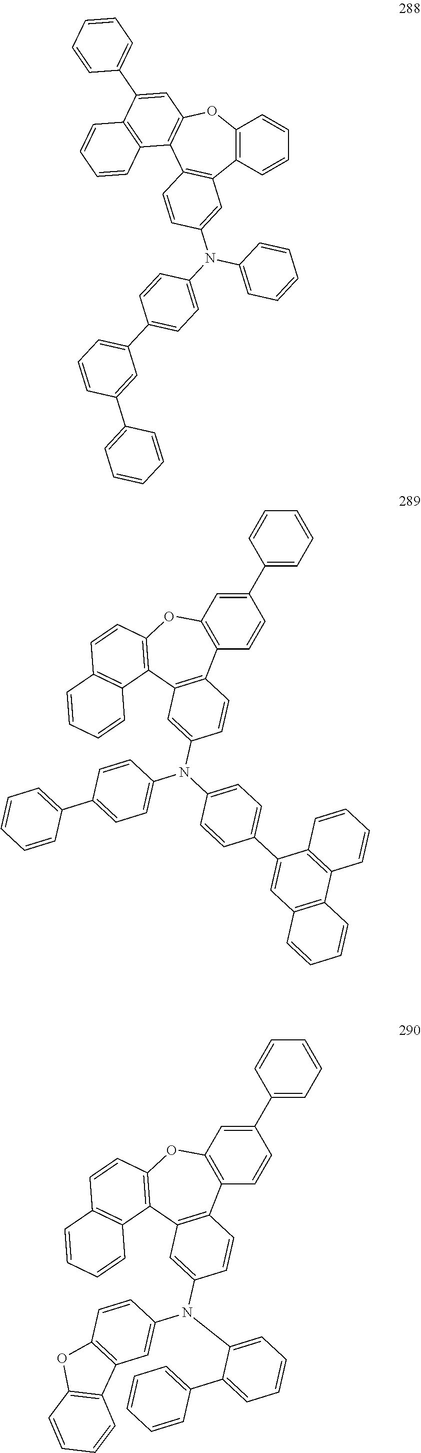 [SMILES: c1ccc(-c2cccc(-c3ccc(N(c4ccccc4)c4ccc5c(c4)-c4ccccc4Oc4cc(-c6ccccc6)c6ccccc6c4-5)cc3)c2)cc1 ; c1ccc(-c2ccc(N(c3ccc(-c4cc5ccccc5c5ccccc45)cc3)c3ccc4c(c3)-c3c(ccc5ccccc35)Oc3cc(-c5ccccc5)ccc3-4)cc2)cc1 ; c1ccc(-c2ccc3c(c2)Oc2ccc4ccccc4c2-c2cc(N(c4ccc5oc6ccccc6c5c4)c4ccccc4-c4ccccc4)ccc2-3)cc1]

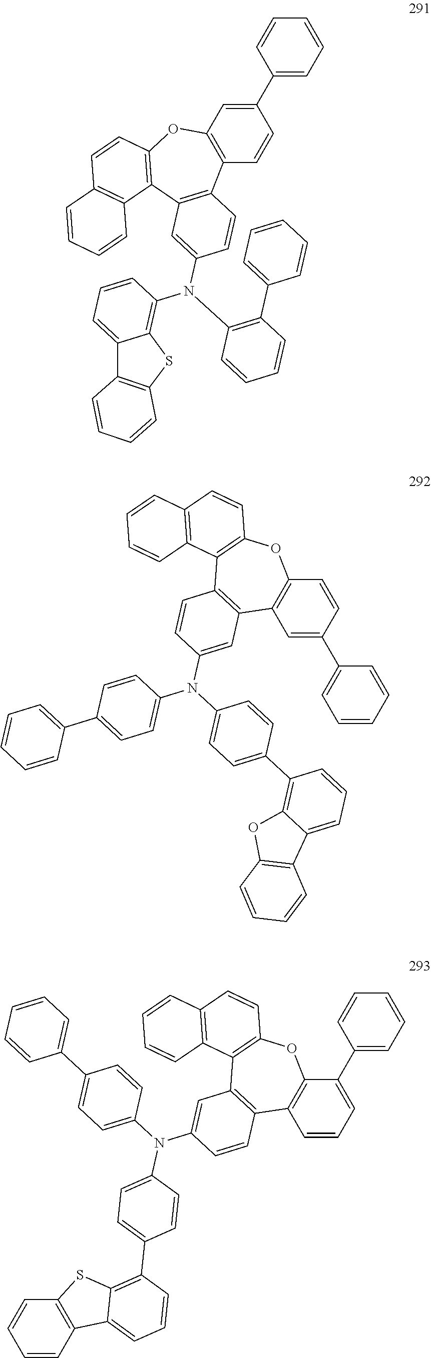 [SMILES: c1ccc(-c2ccc3c(c2)Oc2ccc4ccccc4c2-c2cc(N(c4ccccc4-c4ccccc4)c4cccc5c4sc4ccccc45)ccc2-3)cc1 ; c1ccc(-c2ccc(N(c3ccc(-c4cccc5c4oc4ccccc45)cc3)c3ccc4c(c3)-c3cc(-c5ccccc5)ccc3Oc3ccc5ccccc5c3-4)cc2)cc1 ; c1ccc(-c2ccc(N(c3ccc(-c4cccc5c4sc4ccccc45)cc3)c3ccc4c(c3)-c3c(ccc5ccccc35)Oc3c(-c5ccccc5)cccc3-4)cc2)cc1]

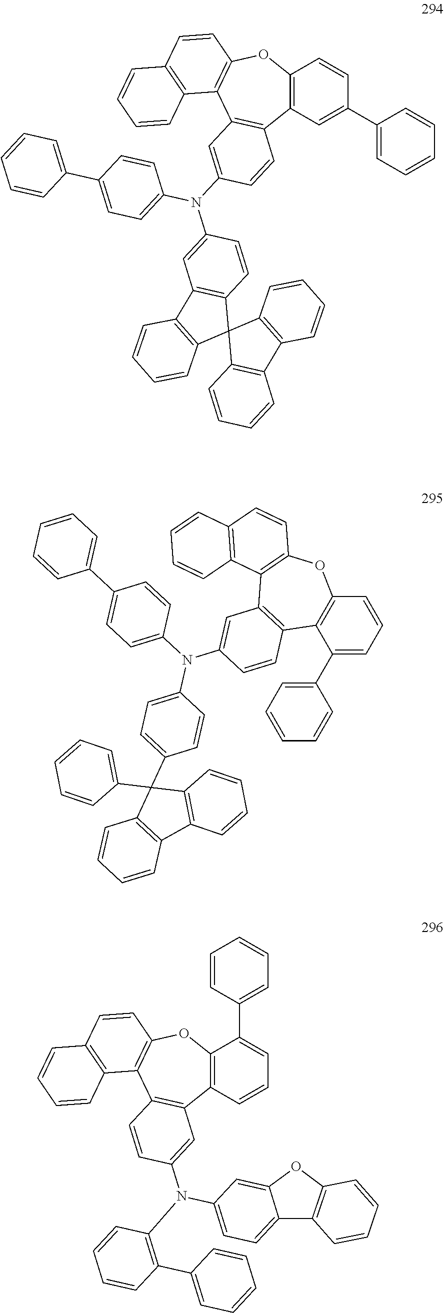 [SMILES: c1ccc(-c2ccc(N(c3ccc4c(c3)-c3c(ccc5ccccc35)Oc3ccc(-c5ccccc5)cc3-4)c3ccc4c(c3)-c3ccccc3C43c4ccccc4-c4ccccc43)cc2)cc1 ; c1ccc(-c2ccc(N(c3ccc(C4(c5ccccc5)c5ccccc5-c5ccccc54)cc3)c3ccc4c(c3)-c3c(ccc5ccccc35)Oc3cccc(-c5ccccc5)c3-4)cc2)cc1 ; c1ccc(-c2ccccc2N(c2ccc3c(c2)-c2cccc(-c4ccccc4)c2Oc2ccc4ccccc4c2-3)c2ccc3c(c2)oc2ccccc23)cc1]

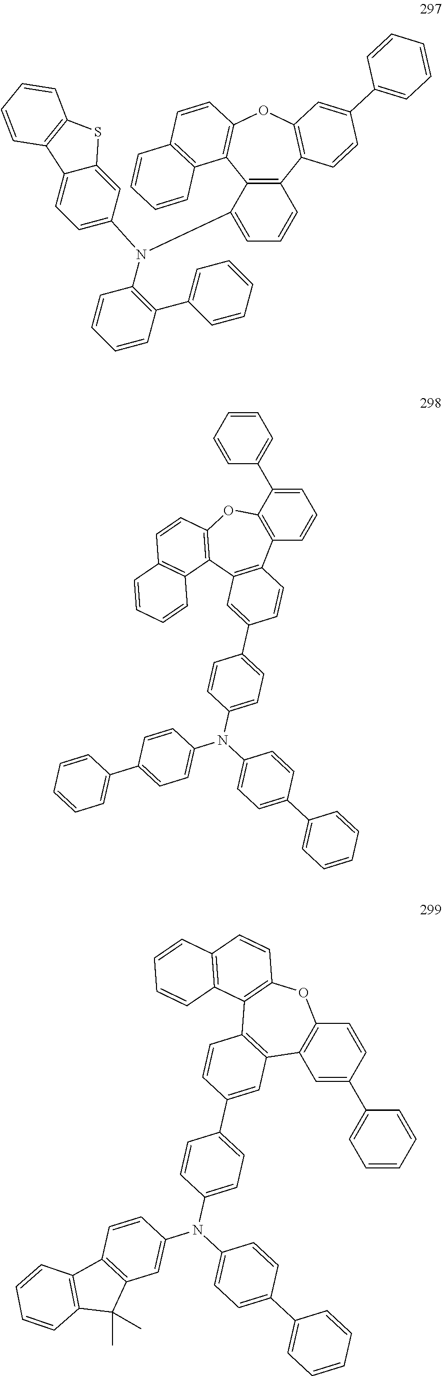 [SMILES: c1ccc(-c2ccc3c(c2)Oc2ccc4ccccc4c2-c2c-3cccc2N(c2ccc3c(c2)sc2ccccc23)c2ccccc2-c2ccccc2)cc1 ; c1ccc(-c2ccc(N(c3ccc(-c4ccccc4)cc3)c3ccc(-c4ccc5c(c4)-c4c(ccc6ccccc46)Oc4c(-c6ccccc6)cccc4-5)cc3)cc2)cc1 ; CC1(C)c2ccccc2-c2ccc(N(c3ccc(-c4ccccc4)cc3)c3ccc(-c4ccc5c(c4)-c4cc(-c6ccccc6)ccc4Oc4ccc6ccccc6c4-5)cc3)cc21]

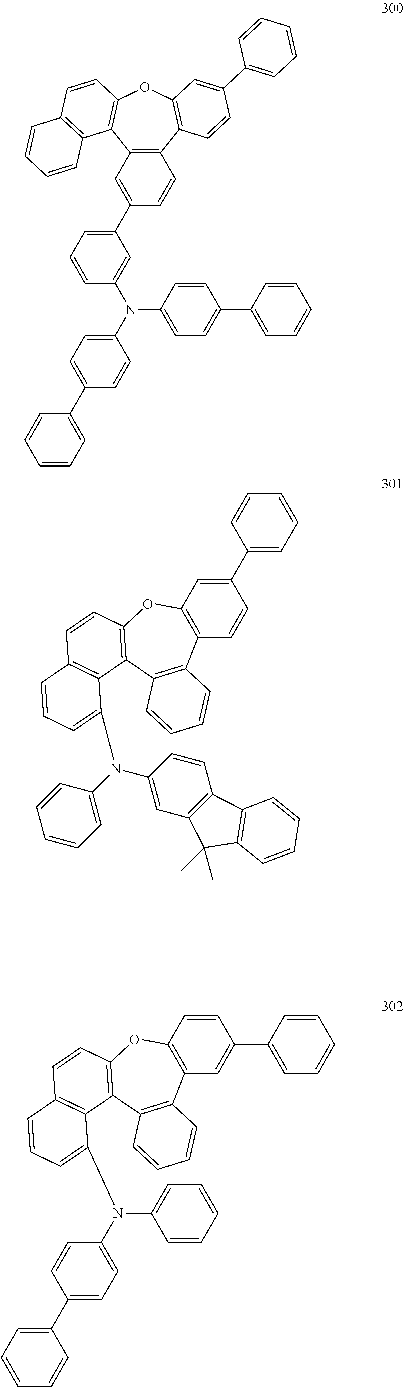 [SMILES: c1ccc(-c2ccc(N(c3ccc(-c4ccccc4)cc3)c3cccc(-c4ccc5c(c4)-c4c(ccc6ccccc46)Oc4cc(-c6ccccc6)ccc4-5)c3)cc2)cc1 ; CC1(C)c2ccccc2-c2ccc(N(c3ccccc3)c3cccc4ccc5c(c34)-c3ccccc3-c3ccc(-c4ccccc4)cc3O5)cc21 ; c1ccc(-c2ccc(N(c3ccccc3)c3cccc4ccc5c(c34)-c3ccccc3-c3cc(-c4ccccc4)ccc3O5)cc2)cc1]

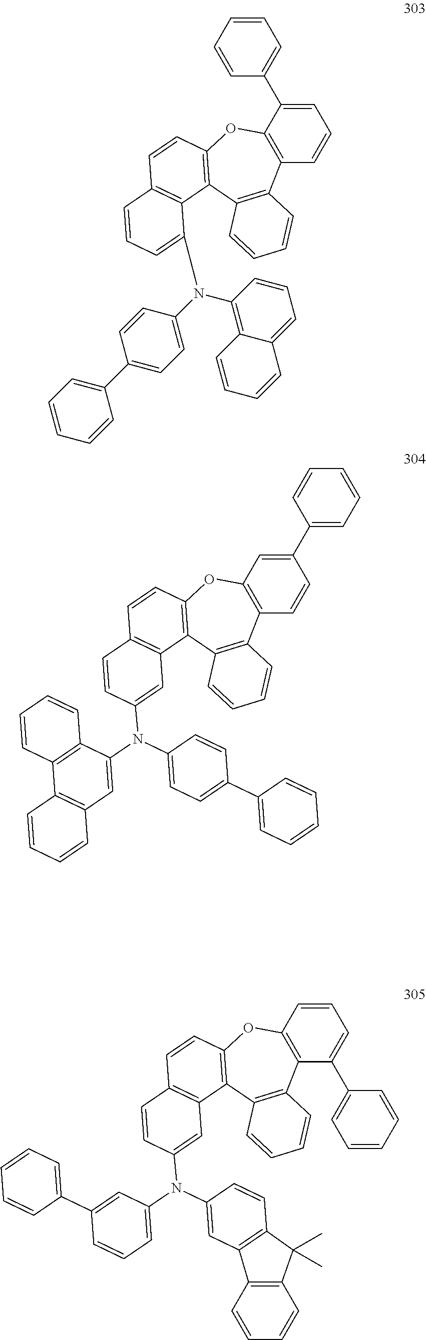 [SMILES: c1ccc(-c2ccc(N(c3cccc4ccccc34)c3cccc4ccc5c(c34)-c3ccccc3-c3cccc(-c4ccccc4)c3O5)cc2)cc1 ; c1ccc(-c2ccc(N(c3ccc4ccc5c(c4c3)-c3ccccc3-c3ccc(-c4ccccc4)cc3O5)c3cc4ccccc4c4ccccc34)cc2)cc1 ; CC1(C)c2ccccc2-c2cc(N(c3cccc(-c4ccccc4)c3)c3ccc4ccc5c(c4c3)-c3ccccc3-c3c(cccc3-c3ccccc3)O5)ccc21]

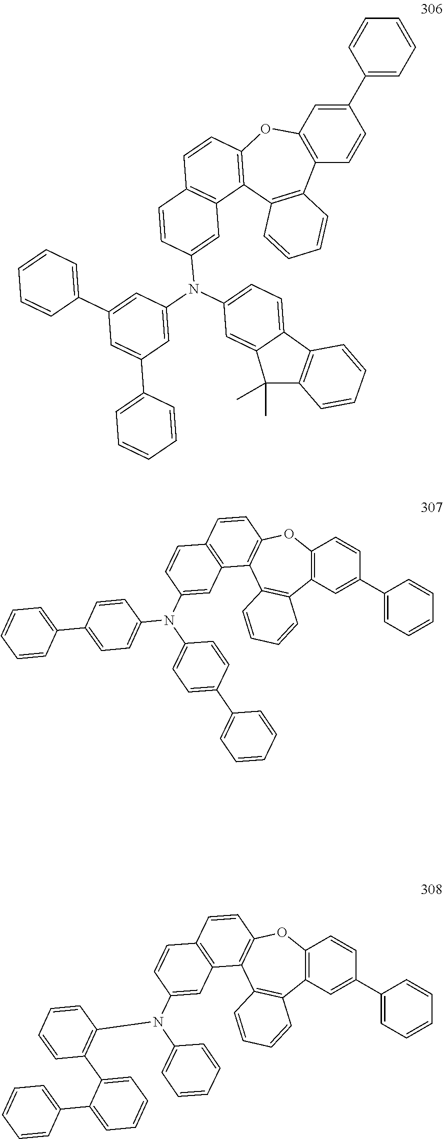 [SMILES: CC1(C)c2ccccc2-c2ccc(N(c3cc(-c4ccccc4)cc(-c4ccccc4)c3)c3ccc4ccc5c(c4c3)-c3ccccc3-c3ccc(-c4ccccc4)cc3O5)cc21 ; c1ccc(-c2ccc(N(c3ccc(-c4ccccc4)cc3)c3ccc4ccc5c(c4c3)-c3ccccc3-c3cc(-c4ccccc4)ccc3O5)cc2)cc1 ; c1ccc(-c2ccc3c(c2)-c2ccccc2-c2c(ccc4ccc(N(c5ccccc5)c5ccccc5-c5ccccc5-c5ccccc5)cc24)O3)cc1]

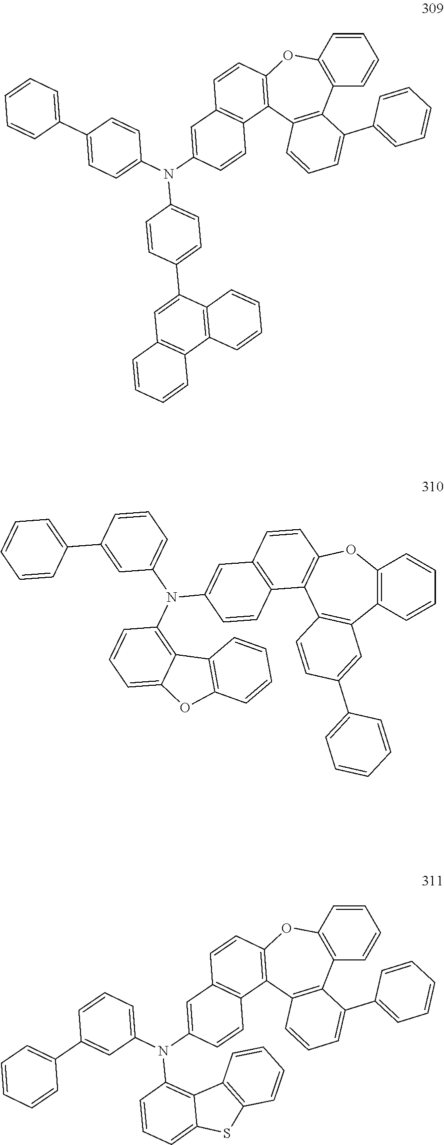 [SMILES: c1ccc(-c2ccc(N(c3ccc(-c4cc5ccccc5c5ccccc45)cc3)c3ccc4c5c(ccc4c3)Oc3ccccc3-c3c(-c4ccccc4)cccc3-5)cc2)cc1 ; c1ccc(-c2cccc(N(c3ccc4c5c(ccc4c3)Oc3ccccc3-c3cc(-c4ccccc4)ccc3-5)c3cccc4oc5ccccc5c34)c2)cc1 ; c1ccc(-c2cccc(N(c3ccc4c5c(ccc4c3)Oc3ccccc3-c3c(-c4ccccc4)cccc3-5)c3cccc4sc5ccccc5c34)c2)cc1]

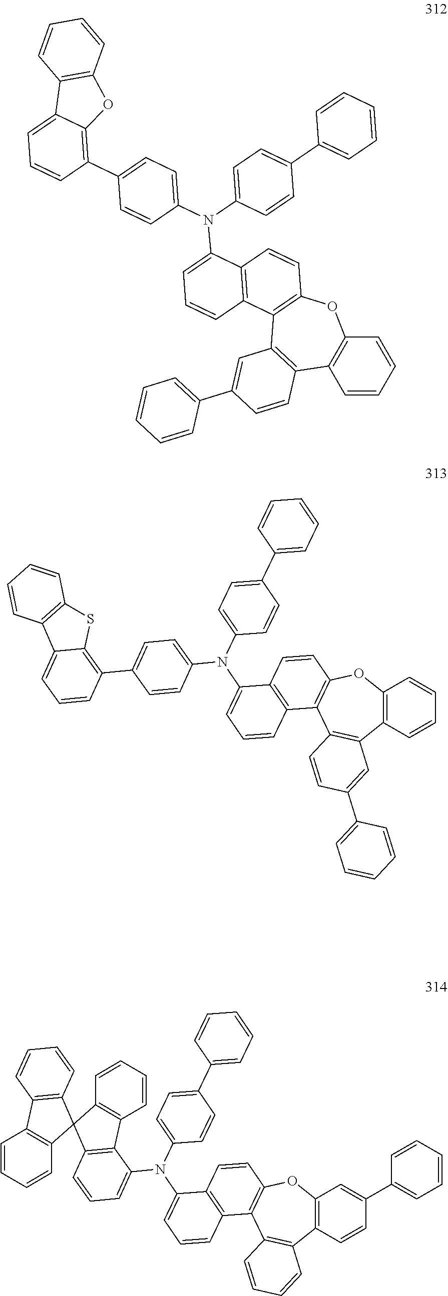 [SMILES: c1ccc(-c2ccc(N(c3ccc(-c4cccc5c4oc4ccccc45)cc3)c3cccc4c5c(ccc34)Oc3ccccc3-c3ccc(-c4ccccc4)cc3-5)cc2)cc1 ; c1ccc(-c2ccc(N(c3ccc(-c4cccc5c4sc4ccccc45)cc3)c3cccc4c5c(ccc34)Oc3ccccc3-c3cc(-c4ccccc4)ccc3-5)cc2)cc1 ; c1ccc(-c2ccc(N(c3cccc4c3-c3ccccc3C43c4ccccc4-c4ccccc43)c3cccc4c5c(ccc34)Oc3cc(-c4ccccc4)ccc3-c3ccccc3-5)cc2)cc1]

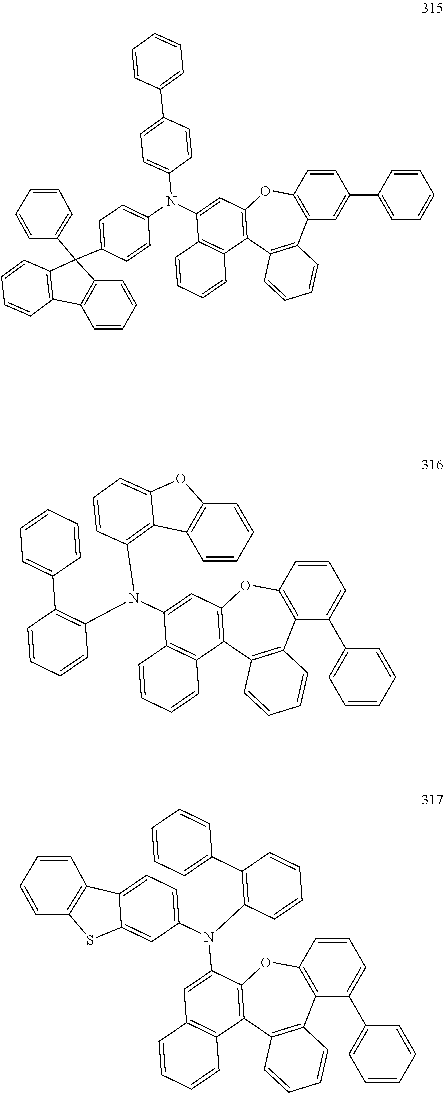 [SMILES: c1ccc(-c2ccc(N(c3ccc(C4(c5ccccc5)c5ccccc5-c5ccccc54)cc3)c3cc4c(c5ccccc35)-c3ccccc3-c3cc(-c5ccccc5)ccc3O4)cc2)cc1 ; c1ccc(-c2ccccc2N(c2cc3c(c4ccccc24)-c2ccccc2-c2c(cccc2-c2ccccc2)O3)c2cccc3oc4ccccc4c23)cc1 ; c1ccc(-c2ccccc2N(c2ccc3c(c2)sc2ccccc23)c2cc3ccccc3c3c2Oc2cccc(-c4ccccc4)c2-c2ccccc2-3)cc1]

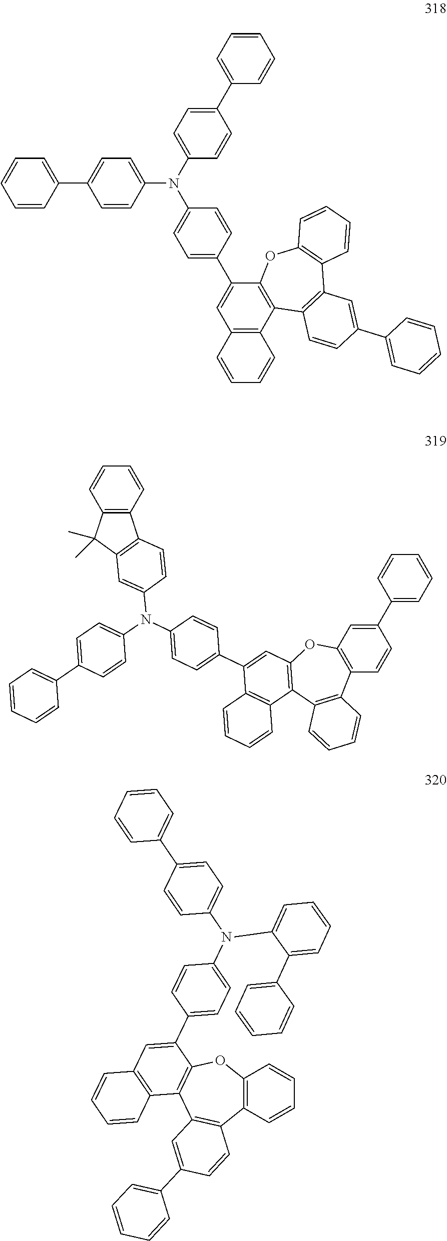 [SMILES: c1ccc(-c2ccc(N(c3ccc(-c4ccccc4)cc3)c3ccc(-c4cc5ccccc5c5c4Oc4ccccc4-c4cc(-c6ccccc6)ccc4-5)cc3)cc2)cc1 ; CC1(C)c2ccccc2-c2ccc(N(c3ccc(-c4ccccc4)cc3)c3ccc(-c4cc5c(c6ccccc46)-c4ccccc4-c4ccc(-c6ccccc6)cc4O5)cc3)cc21 ; c1ccc(-c2ccc(N(c3ccc(-c4cc5ccccc5c5c4Oc4ccccc4-c4ccc(-c6ccccc6)cc4-5)cc3)c3ccccc3-c3ccccc3)cc2)cc1]

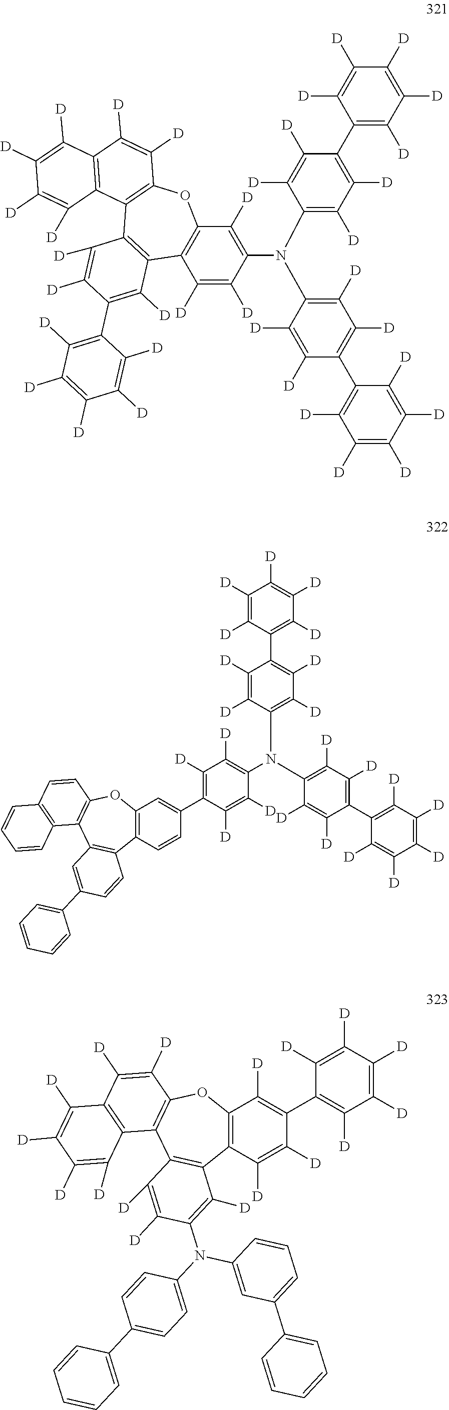

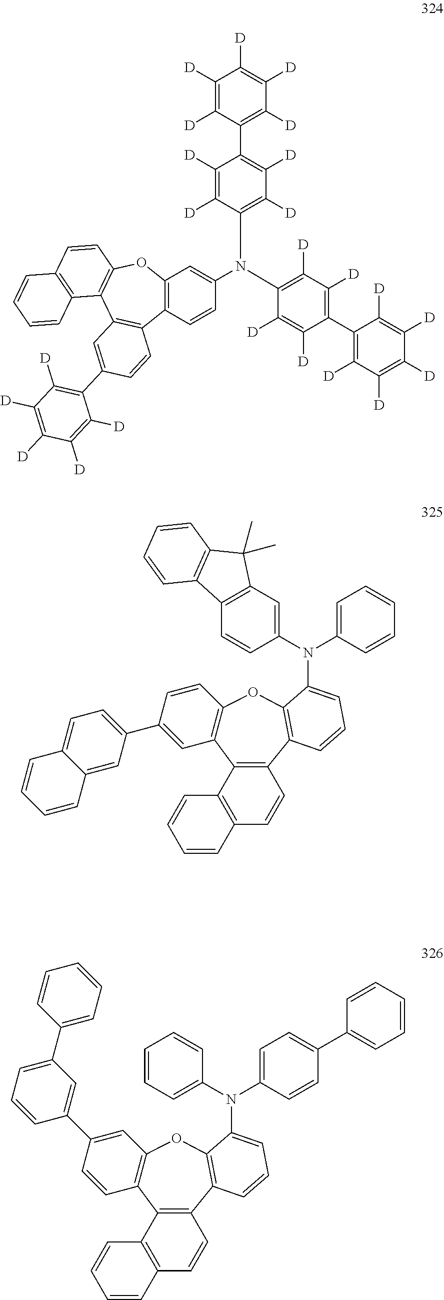 [SMILES: [2H]c1c([2H])c([2H])c(-c2ccc3c(c2)-c2c(ccc4ccccc24)Oc2cc(N(c4c([2H])c([2H])c(-c5c([2H])c([2H])c([2H])c([2H])c5[2H])c([2H])c4[2H])c4c([2H])c([2H])c(-c5c([2H])c([2H])c([2H])c([2H])c5[2H])c([2H])c4[2H])ccc2-3)c([2H])c1[2H] ; CC1(C)c2ccccc2-c2ccc(N(c3ccccc3)c3cccc4c3Oc3ccc(-c5ccc6ccccc6c5)cc3-c3c-4ccc4ccccc34)cc21 ; c1ccc(-c2ccc(N(c3ccccc3)c3cccc4c3Oc3cc(-c5cccc(-c6ccccc6)c5)ccc3-c3c-4ccc4ccccc34)cc2)cc1]

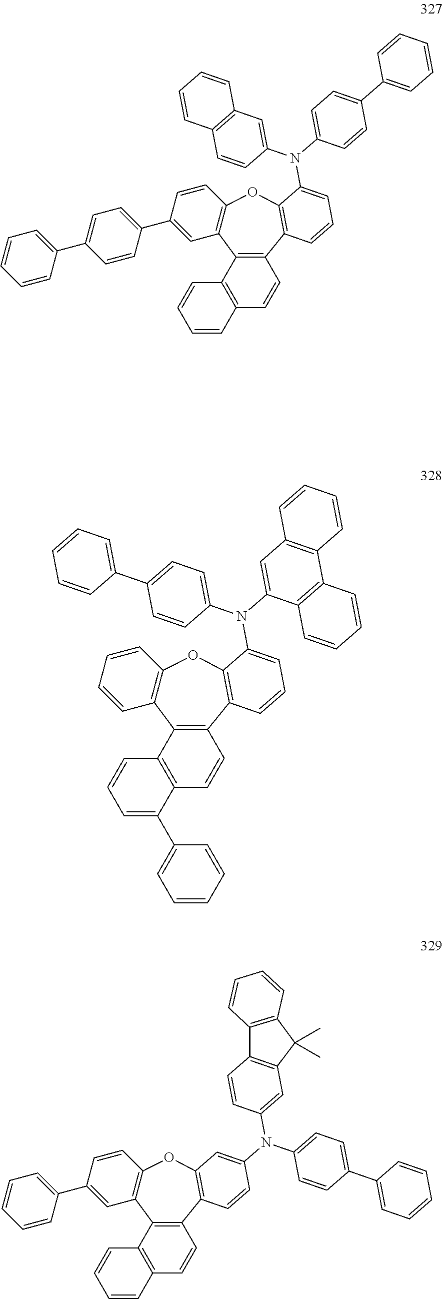 [SMILES: c1ccc(-c2ccc(-c3ccc4c(c3)-c3c(ccc5ccccc35)-c3cccc(N(c5ccc(-c6ccccc6)cc5)c5ccc6ccccc6c5)c3O4)cc2)cc1 ; c1ccc(-c2ccc(N(c3cccc4c3Oc3ccccc3-c3c-4ccc4c(-c5ccccc5)cccc34)c3cc4ccccc4c4ccccc34)cc2)cc1 ; CC1(C)c2ccccc2-c2ccc(N(c3ccc(-c4ccccc4)cc3)c3ccc4c(c3)Oc3ccc(-c5ccccc5)cc3-c3c-4ccc4ccccc34)cc21]

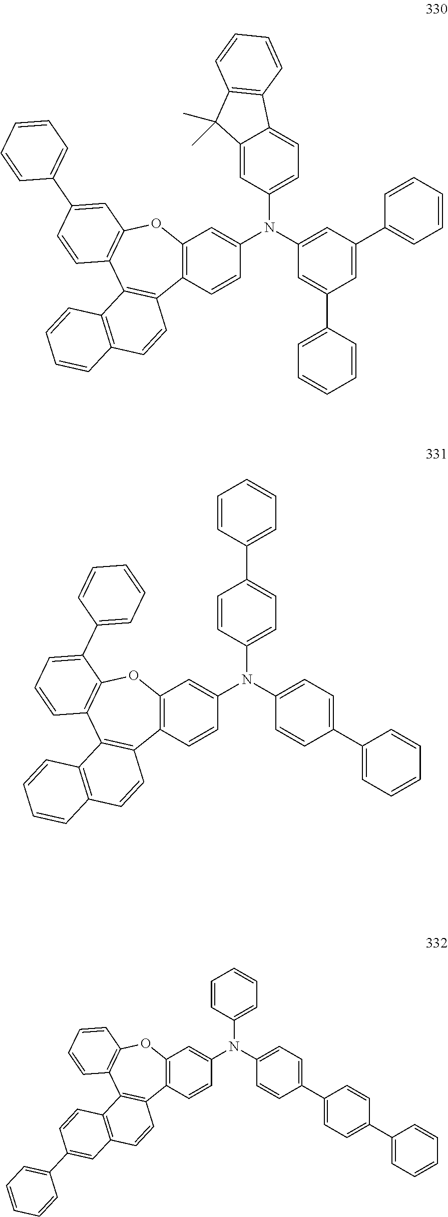 [SMILES: CC1(C)c2ccccc2-c2ccc(N(c3cc(-c4ccccc4)cc(-c4ccccc4)c3)c3ccc4c(c3)Oc3cc(-c5ccccc5)ccc3-c3c-4ccc4ccccc34)cc21 ; c1ccc(-c2ccc(N(c3ccc(-c4ccccc4)cc3)c3ccc4c(c3)Oc3c(-c5ccccc5)cccc3-c3c-4ccc4ccccc34)cc2)cc1 ; c1ccc(-c2ccc(-c3ccc(N(c4ccccc4)c4ccc5c(c4)Oc4ccccc4-c4c-5ccc5cc(-c6ccccc6)ccc45)cc3)cc2)cc1]

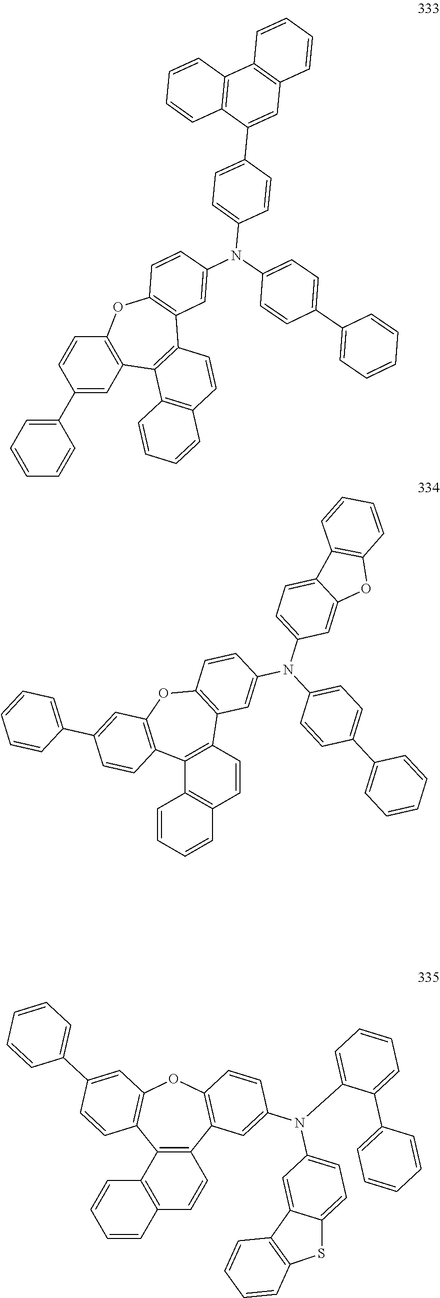 [SMILES: c1ccc(-c2ccc(N(c3ccc(-c4cc5ccccc5c5ccccc45)cc3)c3ccc4c(c3)-c3ccc5ccccc5c3-c3cc(-c5ccccc5)ccc3O4)cc2)cc1 ; c1ccc(-c2ccc(N(c3ccc4c(c3)-c3ccc5ccccc5c3-c3ccc(-c5ccccc5)cc3O4)c3ccc4c(c3)oc3ccccc34)cc2)cc1 ; c1ccc(-c2ccc3c(c2)Oc2ccc(N(c4ccc5sc6ccccc6c5c4)c4ccccc4-c4ccccc4)cc2-c2ccc4ccccc4c2-3)cc1]

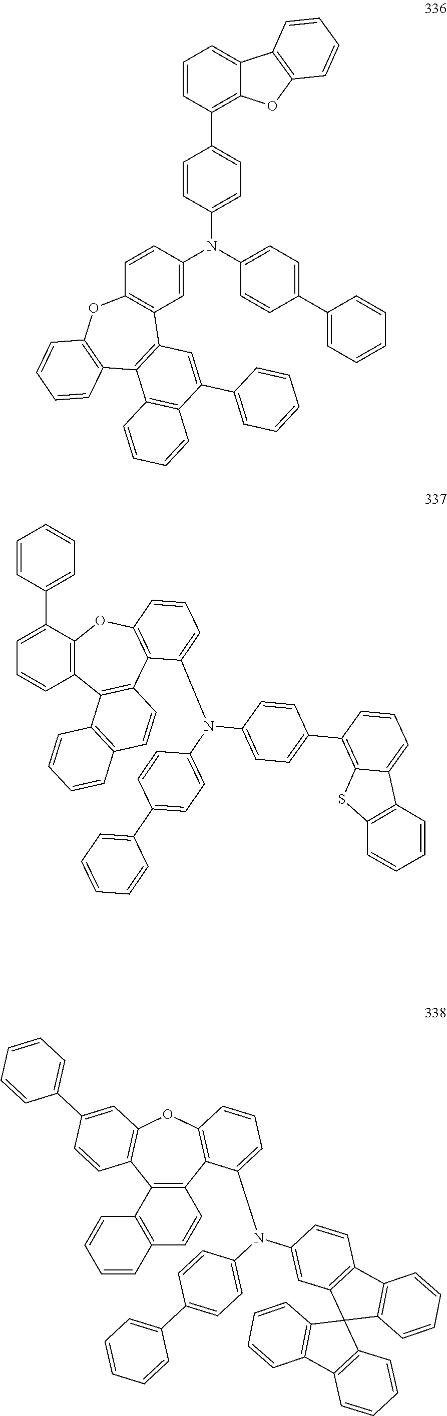 [SMILES: c1ccc(-c2ccc(N(c3ccc(-c4cccc5c4oc4ccccc45)cc3)c3ccc4c(c3)-c3cc(-c5ccccc5)c5ccccc5c3-c3ccccc3O4)cc2)cc1 ; c1ccc(-c2ccc(N(c3ccc(-c4cccc5c4sc4ccccc45)cc3)c3cccc4c3-c3ccc5ccccc5c3-c3cccc(-c5ccccc5)c3O4)cc2)cc1 ; c1ccc(-c2ccc(N(c3ccc4c(c3)C3(c5ccccc5-c5ccccc53)c3ccccc3-4)c3cccc4c3-c3ccc5ccccc5c3-c3ccc(-c5ccccc5)cc3O4)cc2)cc1]

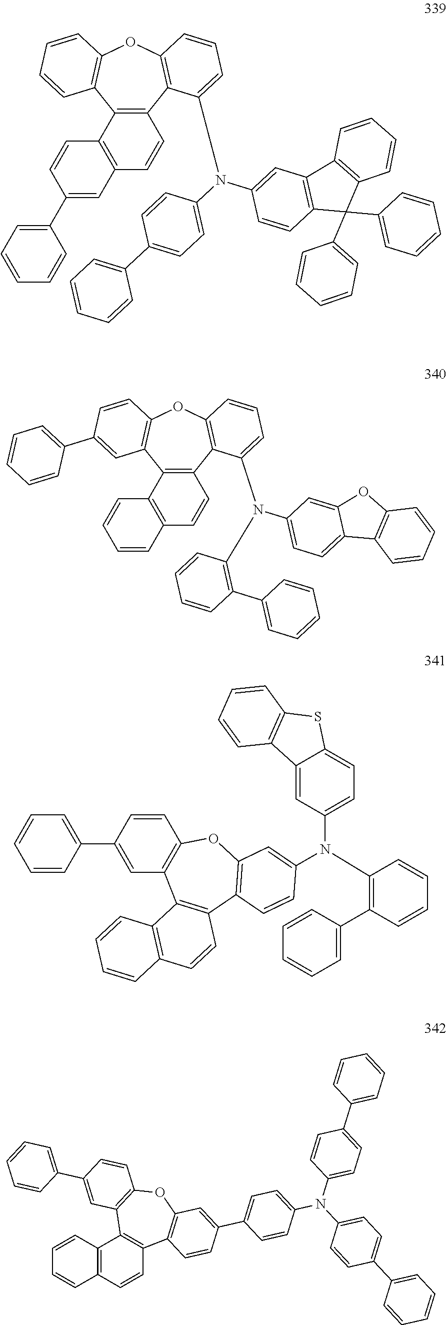 [SMILES: c1ccc(-c2ccc(N(c3ccc4c(c3)-c3ccccc3C4(c3ccccc3)c3ccccc3)c3cccc4c3-c3ccc5cc(-c6ccccc6)ccc5c3-c3ccccc3O4)cc2)cc1 ; c1ccc(-c2ccc3c(c2)-c2c(ccc4ccccc24)-c2c(cccc2N(c2ccc4c(c2)oc2ccccc24)c2ccccc2-c2ccccc2)O3)cc1 ; c1ccc(-c2ccc3c(c2)-c2c(ccc4ccccc24)-c2ccc(N(c4ccc5sc6ccccc6c5c4)c4ccccc4-c4ccccc4)cc2O3)cc1 ; c1ccc(-c2ccc(N(c3ccc(-c4ccccc4)cc3)c3ccc(-c4ccc5c(c4)Oc4ccc(-c6ccccc6)cc4-c4c-5ccc5ccccc45)cc3)cc2)cc1]

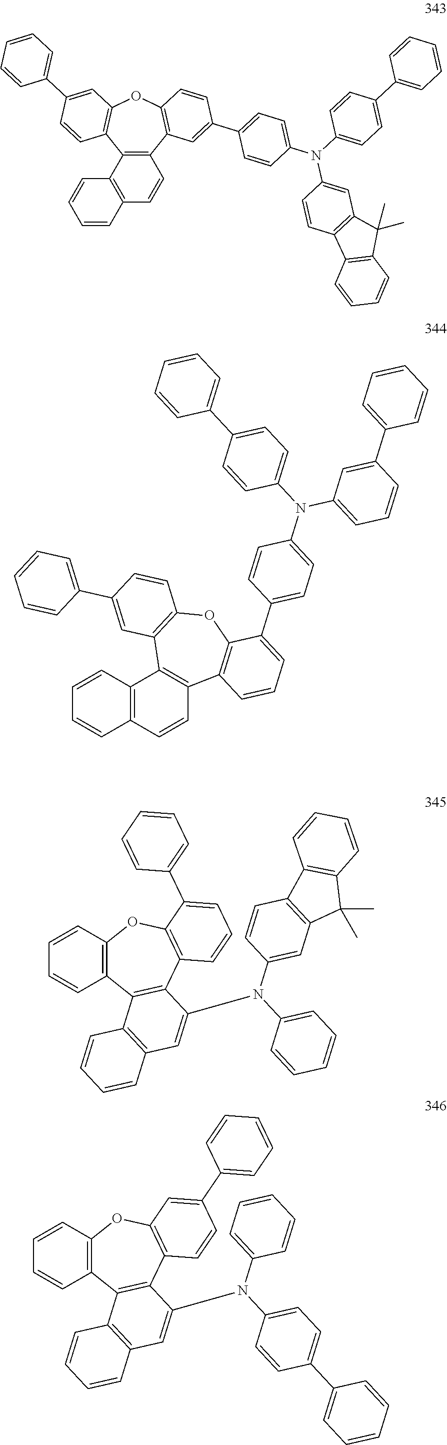 [SMILES: CC1(C)c2ccccc2-c2ccc(N(c3ccc(-c4ccccc4)cc3)c3ccc(-c4ccc5c(c4)-c4ccc6ccccc6c4-c4ccc(-c6ccccc6)cc4O5)cc3)cc21 ; c1ccc(-c2ccc(N(c3ccc(-c4cccc5c4Oc4ccc(-c6ccccc6)cc4-c4c-5ccc5ccccc45)cc3)c3cccc(-c4ccccc4)c3)cc2)cc1 ; CC1(C)c2ccccc2-c2ccc(N(c3ccccc3)c3cc4ccccc4c4c3-c3cccc(-c5ccccc5)c3Oc3ccccc3-4)cc21 ; c1ccc(-c2ccc(N(c3ccccc3)c3cc4ccccc4c4c3-c3ccc(-c5ccccc5)cc3Oc3ccccc3-4)cc2)cc1]

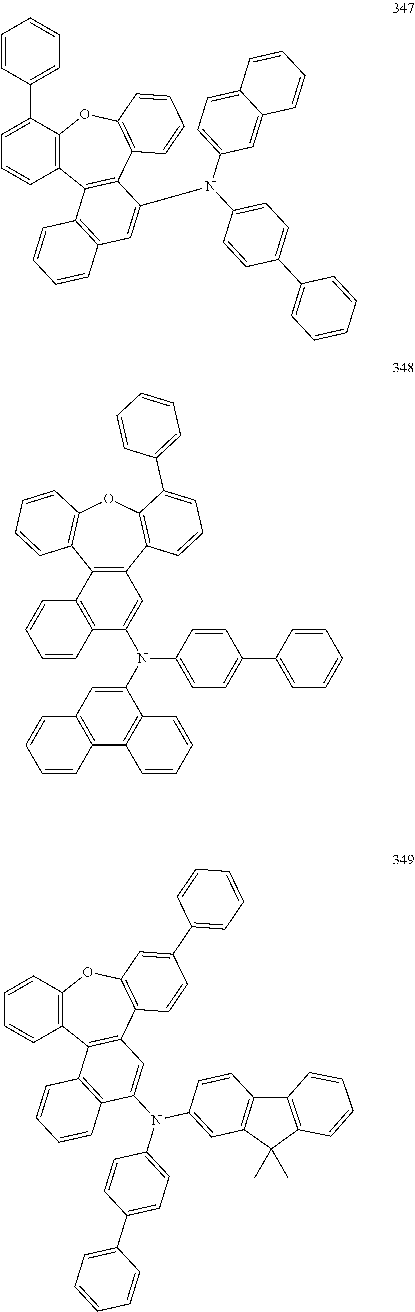 [SMILES: c1ccc(-c2ccc(N(c3ccc4ccccc4c3)c3cc4ccccc4c4c3-c3ccccc3Oc3c(-c5ccccc5)cccc3-4)cc2)cc1 ; c1ccc(-c2ccc(N(c3cc4c(c5ccccc35)-c3ccccc3Oc3c(-c5ccccc5)cccc3-4)c3cc4ccccc4c4ccccc34)cc2)cc1 ; CC1(C)c2ccccc2-c2ccc(N(c3ccc(-c4ccccc4)cc3)c3cc4c(c5ccccc35)-c3ccccc3Oc3cc(-c5ccccc5)ccc3-4)cc21]

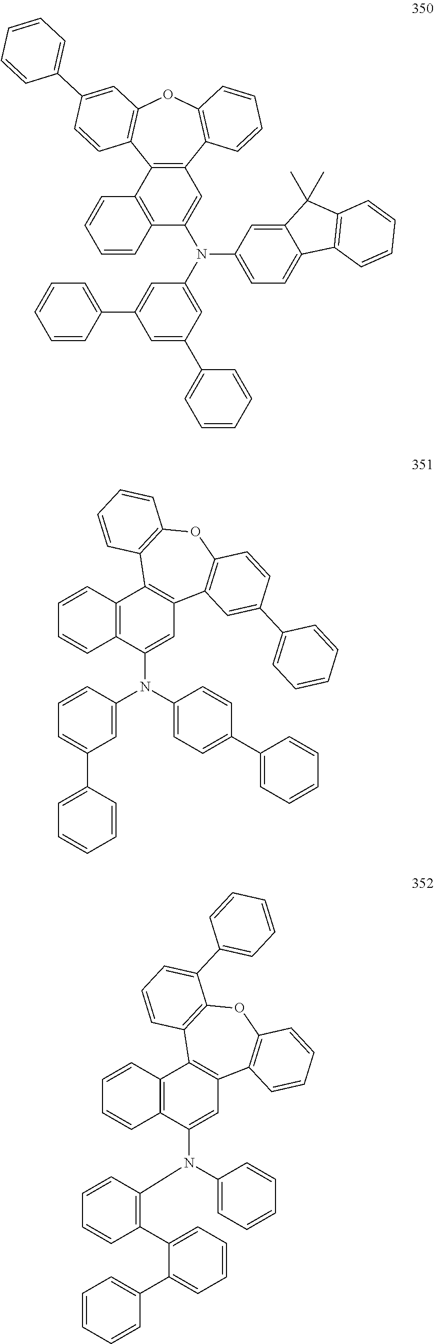 [SMILES: CC1(C)c2ccccc2-c2ccc(N(c3cc(-c4ccccc4)cc(-c4ccccc4)c3)c3cc4c(c5ccccc35)-c3ccc(-c5ccccc5)cc3Oc3ccccc3-4)cc21 ; c1ccc(-c2ccc(N(c3cccc(-c4ccccc4)c3)c3cc4c(c5ccccc35)-c3ccccc3Oc3ccc(-c5ccccc5)cc3-4)cc2)cc1 ; c1ccc(-c2ccccc2-c2ccccc2N(c2ccccc2)c2cc3c(c4ccccc24)-c2cccc(-c4ccccc4)c2Oc2ccccc2-3)cc1]

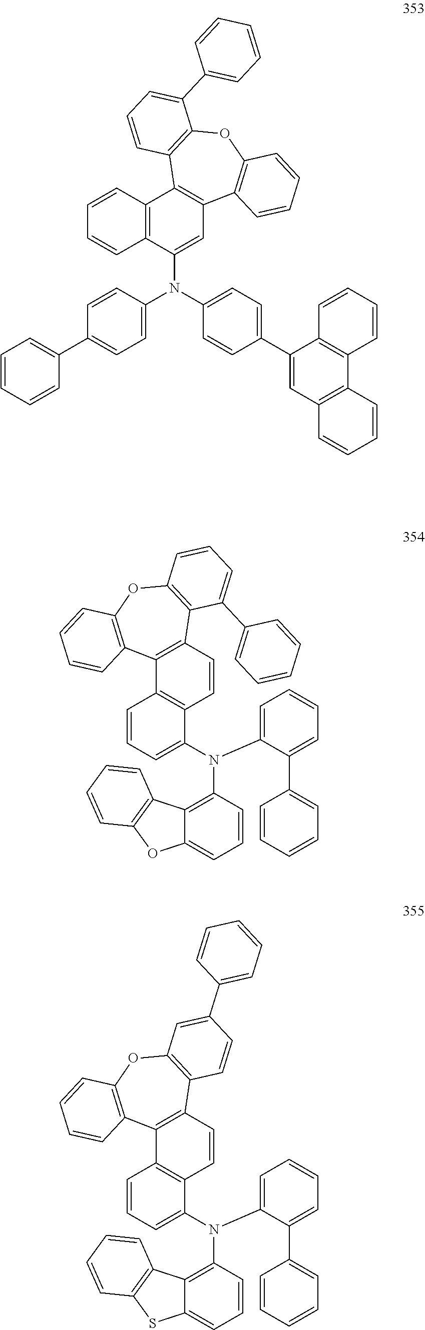 [SMILES: c1ccc(-c2ccc(N(c3ccc(-c4cc5ccccc5c5ccccc45)cc3)c3cc4c(c5ccccc35)-c3cccc(-c5ccccc5)c3Oc3ccccc3-4)cc2)cc1 ; c1ccc(-c2ccccc2N(c2cccc3c4c(ccc23)-c2c(cccc2-c2ccccc2)Oc2ccccc2-4)c2cccc3oc4ccccc4c23)cc1 ; c1ccc(-c2ccc3c(c2)Oc2ccccc2-c2c-3ccc3c(N(c4ccccc4-c4ccccc4)c4cccc5sc6ccccc6c45)cccc23)cc1]

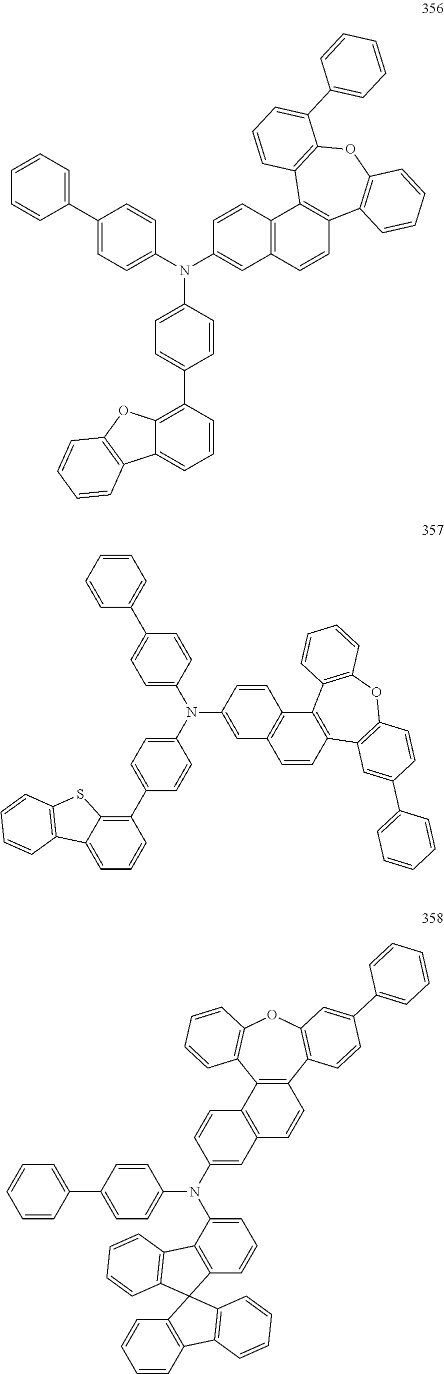 [SMILES: c1ccc(-c2ccc(N(c3ccc(-c4cccc5c4oc4ccccc45)cc3)c3ccc4c5c(ccc4c3)-c3ccccc3Oc3c(-c4ccccc4)cccc3-5)cc2)cc1 ; c1ccc(-c2ccc(N(c3ccc(-c4cccc5c4sc4ccccc45)cc3)c3ccc4c5c(ccc4c3)-c3cc(-c4ccccc4)ccc3Oc3ccccc3-5)cc2)cc1 ; c1ccc(-c2ccc(N(c3ccc4c5c(ccc4c3)-c3ccc(-c4ccccc4)cc3Oc3ccccc3-5)c3cccc4c3-c3ccccc3C43c4ccccc4-c4ccccc43)cc2)cc1]

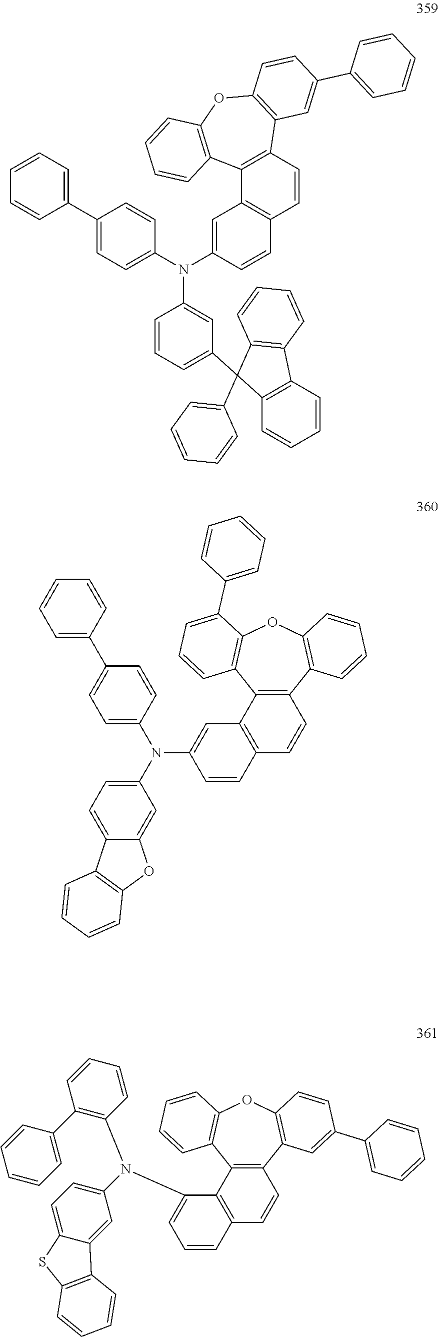 [SMILES: c1ccc(-c2ccc(N(c3cccc(C4(c5ccccc5)c5ccccc5-c5ccccc54)c3)c3ccc4ccc5c(c4c3)-c3ccccc3Oc3ccc(-c4ccccc4)cc3-5)cc2)cc1 ; c1ccc(-c2ccc(N(c3ccc4c(c3)oc3ccccc34)c3ccc4ccc5c(c4c3)-c3cccc(-c4ccccc4)c3Oc3ccccc3-5)cc2)cc1 ; c1ccc(-c2ccc3c(c2)-c2ccc4cccc(N(c5ccc6sc7ccccc7c6c5)c5ccccc5-c5ccccc5)c4c2-c2ccccc2O3)cc1]

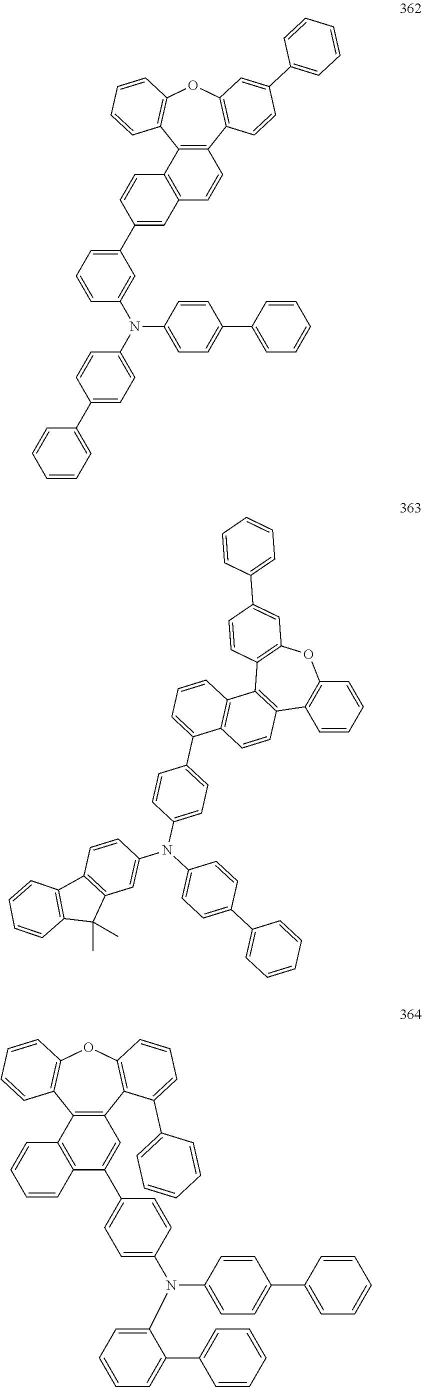 [SMILES: c1ccc(-c2ccc(N(c3ccc(-c4ccccc4)cc3)c3cccc(-c4ccc5c6c(ccc5c4)-c4ccc(-c5ccccc5)cc4Oc4ccccc4-6)c3)cc2)cc1 ; CC1(C)c2ccccc2-c2ccc(N(c3ccc(-c4ccccc4)cc3)c3ccc(-c4cccc5c6c(ccc45)-c4ccccc4Oc4cc(-c5ccccc5)ccc4-6)cc3)cc21 ; c1ccc(-c2ccc(N(c3ccc(-c4cc5c(c6ccccc46)-c4ccccc4Oc4cccc(-c6ccccc6)c4-5)cc3)c3ccccc3-c3ccccc3)cc2)cc1]

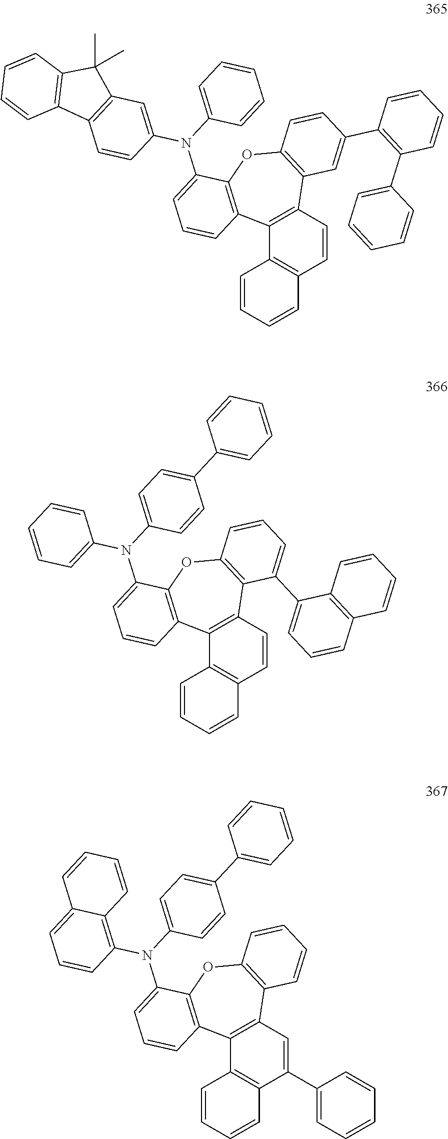 [SMILES: CC1(C)c2ccccc2-c2ccc(N(c3ccccc3)c3cccc4c3Oc3ccc(-c5ccccc5-c5ccccc5)cc3-c3ccc5ccccc5c3-4)cc21 ; c1ccc(-c2ccc(N(c3ccccc3)c3cccc4c3Oc3cccc(-c5cccc6ccccc56)c3-c3ccc5ccccc5c3-4)cc2)cc1 ; c1ccc(-c2ccc(N(c3cccc4c3Oc3ccccc3-c3cc(-c5ccccc5)c5ccccc5c3-4)c3cccc4ccccc34)cc2)cc1]

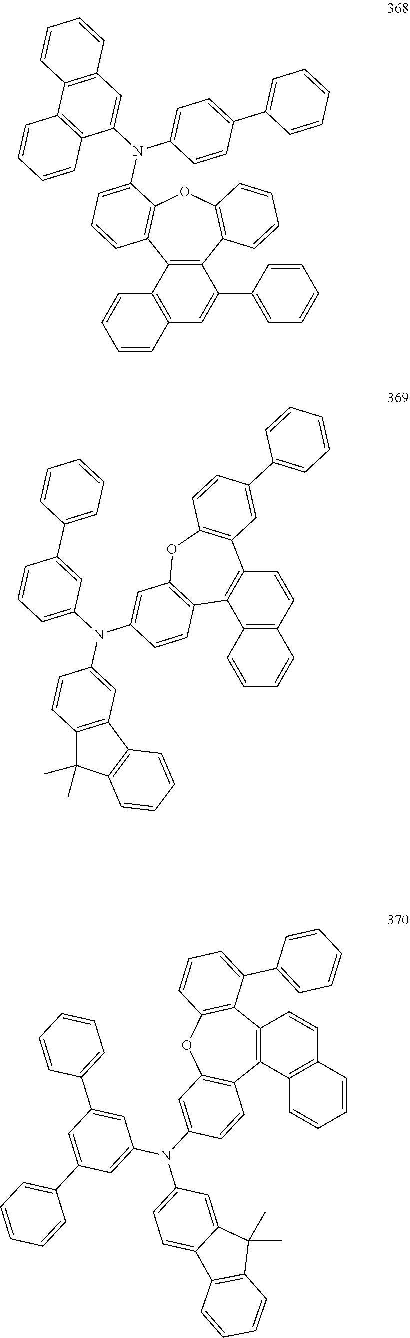 [SMILES: c1ccc(-c2ccc(N(c3cccc4c3Oc3ccccc3-c3c(-c5ccccc5)cc5ccccc5c3-4)c3cc4ccccc4c4ccccc34)cc2)cc1 ; CC1(C)c2ccccc2-c2cc(N(c3cccc(-c4ccccc4)c3)c3ccc4c(c3)Oc3ccc(-c5ccccc5)cc3-c3ccc5ccccc5c3-4)ccc21 ; CC1(C)c2ccccc2-c2ccc(N(c3cc(-c4ccccc4)cc(-c4ccccc4)c3)c3ccc4c(c3)Oc3cccc(-c5ccccc5)c3-c3ccc5ccccc5c3-4)cc21]

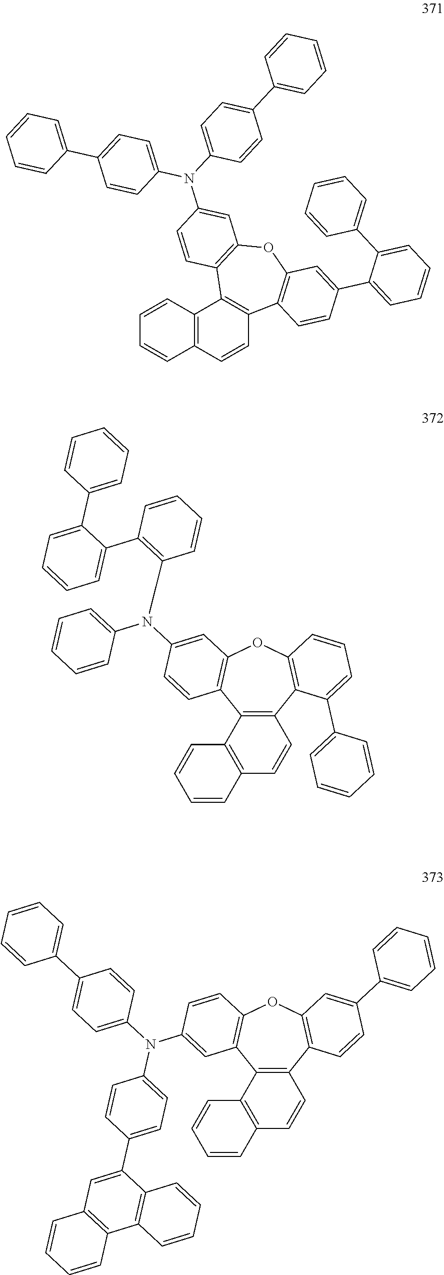 [SMILES: c1ccc(-c2ccc(N(c3ccc(-c4ccccc4)cc3)c3ccc4c(c3)Oc3cc(-c5ccccc5-c5ccccc5)ccc3-c3ccc5ccccc5c3-4)cc2)cc1 ; c1ccc(-c2ccccc2-c2ccccc2N(c2ccccc2)c2ccc3c(c2)Oc2cccc(-c4ccccc4)c2-c2ccc4ccccc4c2-3)cc1 ; c1ccc(-c2ccc(N(c3ccc(-c4cc5ccccc5c5ccccc45)cc3)c3ccc4c(c3)-c3c(ccc5ccccc35)-c3ccc(-c5ccccc5)cc3O4)cc2)cc1]

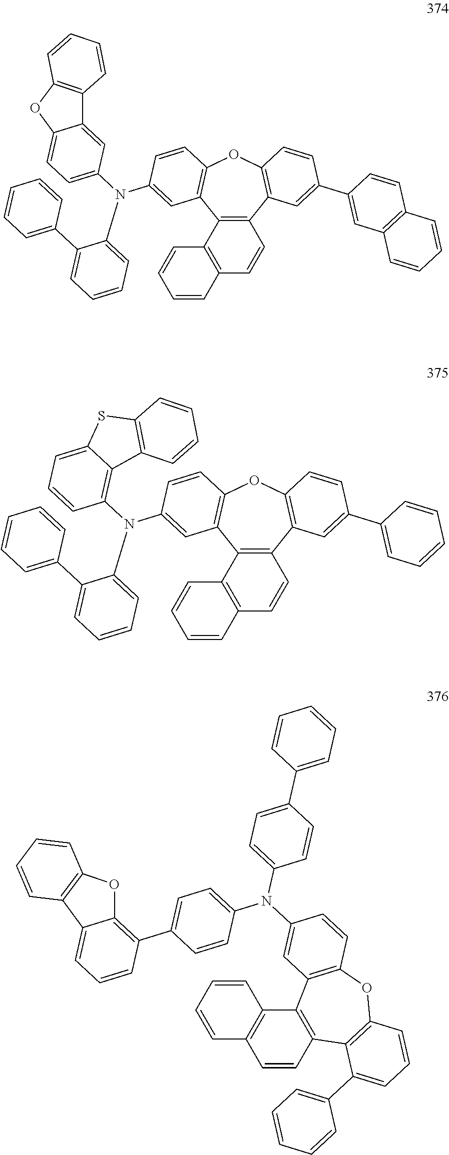 [SMILES: c1ccc(-c2ccccc2N(c2ccc3c(c2)-c2c(ccc4ccccc24)-c2cc(-c4ccc5ccccc5c4)ccc2O3)c2ccc3oc4ccccc4c3c2)cc1 ; c1ccc(-c2ccc3c(c2)-c2ccc4ccccc4c2-c2cc(N(c4ccccc4-c4ccccc4)c4cccc5sc6ccccc6c45)ccc2O3)cc1 ; c1ccc(-c2ccc(N(c3ccc(-c4cccc5c4oc4ccccc45)cc3)c3ccc4c(c3)-c3c(ccc5ccccc35)-c3c(cccc3-c3ccccc3)O4)cc2)cc1]

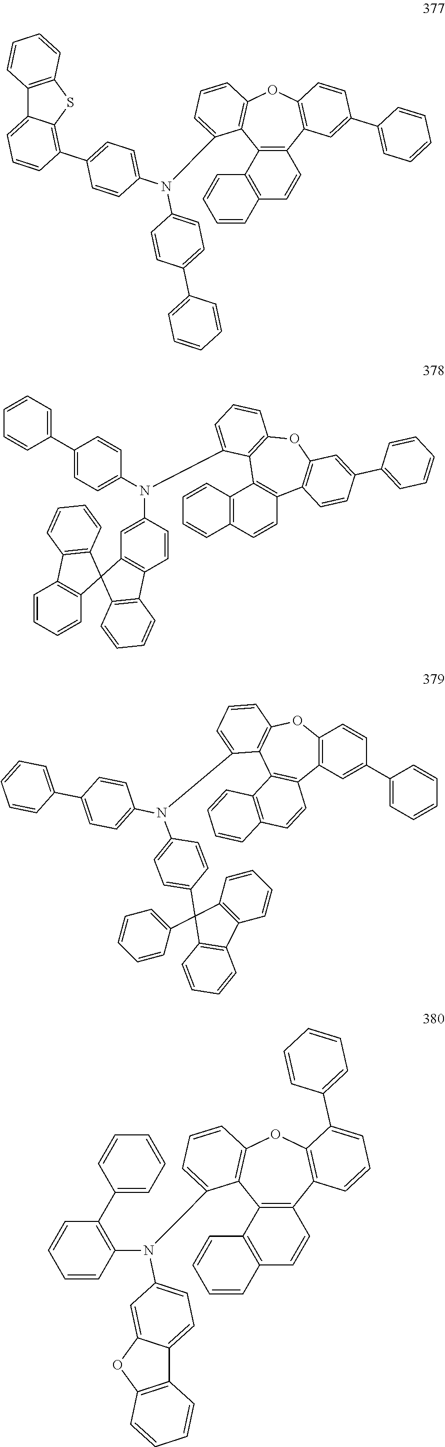 [SMILES: c1ccc(-c2ccc(N(c3ccc(-c4cccc5c4sc4ccccc45)cc3)c3cccc4c3-c3c(ccc5ccccc35)-c3cc(-c5ccccc5)ccc3O4)cc2)cc1 ; c1ccc(-c2ccc(N(c3ccc4c(c3)C3(c5ccccc5-c5ccccc53)c3ccccc3-4)c3cccc4c3-c3c(ccc5ccccc35)-c3ccc(-c5ccccc5)cc3O4)cc2)cc1 ; c1ccc(-c2ccc(N(c3ccc(C4(c5ccccc5)c5ccccc5-c5ccccc54)cc3)c3cccc4c3-c3c(ccc5ccccc35)-c3cc(-c5ccccc5)ccc3O4)cc2)cc1 ; c1ccc(-c2ccccc2N(c2ccc3c(c2)oc2ccccc23)c2cccc3c2-c2c(ccc4ccccc24)-c2cccc(-c4ccccc4)c2O3)cc1]

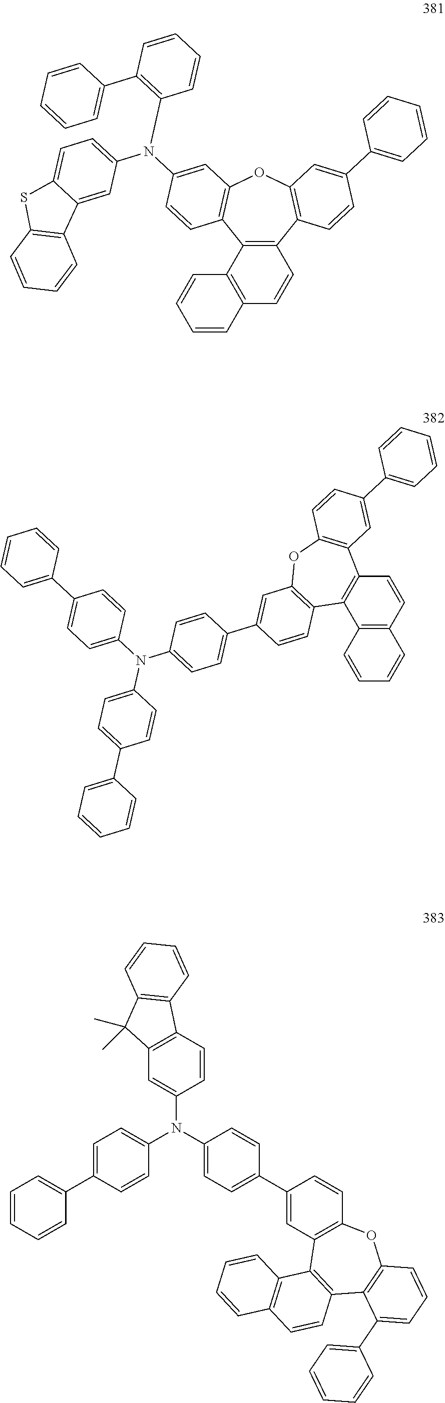 [SMILES: c1ccc(-c2ccc3c(c2)Oc2cc(N(c4ccc5sc6ccccc6c5c4)c4ccccc4-c4ccccc4)ccc2-c2c-3ccc3ccccc23)cc1 ; c1ccc(-c2ccc(N(c3ccc(-c4ccccc4)cc3)c3ccc(-c4ccc5c(c4)Oc4ccc(-c6ccccc6)cc4-c4ccc6ccccc6c4-5)cc3)cc2)cc1 ; CC1(C)c2ccccc2-c2ccc(N(c3ccc(-c4ccccc4)cc3)c3ccc(-c4ccc5c(c4)-c4c(ccc6ccccc46)-c4c(cccc4-c4ccccc4)O5)cc3)cc21]

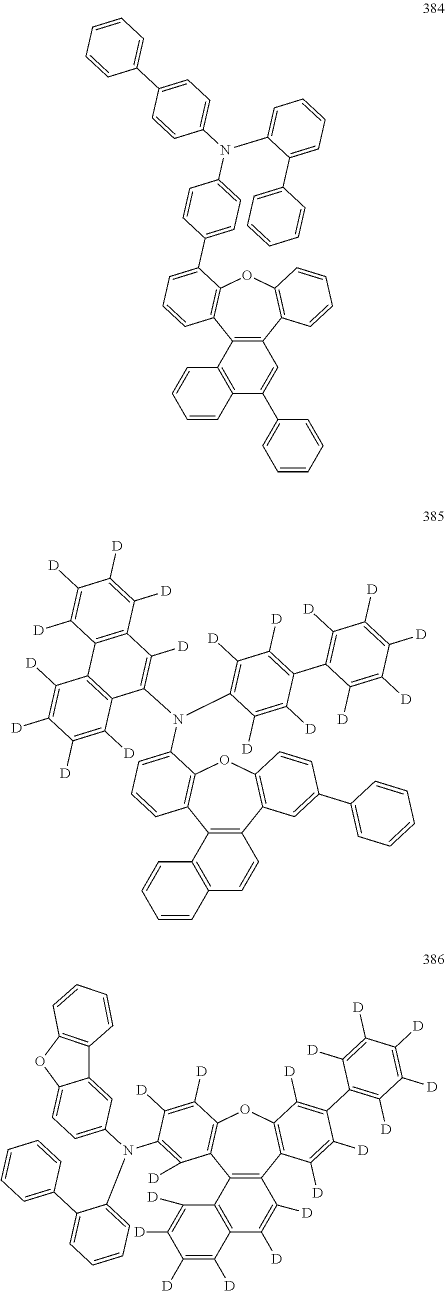 [SMILES: c1ccc(-c2ccc(N(c3ccc(-c4cccc5c4Oc4ccccc4-c4cc(-c6ccccc6)c6ccccc6c4-5)cc3)c3ccccc3-c3ccccc3)cc2)cc1 ; [2H]c1c([2H])c([2H])c(-c2c([2H])c([2H])c(N(c3cccc4c3Oc3ccc(-c5ccccc5)cc3-c3ccc5ccccc5c3-4)c3c([2H])c4c([2H])c([2H])c([2H])c([2H])c4c4c([2H])c([2H])c([2H])c([2H])c34)c([2H])c2[2H])c([2H])c1[2H] ; [2H]c1c([2H])c([2H])c(-c2c([2H])c([2H])c3c(c2[2H])Oc2c([2H])c([2H])c(N(c4ccc5oc6ccccc6c5c4)c4ccccc4-c4ccccc4)c([2H])c2-c2c-3c([2H])c([2H])c3c([2H])c([2H])c([2H])c([2H])c23)c([2H])c1[2H]]

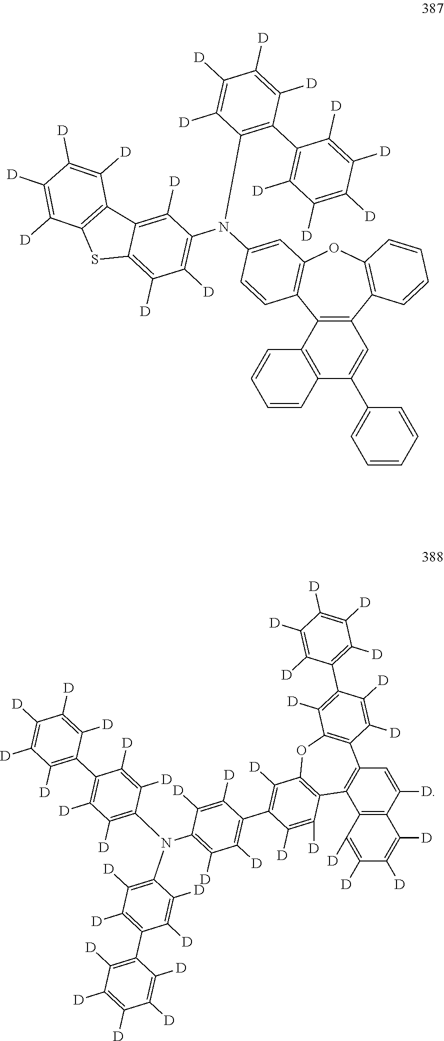

In addition, by introducing various substituents to the structure of Chemical Formula 1, compounds having unique properties of the introduced substituents may be synthesized. For example, by introducing substituents normally used for a hole injection layer material, a hole transport layer material, a hole transport auxiliary layer material, an electron blocking layer material, a light emitting layer material, an electron transport layer material, a hole blocking layer material and an electron injection layer material used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials may be enhanced, and material applications may become diverse.

One embodiment of the present disclosure provides an organic light emitting device including the heterocyclic compound represented by Chemical Formula 1. The “organic light emitting device” may be expressed in terms such as an “organic light emitting diode”, an “OLED”, an “OLED device” and an “organic electroluminescent device”.

In addition, one embodiment of the present disclosure provides an organic light emitting device including: a first electrode; a second electrode provided opposite to the first electrode; and one or more organic material layers provided between the first electrode and the second electrode, wherein one or more layers of the organic material layers include the heterocyclic compound represented by Chemical Formula 1.

In one embodiment of the present disclosure, the first electrode may be a positive electrode, and the second electrode may be a negative electrode.

In one embodiment of the present disclosure, the first electrode may be a negative electrode, and the second electrode may be a positive electrode.

In one embodiment of the present disclosure, the organic material layer may include one or more types selected from the group consisting of an electron injection layer, an electron transport layer, a hole blocking layer, a light emitting layer, an electron blocking layer, a hole transport layer and a hole injection layer, and the one or more types of layers selected from the group consisting of an electron injection layer, an electron transport layer, a hole blocking layer, a light emitting layer, an electron blocking layer, a hole transport layer and a hole injection layer may include the heterocyclic compound represented by Chemical Formula 1.

In one embodiment of the present disclosure, the organic material layer may include a hole transport layer, and the hole transport layer may include the heterocyclic compound represented by Chemical Formula 1.

In one embodiment of the present disclosure, the organic material layer may include an electron blocking layer, and the electron blocking layer may include the heterocyclic compound represented by Chemical Formula 1.

In one embodiment of the present disclosure, the organic light emitting device may be a red organic light emitting device, and the heterocyclic compound represented by Chemical Formula 1 may be used as a material of the red organic light emitting device.

In one embodiment of the present disclosure, the organic light emitting device may be a green organic light emitting device, and the heterocyclic compound represented by Chemical Formula 1 may be used as a material of the green organic light emitting device.

In one embodiment of the present disclosure, the organic light emitting device may be a blue organic light emitting device, and the heterocyclic compound represented by Chemical Formula 1 may be used as a material of the blue organic light emitting device.

In one embodiment of the present disclosure, the organic light emitting device may be a red organic light emitting device, and the heterocyclic compound represented by Chemical Formula 1 may be used as a material of a hole transport layer or an electron blocking layer of the red organic light emitting device.

In one embodiment of the present disclosure, the organic light emitting device may be a green organic light emitting device, and the heterocyclic compound represented by Chemical Formula 1 may be used as a material of a hole transport layer or an electron blocking layer of the green organic light emitting device.

In one embodiment of the present disclosure, the organic light emitting device may be a blue organic light emitting device, and the heterocyclic compound represented by Chemical Formula 1 may be used as a material of a hole transport layer or an electron blocking layer of the blue organic light emitting device.

Specific descriptions on the heterocyclic compound represented by Chemical Formula 1 are the same as the descriptions provided above.

In the organic light emitting device according to one embodiment of the present disclosure, the organic material layer includes an electron injection layer or an electron transport layer, and the electron injection layer or the electron transport layer may include the heterocyclic compound.

In the organic light emitting device according to one embodiment of the present disclosure, the organic material layer includes an electron blocking layer or a hole blocking layer, and the electron blocking layer or a hole blocking layer may include the heterocyclic compound.

In the organic light emitting device according to one embodiment of the present disclosure, the organic material layer includes a light emitting layer, and the light emitting layer may include the heterocyclic compound.

In the organic light emitting device according to one embodiment of the present disclosure, the organic material layer includes a hole injection layer or a hole transport layer, and the hole transport layer or the electron blocking layer may include the heterocyclic compound.

The organic light emitting device of the present disclosure may be manufactured using common organic light emitting device manufacturing methods and materials except that one or more organic material layers are formed using the heterocyclic compound described above.

The heterocyclic compound may form the organic material layer using a solution coating method as well as a vacuum deposition method when the organic light emitting device is manufactured. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, a hole transport layer, a hole transport auxiliary layer, a light emitting layer, an electron injection layer, an electron transport layer, an electron blocking layer, a hole blocking layer and the like as the organic material layer. However, the structure of the organic light emitting device is not limited thereto, and may include a smaller number of organic material layers.

In the organic light emitting device of the present disclosure, the organic material layer includes a hole transport layer or an electron blocking layer, and the hole transport layer or the electron blocking layer may include the heterocyclic compound represented by Chemical Formula 1. When the heterocyclic compound is used in the hole transport layer or the electron blocking layer, electron migration may be effectively controlled due to structural characteristics of the compound having a LUMO level and a triplet energy level, and as a result, driving efficiency and lifetime of the organic light emitting device may become superior.

In one embodiment of the present disclosure, the organic light emitting device may include one or more organic material layers, the organic material layer may include a hole transport layer, and the hole transport layer may include the heterocyclic compound represented by Chemical Formula 1.

In one embodiment of the present disclosure, the organic light emitting device may include one or more organic material layers, the organic material layer may include an electron blocking layer, and the electron blocking layer may include the heterocyclic compound represented by Chemical Formula 1.

In one embodiment of the present disclosure, the organic light emitting device may further include one, two or more layers selected from the group consisting of a light emitting layer, a hole injection layer, a hole blocking layer, an electron injection layer, an electron transport layer and a hole blocking layer.

In one embodiment of the present disclosure, the organic material layer includes the heterocyclic compound represented by Chemical Formula 1, and a phosphorescent dopant may be used therewith.

As the phosphorescent dopant material, those known in the art may be used. For example, phosphorescent dopant materials represented by LL′MX′, LL′ L″M, LMX′X″, L₂MX′ and L₃M may be used, however, the scope of the present disclosure is not limited by these examples.

M may be iridium, platinum, osmium or the like.

L is an anionic bidentate ligand coordinated to M by sp² carbon and heteroatom, and X may function to trap electrons or holes. Nonlimiting examples of L, L′ and L″ may include 2-(1-naphthyl)benzoxazole, 2-phenylbenzoxazole, 2-phenylbenzothiazole, 7,8-benzoquinoline, phenylpyridine, benzothiophenylpyridine, 3-methoxy-2-phenylpyridine, thiophenylpyridine, tolylpyridine and the like. Nonlimiting examples of X′ and X″ may include acetylacetonate (acac), picolinate, 8-10 hexafluoroacetylacetonate, salicylidene, hydroxyquinolinate and the like.

Specific examples of the phosphorescent dopant are shown below, however, the phosphorescent dopant is not limited to these examples:

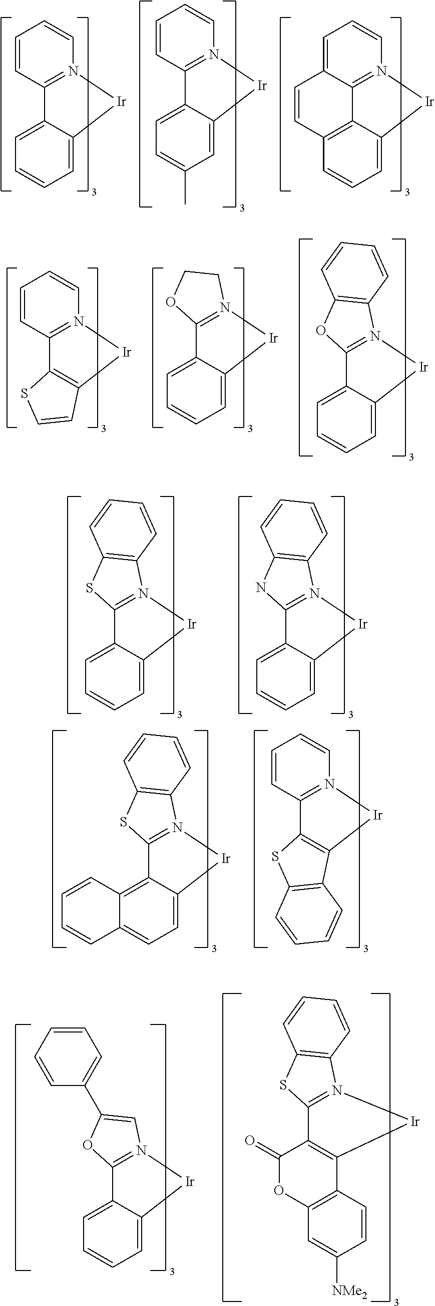

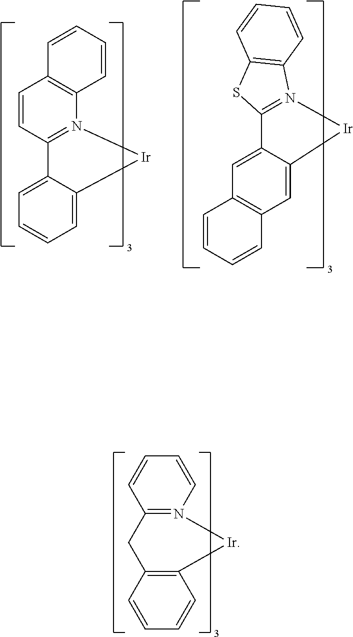

In one embodiment of the present disclosure, the organic material layer includes the heterocyclic compound represented by Chemical Formula 1, and an iridium-based dopant may be used therewith.

In one embodiment of the present disclosure, a content of the dopant may be from 1% to 15%, preferably from 2% to 10% and more preferably from 3% to 7% based on the total weight of the light emitting layer.

In the organic light emitting device according to one embodiment of the present disclosure, the organic material layer includes a hole transport layer or an electron blocking layer, and the hole transport layer or the electron blocking layer may include the heterocyclic compound represented by Chemical Formula 1.

The organic light emitting device according to one embodiment of the present disclosure may further include one, two or more layers selected from the group consisting of a light emitting layer, a hole injection layer, a hole blocking layer, an electron injection layer, an electron transport layer and a hole blocking layer.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, a hole transport layer, a light emitting layer, an electron transport layer, an electron injection layer and the like as the organic material layer. However, the structure of the organic light emitting device is not limited thereto, and may include a smaller number of organic material layers.

One embodiment of the present disclosure provides an organic light emitting device, wherein the organic material layer includes at least one of a hole transport layer, an electron blocking layer and an electron transport layer, and at least one of the hole transport layer, the electron blocking layer and the electron transport layer includes the heterocyclic compound represented by Chemical Formula 1.

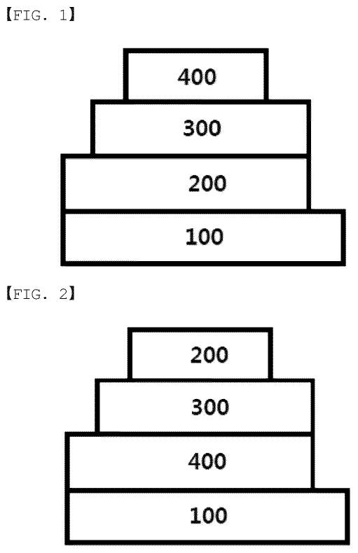

FIGS. 1 to 3 illustrate a lamination order of electrodes and organic material layers of the organic light emitting device according to one embodiment of the present disclosure. However, it is not intended that the scope of the present application be limited by these drawings, and structures of organic light emitting devices known in the art may also be applied to the present application.

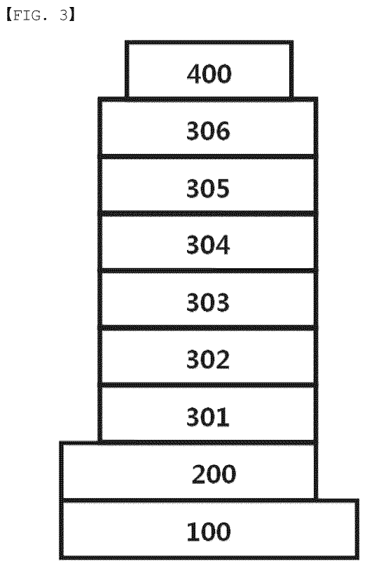

FIG. 1 illustrates an organic light emitting device in which a positive electrode 200, an organic material layer 300 and a negative electrode 400 are sequentially laminated on a substrate 100. However, the structure is not limited only to such a structure, and as illustrated in FIG. 2, an organic light emitting device in which a negative electrode, an organic material layer and a positive electrode are sequentially laminated on a substrate may also be obtained.

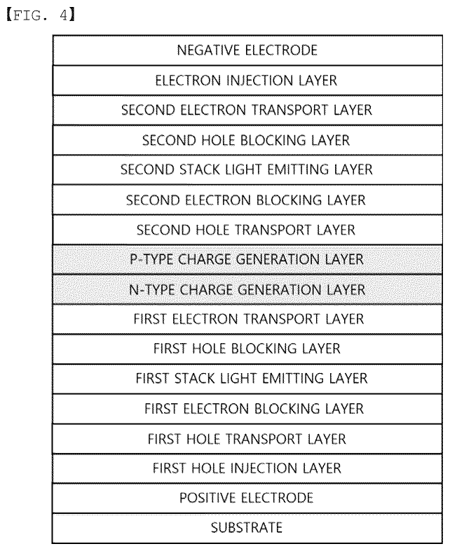

FIG. 3 illustrates a case of the organic material layer being a multilayer. An organic light emitting device according to FIG. 3 includes a hole injection layer 301, a hole transport layer 302, a light emitting layer 303, a hole blocking layer 304, an electron transport layer 305 and an electron injection layer 306. However, the scope of the present application is not limited by such a lamination structure, and as necessary, the layers other than the light emitting layer may not be included, and other necessary functional layers may be further added.

As the organic light emitting device according to one embodiment of the present application, an organic light emitting device having a 2-stack tandem structure is schematically illustrated in FIG. 4.

Herein, a first electron blocking layer, a first hole blocking layer, a second hole blocking layer and the like described in FIG. 4 may not be included in some cases.

One embodiment of the present disclosure provides a method for manufacturing an organic light emitting device, the method including: preparing a substrate; forming a first electrode on the substrate; forming one or more organic material layers on the first electrode; and forming a second electrode on the organic material layers, wherein the forming of organic material layers includes forming one or more organic material layers using the composition for an organic material layer according to one embodiment of the present disclosure.

The organic material layer including the heterocyclic compound represented by Chemical Formula 1 may further include other materials as necessary.

In the organic light emitting device according to one embodiment of the present disclosure, materials other than the heterocyclic compound represented by Chemical Formula 1 are illustrated below, however, these are for illustrative purposes only and not for limiting the scope of the present application, and these materials may be replaced by materials known in the art.

As the positive electrode material, materials each having a relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the negative electrode material, materials each having a relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′, 4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], conductive polymers having solubility such as polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, may be used.

As the hole transport layer material, pyrazoline derivatives, arylamine-based derivatives, stilbene derivatives, triphenyldiamine derivatives and the like may be used, and low molecular or high molecular materials may also be used.

As the electron transport layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials as well as low molecular materials may also be used.

As examples of the electron injection layer material, LiF is typically used in the art, however, the present application is not limited thereto.

As the light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light alone by binding holes and electrons injected from a positive electrode and a negative electrode, respectively, may be used, however, materials having a host material and a dopant material involved in light emission together may also be used.

When hosts of the light emitting layer material are mixed and used, same series hosts may be mixed and used, or different series hosts may be mixed and used. For example, any two or more types of materials among n-type host materials and p-type host materials may be selected and used as a host material of a light emitting layer.

The organic light emitting device according to one embodiment of the present disclosure may be a top-emission type, a bottom-emission type or a dual-emission type depending on the materials used.

The heterocyclic compound according to one embodiment of the present disclosure may also be used in an organic electronic device including an organic solar cell, an organic photo conductor, an organic transistor and the like under a principle similar to that in the organic light emitting device.

Hereinafter, preferred examples are provided to help to understand the present disclosure, however, the following examples are only provided to more readily understand the present disclosure, and the present disclosure is not limited thereto.

### Preparation Example

### Preparation Example 1: Preparation of Compound 1

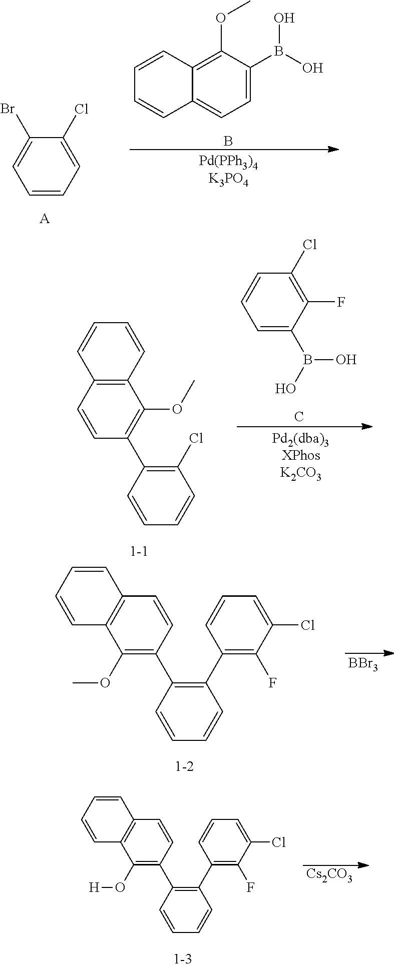

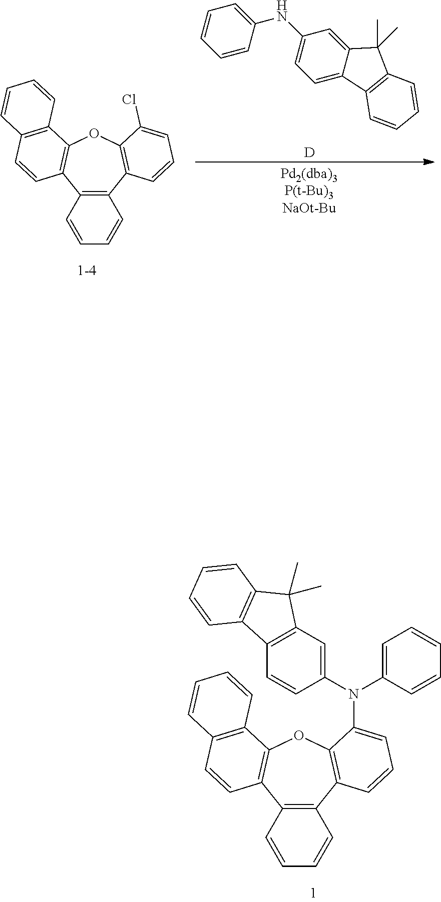

### 1) Preparation of Compound 1-1

1-bromo-2-chlorobenzene (A) (30 g, 0.157 mol, 1 eq.), (1-methoxynaphthalen-2-yl)boronic acid (B) (34.9 g, 0.173 mol, 1.1 eq.), tetrakis(triphenylphosphine) palladium (Pd(PPh₃)₄) (9.2 g, 0.008 mol, 0.05 eq.) and tri-potassium phosphate (K₃PO₄) (73.2 g, 0.345 mol, 2.2 eq.) were introduced to 1,4-dioxane (360 ml) and water (60 ml), and the mixture was stirred for 12 hours at 100° C. Water was introduced thereto to terminate the reaction, and then the result was extracted using methylene chloride (MC) and water. After that, moisture was removed using magnesium sulfate (MgSO₄), and then the result was separated using a silica gel column to obtain Compound 1-1 (40 g, yield 95%).

### 2) Preparation of Compound 1-2

Compound 1-1 (40 g, 0.149 mol, 1 eq.), (3-chloro-2-fluorophenyl)boronic acid (C) (39.1 g, 0.224 mol, 1.5 eq.), tris(dibenzylideneacetone) dipalladium (Pd₂(dba)₃) (6.4 g, 0.007 mol, 0.05 eq.), XPhos (7.2 g, 0.015 mol, 0.1 eq.) and potassium carbonate (K₂CO₃) (61.8 g, 0.447 mol, 3.0 eq.) were introduced to 1,4-dioxane (480 ml) and water (H₂O) (120 ml), and the mixture was stirred for 24 hours at 100° C. Water was introduced thereto to terminate the reaction, and then the result was extracted using methylene chloride (MC) and water. After that, moisture was removed using magnesium sulfate (MgSO₄), and then the result was separated using a silica gel column to obtain Compound 1-2 (50.4 g, yield 93%).

### 3) Preparation of Compound 1-3

Compound 1-2 (35 g, 0.096 mol, 1 eq.) was introduced to methylene chloride (MC) (350 ml), and the mixture was cooled and stirred at 0° ° C. After that, boron tribromide (BBr₃) (48.1 g, 0.192 mol, 2.0 eq.) was slowly added dropwise thereto, and then the result was stirred for 1 hour at room temperature. Water was introduced thereto to terminate the reaction, and then the result was extracted using methylene chloride (MC) and water. After that, moisture was removed using magnesium sulfate (MgSO₄), and then the result was separated using a silica gel column to obtain Compound 1-3 (30.7 g, yield 92%).

### 4) Preparation of Compound 1-4

Compound 1-3 (30 g, 0.086 mol, 1 eq.) and cesium carbonate (Cs₂CO₃) (56.0 g, 0.172 mol, 2.0 eq.) were introduced to dimethylacetamide (DMA) (300 ml), and the mixture was stirred for 1 hour under reflux. Water was introduced thereto to terminate the reaction, and then the result was extracted using methylene chloride (MC) and water. After that, moisture was removed using magnesium sulfate (MgSO₄), and then the result was separated using a silica gel column to obtain Compound 1-4 (25.3 g, yield 90%).

### 5) Preparation of Compound 1

9,9-dimethyl-N-phenyl-9H-fluoren-2-amine (D) (8 g, 0.028 mol, 1 eq.), Compound 1-4 (10.2 g, 0.031 mol, 1.1 eq.), tris(dibenzylideneacetone) dipalladium (Pd₂(dba)₃) (0.9 g, 0.001 mol, 0.05 eq.), XPhos (1.4 g, 0.003 mol, 0.1 eq.) and sodium tert-butoxide (t-BuONa) (4.0 g, 0.042 mol, 1.5 eq.) were introduced to toluene (80 ml), and the mixture was stirred for 2 hours at 100° C. Water was introduced thereto to terminate the reaction, and then the result was extracted using methylene chloride (MC) and water. After that, moisture was removed using magnesium sulfate (MgSO₄), and then the result was separated using a silica gel column to obtain Compound 1 (13.4 g, yield 83%).

Compounds were synthesized in the same manner as in Preparation Example 1 except that Intermediate A, Intermediate B, Intermediate C and Intermediate D of the following Table 1 were used instead of Compound A, Compound B, Compound C and Compound D in Preparation Example 1, respectively.

TABLE 1

| Compound | Intermediate | Intermediate | Intermediate | Intermediate | |
|---|---|---|---|---|---|
| No. | A | B | C | D | Yield |
| 1 | 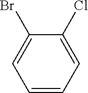 | 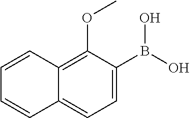 | 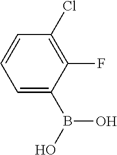 | 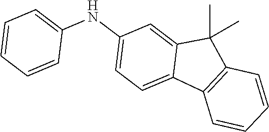 | 61% |
| 5 | 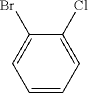 | 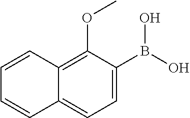 | 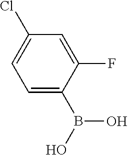 | 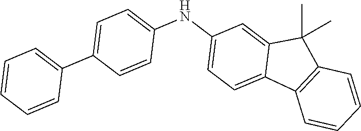 | 65% |
| 7 | 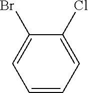 | 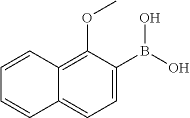 | 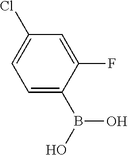 | 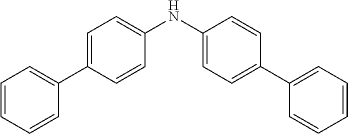 | 67% |
| 9 | 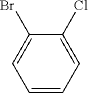 | 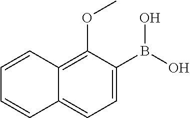 | 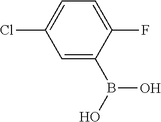 | 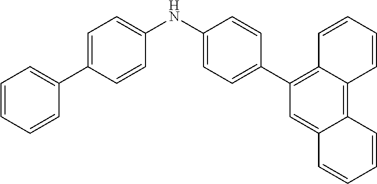 | 65% |
| 16 | 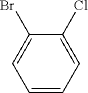 | 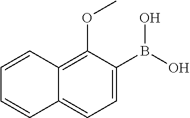 | 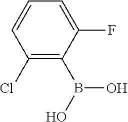 | 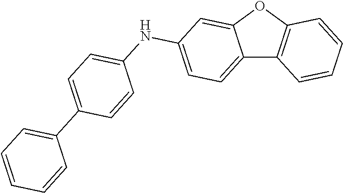 | 58% |
| 17 | 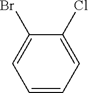 | 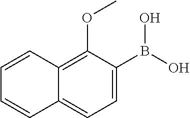 | 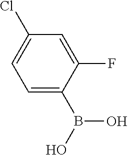 | 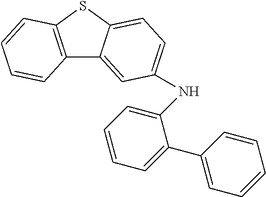 | 60% |
| 25 | 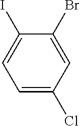 | 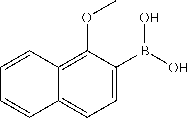 | 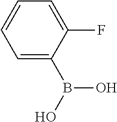 | 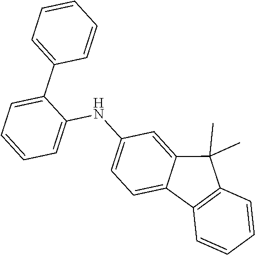 | 57% |
| 28 | 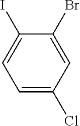 | 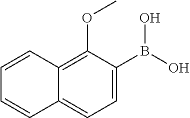 | 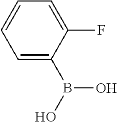 | 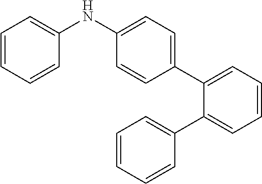 | 60% |
| 30 | 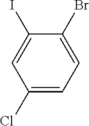 | 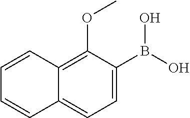 | 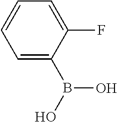 | 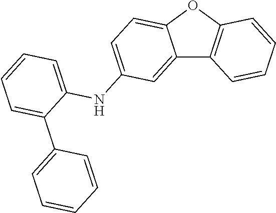 | 58% |
| 33 | 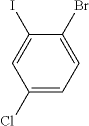 | 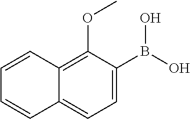 | 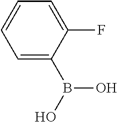 | 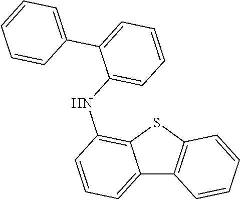 | 57% |
| 45 | 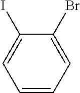 | 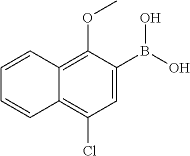 | 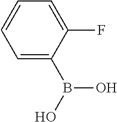 | 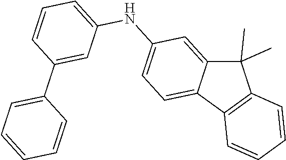 | 55% |
| 53 | 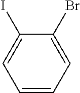 | 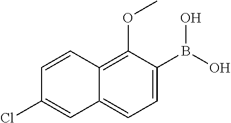 | 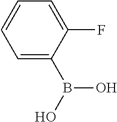 | 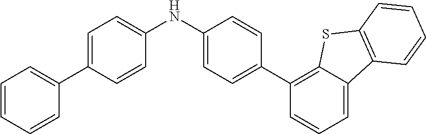 | 57% |
| 56 | 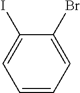 | 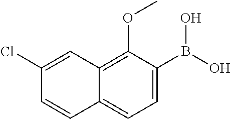 | 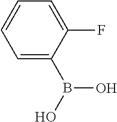 | 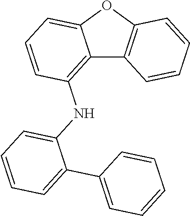 | 56% |
| 74 | 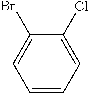 | 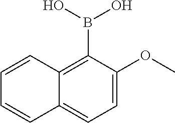 | 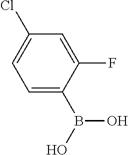 | 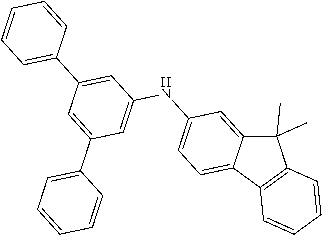 | 63% |
| 75 | 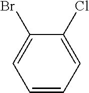 | 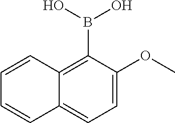 | 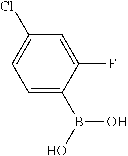 | 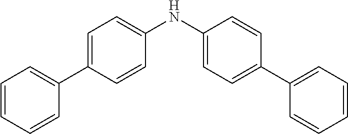 | 63% |
| 77 | 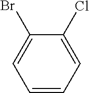 | 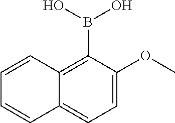 | 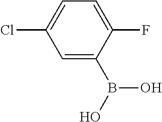 | 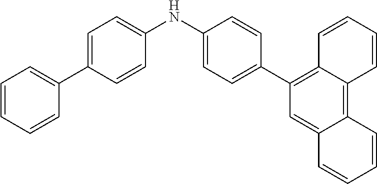 | 64% |
| 78 | 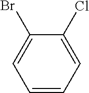 | 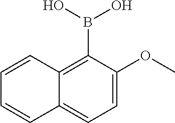 | 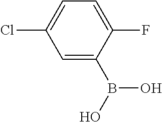 | 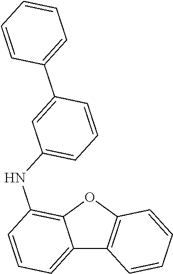 | 56% |
| 79 | 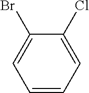 | 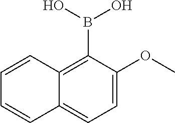 | 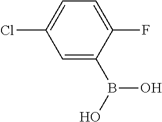 | 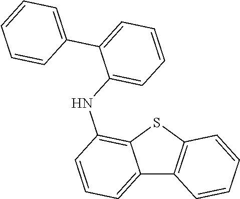 | 56% |
| 93 | 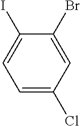 | 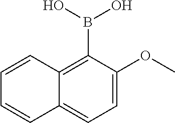 | 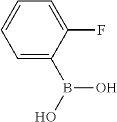 | 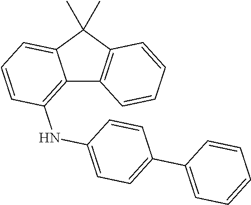 | 60% |
| 99 | 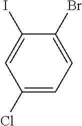 | 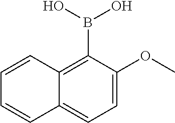 | 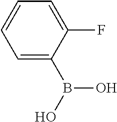 | 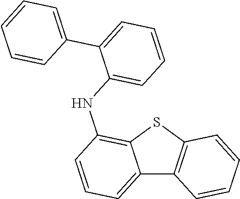 | 58% |
| 100 | 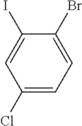 | 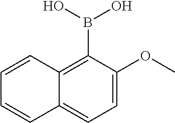 | 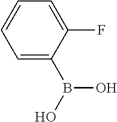 | 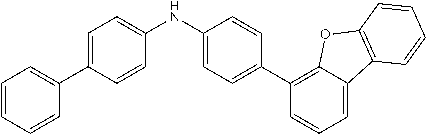 | 61% |
| 113 | 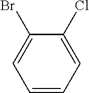 | 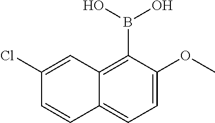 | 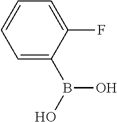 | 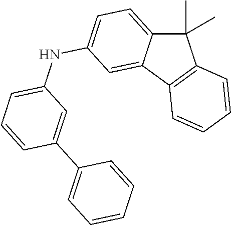 | 57% |
| 119 | 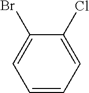 | 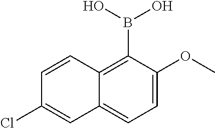 | 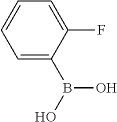 | 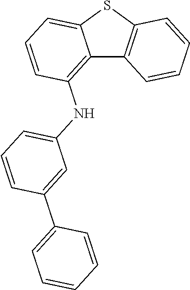 | 57% |
| 124 | 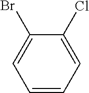 | 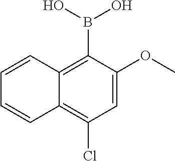 | 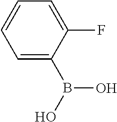 | 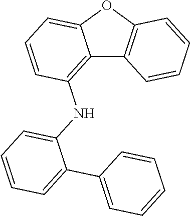 | 55% |
| 135 | 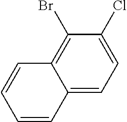 | 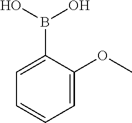 | 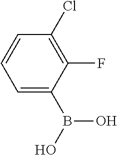 | 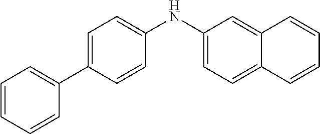 | 62% |
| 138 | 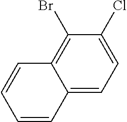 | 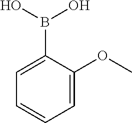 | 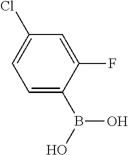 | 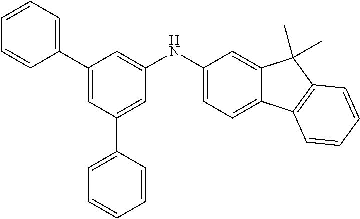 | 60% |
| 140 | 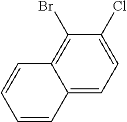 | 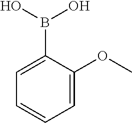 | 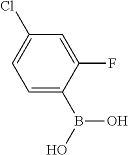 | 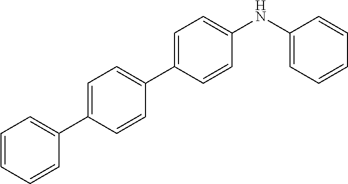 | 63% |
| 142 | 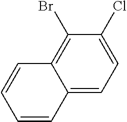 | 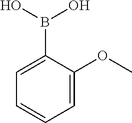 | 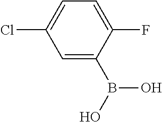 | 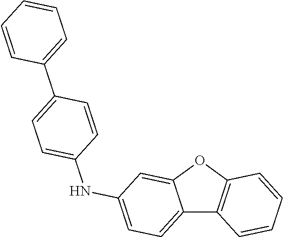 | 62% |
| 143 | 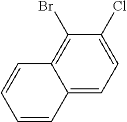 | 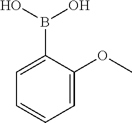 | 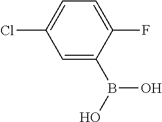 | 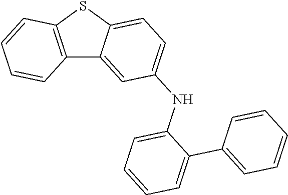 | 59% |
| 157 | 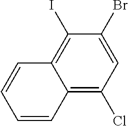 | 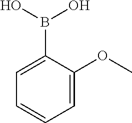 | 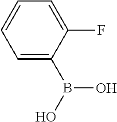 | 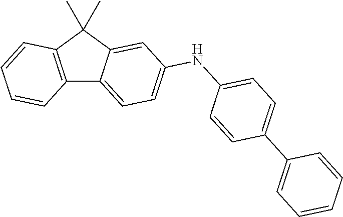 | 62% |
| 163 | 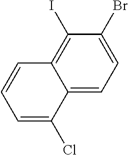 | 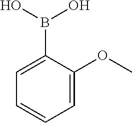 | 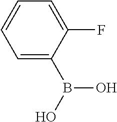 | 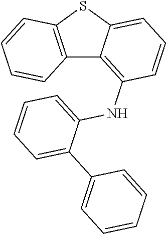 | 58% |
| 164 | 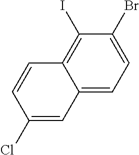 | 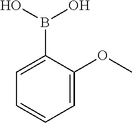 | 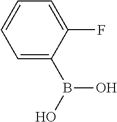 | 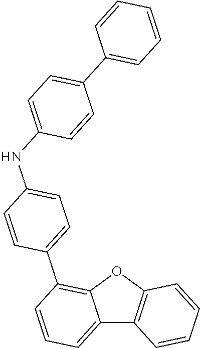 | 60% |
| 177 | 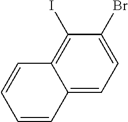 | 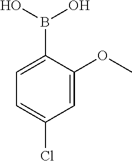 | 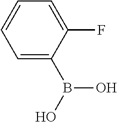 | 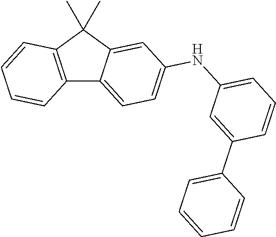 | 60% |
| 182 | 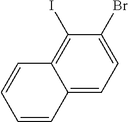 | 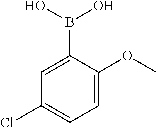 | 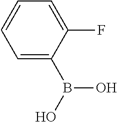 | 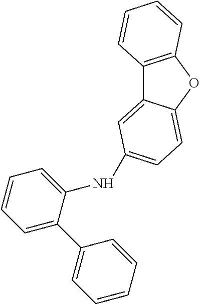 | 59% |
| 183 | 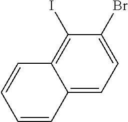 | 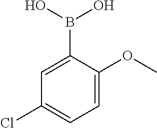 | 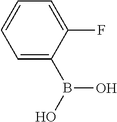 | 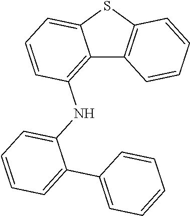 | 58% |
| 198 | 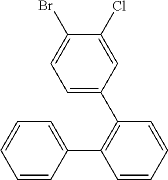 | 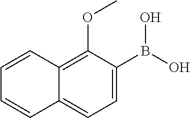 | 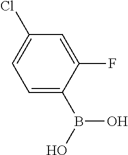 | 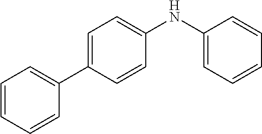 | 60% |
| 205 | 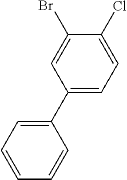 | 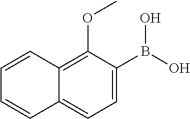 | 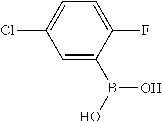 | 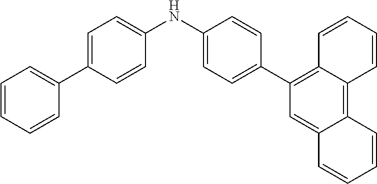 | 64% |
| 212 | 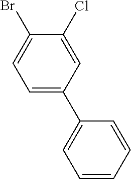 | 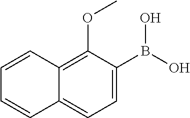 | 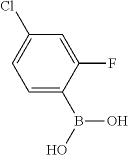 | 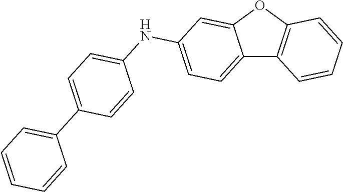 | 61% |
| 230 | 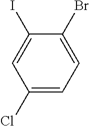 | 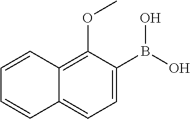 | 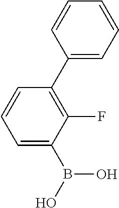 | 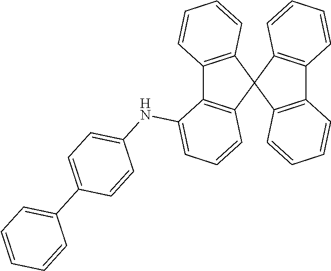 | 63% |
| 231 | 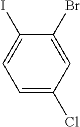 | 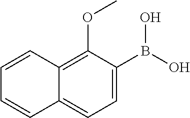 | 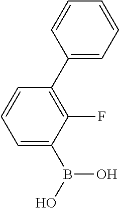 | 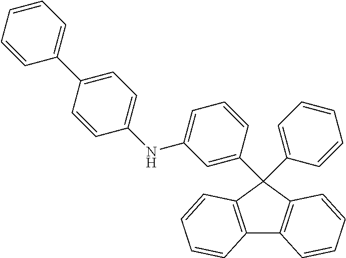 | 62% |
| 238 | 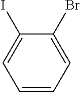 | 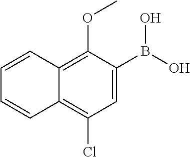 | 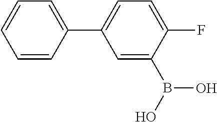 | 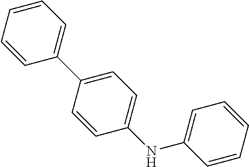 | 58% |
| 252 | 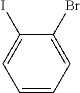 | 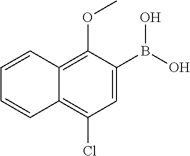 | 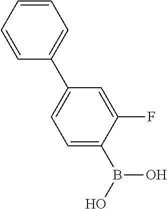 | 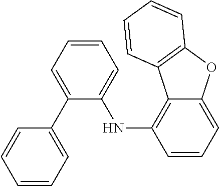 | 57% |
| 267 | 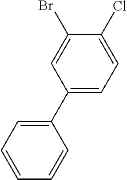 | 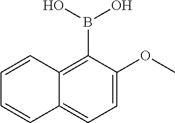 | 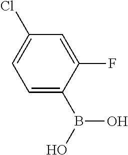 | 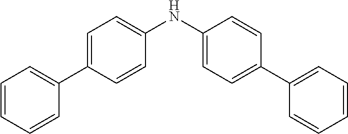 | 63% |
| 304 | 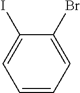 | 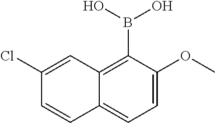 | 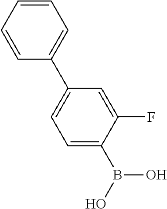 | 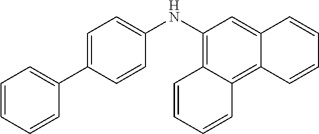 | 60% |
| 329 | 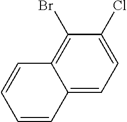 | 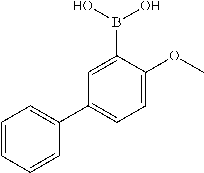 | 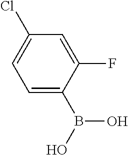 | 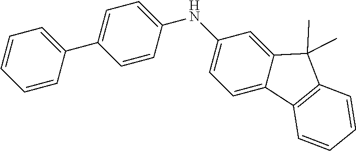 | 63% |
| 351 | 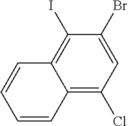 | 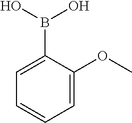 | 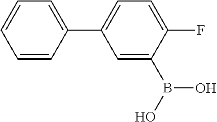 | 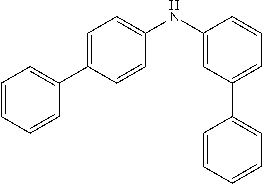 | 58% |
| 355 | 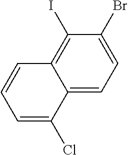 | 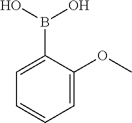 | 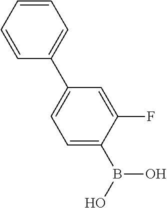 | 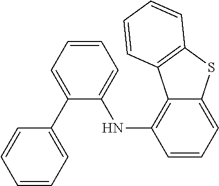 | 57% |

### Preparation Example 2: Preparation of Compound 18

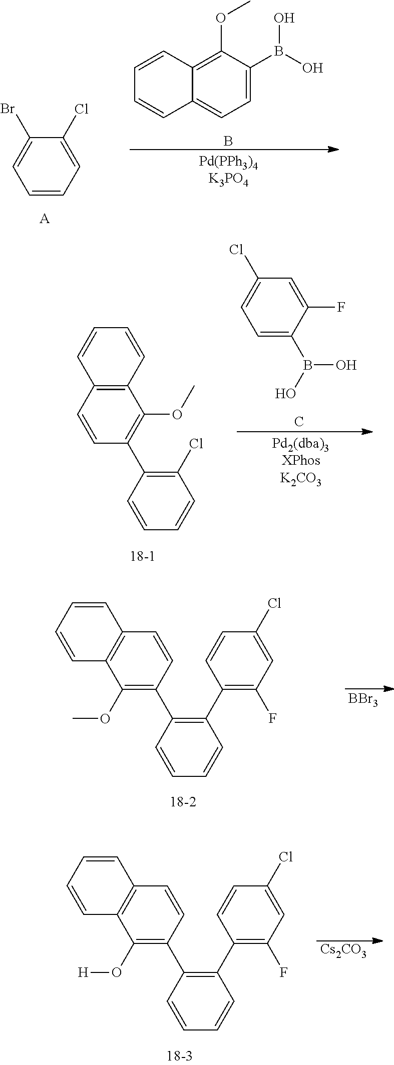

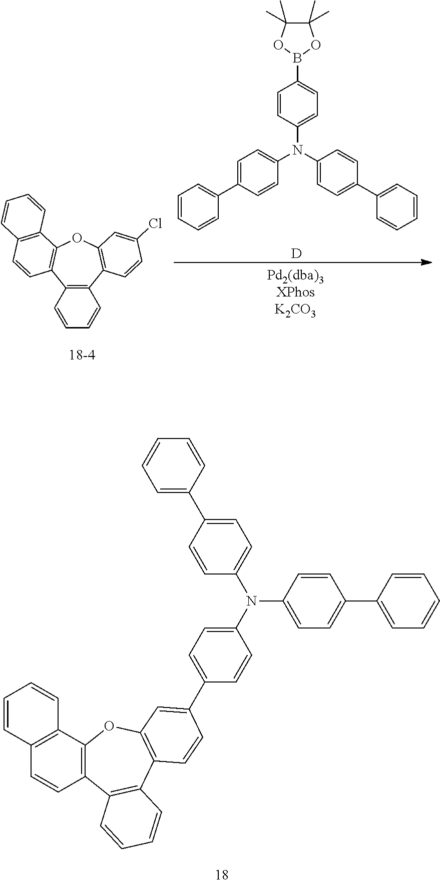

In Preparation Example C, Compound 18-1, Compound 18-2, Compound 18-3 and Compound 18-4 were prepared in the same manner as Compound 1-1, Compound 1-C, Compound 1-3 and Compound 1-4 in Preparation Example 1.

### Preparation of Compound 18

N-([1,1′-biphenyl]-4-yl)-N-(4-(4,4,5,5-tetramethyl-1,3,2-dioxaborolan-2-yl)phenyl)-[1,1′-biphenyl]-4-amine (D) (8 g, 0.015 mol, 1 eq.), Compound 18-4 (5.6 g, 0.017 mol, 1.1 eq.), tris(dibenzylideneacetone) dipalladium (Pd₂(dba)₃) (0.9 g, 0.001 mol, 0.05 eq.), XPhos (1.0 g, 0.002 mol, 0.1 eq.) and potassium carbonate (K₂CO₃) (6.2 g, 0.045 mol, 3.0 eq.) were introduced to toluene (80 ml), and the mixture was stirred for 6 hours at 100° C. Water was introduced thereto to terminate the reaction, and then the result was extracted using methylene chloride (MC) and water. After that, moisture was removed using magnesium sulfate (MgSO₄), and then the result was separated using a silica gel column to obtain Compound 18 (7.6 g, yield 70%).

Compounds were synthesized in the same manner as in Preparation Example 2 except that Intermediate A, Intermediate B, Intermediate C and Intermediate D of the following Table 2 were used instead of Compound A, Compound B, Compound C and Compound D in Preparation Example 2, respectively.

TABLE 2

| Com- | | | |
|---|---|---|---|
| pound | Intermediate | Intermediate | Intermediate |
| No. | A | B | C |
| 18 | 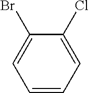 | 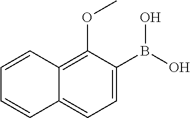 | 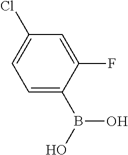 [SMILES: OB(O)c1ccc(Cl)cc1F] |
| 20 | 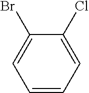 | 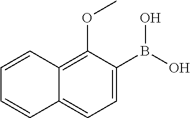 | 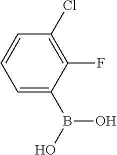 [SMILES: OB(O)c1cccc(Cl)c1F] |
| 38 | 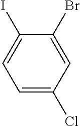 | 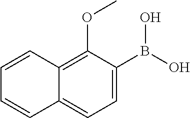 | 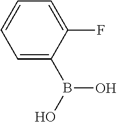 |
| 39 | 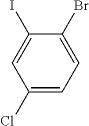 | 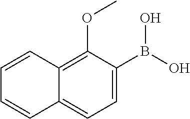 | 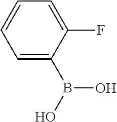 |
| 88 | 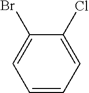 | 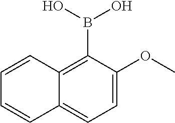 | 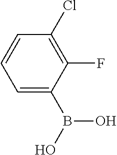 [SMILES: OB(O)c1cccc(Cl)c1F] |
| 152 | 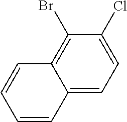 [SMILES: Clc1ccc2ccccc2c1Br] | 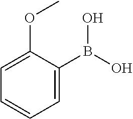 | 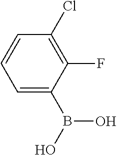 [SMILES: OB(O)c1cccc(Cl)c1F] |
| 214 | 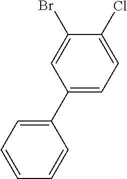 [SMILES: Clc1ccc(-c2ccccc2)cc1Br] | 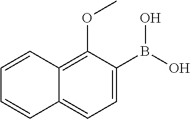 | 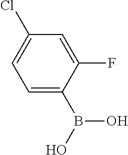 [SMILES: OB(O)c1ccc(Cl)cc1F] |
| 216 | 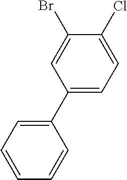 [SMILES: Clc1ccc(-c2ccccc2)cc1Br] | 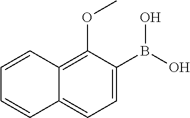 | 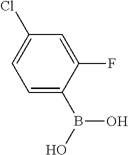 [SMILES: OB(O)c1ccc(Cl)cc1F] |
| 298 | 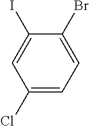 | 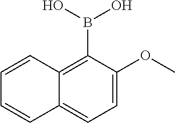 | 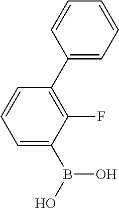 [SMILES: OB(O)c1cccc(-c2ccccc2)c1F] |
| 320 | 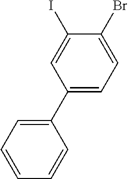 [SMILES: Brc1ccc(-c2ccccc2)cc1I] | 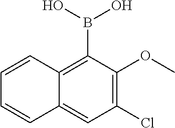 [SMILES: COc1c(Cl)cc2ccccc2c1B(O)O] | 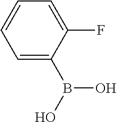 |
| 382 | 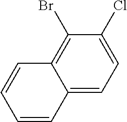 [SMILES: Clc1ccc2ccccc2c1Br] | 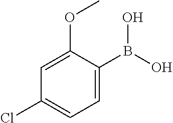 | 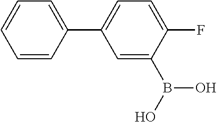 [SMILES: OB(O)c1cc(-c2ccccc2)ccc1F] |

| Com- | | |
|---|---|---|
| pound | Intermediate | |
| No. | D | Yield |
| 18 | 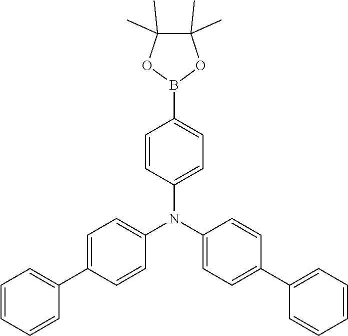 [SMILES: CC1(C)OB(c2ccc(N(c3ccc(-c4ccccc4)cc3)c3ccc(-c4ccccc4)cc3)cc2)OC1(C)C] | 53% |
| 20 | 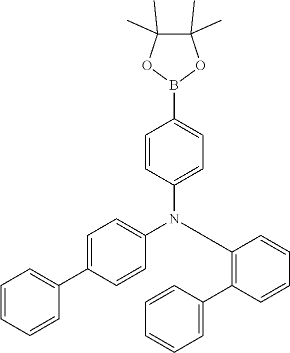 [SMILES: CC1(C)OB(c2ccc(N(c3ccc(-c4ccccc4)cc3)c3ccccc3-c3ccccc3)cc2)OC1(C)C] | 49% |
| 38 | 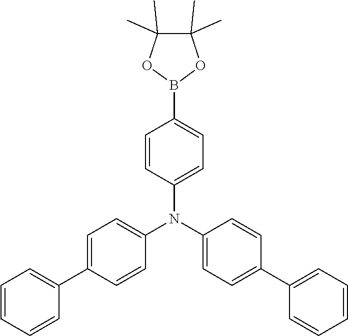 [SMILES: CC1(C)OB(c2ccc(N(c3ccc(-c4ccccc4)cc3)c3ccc(-c4ccccc4)cc3)cc2)OC1(C)C] | 51% |
| 39 | 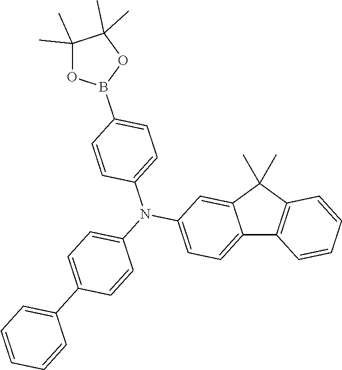 [SMILES: CC1(C)c2ccccc2-c2ccc(N(c3ccc(B4OC(C)(C)C(C)(C)O4)cc3)c3ccc(-c4ccccc4)cc3)cc21] | 47% |
| 88 | 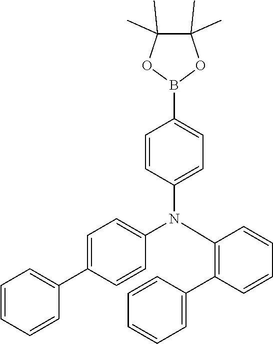 [SMILES: CC1(C)OB(c2ccc(N(c3ccc(-c4ccccc4)cc3)c3ccccc3-c3ccccc3)cc2)OC1(C)C] | 50% |
| 152 | 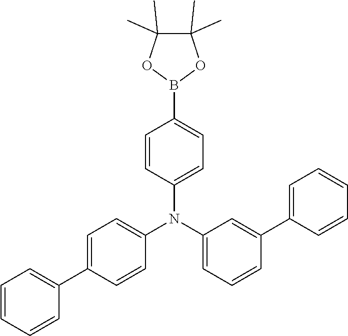 [SMILES: CC1(C)OB(c2ccc(N(c3ccc(-c4ccccc4)cc3)c3cccc(-c4ccccc4)c3)cc2)OC1(C)C] | 50% |
| 214 | 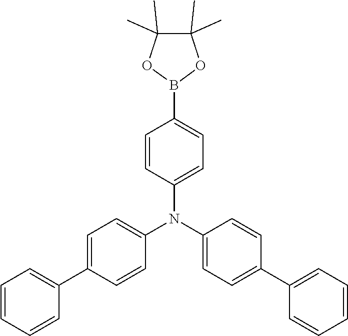 [SMILES: CC1(C)OB(c2ccc(N(c3ccc(-c4ccccc4)cc3)c3ccc(-c4ccccc4)cc3)cc2)OC1(C)C] | 53% |
| 216 | 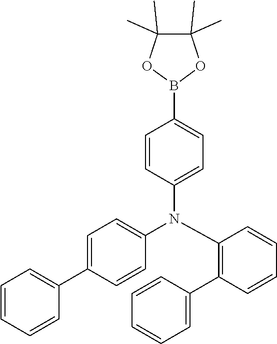 [SMILES: CC1(C)OB(c2ccc(N(c3ccc(-c4ccccc4)cc3)c3ccccc3-c3ccccc3)cc2)OC1(C)C] | 52% |
| 298 | 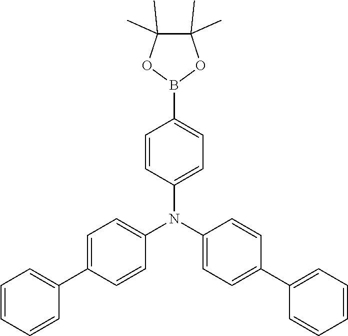 [SMILES: CC1(C)OB(c2ccc(N(c3ccc(-c4ccccc4)cc3)c3ccc(-c4ccccc4)cc3)cc2)OC1(C)C] | 49% |
| 320 | 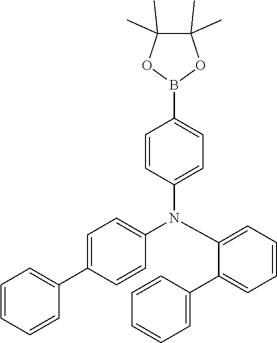 [SMILES: CC1(C)OB(c2ccc(N(c3ccc(-c4ccccc4)cc3)c3ccccc3-c3ccccc3)cc2)OC1(C)C] | 47% |
| 382 | 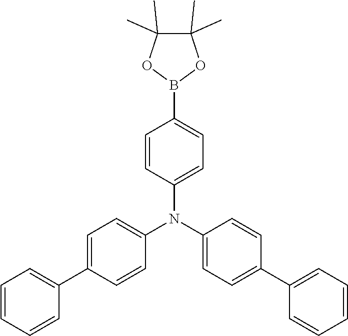 [SMILES: CC1(C)OB(c2ccc(N(c3ccc(-c4ccccc4)cc3)c3ccc(-c4ccccc4)cc3)cc2)OC1(C)C] | 49% |

### Preparation Example 3: Preparation of Compound 60

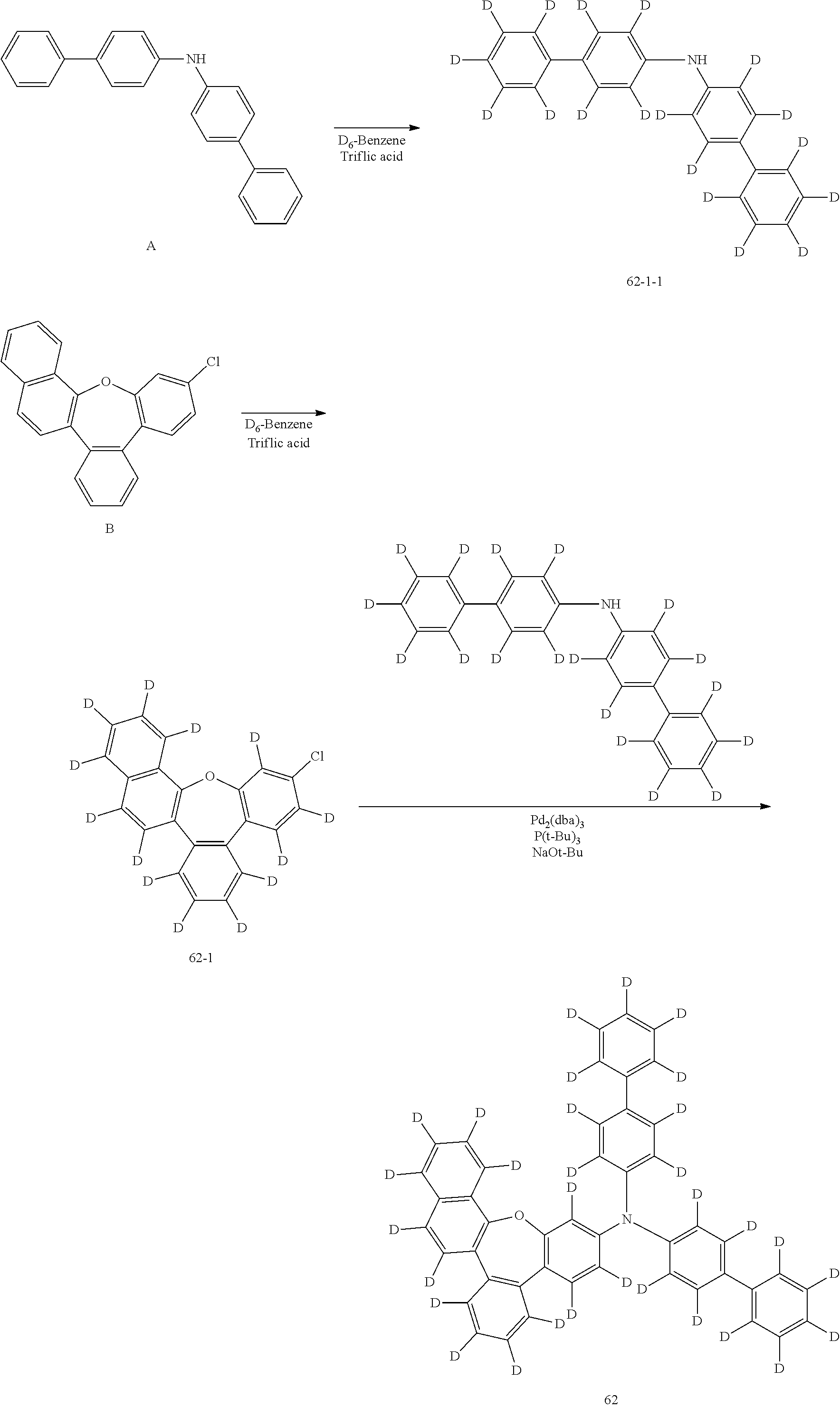

In Preparation Example 3, Compound B is the compound prepared in Preparation Example 2 (Compound 18-4), Compound 62-1 was synthesized in the same manner as the following Compound 62-1-1, and Compound 62 was synthesized in the same manner as Compound 1 of Preparation Example 1.

### Preparation of Compound 62-1-1

Di([1,1′-biphenyl]-4-yl)amine (A) (30 g, 0.093 mol, 1 eq.) was introduced to D6-benzene (300 ml) and triflic acid (57.5 ml, 0.651 mol, 7.0 eq.), and the mixture was stirred for 1 hour at 60° C. Water was introduced thereto to terminate the reaction, and then the result was extracted using methylene chloride (MC) and water. After that, moisture was removed using magnesium sulfate (MgSO₄), and then the result was separated using a silica gel column to obtain Compound 62-1-1 (29.2 g, yield 92%).

Compounds were synthesized in the same manner as in Preparation Example 3 except that Intermediate A and Intermediate B of the following Table 3 were used instead of Compound A and Compound B in Preparation Example 3, respectively.

TABLE 3

| Compound | | | |
|---|---|---|---|
| No. | Intermediate A | Intermediate B | Yield |
| 62 | 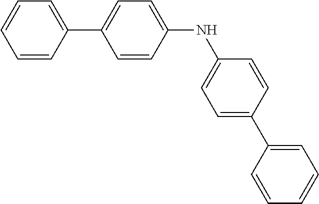 [SMILES: c1ccc(-c2ccc(Nc3ccc(-c4ccccc4)cc3)cc2)cc1] | 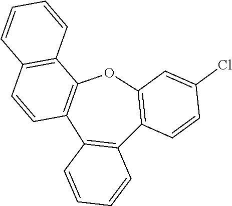 [SMILES: Clc1ccc2c(c1)Oc1c(ccc3ccccc13)-c1ccccc1-2] | 70% |
| 64 | 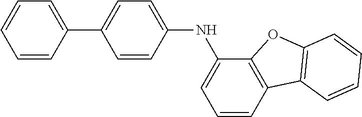 [SMILES: c1ccc(-c2ccc(Nc3cccc4c3oc3ccccc34)cc2)cc1] | 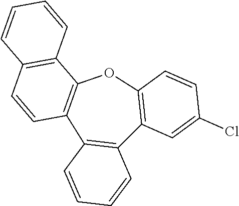 [SMILES: Clc1ccc2c(c1)-c1ccccc1-c1ccc3ccccc3c1O2] | 73% |
| 65 | 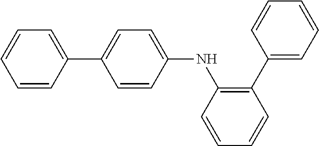 [SMILES: c1ccc(-c2ccc(Nc3ccccc3-c3ccccc3)cc2)cc1] | 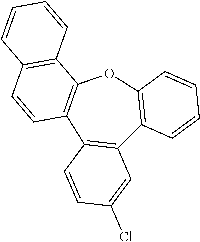 [SMILES: Clc1ccc2c(c1)-c1ccccc1Oc1c-2ccc2ccccc12] | 76% |
| 129 | 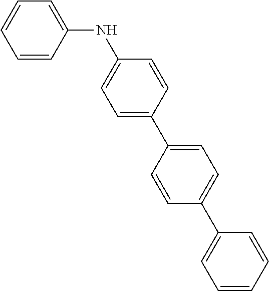 [SMILES: c1ccc(Nc2ccc(-c3ccc(-c4ccccc4)cc3)cc2)cc1] | 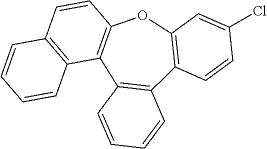 [SMILES: Clc1ccc2c(c1)Oc1ccc3ccccc3c1-c1ccccc1-2] | 70% |
| 195 | 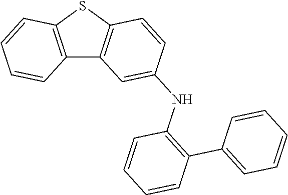 [SMILES: c1ccc(-c2ccccc2Nc2ccc3sc4ccccc4c3c2)cc1] | 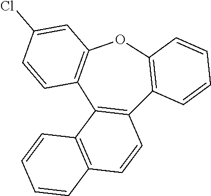 [SMILES: Clc1ccc2c(c1)Oc1ccccc1-c1ccc3ccccc3c1-2] | 71% |
| 257 | 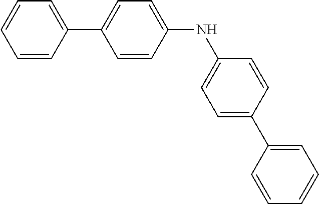 [SMILES: c1ccc(-c2ccc(Nc3ccc(-c4ccccc4)cc3)cc2)cc1] | 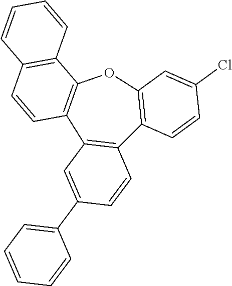 [SMILES: Clc1ccc2c(c1)Oc1c(ccc3ccccc13)-c1cc(-c3ccccc3)ccc1-2] | 71% |

### Preparation Example 4: Preparation of Compound 63

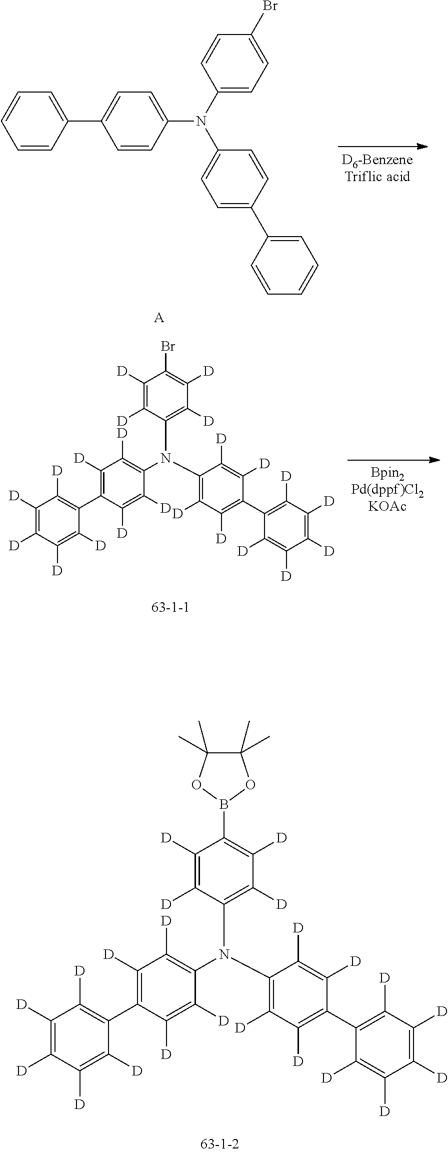

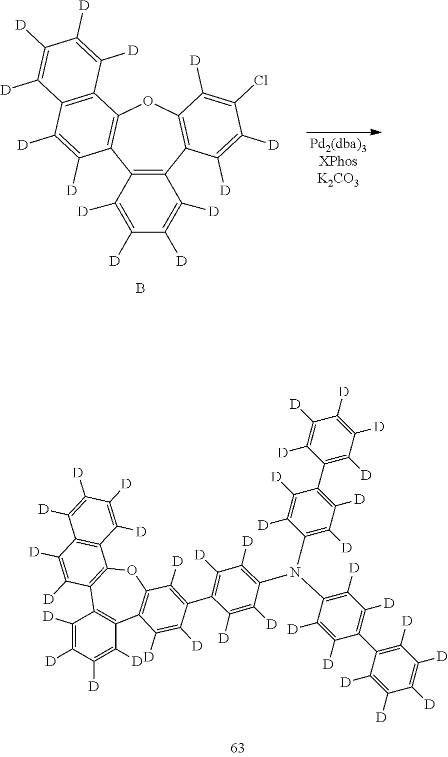

In Preparation Example 4, Compound 63-1-1 was synthesized in the same manner as in Preparation Example 3, Compound B is the compound prepared in Preparation Example 3 (Compound 62-1), and Compound 63 was synthesized in the same manner as in Preparation Example 2.

### Preparation of Compound 63-1-2

N-([1,1′-biphenyl]-4-yl-d9)-N-(4-bromophenyl-2,3,5,6-d4)-[1,1′-biphenyl]-4-amine-d9 (A) (30 g, 0.060 mol, 1 eq.), bis(pinacolato) diboron (B₂pin₂) (22.9 g, 0.090 mol, 1.5 eq.), [1,1′-bis(diphenylphosphino) ferrocene]palladium (II) dichloride (PdCl₂(dppf)) (2.2 g, 0.003 mol, 0.05 eq.) and potassium acetate (KOAc) (17.7 g, 0.180 mol, 3.0 eq.) were introduced to 1,4-dioxane (300 ml), and the mixture was stirred for 12 hours at 100° C. Water was introduced thereto to terminate the reaction, and then the result was extracted using methylene chloride (MC) and water. After that, moisture was removed using magnesium sulfate (MgSO₄), and then the result was separated using a silica gel column to obtain Compound 63-1-2 (27.8 g, yield 85%).

Compounds were synthesized in the same manner as in Preparation Example 4 except that Intermediate A and Intermediate B of the following Table 4 were used instead of Compound A and Compound B in Preparation Example 4, respectively.

TABLE 4

| Compound | | | |
|---|---|---|---|
| No. | Intermediate A | Intermediate B | Yield |
| 63 | 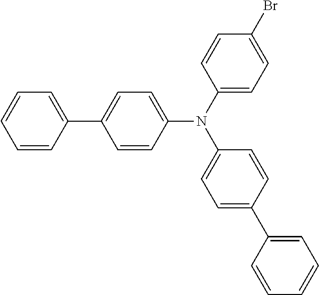 [SMILES: Brc1ccc(N(c2ccc(-c3ccccc3)cc2)c2ccc(-c3ccccc3)cc2)cc1] | 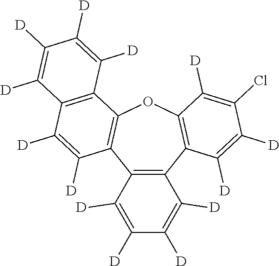 [SMILES: [2H]c1c([2H])c([2H])c2c(c1[2H])-c1c([2H])c([2H])c(Cl)c([2H])c1Oc1c-2c([2H])c([2H])c2c([2H])c([2H])c([2H])c([2H])c12] | 65% |
| 130 | 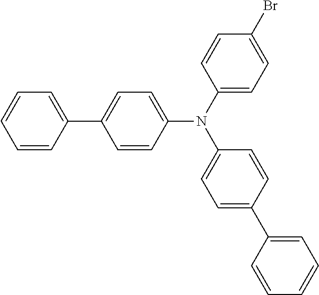 [SMILES: Brc1ccc(N(c2ccc(-c3ccccc3)cc2)c2ccc(-c3ccccc3)cc2)cc1] | 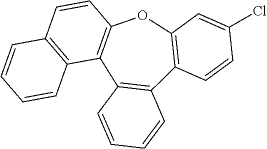 [SMILES: Clc1ccc2c(c1)Oc1ccc3ccccc3c1-c1ccccc1-2] | 67% |
| 196 | 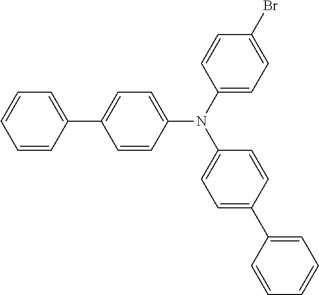 [SMILES: Brc1ccc(N(c2ccc(-c3ccccc3)cc2)c2ccc(-c3ccccc3)cc2)cc1] | 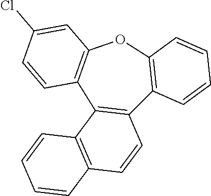 [SMILES: Clc1ccc2c(c1)Oc1ccccc1-c1ccc3ccccc3c1-2] | 67% |
| 258 | 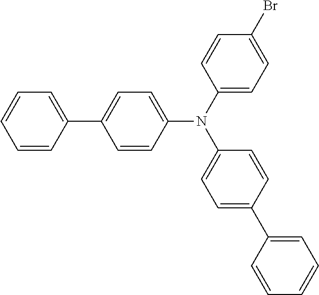 [SMILES: Brc1ccc(N(c2ccc(-c3ccccc3)cc2)c2ccc(-c3ccccc3)cc2)cc1] | 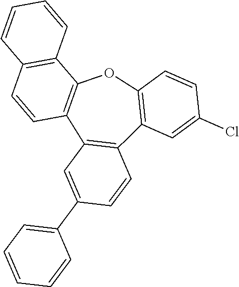 [SMILES: Clc1ccc2c(c1)-c1ccc(-c3ccccc3)cc1-c1ccc3ccccc3c1O2] | 70% |

The rest of compounds other than the compounds described in Preparation Examples 1 to 4 and Tables 1 to 4 were also prepared in the same manner as in the preparation examples described above, and the synthesis results are shown in the following Table 5 and Table 6. The following Table 5 shows measurement values of 1H NMR (CDCl₃, 200 Mz) of the compounds, and the following Table 6 shows measurement values of FD-mass spectrometry (FD-MS: field desorption mass spectrometry).

TABLE 5

| Com- | |
|---|---|
| pound | ¹H NMR (CDCl₃, 200 Mz) |
| | δ = 8.35~8.33(2H, m), 8.15(1H, d), 7.96~7.86(4H, |
| | 7.76~7.55(7H, m), 7.33~7.16(8H, m), 7.08~7.00(3H, m), |
| | 1.69(6H, s) |
| 5 | δ = 8.35~8.33(2H, m), 8.15(1H, d), 7.96~7.86(4H, m), |
| | 7.76~7.55(11H, m), 7.49~7.28(8H, m), 7.16~7.05(3H, m), |
| | 1.69(6H, s) |
| 7 | δ = 8.35~8.33(2H, m), 8.15(1H, d), 7.96~7.93(2H, m), |
| | 7.76~7.55(14H, m), 7.49~7.37(10H, m), 7.09~7.05(2H, m) |
| 9 | δ = 9.08(1H, d), 8.84(1H, d), 8.35~8.27(3H, m), 8.15(1H, d), |
| | 8.05(1H, s), 7.96~7.90(3H, m), 7.76~7.55(15H, m), |
| | 7.49~7.37(7H, m), 7.25~7.22(2H, m), 7.15(1H, d) |
| 16 | δ = 8.35~8.33(2H, m), 8.15(1H, d), 8.03~7.96(4H, m), |
| | 7.80~7.54(11H, m), 7.49~7.27(8H, m), 7.09(1H, d), |
| | 6.91~6.85(2H, m) |
| 17 | δ = 8.45(1H, d), 8.35~8.33(2H, m), 8.15~8.10(2H, m), |
| | 7.96~7.93(4H, m), 7.85(1H, d), 7.76~7.56(7H, m), |
| | 7.49~7.37(7H, m), 7.14~7.05(5H, m) |
| 18 | δ = 8.35~8.33(2H, m), 8.15(1H, d), 7.96~7.92(3H, |
| | 7.76~7.55(15H, m), 7.52~7.37(13H, m), 6.97(1H, d) |
| 20 | δ = 8.35~8.33(2H, m), 8.15~8.05(4H, m), 7.96~7.93(2H, m), |
| | 7.76~7.55(11H, m), 7.49~7.37(13H, m), 7.14~7.08(3H, m) |
| 25 | δ = 8.35~8.33(2H, m), 8.15~8.10(2H, m), 7.95~7.86(4H, m), |
| | 7.76~7.66(3H, m), 7.55(1H, d), 7.47~7.28(11H, m), |
| | 7.19~7.08(6H, m), 1.69(6H, s) |
| 28 | δ = 8.35~8.33(2H, m), 8.15(1H, d), 7.96~7.92(4H, m), |
| | 7.79~7.66(5H, m), 7.60~7.55(4H, m), 7.47~7.37(8H, m), |
| | 7.24~7.18(4H, m), 7.08~7.00(3H, m) |
| 30 | δ = 8.35~8.33(2H, m), 8.22(1H, s), 8.15~8.10(2H, m), |
| | 7.98~7.92(3H, m), 7.76~7.66(3H, m), 7.56~7.31(12H, m), |
| | 7.19~7.08(5H, m), 6.97(1H, d) |
| 33 | δ = 8.45(1H, d), 8.35~8.33(2H, m), 8.15~8.10(3H, m), |
| | 7.95~7.92(3H, m), 7.76~7.66(3H, m), 7.56~7.37(12H, m), |
| | 7.19~7.08(5H, m) |
| 38 | δ = 8.35~8.33(2H, m), 8.15~8.12(3H, 7.95(1H, d), |
| | 7.76~7.66(7H, m), 7.55~7.35(20H, m), 7.19~715(2H, m) |
| 39 | δ = 8.35~8.33(2H, m), 8.15~8.13(3H, m), 7.95~7.86(3H, m), |
| | 7.76~7.66(5H, m), 7.55~7.28(17H, m), 7.19~7.15(3H, m), |
| | 1.69(6H, s) |
| 45 | δ = 8.35~8.31(2H, m), 7.96~7.90(4H, m), 7.75~7.60(6H, m), |
| | 7.55~7.38(9H, m), 7.28~7.25(2H, m), 7.19~7.15(4H, m), |
| | 7.08~7.05(2H, m), 1.69(6H, s) |
| 53 | δ = 8.55(1H, s), 8.45(1H, d), 8.32(1H, d), 8.00~7.93(5H, m), |
| | 7.85~7.70(5H, m), 7.60~7.55(7H, m), 7.49~7.37(10H, m), |
| | 7.19~7.17(2H, m), 6.92(1H, s) |
| 56 | δ = 8.10~7.85(8H, m), 7.60~7.54(3H, m), 7.47~7.31(10H, m), |
| | 7.25~7.08(7H, m), 6.91(1H, d) |
| 62 | Structure fully substituted with D |
| 63 | Structure fully substituted with D |
| 64 | δ = 8.35~8.33(2H, m), 8.15(1H, d), 7.96~7.93(2H, m), |
| | 7.76~7.60(5H, m), 7.24~7.15(3H, m) |
| 65 | δ = 8.35~8.33(2H, m), 8.15(1H, d), 7.95~7.92(2H, m), |
| | 7.76~7.66(3H, m), 7.47~7.43(3H, m), 7.19~7.15(2H, m) |
| 74 | δ = 8.91(1H, d), 8.19(1H, d), 7.98~7.86(5H, m), 7.75~7.69(5H, |
| | m), 7.55~7.28(17H, m), 7.16~6.99(4H, m), 1.69(6H, s) |
| 75 | δ = 8.91(1H, d), 8.19(1H, d), 7.98~7.95(3H, m), 7.75~7.69(5H, |
| | m), 7.55~7.28(18H, m), 7.09~6.99(3H, m) |
| 77 | δ = 9.08(1H, d), 8.91~8.84(2H, m), 8.27(1H, d), 8.19(1H, d), |
| | 8.05(1H, s), 7.98~7.90(4H, m), 7.75~7.60(8H, m), |
| | 7.55~7.37(13H, m), 7.28~7.24(2H, m), 7.15(1H, d), 6.99(1H, |
| | d) |
| 78 | δ = 8.91(1H, d), 8.19(1H, d), 8.00~7.95(4H, m), 7.75~7.70(2H, |
| | m), 7.64~7.54(5H, m), 7.49~7.37(6H, m), 7.31~7.15(8H, m), |
| | 6.99~6.95(2H, m) |
| 79 | δ = 8.91(1H, d), 8.45(1H, d), 8.19(1H, d), 8.12~8.10(2H, m), |
| | 7.98~7.93(4H, m), 7.60~7.56(3H, m), 7.49~7.37(10H, m), |
| | 7.25~7.22(2H, m), 7.15~7.08(4H, m), 6.99(1H, d) |
| 88 | δ = 8.91(1H, d), 8.19(1H, d), 8.10~7.96(6H, m), 7.75~7.73(2H, |
| | m), 7.60~7.55(6H, m), 7.49~7.37(15H, m), 7.14~7.08(3H, m), |
| | 6.99(1H, d) |
| 93 | δ = 8.91(1H, d), 8.19(1H, d), 7.98~7.90(4H, m), 7.75~7.72(2H, |
| | m), 7.55~7.49(5H, m), 7.47~7.37(9H, m), 7.28~7.16(6H, m), |
| | 6.99(1H, d), 1.69(6H, s) |
| 99 | δ = 8.91(1H, d), 8.45(1H, d), 8.19(1H, d), 8.12~8.10(2H, m), |
| | 7.98~7.92(4H, m), 7.56~7.37(14H, m), 7.19~7.08(5H, m), |
| | 6.99(1H, d) |
| 100 | δ = 8.91(1H, d), 8.19(1H, d), 8.08(1H, d), 8.02~7.92(5H, m), |
| | 7.75(2H, d), 7.55~7.49(8H, m), 7.45~7.31(12H, m) |
| | 7.19~7.17(2H, m), 6.99(1H, d) |
| 113 | δ = 7.99~7.95(4H, m), 7.90(1H, d), 7.77~7.75(3H, m), |
| | 7.65~7.60(4H, m), 7.55~7.38(8H, m), 7.29~7.27(2H, m), |
| | 7.19~7.17(4H, m), 7.06(1H, d), 6.80~6.77(2H, m), 1.68(6H, |
| | s) |
| 119 | δ = 8.45(1H, d), 7.96~7.93(4H, m), 7.83(1H, d), 7.77~7.75(2H, |
| | m), 7.65~7.59(4H, m), 7.56~7.41(10H, m), 7.27(1H, s), |
| | 7.19~7.17(4H, m), 6.87(1H, s), 6.72(1H, d) |
| 124 | δ = 8.91(1H, d), 8.26(1H, d), 8.10(1H, d), 7.98~7.95(4H, m), |
| | 7.60~7.54(3H, m), 7.45~7.39(7H, m), 7.35~7.25(5H, m), |
| | 7.22~7.18(3H, m), 7.14~7.08(3H, m), 6.91(1H, d) |
| 129 | Structure fully substituted with D |
| 130 | δ = 8.91(1H, d), 8.19(1H, d), 7.98~7.92(4H, m), 7.60~7.52(3H, |
| | m), 7.44~7.37(2H, m), 6.99~6.97(2H, m) |
| 135 | δ = 8.93(1H, d), 8.31~8.26(2H, m), 7.95(1H, d), 7.78~7.71(5H, |
| | m), 7.61~7.54(5H, m), 7.49~7.37(10H, m), 7.27~7.11(5H, m) |
| 138 | δ = 8.93(1H, d), 8.31~8.26(2H, m), 7.95(1H, d), 7.90(1H, d), |
| | 7.86(1H, d), 7.75~7.69(6H, m), 7.60~7.54(4H, m), |
| | 7.49~7.37(10H, m), 7.33(1H, s), 7.28(1H, t), 7.19~7.16(3H, |
| | m), 7.09(1H, d), 7.05(1H, s), 1.69(6H, s) |
| 140 | δ = 8.93(1H, d), 8.31~8.26(2H, m), 7.95(1H, d), 7.75~7.69(4H, |
| | m), 7.55~7.37(10H, m), 7.25~7.18(8H, m), 7.09~7.00(5H, m) |
| 142 | δ = 8.93(1H, d), 8.31-8.26(2H, m), 8.03(1H, s), 7.98~7.95(2H, |
| | m), 7.80~7.72(4H, m), 7.55~7.39(10H, m), 7.37~7.31(3H, m), |
| | 7.24~7.15(5H, m), 6.91(1H, d) |
| 143 | δ = 8.93(1H, d), 8.45(1H, d), 8.31~8.26(2H, m), 8.10(1H, d), |
| | 7.95~7.93(3H, m), 7.85(1H, d), 7.72(1H, d), 7.54~7.37(11H, |
| | m), 7.19~7.08(8H, m) |
| 152 | δ = 8.93(1H, d), 8.31~8.26(2H, m), 8.05~8.03(2H, m), 7.95(1H, |
| | d), 7.75~7.72(5H, m), 7.55~7.37(19H, m), 7.27(1H, s), |
| | 7.19~7.17(4H, m) |
| 157 | δ = 8.93(1H, d), 8.33(1H, d), 7.95~7.86(4H, m), 7.75~7.73(2H, |
| | m), 7.55~7.28(15H, m), 7.19~7.14(6H, m), 1.69(6H, s) |
| 163 | δ = 8.59(1H, d), 8.45~8.41(2H, m), 8.10(1H, d), 7.95~7.92(3H, |
| | m), 7.72(1H, d), 7.59~7.39(14H, m), 7.19~7.08(7H, m) |
| 164 | δ = 8.16(1H, d), 8.08(1H, d), 8.02~7.95(4H, m), 7.75~7.72(3H, |
| | m), 7.55~7.45(12H, m), 7.41~7.31(7H, m), 7.19~7.16(4H, m), |
| | 6.94(1H, s) |
| 177 | δ = 8.93(1H, d), 8.31~8.26(2H, m), 7.95~7.90(2H, m), |
| | 7.75~7.69(4H, m), 7.62~7.40(10H, m), 7.38~7.17(7H, m), |
| | 7.09~7.05(3H, m), 1.69(6H, s) |
| 182 | δ = 8.93(1H, d), 8.31~8.22(3H, m), 8.10(1H, d), 7.98~7.95(2H, |
| | m), 7.72(1H, d), 7.56~7.37(11H, m), 7.31~7.23(3H, m), |
| | 7.19~7.08(6H, m), 6.97(1H, d) |
| 183 | δ = 8.93(1H, d), 8.45(1H, d), 8.31~8.26(2H, m), 8.10(1H, d), |
| | 7.95~7.93(2H, m), 7.72(1H, d), 7.59~7.37(13H, m), |
| | 7.24~7.08(8H, m) |
| 195 | δ = 8.93(1H, d), 8.31~8.26(2H, m), 7.95(1H, d), 7.72~7.69(2H, |
| | m), 7.54~7.40(3H, m), 7.19~7.05(4H, m) |
| 196 | Structure fully substituted with D |
| 198 | δ = 8.35~8.33(2H, m), 8.15~8.13(3H, m), 7.96~7.92(2H, m), |
| | 7.79~7.70(10H, m), 7.55~7.35(11H, m), 7.24~7.21(2H, m), |
| | 7.09~7.00(5H, m) |
| 205 | δ = 9.08(1H, d), 8.84(1H, d), 8.35~8.27(3H, m), 8.15~8.13(3H, |
| | m), 8.05(1H, s), 7.90(1H, d), 7.76~7.63(10H, m), |
| | 7.55~7.37(16H, m), 7.24~7.23(2H, m), 7.15(1H, d), |
| 212 | δ = 8.35~8.33(2H, m), 8.15~8.13(3H, m), 8.03~7.98(2H, m), |
| | 7.80~7.66(9H, m), 7.55~7.31(14H, m), 7.09~7.05(2H, m), |
| | 6.91(1H, d), |
| 214 | δ = 8.35~8.33(2H, m), 8.15~8.13(3H, m), 7.92(1H, d), |
| | 7.76~7.66(9H, m), 7.55~7.35(23H, m), 6.97(1H, d), |
| 216 | δ = 8.35~8.33(2H, m), 8.15~8.10(4H, m), 7.92(1H, d), |
| | 7.75~7.66(7H, m), 7.55~7.35(21H, m), 7.14~7.08(3H, m), |
| | 6.97(1H, d), |
| 230 | δ = 8.35~8.32(2H, m), 8.15(1H, d), 8.05~8.02(2H, m), |
| | 7.92~7.89(4H, m), 7.75~7.66(5H, m), 7.55~7.37(19H, m), |
| | 7.28~7.16(8H, m) |
| 231 | δ = 8.35~8.32(2H, m), 8.15(1H, d), 8.05~8.02(2H, m), |
| | 7.92~7.90(3H, m), 7.76~7.66(5H, m), 7.55~7.37(19H, m), |
| | 7.28~7.10(9H, m), 6.86~6.84(2H, m) |
| 238 | δ = 8.35~8.31(2H, m), 7.96~7.90(3H, m), 7.77~7.60(8H, m), |
| | 7.55~7.37(12H, m), 7.24~7.22(2H, m), 7.08~7.00(4H, m) |
| 252 | δ = 8.35~8.31(2H, m), 8.10(1H, d), 7.98~7.92(4H, m), |
| | 7.75~7.60(5H, m), 7.55~7.25(15H, m), 7.14~7.08(4H, m), |
| | 6.97(1H, d), 6.91(1H, d) |
| 257 | Structure fully substituted with D |
| 258 | Structure fully substituted with D |
| 267 | δ = 8.91(1H, d), 8.19~8.13(3H, m), 7.98(1H, d), 7.75~7.69(7H, |
| | m), 7.55~7.35(20H, m), 7.09~6.99(3H, m) |
| 298 | δ = 8.91(1H, d), 8.19~8.13(3H, m), 8.05~7.98(3H, m), |
| | 7.75~7.72(4H, m), 7.55~7.35(27H, m), 6.99(1H, d) |
| 304 | δ = 8.98(1H, d), 8.84(1H, d), 8.11(1H, d), 7.99~7.90(5H, m), |
| | 7.75~7.60(12H, m), 7.55~7.37(12H, m), 6.97(1H, d), |
| | 6.80~6.77(2H, m) |
| 320 | δ = 8.97(1H, d), 8.17~8.10(4H, m), 7.95(1H, d), 7.76~7.72(5H, |
| | m), 7.55~7.35(23H, m), 7.19~7.08(5H, m) |
| 329 | δ = 8.93(1H, d), 8.31~8.26(2H, m), 7.90~7.86(3H, m), |
| | 7.77~7.69(7H, m), 7.55~7.37(15H, m), 7.33~7.28(2H, m), |
| | 7.16~7.05(3H, m), 1.69(6H, s) |
| 351 | δ = 8.93(1H, d), 8.33(1H, d), 7.95~7.92(2H, m), 7.77~7.73(7H, |
| | m), 7.55~7.37(18H, m), 7.27(1H, s), 7.19~7.14(5H, m) |
| 355 | δ = 8.59(1H, d), 8.45~8.41(2H, m), 8.10(1H, d), 7.95~7.92(3H, |
| | m), 7.75~7.72(3H, m), 7.59~7.52(4H, m), 7.49~7.37(13H, m), |
| | 7.19~7.08(5H, m), 6.97(1H, d) |
| 382 | δ = 8.93(1H, d), 8.31~8.26(2H, m), 7.92~7.90(2H, m), |
| | 7.75~7.70(8H, m), 7.55~7.37(25H, m), 6.97(1H, d) |

TABLE 6

| Compound | FD-MS | Compound | FD-MS |
|---|---|---|---|
| 1 | m/z = 577.24 | 5 | m/z = 653.27 |
| | (C₄₃H₃₁NO = 577.73) | | (C₄₉H₃₅NO = 653.82) |
| 7 | m/z = 613.24 | 9 | m/z = 713.27 |
| | (C₄₆H₃₁NO = 613.76) | | (C₅₄H₃₅NO = 713.88) |
| 16 | m/z = 627.22 | 17 | m/z = 643.20 |
| | (C₄₆H₂₉NO₂ = 627.74) | | (C₄₆H₂₉NOS = 643.80) |
| 18 | m/z = 689.27 | 20 | m/z = 689.27 |
| | (C₅₂H₃₅NO = 689.86) | | (C₅₂H₃₅NO = 689.86) |
| 25 | m/z = 653.27 | 28 | m/z = 613.24 |
| | (C₄₉H₃₅NO = 653.82) | | (C₄₆H₃₁NO = 613.76) |
| 30 | m/z = 627.22 | 33 | m/z = 643.20 |
| | (C₄₆H₂₉NO₂ = 627.74) | | (C₄₆H₂₉NOS = 643.80) |
| 38 | m/z = 689.27 | 39 | m/z = 729.30 |
| | (C₅₂H₃₅NO = 689.86) | | (C₅₅H₃₉NO = 729.92) |
| 45 | m/z = 653.27 | 53 | m/z = 719.23 |
| | (C₄₉H₃₅NO = 653.82) | | (C₅₂H₃₃NOS = 719.90) |
| 56 | m/z = 627.22 | 62 | m/z = 644.44 |
| | (C₄₆H₂₉NO₂ = 627.74) | | (C₄₆D₃₁NO = 644.95) |
| 63 | m/z = 724.49 | 64 | m/z = 643.32 |
| | (C₅₂D₃₅NO = 725.07) | | (C₄₆H₁₃D₁₆NO₂ = 643.84) |
| 65 | m/z = 631.35 | 74 | m/z = 729.30 |
| | (C₄₆H₁₃D₁₈NO = 631.87) | | (C₅₅H₃₉NO = 729.92) |
| 75 | m/z = 613.24 | 77 | m/z = 713.27 |
| | (C₄₆H₃₁NO = 613.76) | | (C₅₄H₃₅NO = 713.88) |
| 78 | m/z = 627.22 | 79 | m/z = 643.20 |
| | (C₄₆H₂₉NO₂ = 627.74) | | (C46H29NOS = 643.80) |
| 88 | m/z = 689.27 | 93 | m/z = 653.27 |
| | (C₅₂H₃₅NO = 689.86) | | (C₄₉H₃₅NO = 653.82) |
| 99 | m/z = 643.20 | 100 | m/z = 703.25 |
| | (C₄₆H₂₉NOS = 643.80) | | (C₅₂H₃₃NO₂ = 703.84) |
| 113 | m/z = 653.27 | 119 | m/z = 643.20 |
| | (C₄₉H₃₅NO = 653.82) | | (C₄₆H₂₉NOS = 643.80) |
| 124 | m/z = 627.22 | 129 | m/z = 644.44 |
| | (C₄₆H₂₉NO₂ = 627.74) | | (C₄₆D₃₁NO = 644.95) |
| 130 | m/z = 711.41 | 135 | m/z = 587.22 |
| | (C₅₂H₁₃D₂₂NO = 711.99) | | (C₄₄H₂₉NO = 587.72) |
| 138 | m/z = 729.30 | 140 | m/z = 613.24 |
| | (C₅₅H₃₉NO = 729.92) | | (C₄₆H₃₁NO = 613.76) |
| 142 | m/z = 627.22 | 143 | m/z = 643.20 |
| | (C₄₆H₂₉NO₂ = 627.74) | | (C₄₆H₂₉NOS = 643.80) |
| 152 | m/z = 689.27 | 157 | m/z = 653.27 |
| | (C₅₂H₃₅NO = 689.86) | | (C₄₉H₃₅NO = 653.82) |
| 163 | m/z = 643.20 | 164 | m/z = 703.25 |
| | (C₄₆H₂₉NOS = 643.80) | | (C₅₂H₃₃NO₂ = 703.84) |
| 177 | m/z = 653.27 | 182 | m/z = 627.22 |
| | (C₄₉H₃₅NO = 653.82) | | (C₄₆H₂₉NO₂ = 627.74) |
| 183 | m/z = 643.20 | 195 | m/z = 659.30 |
| | (C₄₆H₂₉NOS = 643.80) | | (C₄₆H₁₃D₁₆NOS = 659.90) |
| 196 | m/z = 724.49 | 198 | m/z = 689.27 |
| | (C₅₂D₃₅NO = 725.07) | | (C₅₂H₃₅NO = 689.86) |
| 205 | m/z = 789.30 | 212 | m/z = 703.25 |
| | (C₆₀H₃₉NO = 789.98) | | (C₅₂H₃₃NO₂ = 703.84) |
| 214 | m/z = 765.30 | 216 | m/z = 765.30 |
| | (C₅₈H₃₉NO = 765.96) | | (C₅₈H₃₉NO = 765.96) |
| 230 | m/z = 851.32 | 231 | m/z = 853.33 |
| | (C₆₅H₄₁NO = 852.05) | | (C₆₅H₄₃NO = 854.06) |
| 238 | m/z = 613.24 | 252 | m/z = 703.25 |
| | (C46H31NO = 613.76) | | (C₅₂H₃₃NO₂ = 703.84) |
| 257 | m/z = 724.49 | 258 | m/z = 804.55 |
| | (C₅₂D₃₅NO = 725.07) | | (C₅₈D₃₉NO = 805.19) |
| 267 | m/z = 689.27 | 298 | m/z = 765.30 |
| | (C₅₂H₃₅NO = 689.86) | | (C₅₈H₃₉NO = 765.96) |
| 304 | m/z = 713.27 | 320 | m/z = 765.30 |
| | (C₅₄H₃₅NO = 713.88) | | (C₅₈H₃₉NO = 765.96) |
| 329 | m/z = 729.30 | 351 | m/z = 689.27 |
| | (C₅₅H₃₉NO = 729.92) | | (C₅₂H₃₅NO = 689.86) |
| 355 | m/z = 719.23 | 382 | m/z = 765.30 |
| | (C₅₂H₃₃NOS = 719.90) | | (C₅₈H₃₉NO = 765.96) |

### Experimental Example

### Experimental Example 1

### (1) Manufacture of Organic Light Emitting Device

A transparent electrode indium tin oxide (ITO) thin film obtained from glass for an OLED (manufactured by Samsung Corning Advanced Glass) was ultrasonic cleaned using trichloroethylene, acetone, ethanol and distilled water sequentially for 5 minutes each, and then stored in isopropanol before use. Then, the ITO substrate was installed in a substrate folder of a vacuum deposition apparatus, and to a cell in the vacuum deposition apparatus, the following 4,4′,4″-tris(N,N-(2-naphthyl)-phenylamino)triphenylamine (2-TNATA) was introduced.

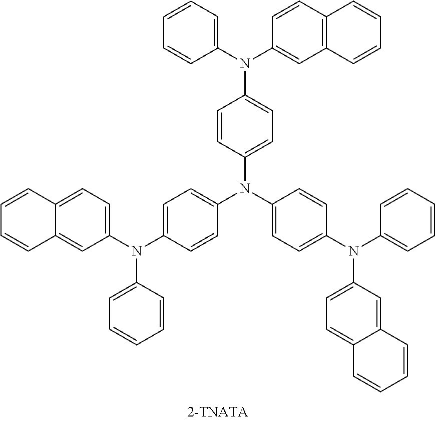 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then the 2-TNATA was evaporated by applying a current to the cell to deposit a hole injection layer having a thickness of 600 Å on the ITO substrate. To another cell in the vacuum deposition apparatus, the following N,N′-bis(α-naphthyl)-N,N′-diphenyl-4,4′-diamine (NPB) was introduced, and evaporated by applying a current to the cell to deposit a hole transport layer having a thickness of 300 Å on the hole injection layer.

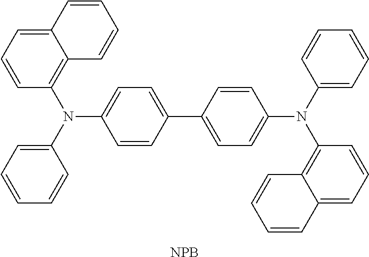 [SMILES: c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transport layer as above, a blue light emitting material having the following structure was deposited thereon as a light emitting layer. Specifically, on one cell in the vacuum deposition apparatus, H1 that is a blue light emitting host material was vacuum deposited to a thickness of 200 Å, and D1 that is a blue light emitting dopant material was vacuum deposited thereon to a thickness of 58 with respect to the host material.

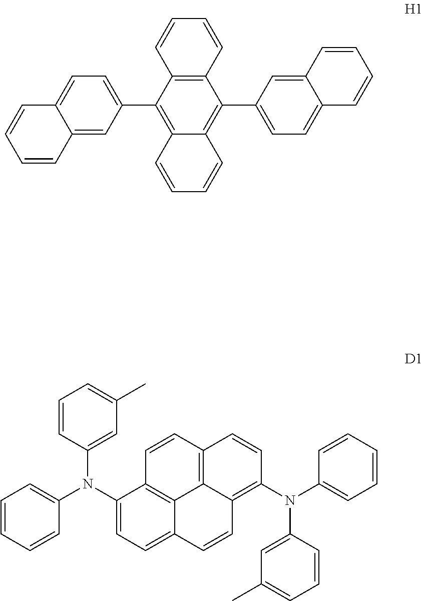

Subsequently, a compound of the following Structural Formula E1 was deposited to a thickness of 300 Å as an electron transport layer.

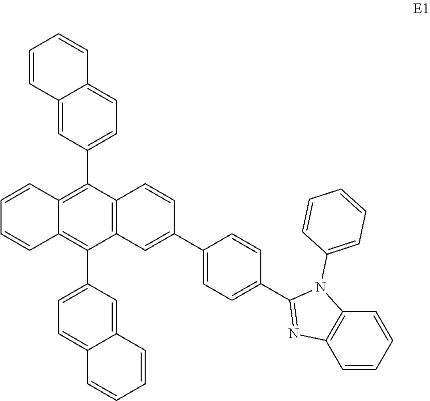 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

Lithium fluoride (LiF) was deposited to a thickness of 10 Å as an electron injection layer, and an aluminum (Al) negative electrode was deposited to a thickness of 1,000 Å, and as a result, an organic light emitting device was manufactured.

Meanwhile, all the organic compounds required to manufacture the organic light emitting device were vacuum sublimation purified under 10⁻⁸ torr to 10⁻⁶ torr for each material to be used in the manufacture of the organic light emitting device.

Organic light emitting devices were manufactured in the same manner as in Experimental Example 1 except that compounds listed in the following Table 7 were used instead of NPB used when forming the hole transport layer in Experimental Example 1.

### (2) Driving Voltage and Light Emission Efficiency of Organic Light Emitting Device

For each of the organic light emitting devices manufactured as above, electroluminescent (EL) properties were measured using M7000 manufactured by McScience Inc., and with the measurement results, a lifetime T₉₅, a time taken for luminance to become 95% with respect to initial luminance, was measured when standard luminance was 700 cd/m2 through a lifetime measurement system (M6000) manufactured by McScience Inc.

Results of measuring driving voltage, light emission efficiency, color coordinate (CIE) and lifetime of the blue organic light emitting devices manufactured according to the present disclosure are shown in the following Table 7.

TABLE 7

| | | | Light | | |
|---|---|---|---|---|---|
| | | | Emis- | | |
| | | | sion | | |
| | | Driving | Effi- | | Life |
| | | Voltage | ciency | CIE | time |
| | Compound | (V) | (cd/A) | (x, y) | (T95) |
| Example 1 | 1 | 4.55 | 6.85 | (0.134, 0.100) | 91 |
| Example 2 | 5 | 4.50 | 6.98 | (0.134, 0.100) | 90 |
| Example 3 | 7 | 4.43 | 6.80 | (0.133, 0.100) | 95 |
| Example 4 | 9 | 4.40 | 6.88 | (0.134, 0.100) | 96 |
| Example 5 | 16 | 4.58 | 6.82 | (0.134, 0.101) | 98 |
| Example 6 | 17 | 4.41 | 6.94 | (0.134, 0.100) | 93 |
| Example 7 | 18 | 4.46 | 6.84 | (0.133, 0.101) | 94 |
| Example 8 | 20 | 4.48 | 6.87 | (0.134, 0.100) | 91 |
| Example 9 | 25 | 4.61 | 6.85 | (0.133, 0.100) | 90 |
| Example 10 | 28 | 4.53 | 6.92 | (0.133, 0.101) | 93 |
| Example 11 | 30 | 4.57 | 6.90 | (0.134, 0.100) | 92 |
| Example 12 | 33 | 4.60 | 6.81 | (0.134, 0.100) | 90 |
| Example 13 | 38 | 4.56 | 6.83 | (0.134, 0.101) | 94 |
| Example 14 | 39 | 4.59 | 6.85 | (0.133, 0.100) | 90 |
| Example 15 | 45 | 4.54 | 6.85 | (0.133, 0.100) | 92 |
| Example 16 | 53 | 4.58 | 6.79 | (0.133, 0.100) | 90 |
| Example 17 | 56 | 4.57 | 6.81 | (0.133, 0.100) | 90 |
| Example 18 | 62 | 4.47 | 6.84 | (0.133, 0.101) | 100 |
| Example 19 | 63 | 4.46 | 6.84 | (0.133, 0.101) | 98 |
| Example 20 | 64 | 4.59 | 6.81 | (0.134, 0.100) | 95 |
| Example 21 | 65 | 4.54 | 6.88 | (0.134, 0.101) | 95 |
| Example 22 | 74 | 4.53 | 6.95 | (0.134, 0.100) | 90 |
| Example 23 | 75 | 4.50 | 6.78 | (0.134, 0.100) | 92 |
| Example 24 | 77 | 4.45 | 6.83 | (0.133, 0.100) | 91 |
| Example 25 | 78 | 4.48 | 6.88 | (0.134, 0.101) | 93 |
| Example 26 | 79 | 4.50 | 6.93 | (0.134, 0.100) | 94 |
| Example 27 | 88 | 4.49 | 6.78 | (0.133, 0.101) | 90 |
| Example 28 | 93 | 4.53 | 6.93 | (0.134, 0.100) | 90 |
| Example 29 | 99 | 4.55 | 6.75 | (0.134, 0.100) | 91 |
| Example 30 | 100 | 4.50 | 6.78 | (0.134, 0.101) | 93 |
| Example 31 | 113 | 4.53 | 6.91 | (0.134, 0.100) | 91 |
| Example 32 | 119 | 4.58 | 6.90 | (0.134, 0.100) | 91 |
| Example 33 | 124 | 4.57 | 6.75 | (0.133, 0.100) | 90 |
| Example 34 | 129 | 4.53 | 6.79 | (0.134, 0.100) | 96 |
| Example 35 | 130 | 4.52 | 6.82 | (0.133, 0.100) | 95 |
| Example 36 | 135 | 4.65 | 6.80 | (0.134, 0.100) | 92 |
| Example 37 | 138 | 4.50 | 6.92 | (0.134, 0.100) | 90 |
| Example 38 | 140 | 4.60 | 6.83 | (0.134, 0.101) | 93 |
| Example 39 | 142 | 4.58 | 6.81 | (0.134, 0.101) | 93 |
| Example 40 | 143 | 4.58 | 6.85 | (0.134, 0.101) | 93 |
| Example 41 | 152 | 4.52 | 6.78 | (0.133, 0.100) | 92 |
| Example 42 | 157 | 4.60 | 6.88 | (0.134, 0.101) | 88 |
| Example 43 | 163 | 4.58 | 6.83 | (0.134, 0.101) | 90 |
| Example 44 | 164 | 4.62 | 6.80 | (0.134, 0.101) | 93 |
| Example 45 | 177 | 4.59 | 6.85 | (0.134, 0.100) | 90 |
| Example 46 | 182 | 4.60 | 6.81 | (0.134, 0.100) | 89 |
| Example 47 | 183 | 4.55 | 6.83 | (0.134, 0.100) | 90 |
| Example 48 | 195 | 4.59 | 6.85 | (0.134, 0.101) | 97 |
| Example 49 | 196 | 4.61 | 6.83 | (0.134, 0.100) | 91 |
| Example 50 | 198 | 4.48 | 6.81 | (0.133, 0.100) | 94 |
| Example 51 | 205 | 4.48 | 6.79 | (0.134, 0.100) | 91 |
| Example 52 | 212 | 4.52 | 6.83 | (0.134, 0.100) | 94 |
| Example 53 | 214 | 4.48 | 6.83 | (0.133, 0.101) | 94 |
| Example 54 | 216 | 4.51 | 6.84 | (0.133, 0.100) | 95 |
| Example 55 | 230 | 4.61 | 6.88 | (0.134, 0.100) | 89 |
| Example 56 | 231 | 4.60 | 6.90 | (0.134, 0.100) | 90 |
| Example 57 | 238 | 4.55 | 6.80 | (0.134, 0.100) | 89 |
| Example 58 | 252 | 4.53 | 6.81 | (0.134, 0.100) | 90 |
| Example 59 | 257 | 4.50 | 6.78 | (0.133, 0.101) | 100 |
| Example 60 | 258 | 4.50 | 6.84 | (0.133, 0.101) | 100 |
| Example 61 | 267 | 4.53 | 6.75 | (0.134, 0.100) | 93 |
| Example 62 | 298 | 4.52 | 6.80 | (0.133, 0.101) | 96 |
| Example 63 | 304 | 4.55 | 6.78 | (0.134, 0.100) | 91 |
| Example 64 | 320 | 4.53 | 6.80 | (0.134, 0.101) | 91 |
| Example 65 | 329 | 4.48 | 6.83 | (0.134, 0.100) | 91 |
| Example 66 | 351 | 4.60 | 6.73 | (0.133, 0.100) | 89 |
| Example 67 | 355 | 4.58 | 6.75 | (0.133, 0.101) | 90 |
| Example 68 | 382 | 4.50 | 6.82 | (0.134, 0.100) | 95 |
| Comparative | NPB | 5.15 | 5.94 | (0.134, 0.100) | 56 |
| Example 1 | | | | | |
| Comparative | Comparative | 5.25 | 6.03 | (0.134, 0.100) | 58 |
| Example 2 | Compound A | | | | |
| Comparative | Comparative | 5.23 | 6.12 | (0.133, 0.100) | 57 |
| Example 3 | Compound B | | | | |
| Comparative | Comparative | 5.31 | 6.21 | (0.134, 0.100) | 68 |
| Example 4 | Compound C | | | | |
| Comparative | Comparative | 4.95 | 6.03 | (0.133, 0.101) | 63 |
| Example 5 | Compound D | | | | |
| Comparative | Comparative | 4.90 | 6.10 | (0.134, 0.100) | 60 |
| Example 6 | Compound E | | | | |
| Comparative | Comparative | 4.91 | 6.30 | (0.133, 0.100) | 72 |
| Example 7 | Compound F | | | | |

The compound of Comparative Example 1 is as follows:

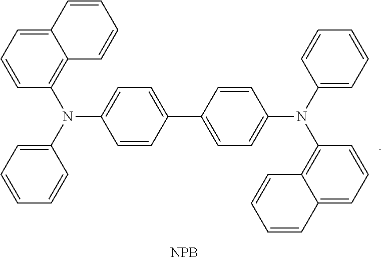 [SMILES: c1ccc(N(c2ccc(-c3ccc(N(c4ccccc4)c4cccc5ccccc45)cc3)cc2)c2cccc3ccccc23)cc1 ; "NPB"]

In addition, the compounds of Comparative Examples 2 to 7 are the same as the following Comparative Compounds A to F:

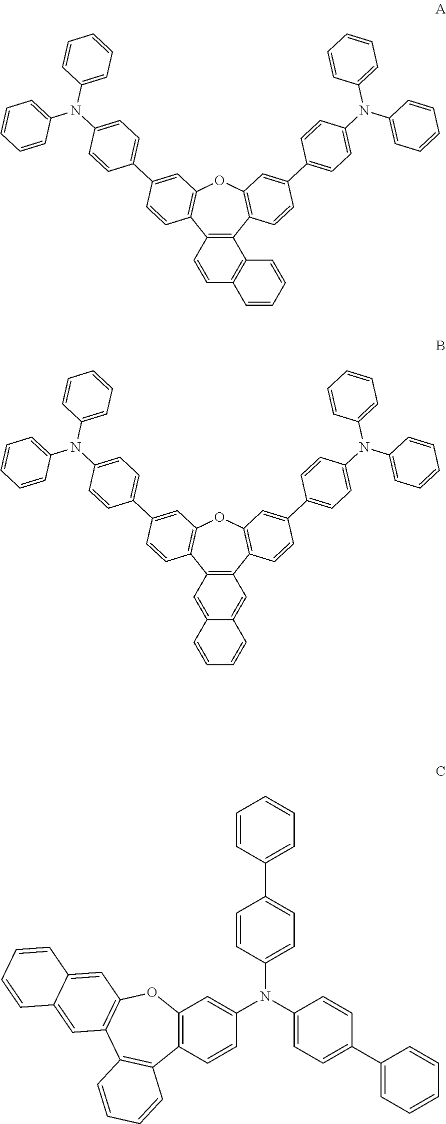 [SMILES: c1ccc(N(c2ccccc2)c2ccc(-c3ccc4c(c3)Oc3cc(-c5ccc(N(c6ccccc6)c6ccccc6)cc5)ccc3-c3c-4ccc4ccccc34)cc2)cc1 ; c1ccc(N(c2ccccc2)c2ccc(-c3ccc4c(c3)Oc3cc(-c5ccc(N(c6ccccc6)c6ccccc6)cc5)ccc3-c3cc5ccccc5cc3-4)cc2)cc1 ; c1ccc(-c2ccc(N(c3ccc(-c4ccccc4)cc3)c3ccc4c(c3)Oc3cc5ccccc5cc3-c3ccccc3-4)cc2)cc1]

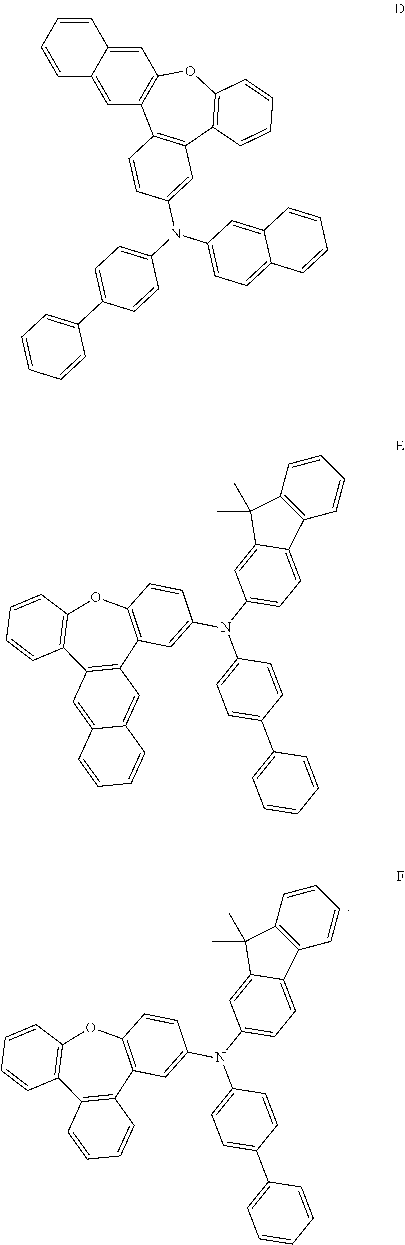 [SMILES: c1ccc(-c2ccc(N(c3ccc4c(c3)-c3ccccc3Oc3cc5ccccc5cc3-4)c3ccc4ccccc4c3)cc2)cc1 ; CC1(C)c2ccccc2-c2ccc(N(c3ccc(-c4ccccc4)cc3)c3ccc4c(c3)-c3cc5ccccc5cc3-c3ccccc3O4)cc21 ; CC1(C)c2ccccc2-c2ccc(N(c3ccc(-c4ccccc4)cc3)c3ccc4c(c3)-c3ccccc3-c3ccccc3O4)cc21]

From the results of Table 7, it was identified that the blue organic light emitting devices of Examples 1 to 68 using the heterocyclic compounds according to the present disclosure as a hole transport layer material had a lower driving voltage, and significantly improved light emission efficiency and lifetime compared to the organic light emitting devices using the compounds described in Comparative Examples 1 to 7. Comparing the structures of the compounds described in Comparative Examples 2 to 7 and the compounds described in Examples 1 to 68, the compounds of Comparative Examples and Examples are similar in that they all have an arylamine group. However, whereas the compounds of Comparative Examples 2 to 7 have, based on the oxepine skeleton, one arylamine group while having a skeleton with no extension of the benzene ring (Comparative Compound F) and a skeleton extended in a linear form (Comparative Compounds B, C, D and E), or two arylamine groups while having a skeleton extended in a bent form (Comparative Compound A), the heterocyclic compounds according to the present disclosure are different in having one arylamine group while having a skeleton extended in a bent form instead of being extended in a linear form. Due to such structural characteristics, there are advantages in that a high triplet energy level (T1 level) is obtained compared to those of Comparative Compounds A to F, and changes in the triplet energy level are not significant even when the arylamine group is changed. Accordingly, by the heterocyclic compound according to the present disclosure having enhanced hole transport properties or enhancing stability, excellent properties are obtained in all aspects of driving voltage, light emission efficiency and lifetime.

### Experimental Example 2

### (1) Manufacture of Organic Light Emitting Device

A transparent electrode indium tin oxide (ITO) thin film obtained from glass for an OLED (manufactured by Samsung Corning Advanced Glass) was ultrasonic cleaned using trichloroethylene, acetone, ethanol and distilled water sequentially for 5 minutes each, and then stored in isopropanol before use. Then, the ITO substrate was installed in a substrate folder of a vacuum deposition apparatus, and to a cell in the vacuum deposition apparatus, the following 4,4′,4″-tris(N,N-(2-naphthyl)-phenylamino)triphenylamine (2-TNATA) was introduced.

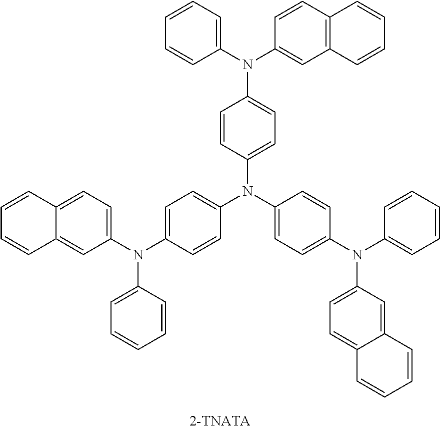 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then the 2-TNATA was evaporated by applying a current to the cell to deposit a hole injection layer having a thickness of 600 Å on the ITO substrate. To another cell in the vacuum deposition apparatus, the following N,N′-bis(α-naphthyl)-N,N′-diphenyl-4,4′-diamine (NPB) was introduced, and evaporated by applying a current to the cell to deposit a hole transport layer having a thickness of 300 Å on the hole injection layer.

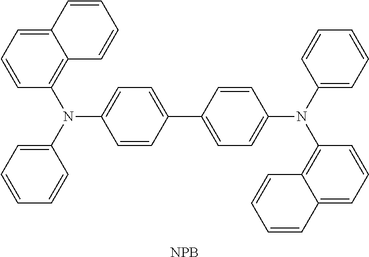 [SMILES: c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transport layer as above, a blue light emitting material having the following structure was deposited thereon as a light emitting layer. Specifically, on one cell in the vacuum deposition apparatus, H1 that is a blue light emitting host material was vacuum deposited to a thickness of 200 Å, and D1 that is a blue light emitting dopant material was vacuum deposited thereon to a thickness of 58 with respect to the host material.

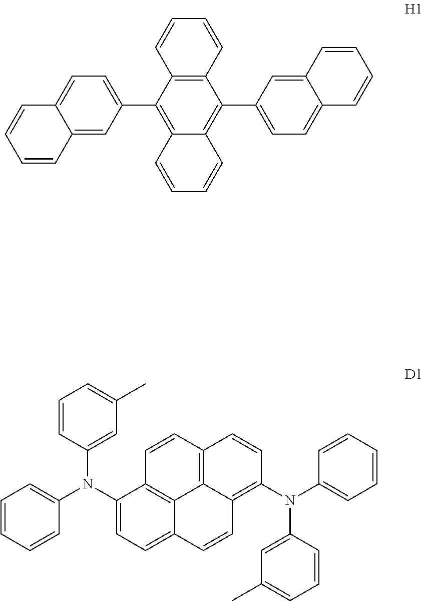

Subsequently, a compound of the following Structural Formula E1 was deposited to a thickness of 300 Å as an electron transport layer.

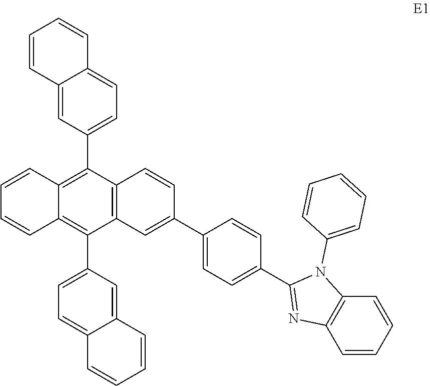 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

Lithium fluoride (LiF) was deposited to a thickness of 10 Å as an electron injection layer, and an aluminum (Al) negative electrode was employed to have a thickness of 1,000 Å, and as a result, an organic light emitting device was manufactured.

Meanwhile, all the organic compounds required to manufacture the organic light emitting device were vacuum sublimation purified under 10⁻⁸ torr to 10⁻⁶ torr for each material to be used in the manufacture of the organic light emitting device.

Organic light emitting devices were manufactured in the same manner as in Experimental Example 2, except that NPB used when forming the hole transport layer in Experimental Example 2 was formed to a thickness of 250 Å and then an electron blocking layer was formed to a thickness of 50 Å on the hole transport layer using compounds listed in the following Table 8.

### (2) Driving Voltage and Light Emission Efficiency of Organic Light Emitting Device

For each of the organic light emitting devices manufactured as above, electroluminescent (EL) properties were measured using M7000 manufactured by McScience Inc., and with the measurement results, a lifetime T₉₅, a time taken for luminance to become 95% with respect to initial luminance, was measured when standard luminance was 700 cd/m2 through a lifetime measurement system (M6000) manufactured by McScience Inc.

Results of measuring driving voltage, light emission efficiency, color coordinate (CIE) and lifetime of the blue organic light emitting devices manufactured according to the present disclosure are shown in the following Table 8.

TABLE 8

| | | | Light | | |
|---|---|---|---|---|---|
| | | | Emis- | | |
| | | | sion | | |
| | | Driving | Effi- | | Life |
| | | Voltage | ciency | CIE | time |
| | Compound | (V) | (cd/A) | (x, y) | (T95) |
| Example 69 | 1 | 4.73 | 7.01 | (0.134, 0.100) | 93 |
| Example 70 | 5 | 4.71 | 7.18 | (0.134, 0.100) | 94 |
| Example 71 | 7 | 4.83 | 7.23 | (0.134, 0.100) | 97 |
| Example 72 | 9 | 4.82 | 7.15 | (0.134, 0.100) | 95 |
| Example 73 | 16 | 4.93 | 7.13 | (0.133, 0.100) | 100 |
| Example 74 | 17 | 4.81 | 7.20 | (0.133, 0.100) | 100 |
| Example 75 | 18 | 4.85 | 7.13 | (0.134, 0.100) | 95 |
| Example 76 | 20 | 4.90 | 7.00 | (0.134, 0.100) | 94 |
| Example 77 | 25 | 4.73 | 7.13 | (0.134, 0.101) | 91 |
| Example 78 | 28 | 4.65 | 7.01 | (0.134, 0.100) | 93 |
| Example 79 | 30 | 4.78 | 7.03 | (0.134, 0.100) | 100 |
| Example 80 | 33 | 4.88 | 6.98 | (0.134, 0.100) | 90 |
| Example 81 | 38 | 4.90 | 7.05 | (0.134, 0.100) | 95 |
| Example 82 | 39 | 4.80 | 7.15 | (0.134, 0.100) | 94 |
| Example 83 | 45 | 4.85 | 7.10 | (0.133, 0.100) | 93 |
| Example 84 | 53 | 4.84 | 7.00 | (0.134, 0.100) | 92 |
| Example 85 | 56 | 4.88 | 7.03 | (0.134, 0.100) | 93 |
| Example 86 | 62 | 4.73 | 7.01 | (0.134, 0.100) | 104 |
| Example 87 | 63 | 4.85 | 7.13 | (0.134, 0.100) | 107 |
| Example 88 | 64 | 4.81 | 7.10 | (0.133, 0.100) | 105 |
| Example 89 | 65 | 4.83 | 7.05 | (0.134, 0.100) | 97 |
| Example 90 | 74 | 4.81 | 7.07 | (0.133, 0.100) | 94 |
| Example 91 | 75 | 4.76 | 7.12 | (0.134, 0.100) | 95 |
| Example 92 | 77 | 4.84 | 7.01 | (0.134, 0.100) | 95 |
| Example 93 | 78 | 4.84 | 6.99 | (0.134, 0.100) | 92 |
| Example 94 | 79 | 4.82 | 7.03 | (0.134, 0.100) | 92 |
| Example 95 | 88 | 4.73 | 7.11 | (0.134, 0.100) | 95 |
| Example 96 | 93 | 4.82 | 7.20 | (0.134, 0.100) | 90 |
| Example 97 | 99 | 4.83 | 7.02 | (0.134, 0.100) | 92 |
| Example 98 | 100 | 4.75 | 7.08 | (0.134, 0.100) | 94 |
| Example 99 | 113 | 4.88 | 7.20 | (0.134, 0.101) | 89 |
| Example 100 | 119 | 4.71 | 7.12 | (0.133, 0.100) | 90 |
| Example 101 | 124 | 4.73 | 7.12 | (0.133, 0.100) | 90 |
| Example 102 | 129 | 4.85 | 7.21 | (0.134, 0.100) | 110 |
| Example 103 | 130 | 4.85 | 7.03 | (0.134, 0.100) | 100 |
| Example 104 | 135 | 4.83 | 7.21 | (0.134, 0.100) | 93 |
| Example 105 | 138 | 4.84 | 7.23 | (0.134, 0.100) | 89 |
| Example 106 | 140 | 4.94 | 7.13 | (0.134, 0.100) | 99 |
| Example 107 | 142 | 4.80 | 7.00 | (0.134, 0.100) | 93 |
| Example 108 | 143 | 4.91 | 7.00 | (0.133, 0.100) | 92 |
| Example 109 | 152 | 4.71 | 7.12 | (0.134, 0.100) | 91 |
| Example 110 | 157 | 4.80 | 7.21 | (0.134, 0.100) | 89 |
| Example 111 | 163 | 4.70 | 6.99 | (0.134, 0.100) | 92 |
| Example 112 | 164 | 4.67 | 7.00 | (0.134, 0.101) | 94 |
| Example 113 | 177 | 4.82 | 7.22 | (0.134, 0.100) | 90 |
| Example 114 | 182 | 4.73 | 7.02 | (0.134, 0.100) | 91 |
| Example 115 | 183 | 4.72 | 7.03 | (0.134, 0.100) | 92 |
| Example 116 | 195 | 4.83 | 7.12 | (0.134, 0.100) | 105 |
| Example 117 | 196 | 4.90 | 7.03 | (0.134, 0.100) | 107 |
| Example 118 | 198 | 4.82 | 7.18 | (0.134, 0.100) | 95 |
| Example 119 | 205 | 4.83 | 7.05 | (0.134, 0.100) | 97 |
| Example 120 | 212 | 4.78 | 7.13 | (0.133, 0.100) | 97 |
| Example 121 | 214 | 4.80 | 7.03 | (0.134, 0.100) | 100 |
| Example 122 | 216 | 4.87 | 7.08 | (0.134, 0.100) | 103 |
| Example 123 | 230 | 4.89 | 7.24 | (0.134, 0.100) | 95 |
| Example 124 | 231 | 4.88 | 7.22 | (0.134, 0.100) | 95 |
| Example 125 | 238 | 4.75 | 7.08 | (0.134, 0.100) | 94 |
| Example 126 | 252 | 4.77 | 7.00 | (0.134, 0.100) | 94 |
| Example 127 | 257 | 4.80 | 7.20 | (0.134, 0.100) | 110 |
| Example 128 | 258 | 4.85 | 7.02 | (0.134, 0.100) | 103 |
| Example 129 | 267 | 4.75 | 7.00 | (0.134, 0.100) | 93 |
| Example 130 | 298 | 4.90 | 6.88 | (0.133, 0.100) | 94 |
| Example 131 | 304 | 4.88 | 7.11 | (0.134, 0.100) | 95 |
| Example 132 | 320 | 4.87 | 7.13 | (0.134, 0.100) | 97 |
| Example 133 | 329 | 4.80 | 7.10 | (0.133, 0.100) | 94 |
| Example 134 | 351 | 4.92 | 7.01 | (0.134, 0.100) | 94 |
| Example 135 | 355 | 4.90 | 7.03 | (0.134, 0.100) | 94 |
| Example 136 | 382 | 4.85 | 7.10 | (0.133, 0.100) | 99 |
| Comparative | NPB | 5.53 | 6.05 | (0.134, 0.100) | 50 |
| Example 8 | | | | | |
| Comparative | Comparative | 5.33 | 6.03 | (0.134, 0.100) | 55 |
| Example 9 | Compound A | | | | |
| Comparative | Comparative | 5.42 | 6.00 | (0.134, 0.100) | 57 |
| Example 10 | Compound B | | | | |
| Comparative | Comparative | 5.34 | 6.20 | (0.133, 0.100) | 60 |
| Example 11 | Compound C | | | | |
| Comparative | Comparative | 5.21 | 6.18 | (0.134, 0.100) | 59 |
| Example 12 | Compound D | | | | |
| Comparative | Comparative | 5.24 | 6.20 | (0.134, 0.100) | 60 |
| Example 13 | Compound E | | | | |
| Comparative | Comparative | 5.23 | 6.33 | (0.134, 0.101) | 73 |
| Example 14 | Compound F | | | | |

The compound of Comparative Example 8 is as follows:

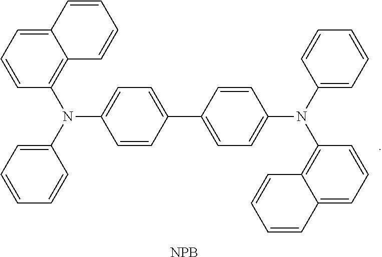 [SMILES: c1ccc(N(c2ccc(-c3ccc(N(c4ccccc4)c4cccc5ccccc45)cc3)cc2)c2cccc3ccccc23)cc1 ; "NPB"]

In addition, the compounds of Comparative Examples 9 to 14 are the same as the following Comparative Compounds A to F:

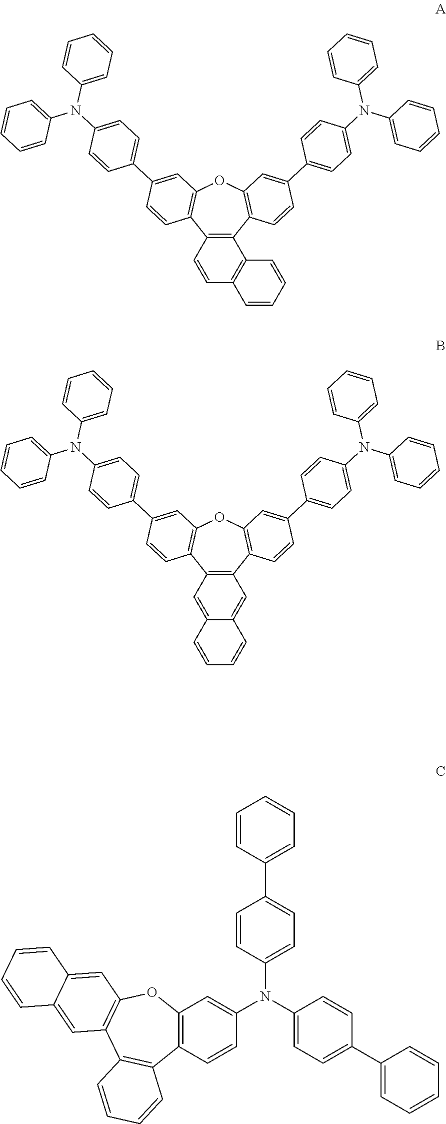 [SMILES: c1ccc(N(c2ccccc2)c2ccc(-c3ccc4c(c3)Oc3cc(-c5ccc(N(c6ccccc6)c6ccccc6)cc5)ccc3-c3c-4ccc4ccccc34)cc2)cc1 ; c1ccc(N(c2ccccc2)c2ccc(-c3ccc4c(c3)Oc3cc(-c5ccc(N(c6ccccc6)c6ccccc6)cc5)ccc3-c3cc5ccccc5cc3-4)cc2)cc1 ; c1ccc(-c2ccc(N(c3ccc(-c4ccccc4)cc3)c3ccc4c(c3)Oc3cc5ccccc5cc3-c3ccccc3-4)cc2)cc1]

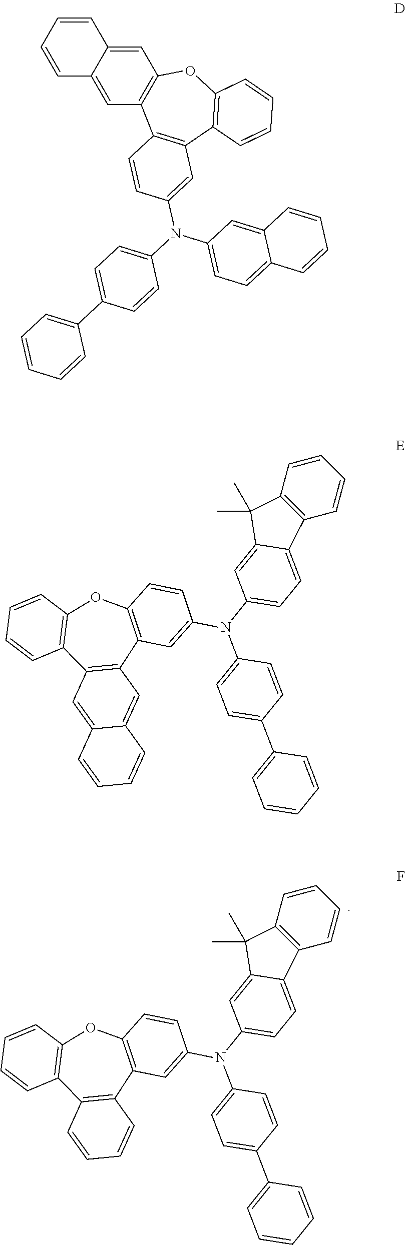 [SMILES: c1ccc(-c2ccc(N(c3ccc4c(c3)-c3ccccc3Oc3cc5ccccc5cc3-4)c3ccc4ccccc4c3)cc2)cc1 ; CC1(C)c2ccccc2-c2ccc(N(c3ccc(-c4ccccc4)cc3)c3ccc4c(c3)-c3cc5ccccc5cc3-c3ccccc3O4)cc21 ; CC1(C)c2ccccc2-c2ccc(N(c3ccc(-c4ccccc4)cc3)c3ccc4c(c3)-c3ccccc3-c3ccccc3O4)cc21]

From the results of Table 8, it was identified that the blue organic light emitting devices of Examples 69 to 136 using the heterocyclic compounds according to the present disclosure as an electron blocking layer material had a lower driving voltage, and significantly improved light emission efficiency and lifetime compared to the organic light emitting devices using the compounds described in Comparative Examples 8 to 14. When electrons migrating in an organic light emitting device pass through a hole transport layer and migrate to a positive electrode without binding in a light emitting layer, a phenomenon of reducing light emission efficiency and lifetime occurs in the organic light emitting device. When a compound having high LUMO level and triplet energy level (T1 level) is used as an electron blocking layer in order to prevent such a phenomenon, electrons attempting to pass through a light emitting layer and migrate to a positive electrode are blocked in the migration by an energy barrier of the electron blocking layer. As a result, probability of holes and electrons forming excitons in the light emitting layer increases, and possibility of them being emitted as light in the light emitting layer increases. Accordingly, when using the heterocyclic compound according to the present disclosure as a material of an electron blocking layer, the heterocyclic compound according to the present disclosure is capable of effectively controlling electron migration due to structural properties of the compound having high LUMO level and triplet energy level, and exhibits excellent properties in all aspects of driving voltage, light emission efficiency and lifetime.

### REFERENCE NUMERAL

100: Substrate
200: Positive Electrode
300: Organic Material Layer
301: Hole Injection Layer
302: Hole Transport Layer
303: Light Emitting Layer
304: Hole Blocking Layer
305: Electron Transport Layer
306: Election Injection Layer
400: Negative Electrode

What is claimed is:

1. A heterocyclic compound represented by the following Chemical Formula 1: 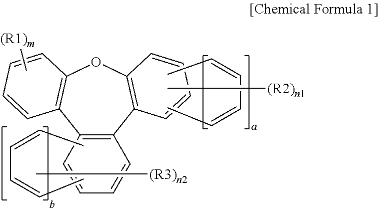
in Chemical Formula 1,
R1 to R3 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; and a group represented by the following Chemical Formula 2;
any one of R1 to R3 is the group represented by the following Chemical Formula 2, however, the remaining two are not the group represented by the following Chemical Formula 2;
a and b are each independently an integer of 0 or 1, and satisfy a+b=1;
m is one of integers of 1 to 4;
when a or b is 0, n1 or n2 is each independently one of integers of 1 to 4; and
when a or b is 1, n1 or n2 is each independently one of integers of 1 to 6, 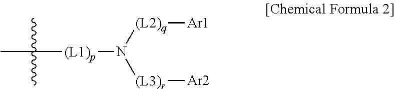
in Chemical Formula 2,
L1, L2 and L3 are the same as or different from each other, and each independently a single bond; a substituted or unsubstituted C6 to C60 arylene group; or a substituted or unsubstituted C2 to C60 heteroarylene group;
Ar1 and Ar2 are the same as or different from each other, and each independently a substituted or unsubstituted C6 to C60 aryl group; or a substituted or unsubstituted C2 to C60 heteroaryl group; and
p, q and r are the same as or different from each other, and each independently an integer of 0 to 3.

2. The heterocyclic compound of claim 1, wherein the compound of Chemical Formula 1 is represented by the following Chemical Formula 1-a, Chemical Formula 1-b or Chemical Formula 1-c: 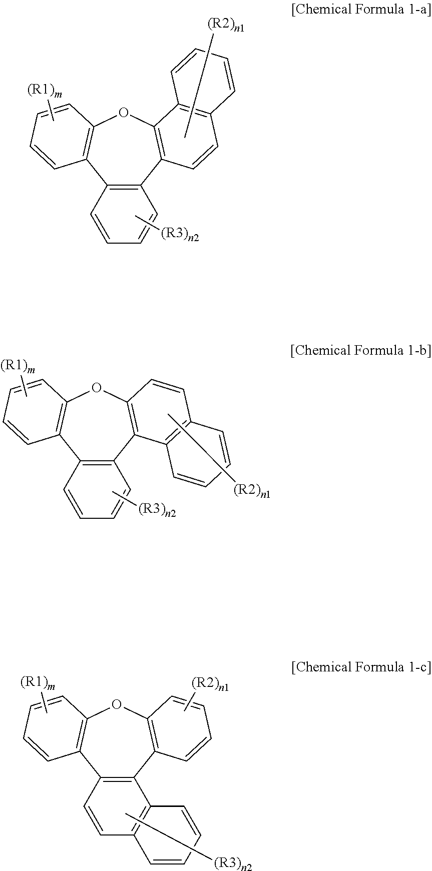
in Chemical Formulae 1-a to 1-c,
R1, R2, R3, m, n1 and n2 have the same definitions as in Chemical Formula 1.

3. The heterocyclic compound of claim 1, wherein the compound of Chemical Formula 1 is represented by the following Chemical Formula 1-a-1, Chemical Formula 1-a-2, Chemical Formula 1-a-3, Chemical Formula 1-b-1, Chemical Formula 1-b-2, Chemical Formula 1-b-3, Chemical Formula 1-c-1, Chemical Formula 1-c-2 or Chemical Formula 1-c-3: 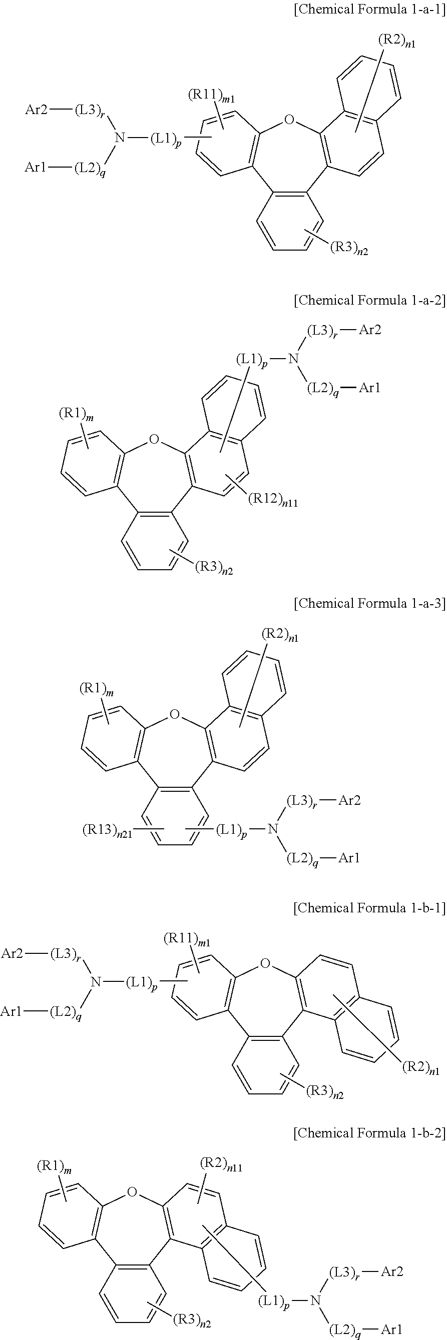 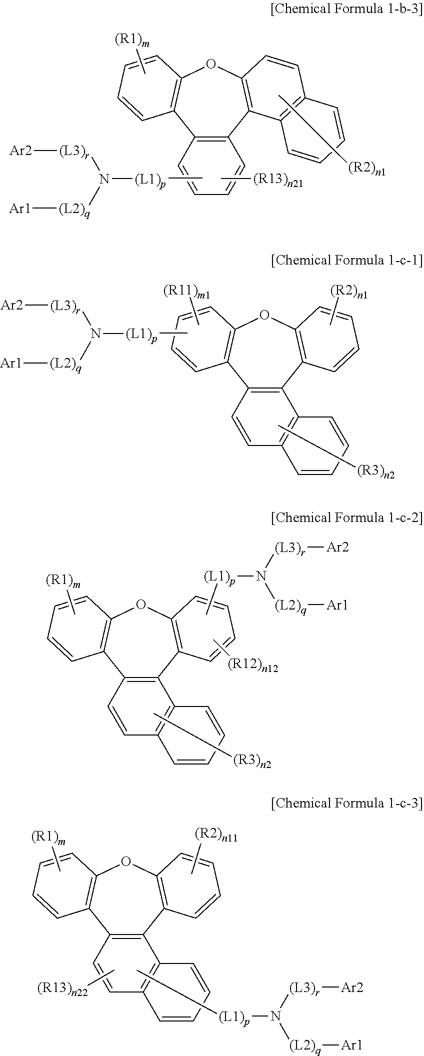
in Chemical Formulae 1-a-1 to 1-c-3,
R1, R2, R3, m, n1, n2, L1, L2, L3, Ar1, Ar2, p, q and r have the same definitions as in Chemical Formula 1;
R11, R12 and R13 are the same as or different from each other, and each independently selected from the group consisting of hydrogen; deuterium; halogen; a cyano group; a substituted or a substituted or C1 unsubstituted C1 to C60 alkyl group; unsubstituted to C60 alkenyl group; a substituted or unsubstituted C2 to C60 alkynyl group; a substituted or unsubstituted C1 to C60 alkoxy group; a substituted or unsubstituted C3 to C60 cycloalkyl group; and a substituted or unsubstituted C2 to C60 heterocycloalkyl group;
m1 is one of integers of 1 to 3;
n11 is one of integers of 1 to 5;
n12 is one of integers of 1 to 3;
n21 is one of integers of 1 to 3; and
n22 is one of integers of 1 to 5.

4. The heterocyclic compound of claim 3, wherein at least one of R1 to R3 is hydrogen; deuterium; or a substituted or unsubstituted C6 to C60 aryl group.

5. The heterocyclic compound of claim 3, wherein, when at least one of R1 to R3 is a C6 to C60 aryl group, the aryl group is selected from the group consisting of the following structural formulae: 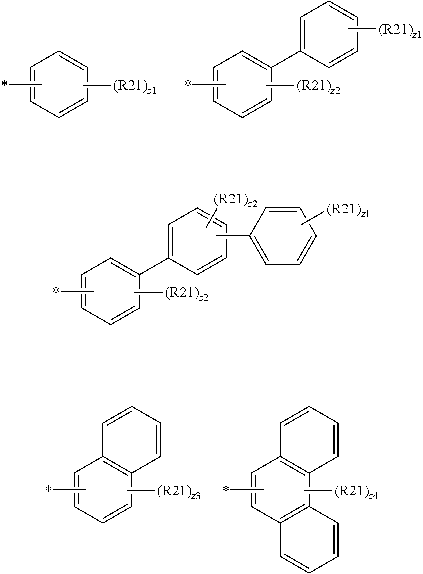
wherein,
R21 is selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; and a substituted or unsubstituted C2 to C60 heterocycloalkyl group;
z1 is one of integers of 1 to 5;
z2 is one of integers of 1 to 4;
z3 is one of integers of 1 to 7;
z4 is one of integers of 1 to 9; and
* is a position bonding to the atom of Chemical Formula 1.

6. The heterocyclic compound of claim 1, wherein, in Chemical Formula 2, Ar1 and Ar2 are each independently selected from the group consisting of a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group; a substituted or unsubstituted fluorenyl group; a substituted or unsubstituted benzofluorenyl group; a substituted or unsubstituted dibenzofluorenyl group; a substituted or unsubstituted spiro-bifluorenyl group; a substituted or unsubstituted benzofuranyl group; a substituted or unsubstituted dibenzofuranyl group; a substituted or unsubstituted benzothiophenyl group; and a substituted or unsubstituted dibenzothiophenyl group.

7. The heterocyclic compound of claim 1, wherein, in Chemical Formula 2, Ar1 and Ar2 are each independently selected from the group consisting of the following structural formulae: 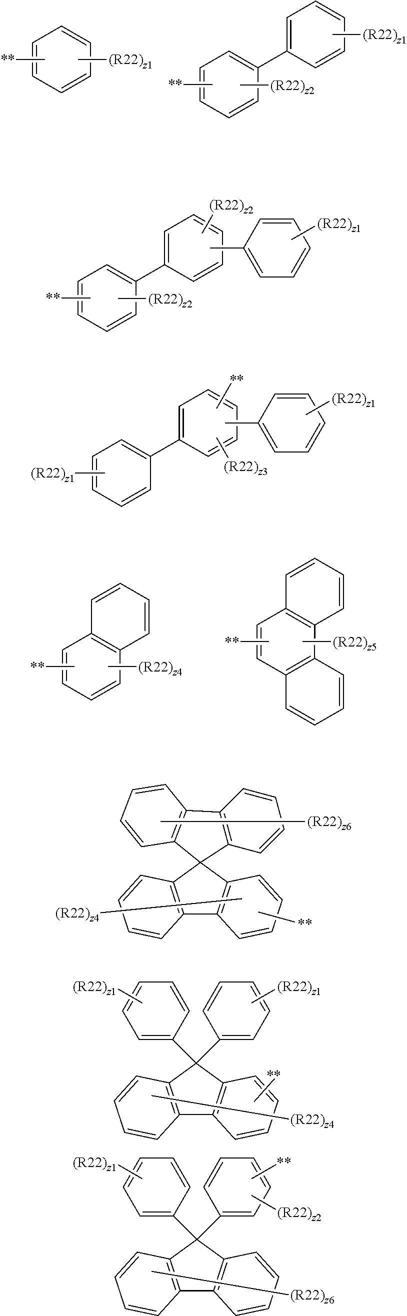 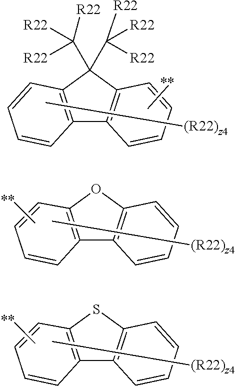
wherein,
R22 is selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; and a substituted or unsubstituted C2 to C60 heterocycloalkyl group;
z1 is one of integers of 1 to 5;
z2 is one of integers of 1 to 4;
z3 is one of integers of 1 to 3;
z4 is one of integers of 1 to 7;
z5 is one of integers of 1 to 9;
z6 is one of integers of 1 to 8; and
** is a position bonding to the N atom, L2 or L3 of Chemical Formula 2.

8. The heterocyclic compound of claim 1, wherein the heterocyclic compound represented by Chemical Formula 1 does not include deuterium as a substituent, or has a deuterium content of 1% to 100% based on a total number of hydrogen atoms and deuterium atoms.

9. The heterocyclic compound of claim 1, wherein the heterocyclic compound represented by Chemical Formula 1 is any one selected from the group consisting of the following compounds:

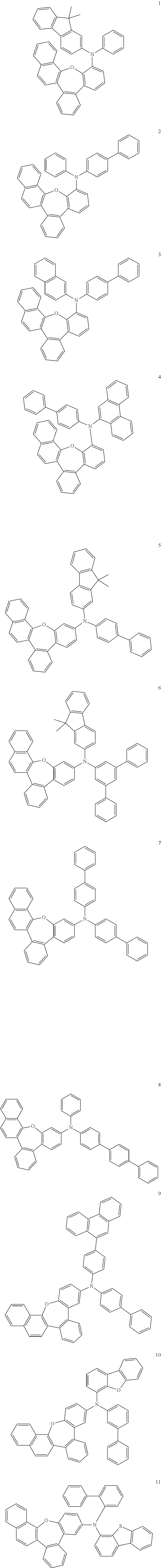

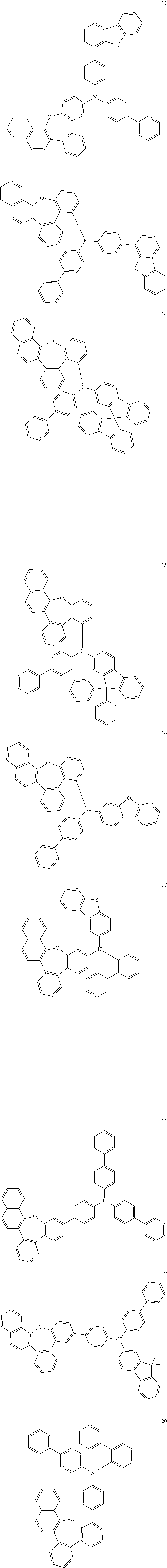

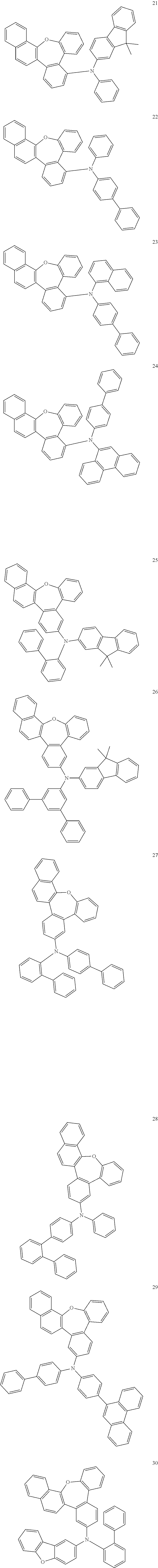

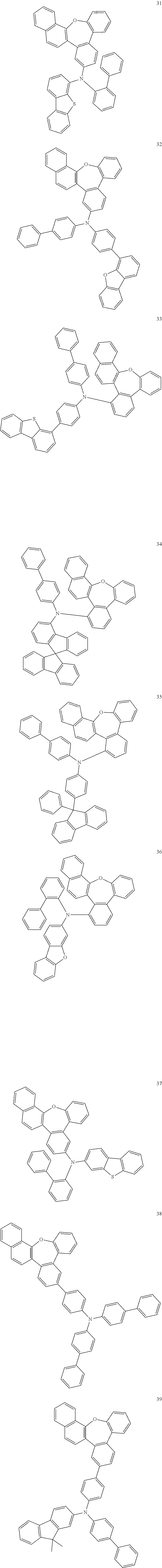

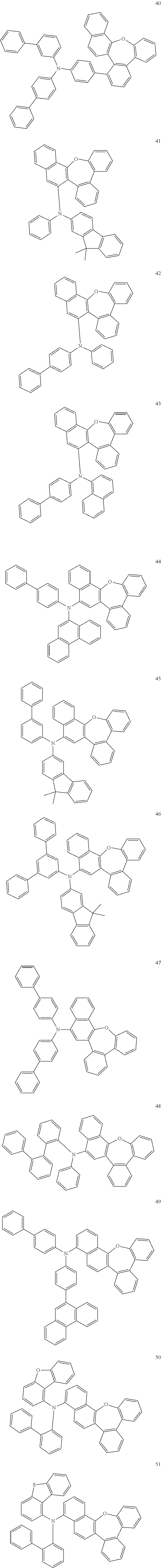

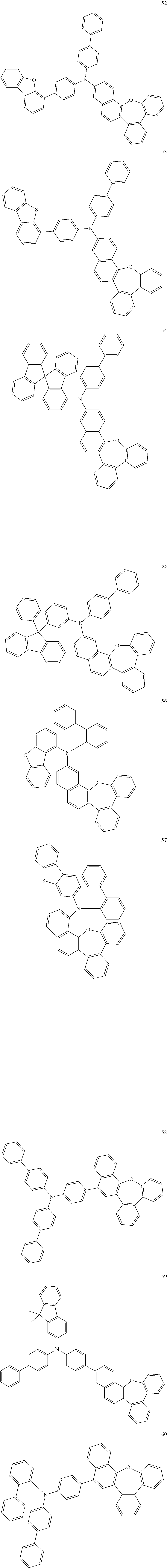

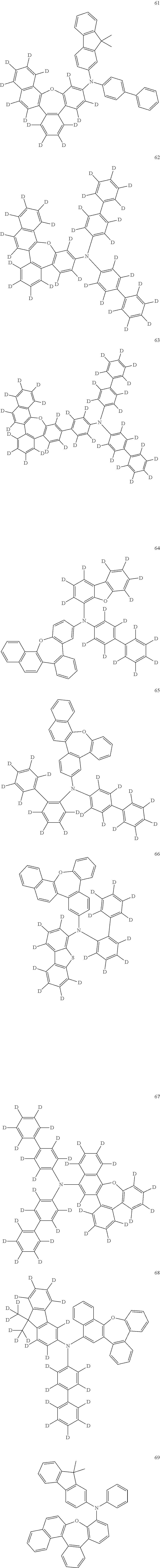

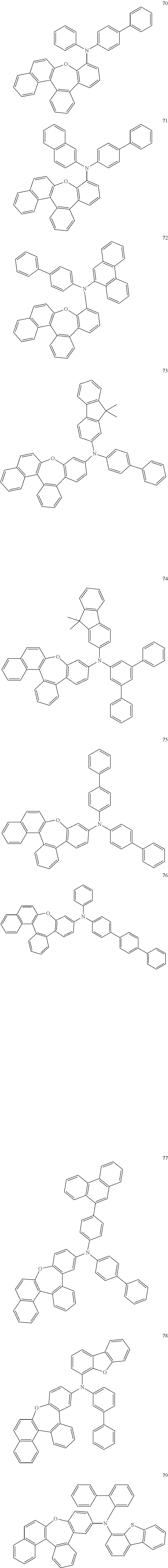

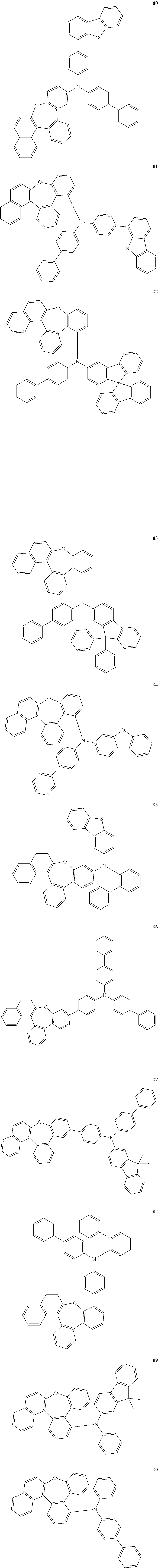

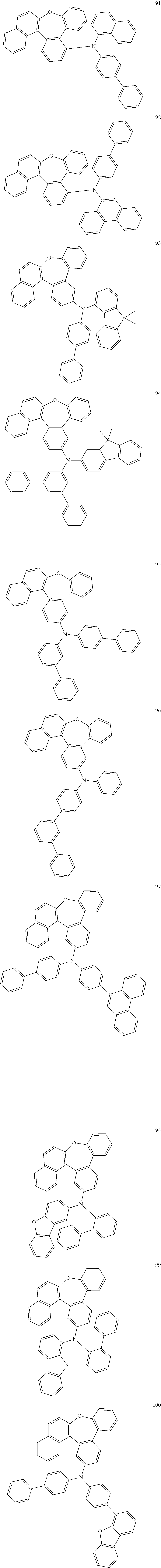

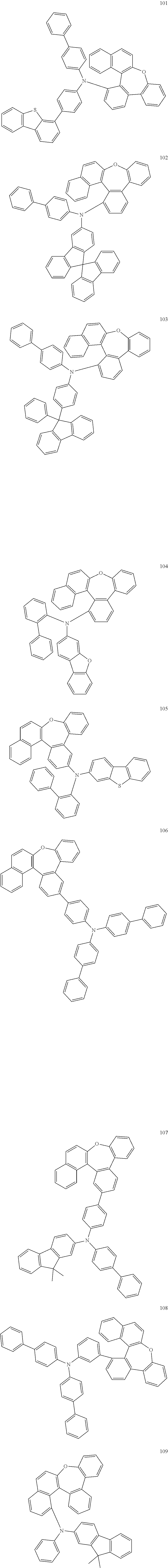

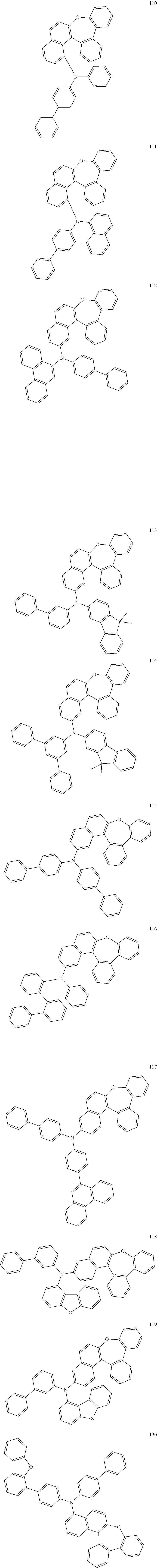

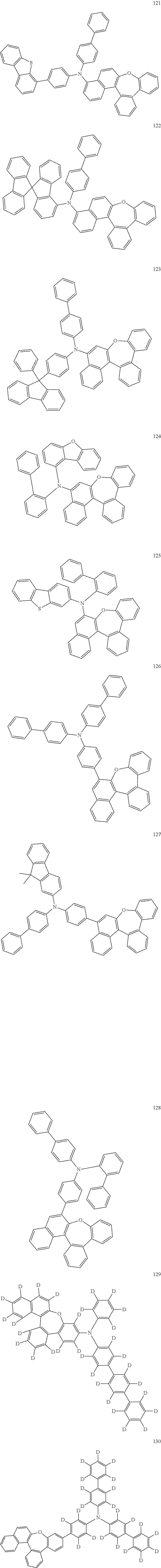

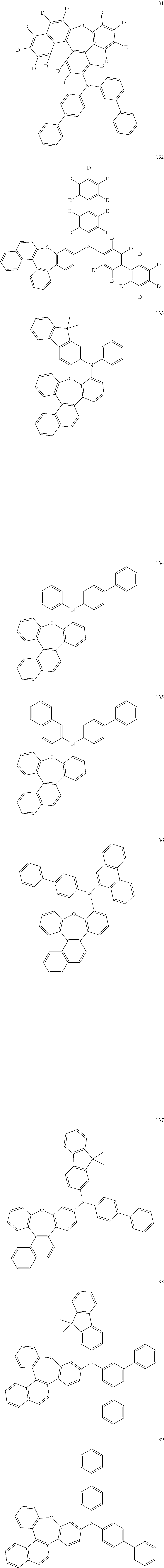

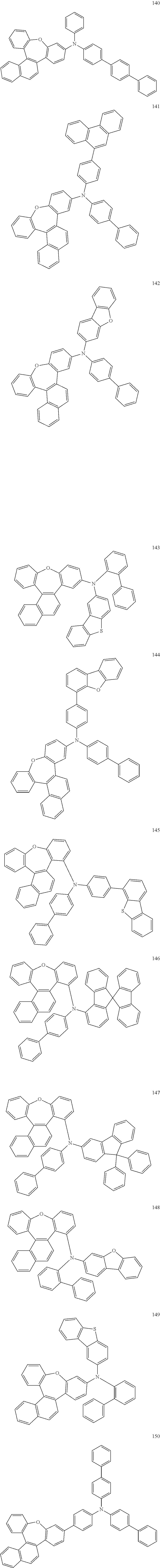

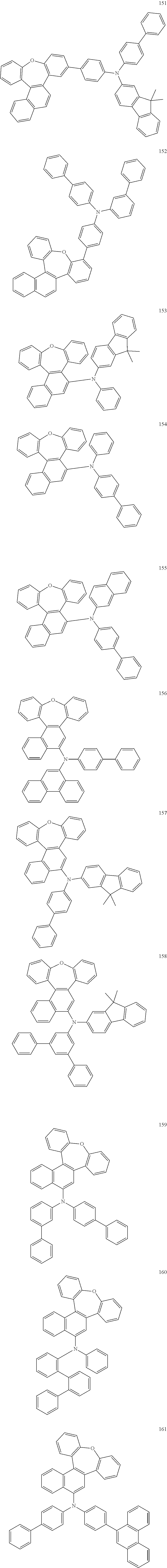

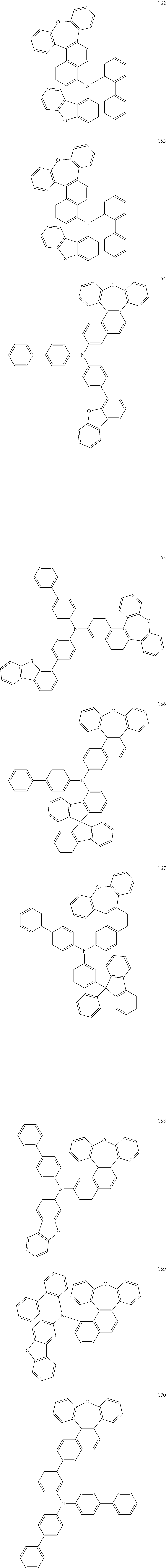

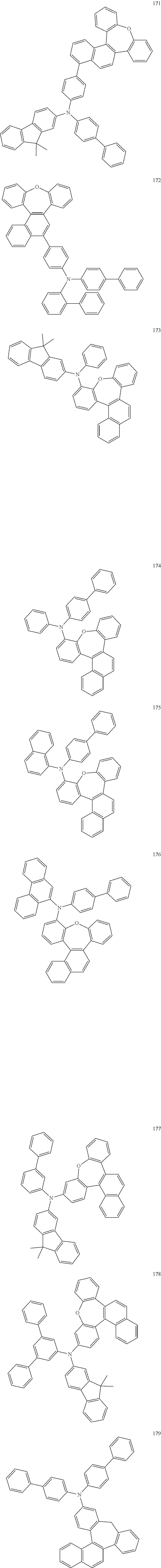

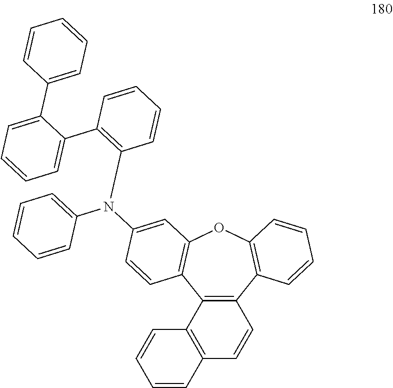 [SMILES: c1ccc(-c2ccccc2-c2ccccc2N(c2ccccc2)c2ccc3c(c2)Oc2ccccc2-c2ccc4ccccc4c2-3)cc1]

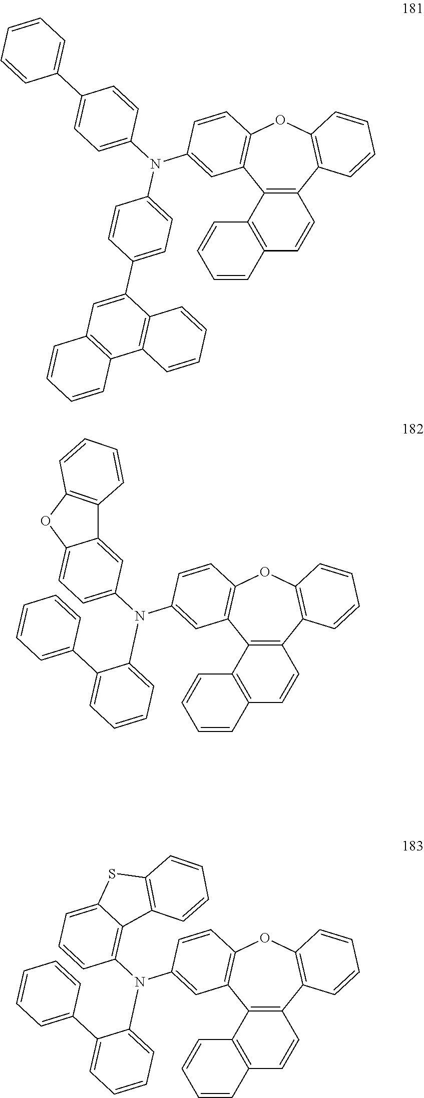

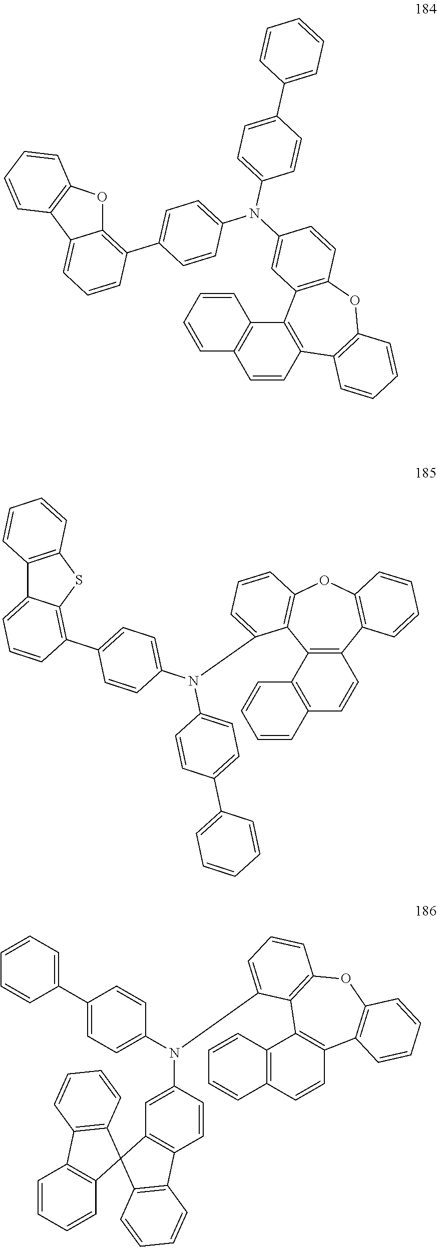 [SMILES: c1ccc(-c2ccc(N(c3ccc(-c4cccc5c4oc4ccccc45)cc3)c3ccc4c(c3)-c3c(ccc5ccccc35)-c3ccccc3O4)cc2)cc1 ; c1ccc(-c2ccc(N(c3ccc(-c4cccc5c4sc4ccccc45)cc3)c3cccc4c3-c3c(ccc5ccccc35)-c3ccccc3O4)cc2)cc1 ; c1ccc(-c2ccc(N(c3ccc4c(c3)C3(c5ccccc5-c5ccccc53)c3ccccc3-4)c3cccc4c3-c3c(ccc5ccccc35)-c3ccccc3O4)cc2)cc1]

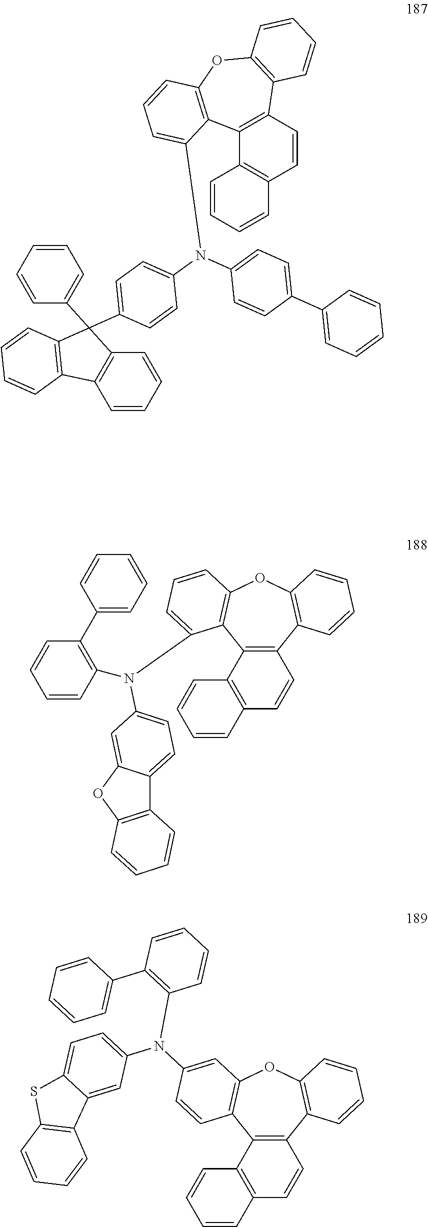 [SMILES: c1ccc(-c2ccc(N(c3ccc(C4(c5ccccc5)c5ccccc5-c5ccccc54)cc3)c3cccc4c3-c3c(ccc5ccccc35)-c3ccccc3O4)cc2)cc1 ; c1ccc(-c2ccccc2N(c2ccc3c(c2)oc2ccccc23)c2cccc3c2-c2c(ccc4ccccc24)-c2ccccc2O3)cc1 ; c1ccc(-c2ccccc2N(c2ccc3c(c2)Oc2ccccc2-c2ccc4ccccc4c2-3)c2ccc3sc4ccccc4c3c2)cc1]

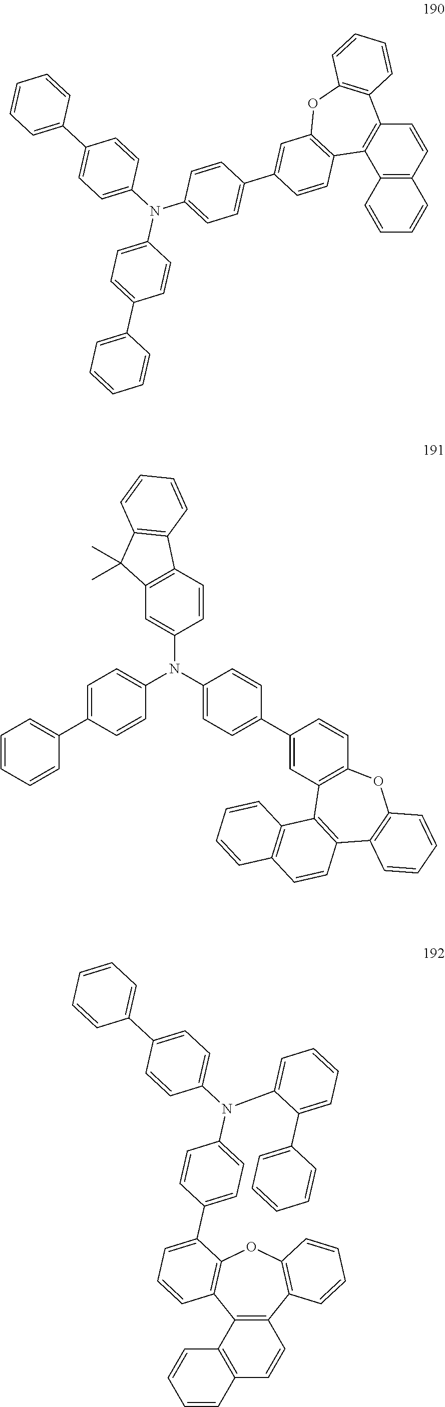 [SMILES: c1ccc(-c2ccc(N(c3ccc(-c4ccccc4)cc3)c3ccc(-c4ccc5c(c4)Oc4ccccc4-c4ccc6ccccc6c4-5)cc3)cc2)cc1 ; CC1(C)c2ccccc2-c2ccc(N(c3ccc(-c4ccccc4)cc3)c3ccc(-c4ccc5c(c4)-c4c(ccc6ccccc46)-c4ccccc4O5)cc3)cc21 ; c1ccc(-c2ccc(N(c3ccc(-c4cccc5c4Oc4ccccc4-c4ccc6ccccc6c4-5)cc3)c3ccccc3-c3ccccc3)cc2)cc1]

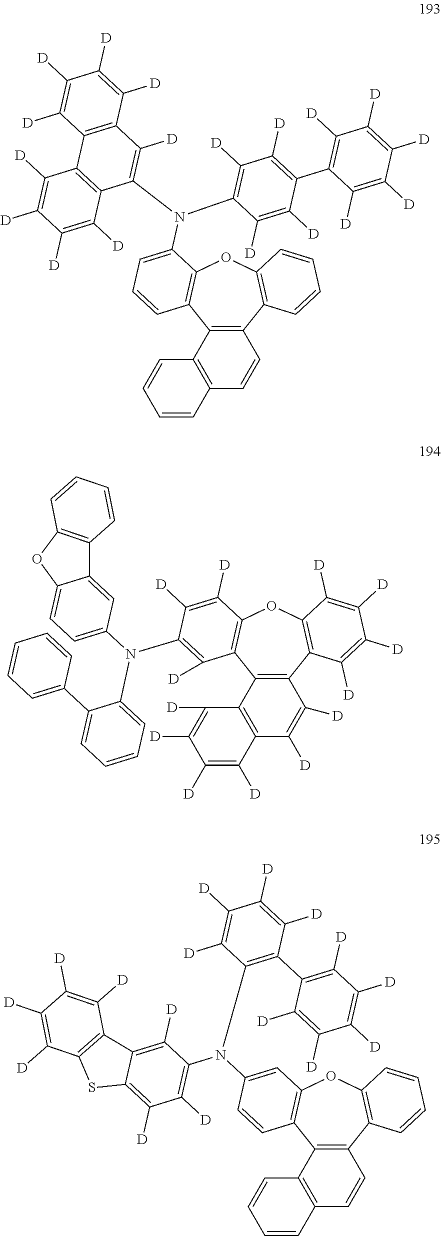

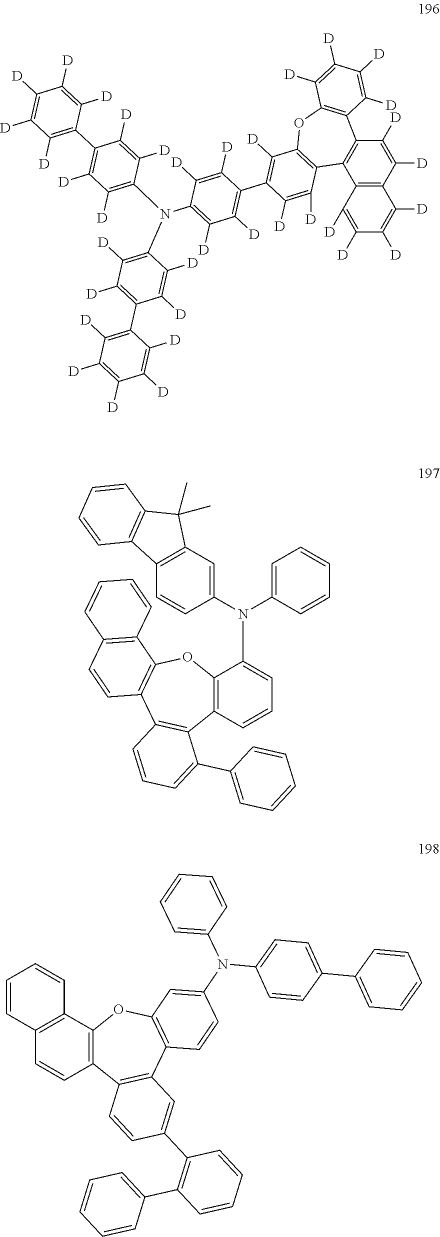 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c(-c4c([2H])c([2H])c5c(c4[2H])Oc4c([2H])c([2H])c([2H])c([2H])c4-c4c([2H])c([2H])c6c([2H])c([2H])c([2H])c([2H])c6c4-5)c([2H])c3[2H])c([2H])c2[2H])c([2H])c1[2H] ; CC1(C)c2ccccc2-c2ccc(N(c3ccccc3)c3cccc4c3Oc3c(ccc5ccccc35)-c3cccc(-c5ccccc5)c3-4)cc21 ; c1ccc(-c2ccc(N(c3ccccc3)c3ccc4c(c3)Oc3c(ccc5ccccc35)-c3ccc(-c5ccccc5-c5ccccc5)cc3-4)cc2)cc1]

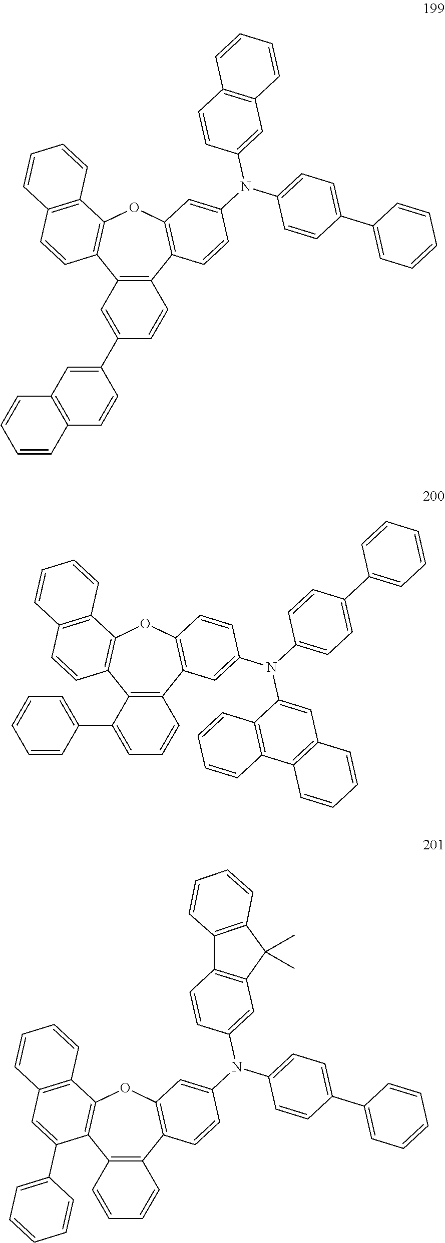 [SMILES: c1ccc(-c2ccc(N(c3ccc4c(c3)Oc3c(ccc5ccccc35)-c3cc(-c5ccc6ccccc6c5)ccc3-4)c3ccc4ccccc4c3)cc2)cc1 ; c1ccc(-c2ccc(N(c3ccc4c(c3)-c3cccc(-c5ccccc5)c3-c3ccc5ccccc5c3O4)c3cc4ccccc4c4ccccc34)cc2)cc1 ; CC1(C)c2ccccc2-c2ccc(N(c3ccc(-c4ccccc4)cc3)c3ccc4c(c3)Oc3c(c(-c5ccccc5)cc5ccccc35)-c3ccccc3-4)cc21]

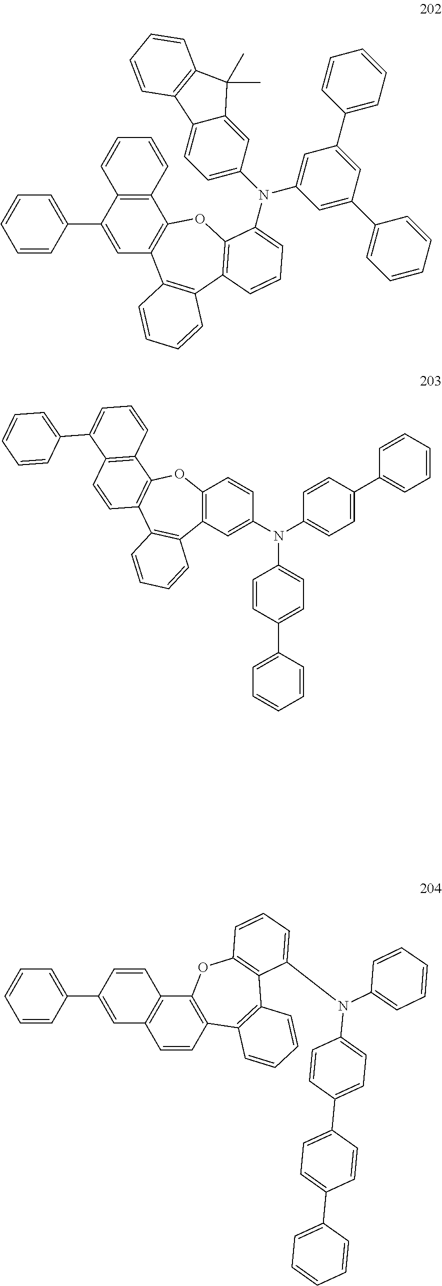 [SMILES: CC1(C)c2ccccc2-c2ccc(N(c3cc(-c4ccccc4)cc(-c4ccccc4)c3)c3cccc4c3Oc3c(cc(-c5ccccc5)c5ccccc35)-c3ccccc3-4)cc21 ; c1ccc(-c2ccc(N(c3ccc(-c4ccccc4)cc3)c3ccc4c(c3)-c3ccccc3-c3ccc5c(-c6ccccc6)cccc5c3O4)cc2)cc1 ; c1ccc(-c2ccc(-c3ccc(N(c4ccccc4)c4cccc5c4-c4ccccc4-c4ccc6cc(-c7ccccc7)ccc6c4O5)cc3)cc2)cc1]

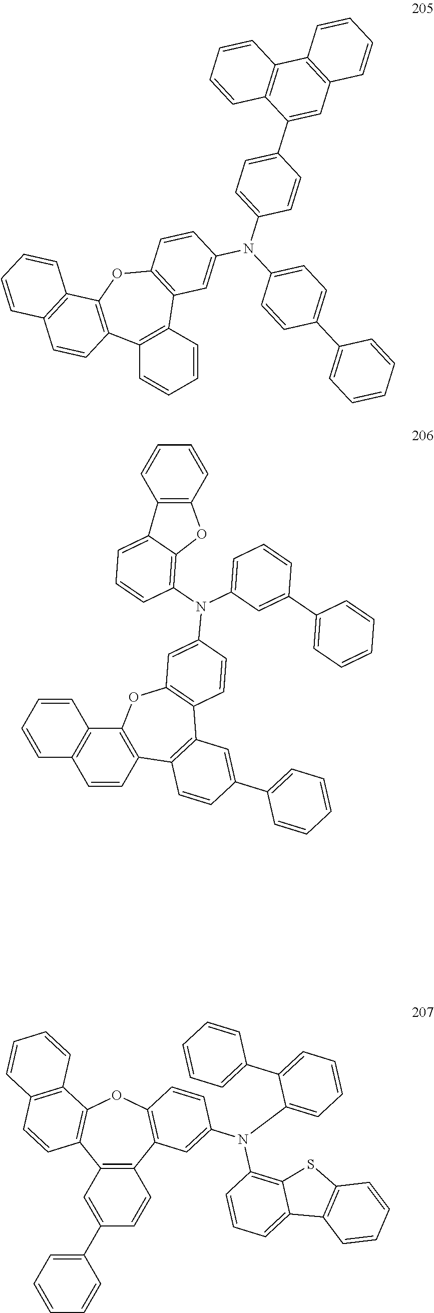 [SMILES: c1ccc(-c2ccc(N(c3ccc(-c4cc5ccccc5c5ccccc45)cc3)c3ccc4c(c3)-c3ccccc3-c3ccc5ccccc5c3O4)cc2)cc1 ; c1ccc(-c2cccc(N(c3ccc4c(c3)Oc3c(ccc5ccccc35)-c3ccc(-c5ccccc5)cc3-4)c3cccc4c3oc3ccccc34)c2)cc1 ; c1ccc(-c2ccc3c(c2)-c2ccc4ccccc4c2Oc2ccc(N(c4ccccc4-c4ccccc4)c4cccc5c4sc4ccccc45)cc2-3)cc1]

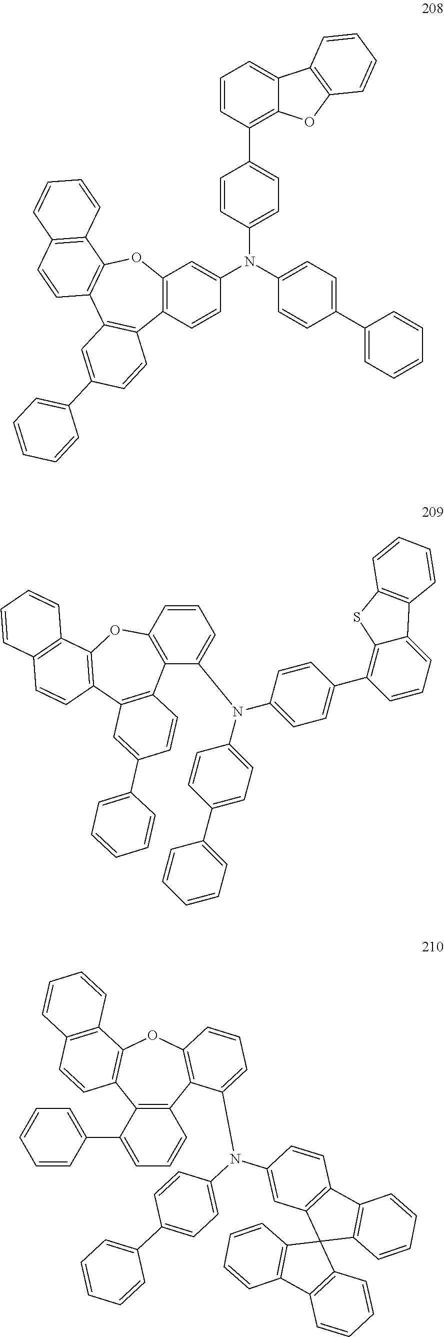 [SMILES: c1ccc(-c2ccc(N(c3ccc(-c4cccc5c4oc4ccccc45)cc3)c3ccc4c(c3)Oc3c(ccc5ccccc35)-c3cc(-c5ccccc5)ccc3-4)cc2)cc1 ; c1ccc(-c2ccc(N(c3ccc(-c4cccc5c4sc4ccccc45)cc3)c3cccc4c3-c3ccc(-c5ccccc5)cc3-c3ccc5ccccc5c3O4)cc2)cc1 ; c1ccc(-c2ccc(N(c3ccc4c(c3)C3(c5ccccc5-c5ccccc53)c3ccccc3-4)c3cccc4c3-c3cccc(-c5ccccc5)c3-c3ccc5ccccc5c3O4)cc2)cc1]

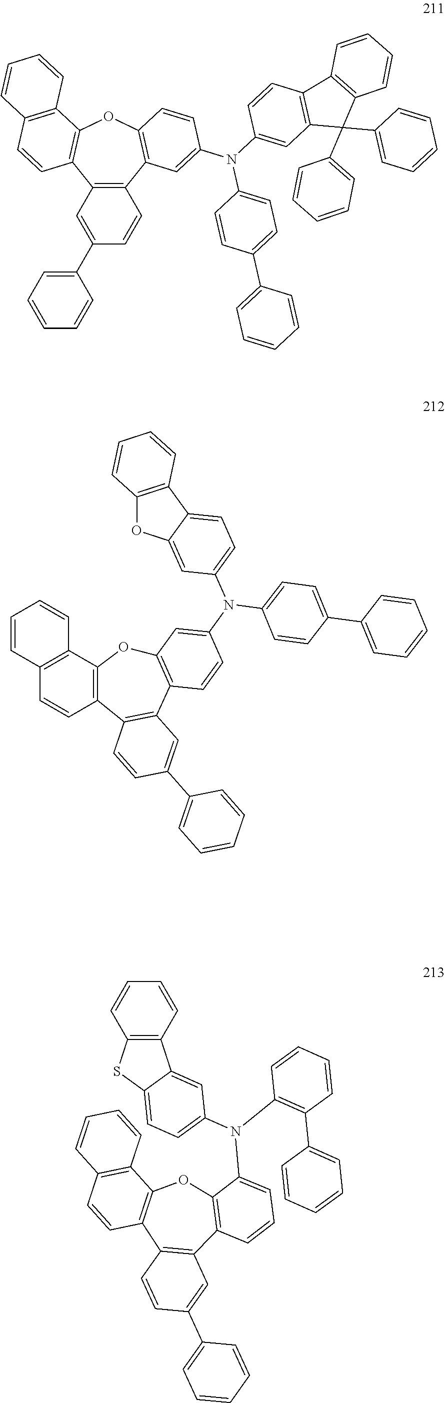 [SMILES: c1ccc(-c2ccc(N(c3ccc4c(c3)-c3ccc(-c5ccccc5)cc3-c3ccc5ccccc5c3O4)c3ccc4c(c3)C(c3ccccc3)(c3ccccc3)c3ccccc3-4)cc2)cc1 ; c1ccc(-c2ccc(N(c3ccc4c(c3)Oc3c(ccc5ccccc35)-c3ccc(-c5ccccc5)cc3-4)c3ccc4c(c3)oc3ccccc34)cc2)cc1 ; c1ccc(-c2ccc3c(c2)-c2cccc(N(c4ccc5sc6ccccc6c5c4)c4ccccc4-c4ccccc4)c2Oc2c-3ccc3ccccc23)cc1]

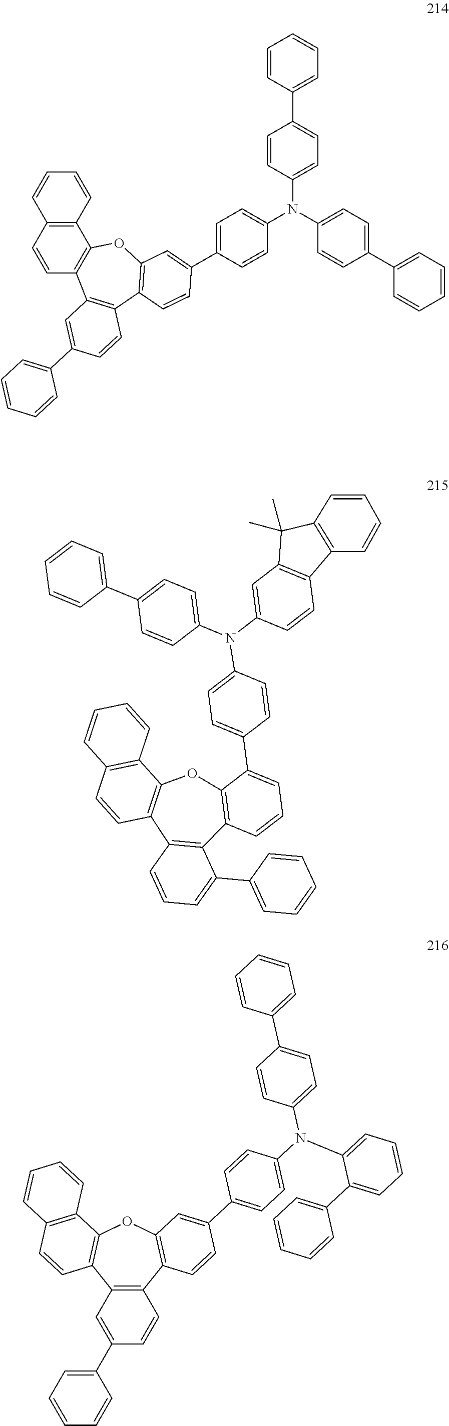 [SMILES: c1ccc(-c2ccc(N(c3ccc(-c4ccccc4)cc3)c3ccc(-c4ccc5c(c4)Oc4c(ccc6ccccc46)-c4cc(-c6ccccc6)ccc4-5)cc3)cc2)cc1 ; CC1(C)c2ccccc2-c2ccc(N(c3ccc(-c4ccccc4)cc3)c3ccc(-c4cccc5c4Oc4c(ccc6ccccc46)-c4cccc(-c6ccccc6)c4-5)cc3)cc21 ; c1ccc(-c2ccc(N(c3ccc(-c4ccc5c(c4)Oc4c(ccc6ccccc46)-c4cc(-c6ccccc6)ccc4-5)cc3)c3ccccc3-c3ccccc3)cc2)cc1]

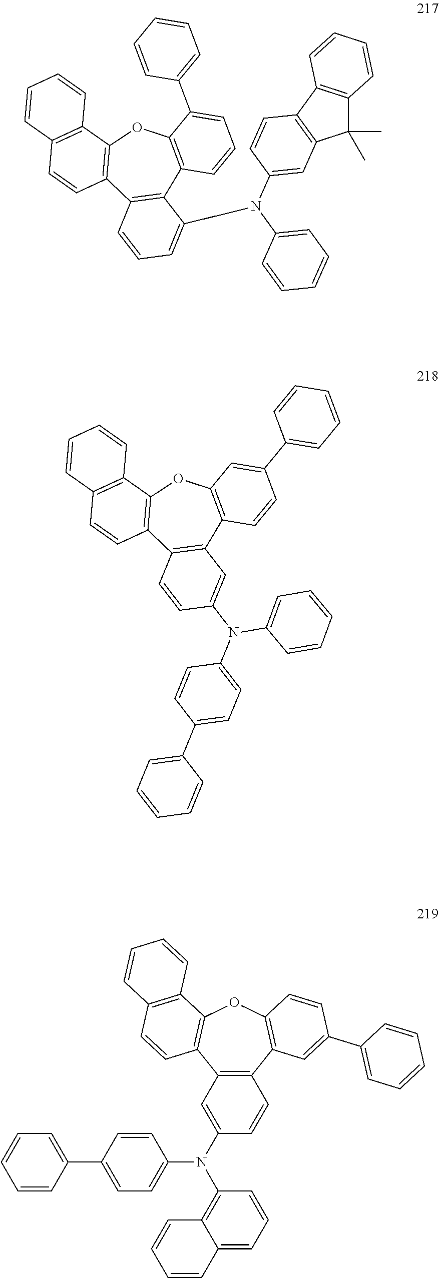 [SMILES: CC1(C)c2ccccc2-c2ccc(N(c3ccccc3)c3cccc4c3-c3cccc(-c5ccccc5)c3Oc3c-4ccc4ccccc34)cc21 ; c1ccc(-c2ccc(N(c3ccccc3)c3ccc4c(c3)-c3ccc(-c5ccccc5)cc3Oc3c-4ccc4ccccc34)cc2)cc1 ; c1ccc(-c2ccc(N(c3ccc4c(c3)-c3ccc5ccccc5c3Oc3ccc(-c5ccccc5)cc3-4)c3cccc4ccccc34)cc2)cc1]

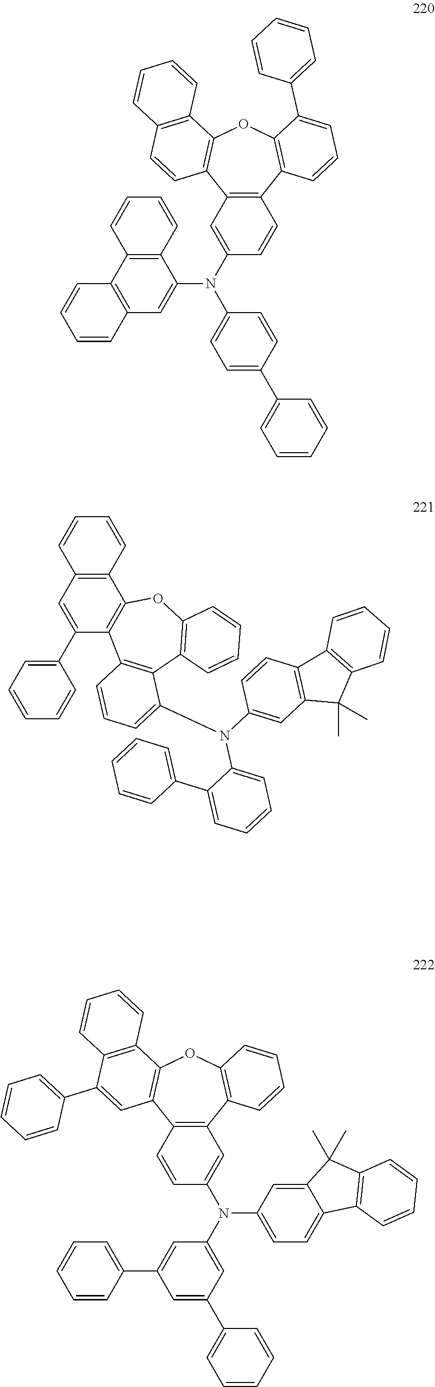 [SMILES: c1ccc(-c2ccc(N(c3ccc4c(c3)-c3ccc5ccccc5c3Oc3c(-c5ccccc5)cccc3-4)c3cc4ccccc4c4ccccc34)cc2)cc1 ; CC1(C)c2ccccc2-c2ccc(N(c3ccccc3-c3ccccc3)c3cccc4c3-c3ccccc3Oc3c-4c(-c4ccccc4)cc4ccccc34)cc21 ; CC1(C)c2ccccc2-c2ccc(N(c3cc(-c4ccccc4)cc(-c4ccccc4)c3)c3ccc4c(c3)-c3ccccc3Oc3c-4cc(-c4ccccc4)c4ccccc34)cc21]

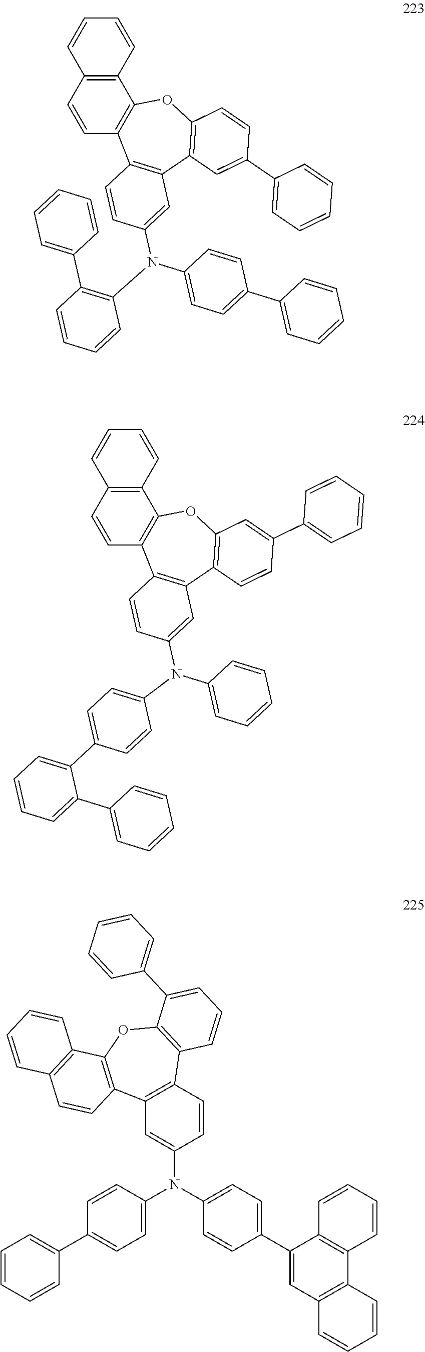 [SMILES: c1ccc(-c2ccc(N(c3ccc4c(c3)-c3cc(-c5ccccc5)ccc3Oc3c-4ccc4ccccc34)c3ccccc3-c3ccccc3)cc2)cc1 ; c1ccc(-c2ccc3c(c2)Oc2c(ccc4ccccc24)-c2ccc(N(c4ccccc4)c4ccc(-c5ccccc5-c5ccccc5)cc4)cc2-3)cc1 ; c1ccc(-c2ccc(N(c3ccc(-c4cc5ccccc5c5ccccc45)cc3)c3ccc4c(c3)-c3ccc5ccccc5c3Oc3c(-c5ccccc5)cccc3-4)cc2)cc1]

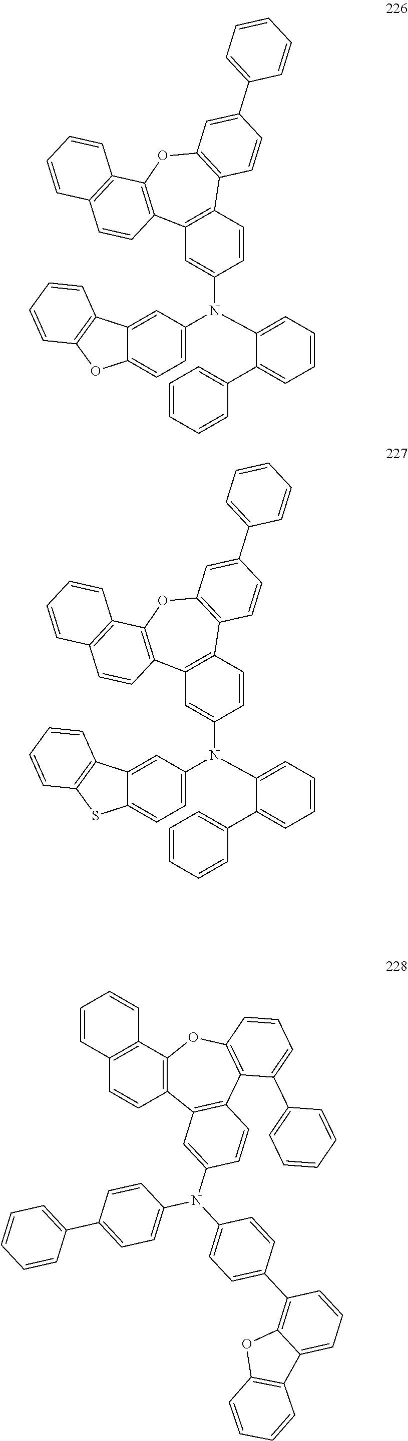 [SMILES: c1ccc(-c2ccc3c(c2)Oc2c(ccc4ccccc24)-c2cc(N(c4ccc5oc6ccccc6c5c4)c4ccccc4-c4ccccc4)ccc2-3)cc1 ; c1ccc(-c2ccc3c(c2)Oc2c(ccc4ccccc24)-c2cc(N(c4ccc5sc6ccccc6c5c4)c4ccccc4-c4ccccc4)ccc2-3)cc1 ; c1ccc(-c2ccc(N(c3ccc(-c4cccc5c4oc4ccccc45)cc3)c3ccc4c(c3)-c3ccc5ccccc5c3Oc3cccc(-c5ccccc5)c3-4)cc2)cc1]

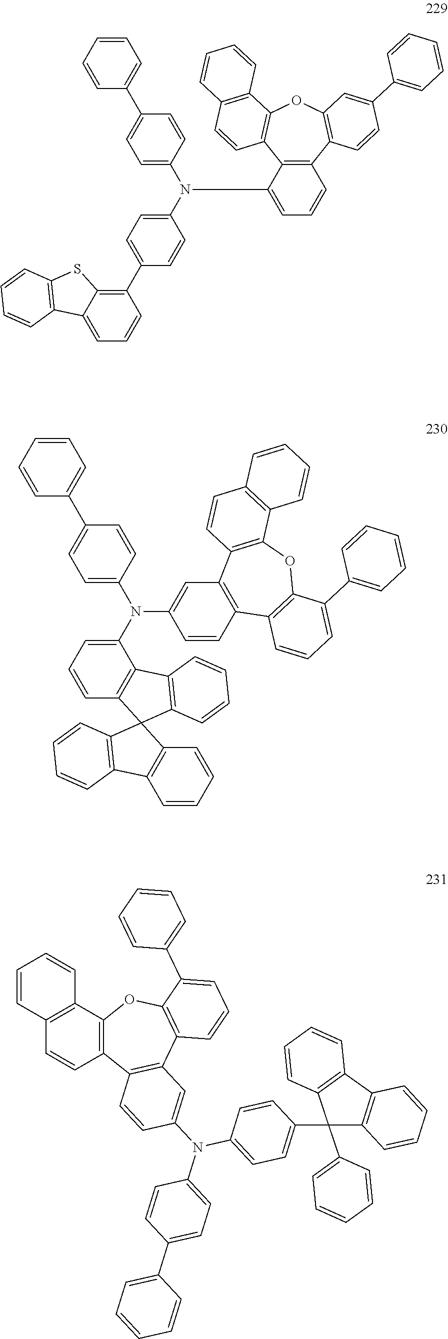 [SMILES: c1ccc(-c2ccc(N(c3ccc(-c4cccc5c4sc4ccccc45)cc3)c3cccc4c3-c3ccc5ccccc5c3Oc3cc(-c5ccccc5)ccc3-4)cc2)cc1 ; c1ccc(-c2ccc(N(c3ccc4c(c3)-c3ccc5ccccc5c3Oc3c(-c5ccccc5)cccc3-4)c3cccc4c3-c3ccccc3C43c4ccccc4-c4ccccc43)cc2)cc1 ; c1ccc(-c2ccc(N(c3ccc(C4(c5ccccc5)c5ccccc5-c5ccccc54)cc3)c3ccc4c(c3)-c3cccc(-c5ccccc5)c3Oc3c-4ccc4ccccc34)cc2)cc1]

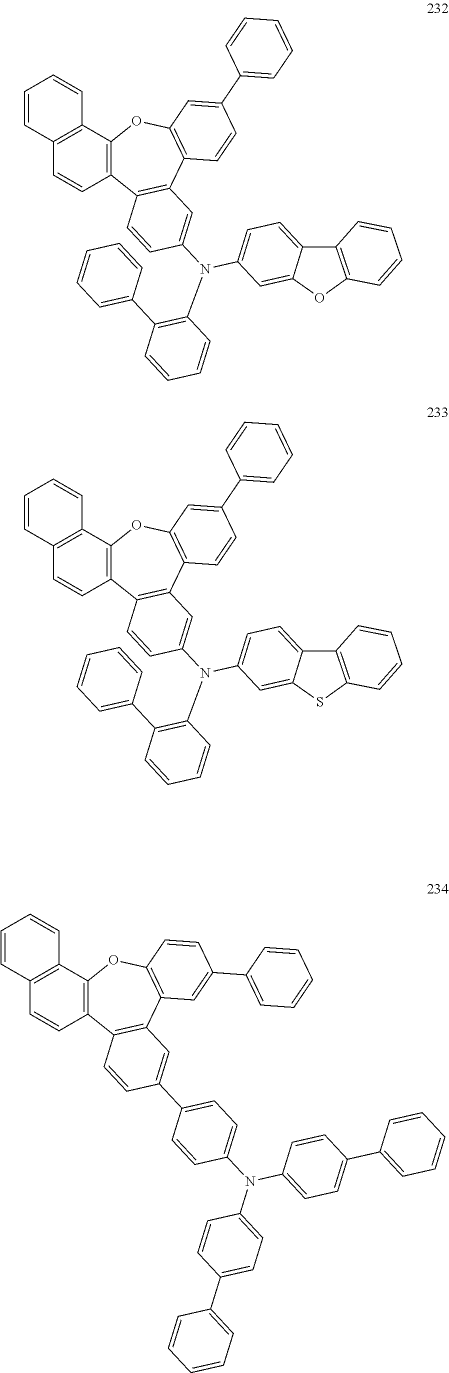 [SMILES: c1ccc(-c2ccc3c(c2)Oc2c(ccc4ccccc24)-c2ccc(N(c4ccc5c(c4)oc4ccccc45)c4ccccc4-c4ccccc4)cc2-3)cc1 ; c1ccc(-c2ccc3c(c2)Oc2c(ccc4ccccc24)-c2ccc(N(c4ccc5c(c4)sc4ccccc45)c4ccccc4-c4ccccc4)cc2-3)cc1 ; c1ccc(-c2ccc(N(c3ccc(-c4ccccc4)cc3)c3ccc(-c4ccc5c(c4)-c4cc(-c6ccccc6)ccc4Oc4c-5ccc5ccccc45)cc3)cc2)cc1]

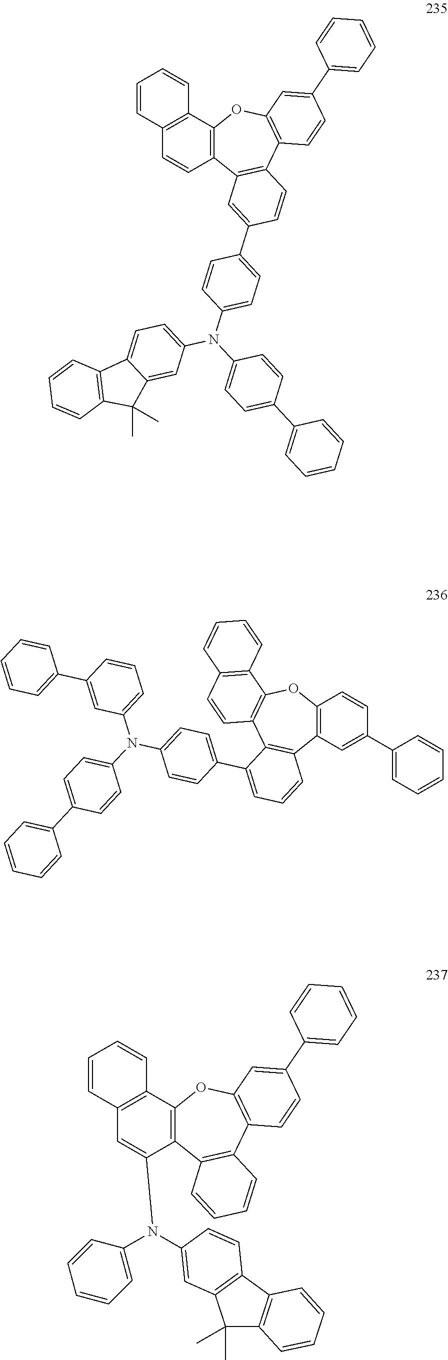 [SMILES: CC1(C)c2ccccc2-c2ccc(N(c3ccc(-c4ccccc4)cc3)c3ccc(-c4ccc5c(c4)-c4ccc6ccccc6c4Oc4cc(-c6ccccc6)ccc4-5)cc3)cc21 ; c1ccc(-c2ccc(N(c3ccc(-c4cccc5c4-c4ccc6ccccc6c4Oc4ccc(-c6ccccc6)cc4-5)cc3)c3cccc(-c4ccccc4)c3)cc2)cc1 ; CC1(C)c2ccccc2-c2ccc(N(c3ccccc3)c3cc4ccccc4c4c3-c3ccccc3-c3ccc(-c5ccccc5)cc3O4)cc21]

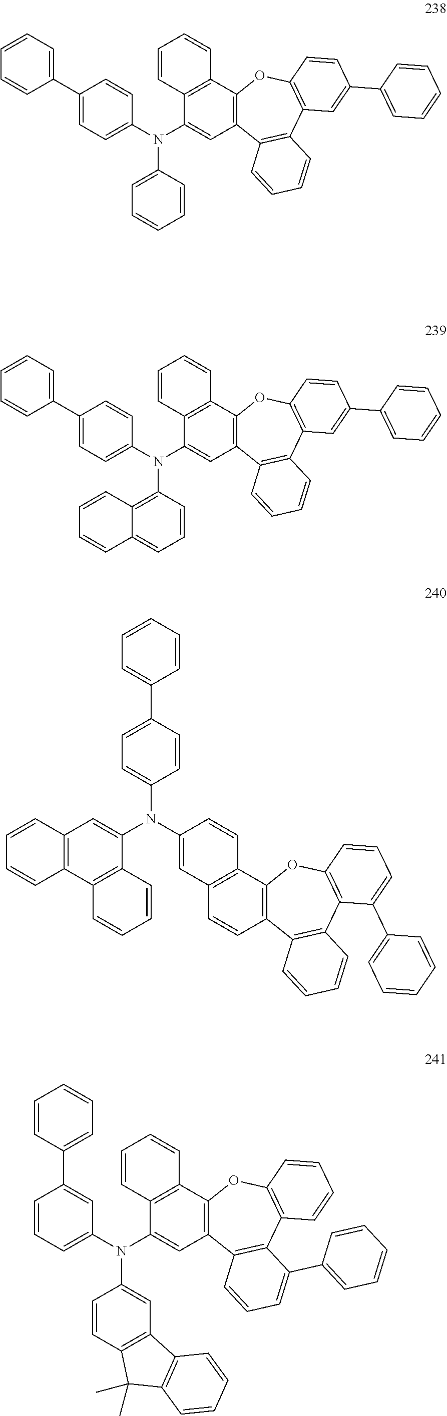 [SMILES: c1ccc(-c2ccc(N(c3ccccc3)c3cc4c(c5ccccc35)Oc3ccc(-c5ccccc5)cc3-c3ccccc3-4)cc2)cc1 ; c1ccc(-c2ccc(N(c3cccc4ccccc34)c3cc4c(c5ccccc35)Oc3ccc(-c5ccccc5)cc3-c3ccccc3-4)cc2)cc1 ; c1ccc(-c2ccc(N(c3ccc4c5c(ccc4c3)-c3ccccc3-c3c(cccc3-c3ccccc3)O5)c3cc4ccccc4c4ccccc34)cc2)cc1 ; CC1(C)c2ccccc2-c2cc(N(c3cccc(-c4ccccc4)c3)c3cc4c(c5ccccc35)Oc3ccccc3-c3c(-c5ccccc5)cccc3-4)ccc21]

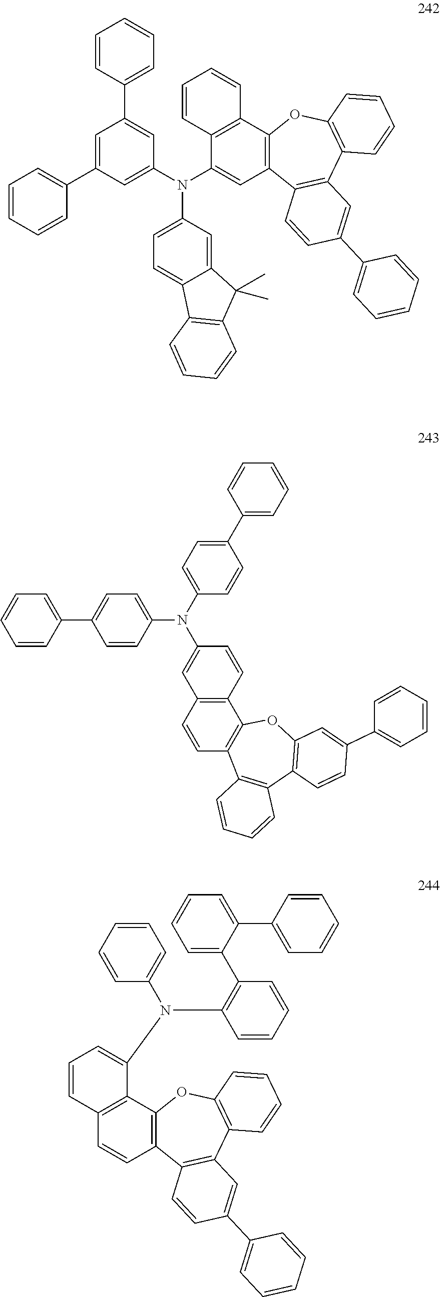 [SMILES: CC1(C)c2ccccc2-c2ccc(N(c3cc(-c4ccccc4)cc(-c4ccccc4)c3)c3cc4c(c5ccccc35)Oc3ccccc3-c3cc(-c5ccccc5)ccc3-4)cc21 ; c1ccc(-c2ccc(N(c3ccc(-c4ccccc4)cc3)c3ccc4c5c(ccc4c3)-c3ccccc3-c3ccc(-c4ccccc4)cc3O5)cc2)cc1 ; c1ccc(-c2ccc3c(c2)-c2ccccc2Oc2c-3ccc3cccc(N(c4ccccc4)c4ccccc4-c4ccccc4-c4ccccc4)c23)cc1]

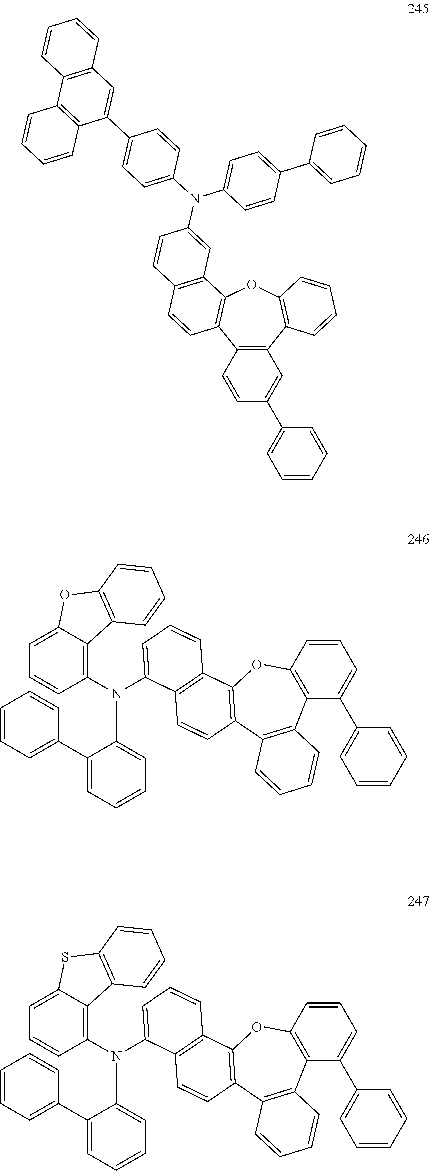 [SMILES: c1ccc(-c2ccc(N(c3ccc(-c4cc5ccccc5c5ccccc45)cc3)c3ccc4ccc5c(c4c3)Oc3ccccc3-c3cc(-c4ccccc4)ccc3-5)cc2)cc1 ; c1ccc(-c2ccccc2N(c2cccc3c4c(ccc23)-c2ccccc2-c2c(cccc2-c2ccccc2)O4)c2cccc3oc4ccccc4c23)cc1 ; c1ccc(-c2ccccc2N(c2cccc3c4c(ccc23)-c2ccccc2-c2c(cccc2-c2ccccc2)O4)c2cccc3sc4ccccc4c23)cc1]

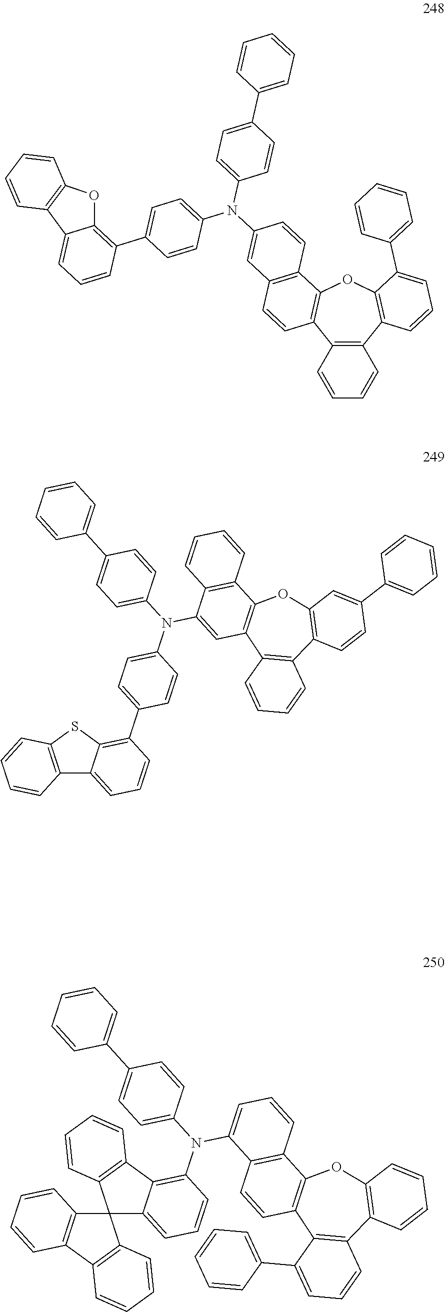 [SMILES: c1ccc(-c2ccc(N(c3ccc(-c4cccc5c4oc4ccccc45)cc3)c3ccc4c5c(ccc4c3)-c3ccccc3-c3cccc(-c4ccccc4)c3O5)cc2)cc1 ; c1ccc(-c2ccc(N(c3ccc(-c4cccc5c4sc4ccccc45)cc3)c3cc4c(c5ccccc35)Oc3cc(-c5ccccc5)ccc3-c3ccccc3-4)cc2)cc1 ; c1ccc(-c2ccc(N(c3cccc4c3-c3ccccc3C43c4ccccc4-c4ccccc43)c3cccc4c5c(ccc34)-c3c(-c4ccccc4)cccc3-c3ccccc3O5)cc2)cc1]

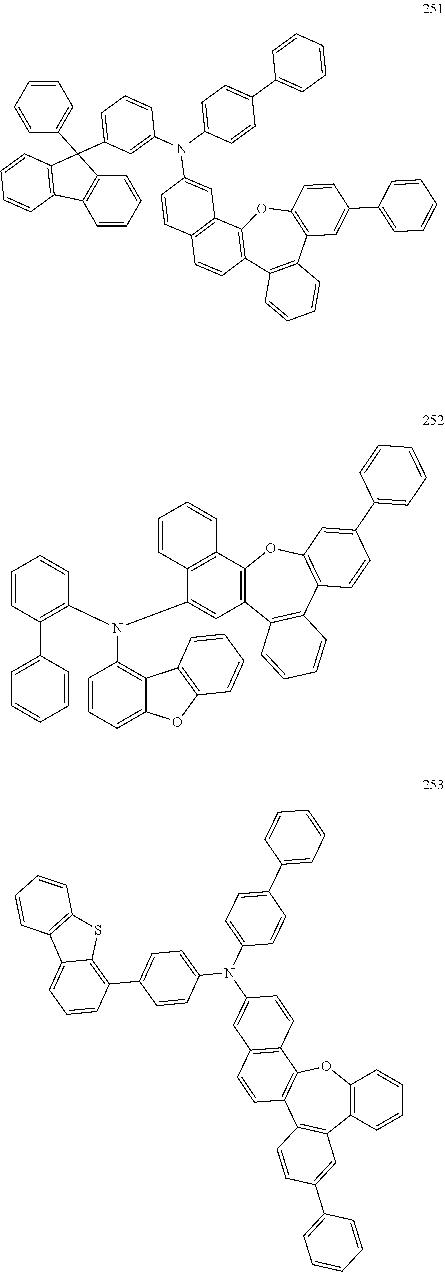 [SMILES: c1ccc(-c2ccc(N(c3cccc(C4(c5ccccc5)c5ccccc5-c5ccccc54)c3)c3ccc4ccc5c(c4c3)Oc3ccc(-c4ccccc4)cc3-c3ccccc3-5)cc2)cc1 ; c1ccc(-c2ccc3c(c2)Oc2c(cc(N(c4ccccc4-c4ccccc4)c4cccc5oc6ccccc6c45)c4ccccc24)-c2ccccc2-3)cc1 ; c1ccc(-c2ccc(N(c3ccc(-c4cccc5c4sc4ccccc45)cc3)c3ccc4c5c(ccc4c3)-c3ccc(-c4ccccc4)cc3-c3ccccc3O5)cc2)cc1]

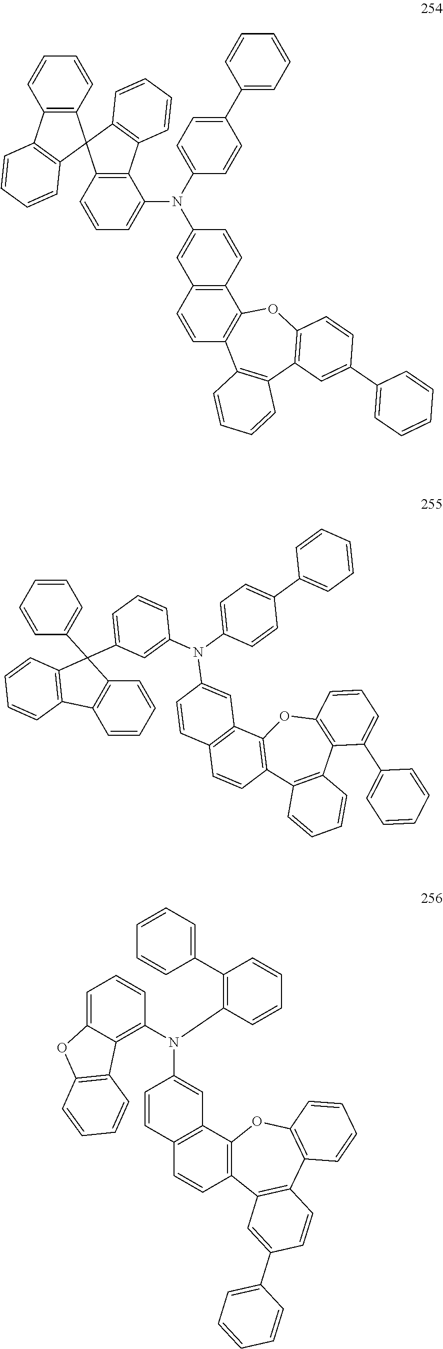 [SMILES: c1ccc(-c2ccc(N(c3ccc4c5c(ccc4c3)-c3ccccc3-c3cc(-c4ccccc4)ccc3O5)c3cccc4c3-c3ccccc3C43c4ccccc4-c4ccccc43)cc2)cc1 ; c1ccc(-c2ccc(N(c3cccc(C4(c5ccccc5)c5ccccc5-c5ccccc54)c3)c3ccc4ccc5c(c4c3)Oc3cccc(-c4ccccc4)c3-c3ccccc3-5)cc2)cc1 ; c1ccc(-c2ccc3c(c2)-c2ccc4ccc(N(c5ccccc5-c5ccccc5)c5cccc6oc7ccccc7c56)cc4c2Oc2ccccc2-3)cc1]

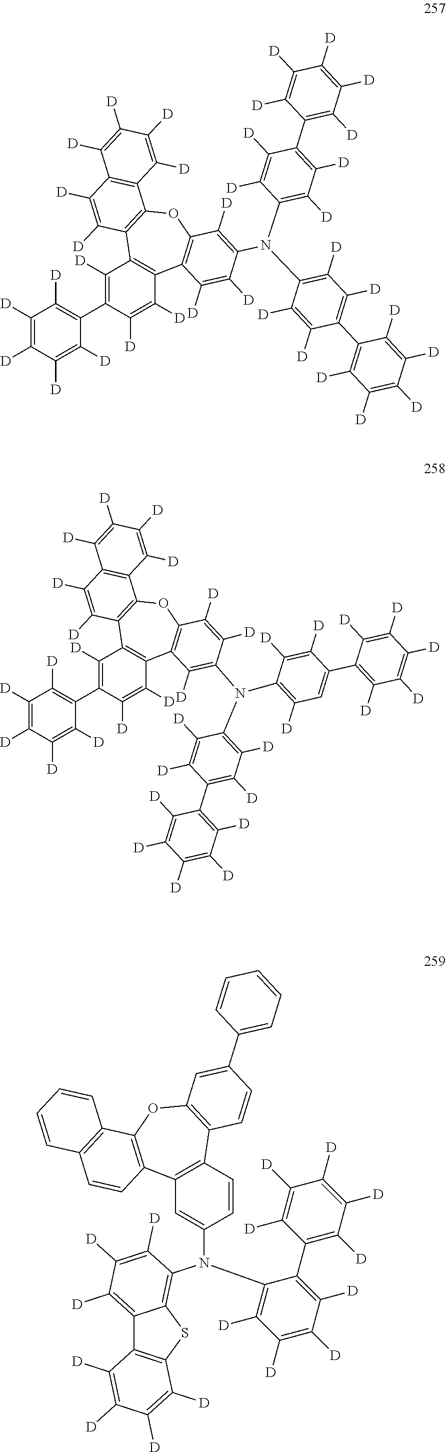

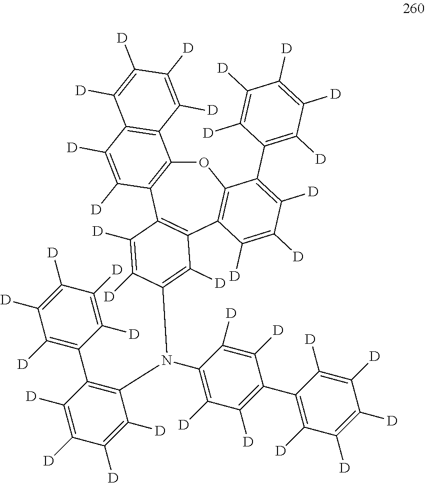 [SMILES: [2H]c1c([2H])c([2H])c(-c2c([2H])c([2H])c(N(c3c([2H])c([2H])c4c(c3[2H])-c3c([2H])c([2H])c([2H])c(-c5c([2H])c([2H])c([2H])c([2H])c5[2H])c3Oc3c-4c([2H])c([2H])c4c([2H])c([2H])c([2H])c([2H])c34)c3c([2H])c([2H])c([2H])c([2H])c3-c3c([2H])c([2H])c([2H])c([2H])c3[2H])c([2H])c2[2H])c([2H])c1[2H]]

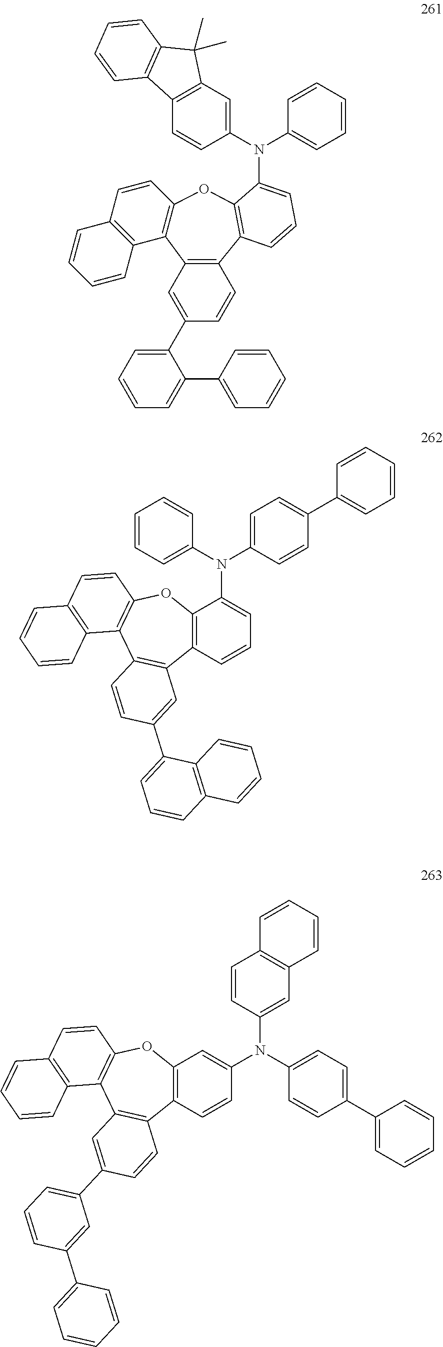

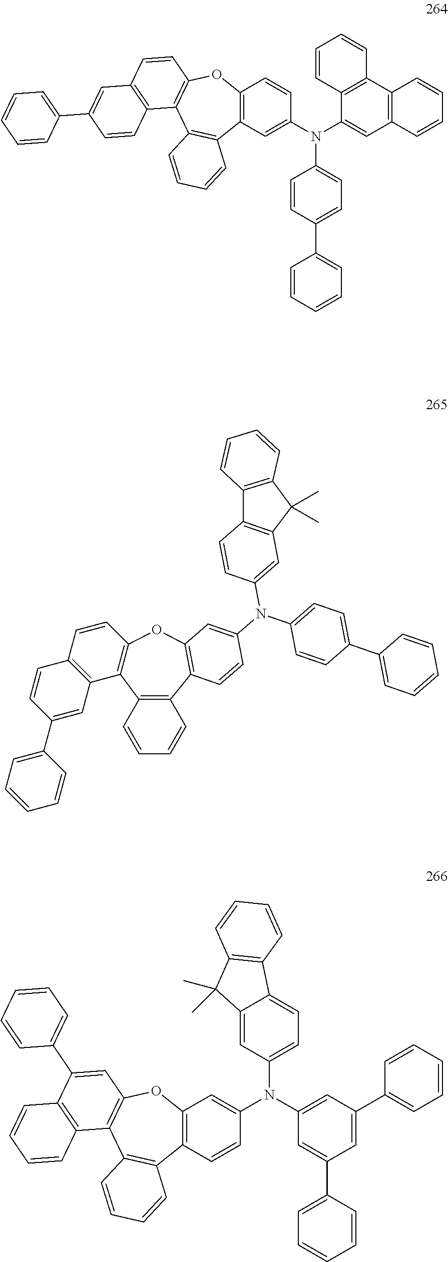

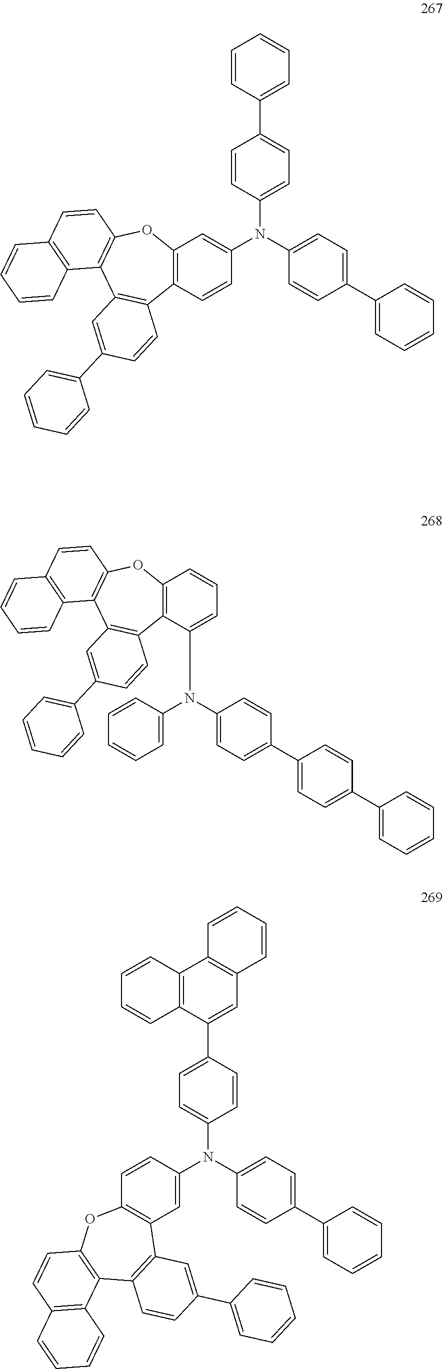 [SMILES: c1ccc(-c2ccc(N(c3ccc(-c4ccccc4)cc3)c3ccc4c(c3)Oc3ccc5ccccc5c3-c3cc(-c5ccccc5)ccc3-4)cc2)cc1 ; c1ccc(-c2ccc(-c3ccc(N(c4ccccc4)c4cccc5c4-c4ccc(-c6ccccc6)cc4-c4c(ccc6ccccc46)O5)cc3)cc2)cc1 ; c1ccc(-c2ccc(N(c3ccc(-c4cc5ccccc5c5ccccc45)cc3)c3ccc4c(c3)-c3cc(-c5ccccc5)ccc3-c3c(ccc5ccccc35)O4)cc2)cc1]

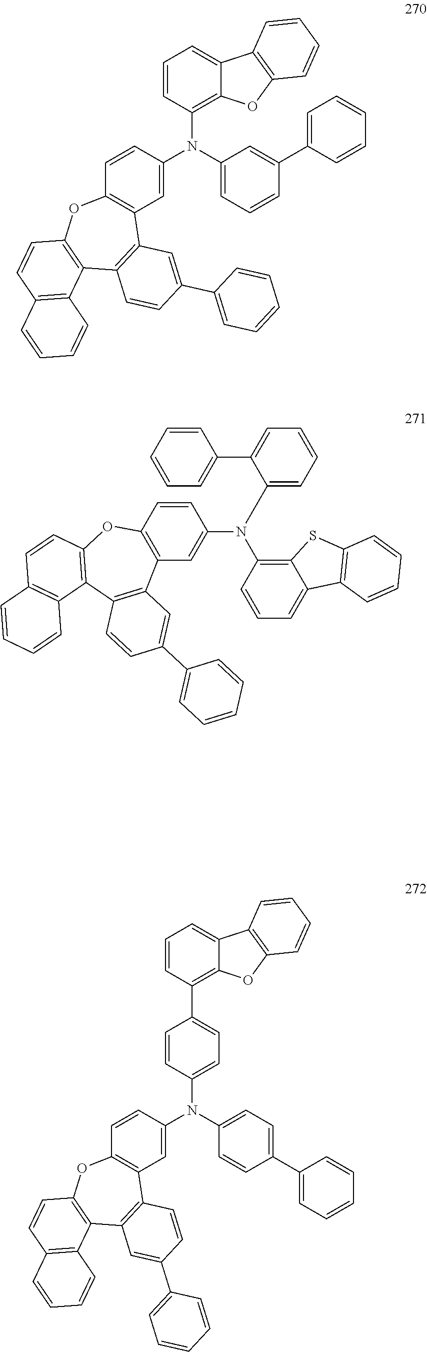 [SMILES: c1ccc(-c2cccc(N(c3ccc4c(c3)-c3cc(-c5ccccc5)ccc3-c3c(ccc5ccccc35)O4)c3cccc4c3oc3ccccc34)c2)cc1 ; c1ccc(-c2ccc3c(c2)-c2cc(N(c4ccccc4-c4ccccc4)c4cccc5c4sc4ccccc45)ccc2Oc2ccc4ccccc4c2-3)cc1 ; c1ccc(-c2ccc(N(c3ccc(-c4cccc5c4oc4ccccc45)cc3)c3ccc4c(c3)-c3ccc(-c5ccccc5)cc3-c3c(ccc5ccccc35)O4)cc2)cc1]

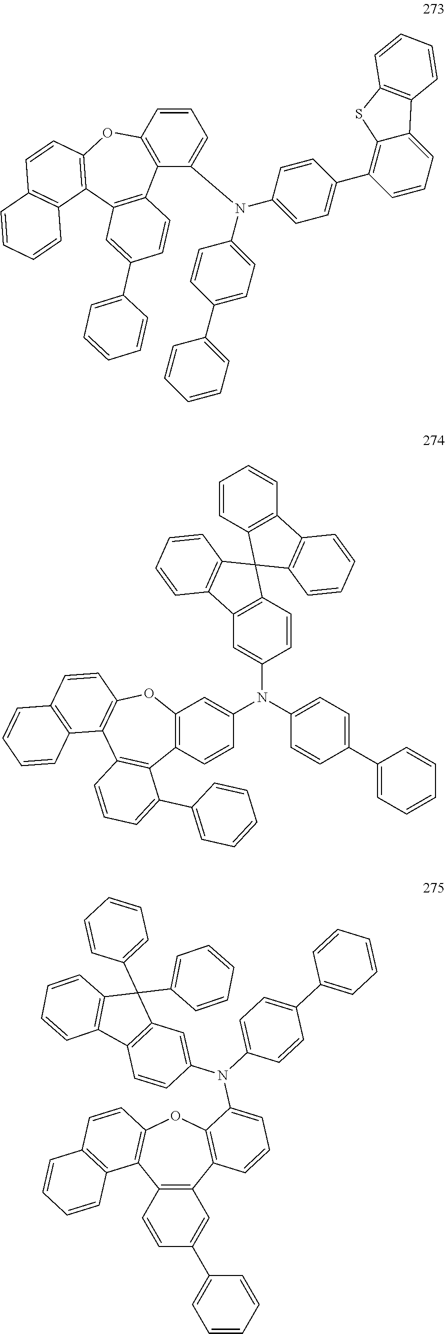 [SMILES: c1ccc(-c2ccc(N(c3ccc(-c4cccc5c4sc4ccccc45)cc3)c3cccc4c3-c3ccc(-c5ccccc5)cc3-c3c(ccc5ccccc35)O4)cc2)cc1 ; c1ccc(-c2ccc(N(c3ccc4c(c3)Oc3ccc5ccccc5c3-c3cccc(-c5ccccc5)c3-4)c3ccc4c(c3)-c3ccccc3C43c4ccccc4-c4ccccc43)cc2)cc1 ; c1ccc(-c2ccc(N(c3ccc4c(c3)C(c3ccccc3)(c3ccccc3)c3ccccc3-4)c3cccc4c3Oc3ccc5ccccc5c3-c3ccc(-c5ccccc5)cc3-4)cc2)cc1]

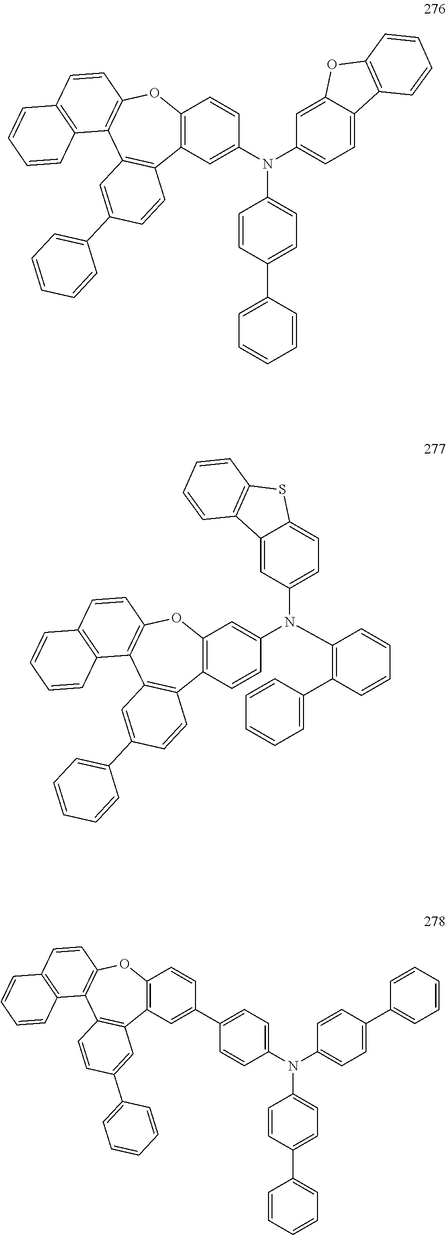

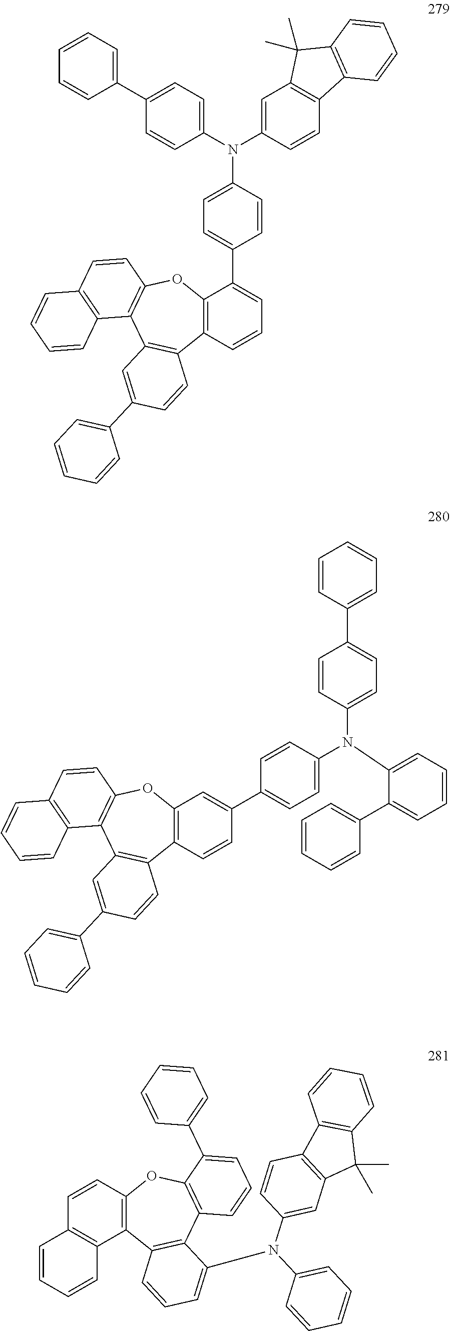 [SMILES: CC1(C)c2ccccc2-c2ccc(N(c3ccc(-c4ccccc4)cc3)c3ccc(-c4cccc5c4Oc4ccc6ccccc6c4-c4cc(-c6ccccc6)ccc4-5)cc3)cc21 ; c1ccc(-c2ccc(N(c3ccc(-c4ccc5c(c4)Oc4ccc6ccccc6c4-c4cc(-c6ccccc6)ccc4-5)cc3)c3ccccc3-c3ccccc3)cc2)cc1 ; CC1(C)c2ccccc2-c2ccc(N(c3ccccc3)c3cccc4c3-c3cccc(-c5ccccc5)c3Oc3ccc5ccccc5c3-4)cc21]

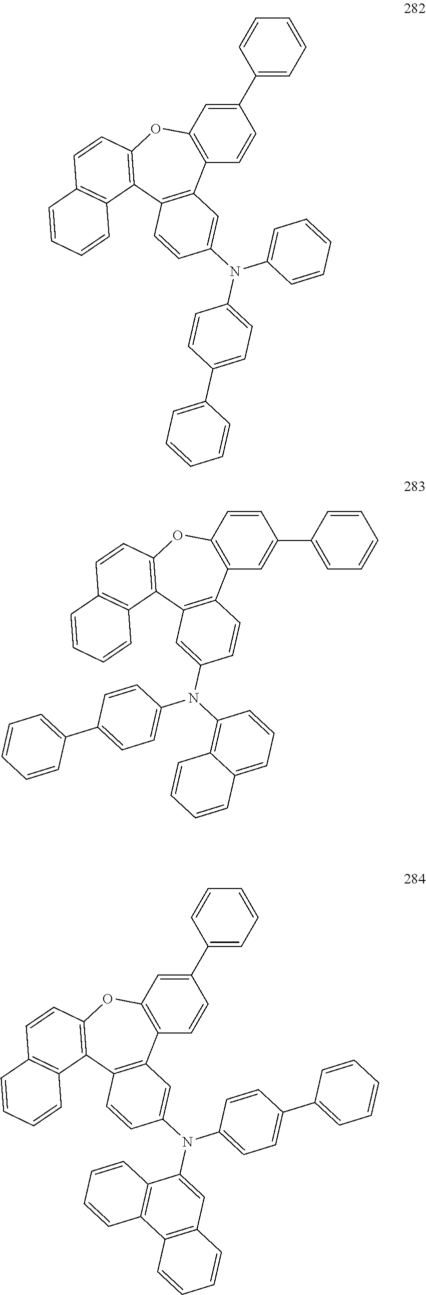 [SMILES: c1ccc(-c2ccc(N(c3ccccc3)c3ccc4c(c3)-c3ccc(-c5ccccc5)cc3Oc3ccc5ccccc5c3-4)cc2)cc1 ; c1ccc(-c2ccc(N(c3ccc4c(c3)-c3c(ccc5ccccc35)Oc3ccc(-c5ccccc5)cc3-4)c3cccc4ccccc34)cc2)cc1 ; c1ccc(-c2ccc(N(c3ccc4c(c3)-c3ccc(-c5ccccc5)cc3Oc3ccc5ccccc5c3-4)c3cc4ccccc4c4ccccc34)cc2)cc1]

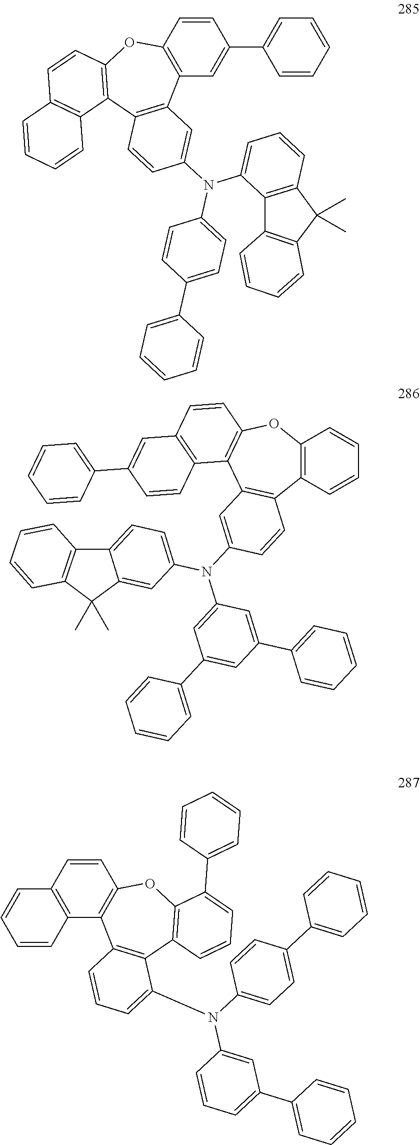

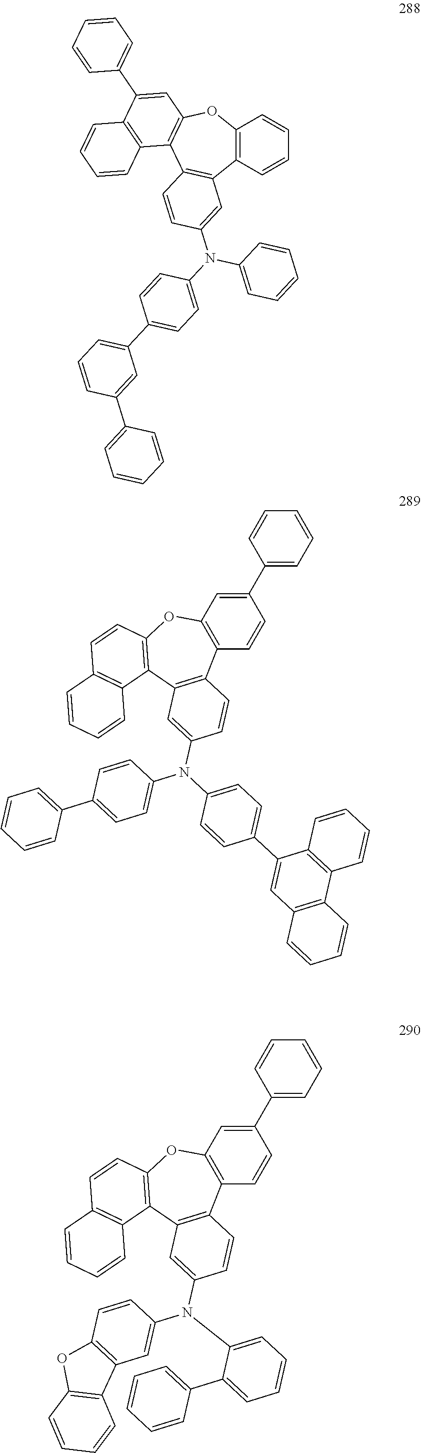 [SMILES: c1ccc(-c2cccc(-c3ccc(N(c4ccccc4)c4ccc5c(c4)-c4ccccc4Oc4cc(-c6ccccc6)c6ccccc6c4-5)cc3)c2)cc1 ; c1ccc(-c2ccc(N(c3ccc(-c4cc5ccccc5c5ccccc45)cc3)c3ccc4c(c3)-c3c(ccc5ccccc35)Oc3cc(-c5ccccc5)ccc3-4)cc2)cc1 ; c1ccc(-c2ccc3c(c2)Oc2ccc4ccccc4c2-c2cc(N(c4ccc5oc6ccccc6c5c4)c4ccccc4-c4ccccc4)ccc2-3)cc1]

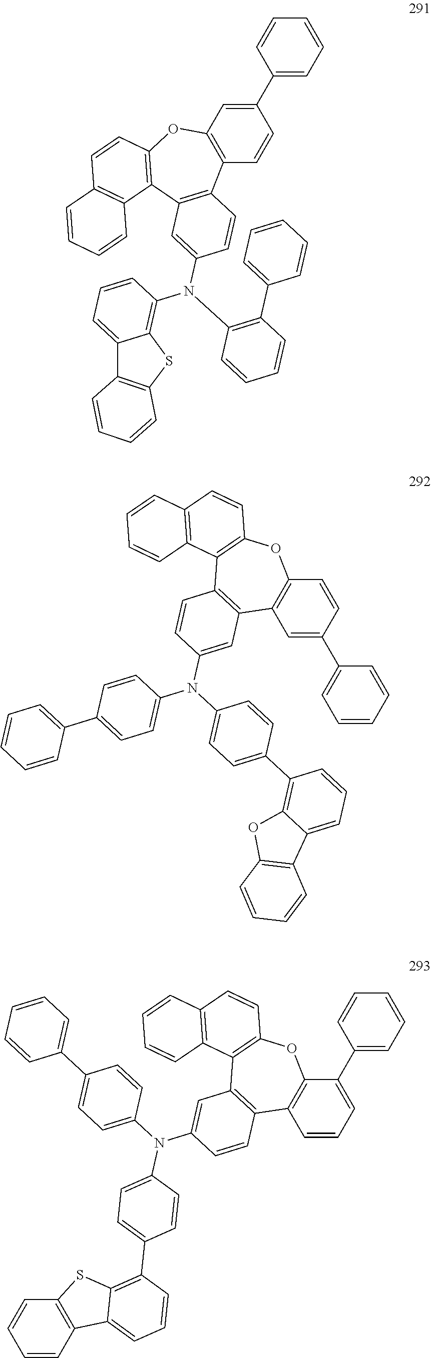 [SMILES: c1ccc(-c2ccc3c(c2)Oc2ccc4ccccc4c2-c2cc(N(c4ccccc4-c4ccccc4)c4cccc5c4sc4ccccc45)ccc2-3)cc1 ; c1ccc(-c2ccc(N(c3ccc(-c4cccc5c4oc4ccccc45)cc3)c3ccc4c(c3)-c3cc(-c5ccccc5)ccc3Oc3ccc5ccccc5c3-4)cc2)cc1 ; c1ccc(-c2ccc(N(c3ccc(-c4cccc5c4sc4ccccc45)cc3)c3ccc4c(c3)-c3c(ccc5ccccc35)Oc3c(-c5ccccc5)cccc3-4)cc2)cc1]

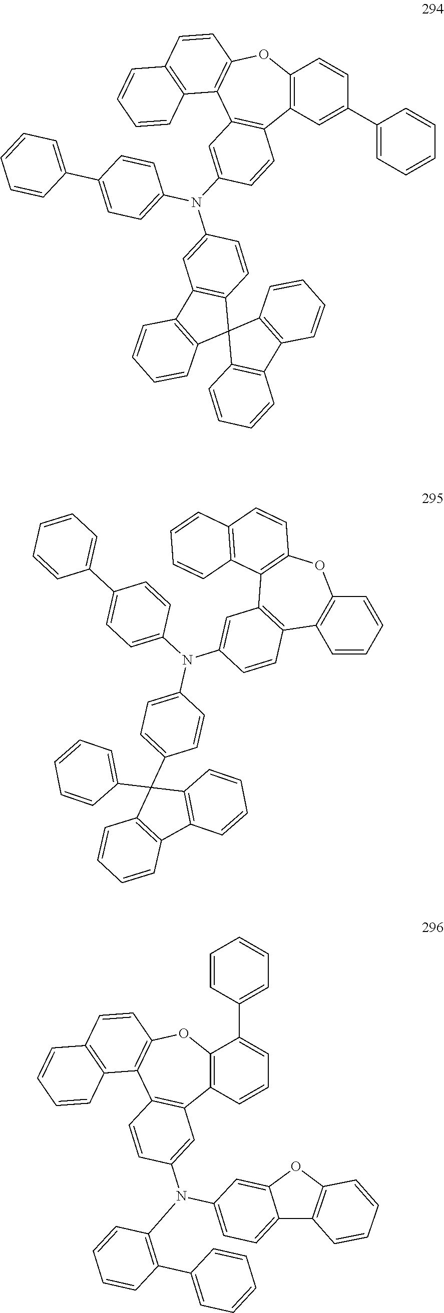 [SMILES: c1ccc(-c2ccc(N(c3ccc4c(c3)-c3c(ccc5ccccc35)Oc3ccc(-c5ccccc5)cc3-4)c3ccc4c(c3)-c3ccccc3C43c4ccccc4-c4ccccc43)cc2)cc1 ; c1ccc(-c2ccc(N(c3ccc(C4(c5ccccc5)c5ccccc5-c5ccccc54)cc3)c3ccc4c(c3)-c3c(ccc5ccccc35)Oc3ccccc3-4)cc2)cc1 ; c1ccc(-c2ccccc2N(c2ccc3c(c2)-c2cccc(-c4ccccc4)c2Oc2ccc4ccccc4c2-3)c2ccc3c(c2)oc2ccccc23)cc1]

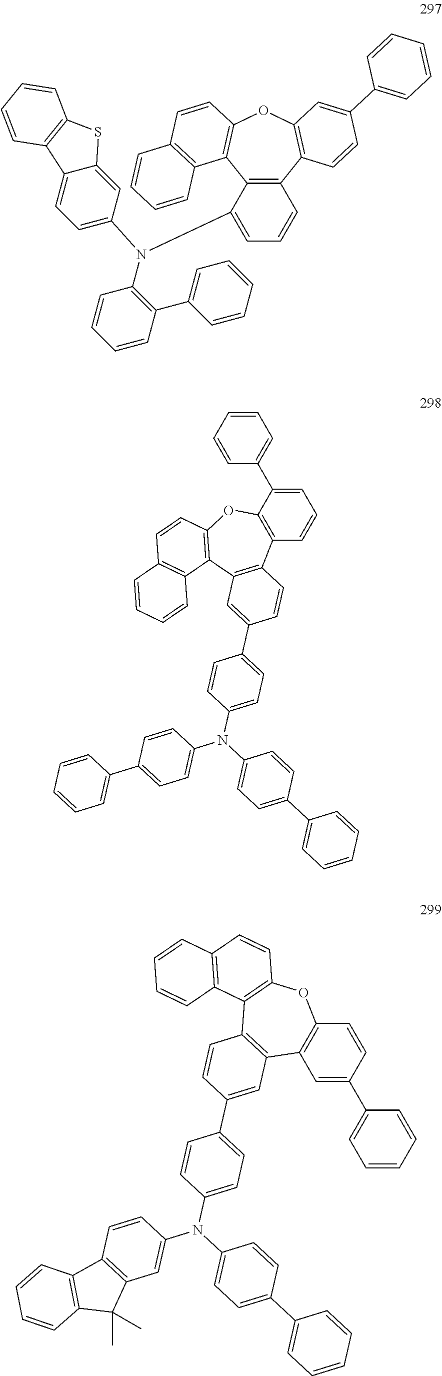 [SMILES: c1ccc(-c2ccc3c(c2)Oc2ccc4ccccc4c2-c2c-3cccc2N(c2ccc3c(c2)sc2ccccc23)c2ccccc2-c2ccccc2)cc1 ; c1ccc(-c2ccc(N(c3ccc(-c4ccccc4)cc3)c3ccc(-c4ccc5c(c4)-c4c(ccc6ccccc46)Oc4c(-c6ccccc6)cccc4-5)cc3)cc2)cc1 ; CC1(C)c2ccccc2-c2ccc(N(c3ccc(-c4ccccc4)cc3)c3ccc(-c4ccc5c(c4)-c4cc(-c6ccccc6)ccc4Oc4ccc6ccccc6c4-5)cc3)cc21]

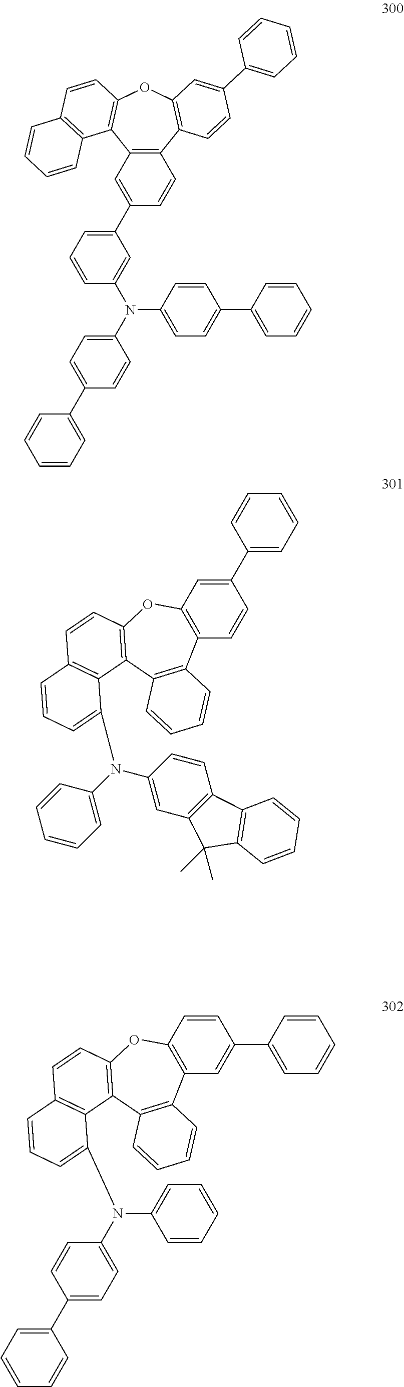 [SMILES: c1ccc(-c2ccc(N(c3ccc(-c4ccccc4)cc3)c3cccc(-c4ccc5c(c4)-c4c(ccc6ccccc46)Oc4cc(-c6ccccc6)ccc4-5)c3)cc2)cc1 ; CC1(C)c2ccccc2-c2ccc(N(c3ccccc3)c3cccc4ccc5c(c34)-c3ccccc3-c3ccc(-c4ccccc4)cc3O5)cc21 ; c1ccc(-c2ccc(N(c3ccccc3)c3cccc4ccc5c(c34)-c3ccccc3-c3cc(-c4ccccc4)ccc3O5)cc2)cc1]

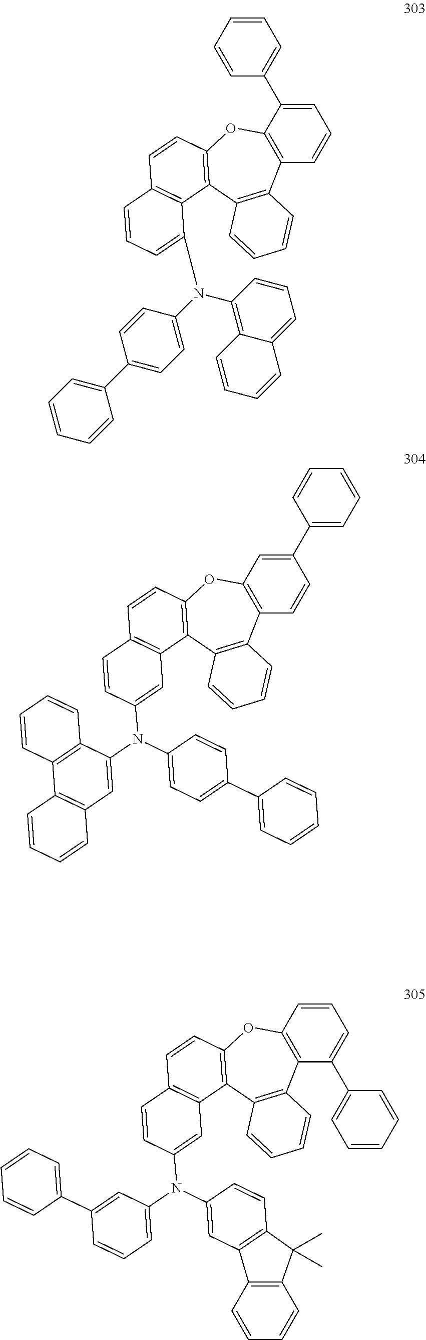 [SMILES: c1ccc(-c2ccc(N(c3cccc4ccccc34)c3cccc4ccc5c(c34)-c3ccccc3-c3cccc(-c4ccccc4)c3O5)cc2)cc1 ; c1ccc(-c2ccc(N(c3ccc4ccc5c(c4c3)-c3ccccc3-c3ccc(-c4ccccc4)cc3O5)c3cc4ccccc4c4ccccc34)cc2)cc1 ; CC1(C)c2ccccc2-c2cc(N(c3cccc(-c4ccccc4)c3)c3ccc4ccc5c(c4c3)-c3ccccc3-c3c(cccc3-c3ccccc3)O5)ccc21]

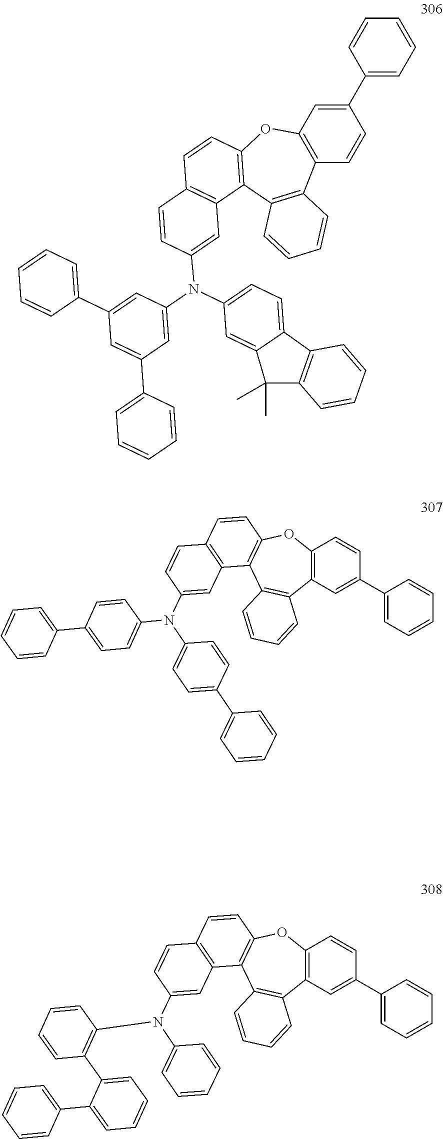 [SMILES: CC1(C)c2ccccc2-c2ccc(N(c3cc(-c4ccccc4)cc(-c4ccccc4)c3)c3ccc4ccc5c(c4c3)-c3ccccc3-c3ccc(-c4ccccc4)cc3O5)cc21 ; c1ccc(-c2ccc(N(c3ccc(-c4ccccc4)cc3)c3ccc4ccc5c(c4c3)-c3ccccc3-c3cc(-c4ccccc4)ccc3O5)cc2)cc1 ; c1ccc(-c2ccc3c(c2)-c2ccccc2-c2c(ccc4ccc(N(c5ccccc5)c5ccccc5-c5ccccc5-c5ccccc5)cc24)O3)cc1]

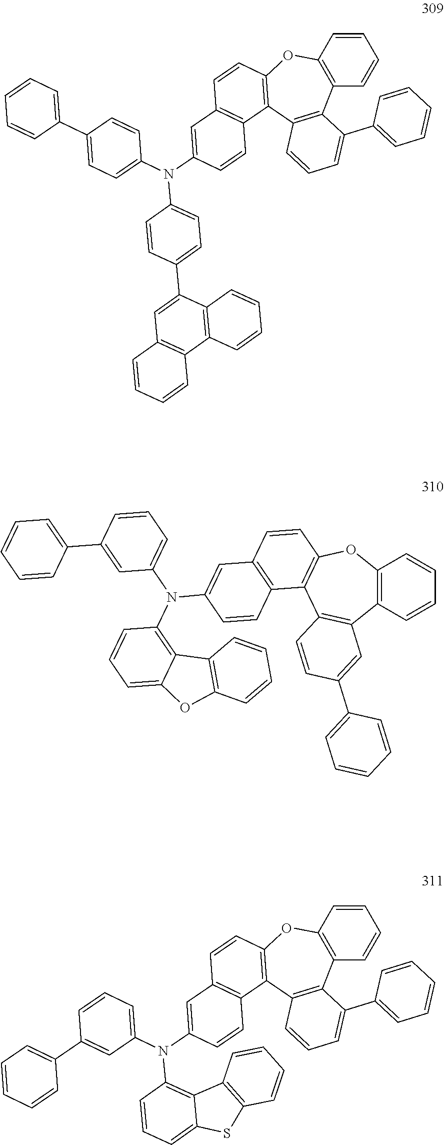 [SMILES: c1ccc(-c2ccc(N(c3ccc(-c4cc5ccccc5c5ccccc45)cc3)c3ccc4c5c(ccc4c3)Oc3ccccc3-c3c(-c4ccccc4)cccc3-5)cc2)cc1 ; c1ccc(-c2cccc(N(c3ccc4c5c(ccc4c3)Oc3ccccc3-c3cc(-c4ccccc4)ccc3-5)c3cccc4oc5ccccc5c34)c2)cc1 ; c1ccc(-c2cccc(N(c3ccc4c5c(ccc4c3)Oc3ccccc3-c3c(-c4ccccc4)cccc3-5)c3cccc4sc5ccccc5c34)c2)cc1]

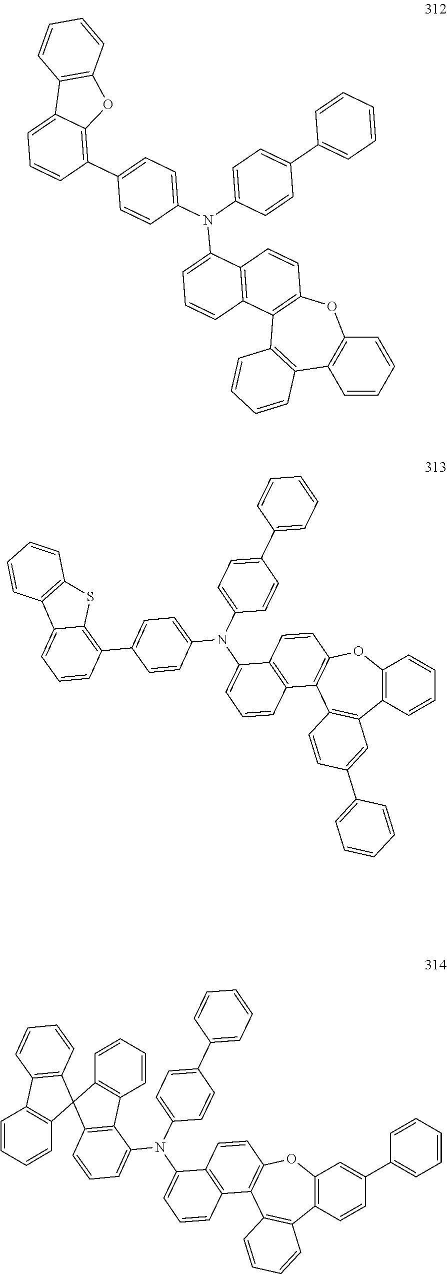 [SMILES: c1ccc(-c2ccc(N(c3ccc(-c4cccc5c4oc4ccccc45)cc3)c3cccc4c5c(ccc34)Oc3ccccc3-c3ccccc3-5)cc2)cc1 ; c1ccc(-c2ccc(N(c3ccc(-c4cccc5c4sc4ccccc45)cc3)c3cccc4c5c(ccc34)Oc3ccccc3-c3cc(-c4ccccc4)ccc3-5)cc2)cc1 ; c1ccc(-c2ccc(N(c3cccc4c3-c3ccccc3C43c4ccccc4-c4ccccc43)c3cccc4c5c(ccc34)Oc3cc(-c4ccccc4)ccc3-c3ccccc3-5)cc2)cc1]

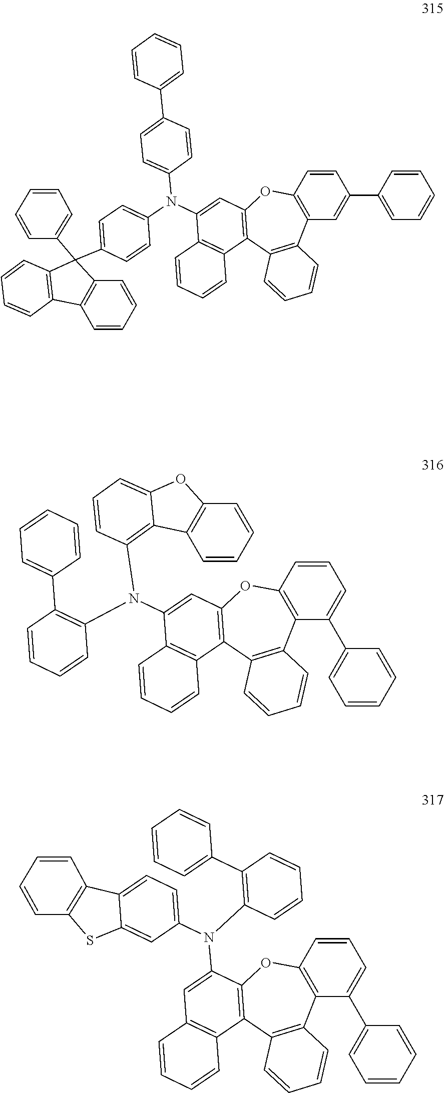 [SMILES: c1ccc(-c2ccc(N(c3ccc(C4(c5ccccc5)c5ccccc5-c5ccccc54)cc3)c3cc4c(c5ccccc35)-c3ccccc3-c3cc(-c5ccccc5)ccc3O4)cc2)cc1 ; c1ccc(-c2ccccc2N(c2cc3c(c4ccccc24)-c2ccccc2-c2c(cccc2-c2ccccc2)O3)c2cccc3oc4ccccc4c23)cc1 ; c1ccc(-c2ccccc2N(c2ccc3c(c2)sc2ccccc23)c2cc3ccccc3c3c2Oc2cccc(-c4ccccc4)c2-c2ccccc2-3)cc1]

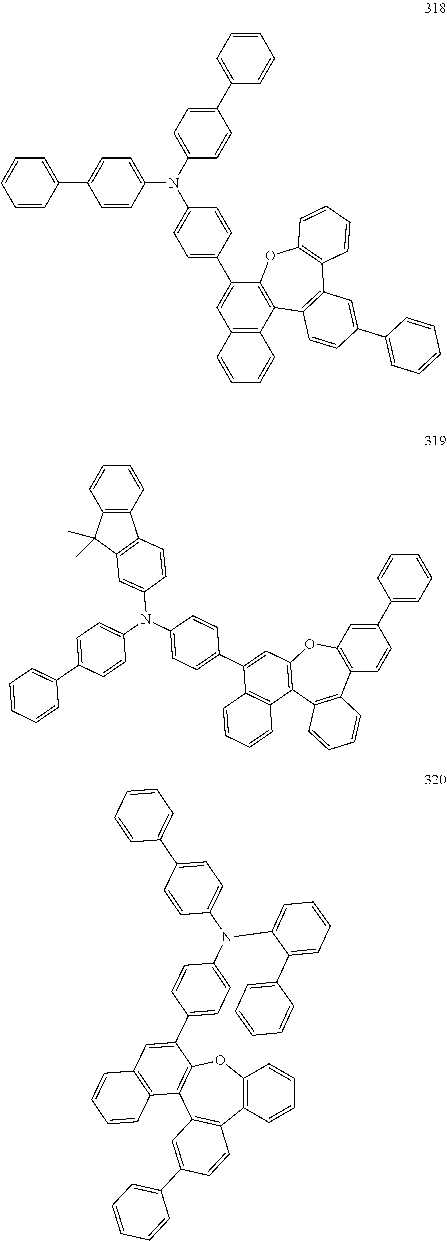 [SMILES: c1ccc(-c2ccc(N(c3ccc(-c4ccccc4)cc3)c3ccc(-c4cc5ccccc5c5c4Oc4ccccc4-c4cc(-c6ccccc6)ccc4-5)cc3)cc2)cc1 ; CC1(C)c2ccccc2-c2ccc(N(c3ccc(-c4ccccc4)cc3)c3ccc(-c4cc5c(c6ccccc46)-c4ccccc4-c4ccc(-c6ccccc6)cc4O5)cc3)cc21 ; c1ccc(-c2ccc(N(c3ccc(-c4cc5ccccc5c5c4Oc4ccccc4-c4ccc(-c6ccccc6)cc4-5)cc3)c3ccccc3-c3ccccc3)cc2)cc1]

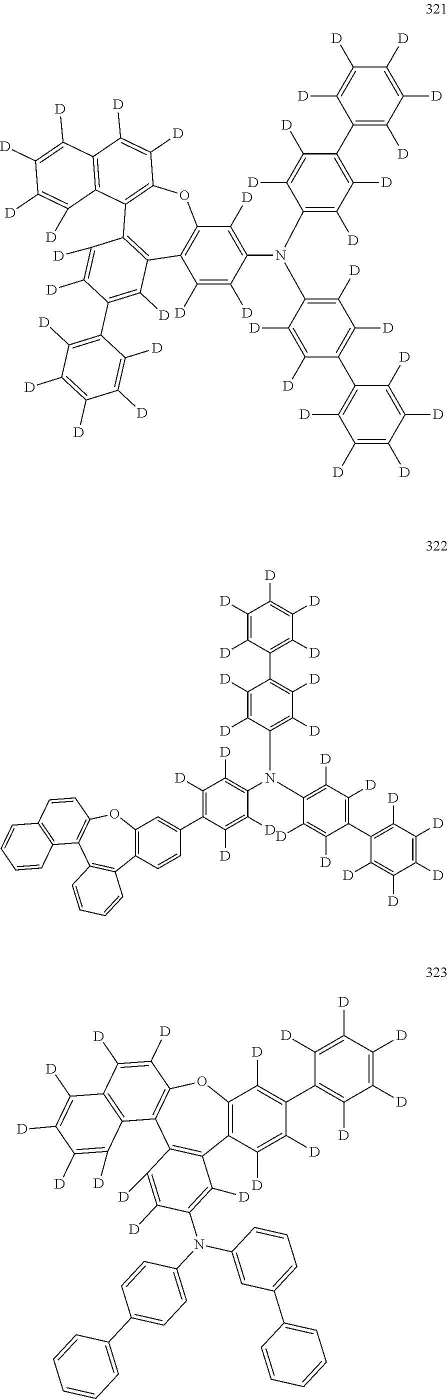

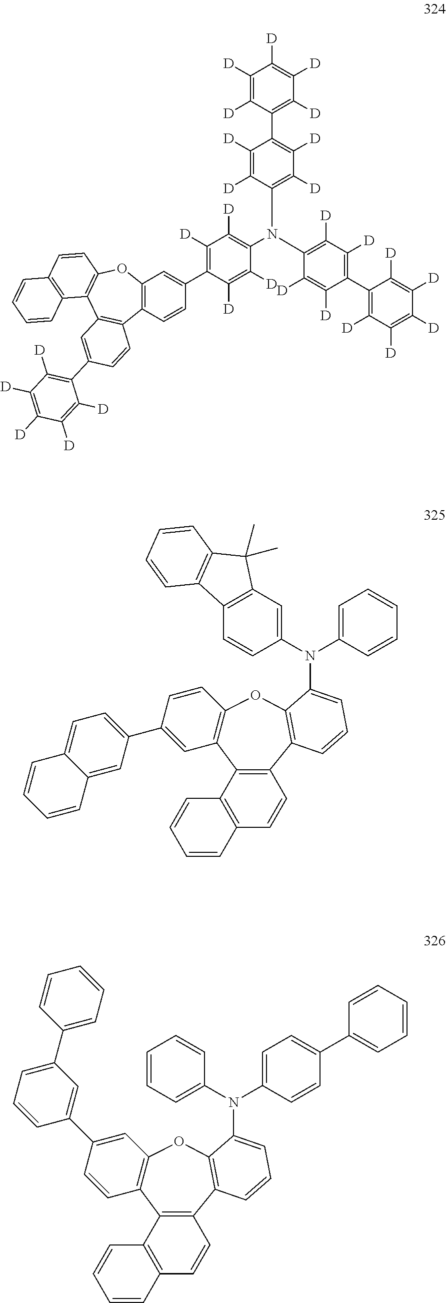 [SMILES: [2H]c1c([2H])c([2H])c(-c2ccc3c(c2)-c2c(ccc4ccccc24)Oc2cc(-c4c([2H])c([2H])c(N(c5c([2H])c([2H])c(-c6c([2H])c([2H])c([2H])c([2H])c6[2H])c([2H])c5[2H])c5c([2H])c([2H])c(-c6c([2H])c([2H])c([2H])c([2H])c6[2H])c([2H])c5[2H])c([2H])c4[2H])ccc2-3)c([2H])c1[2H] ; CC1(C)c2ccccc2-c2ccc(N(c3ccccc3)c3cccc4c3Oc3ccc(-c5ccc6ccccc6c5)cc3-c3c-4ccc4ccccc34)cc21 ; c1ccc(-c2ccc(N(c3ccccc3)c3cccc4c3Oc3cc(-c5cccc(-c6ccccc6)c5)ccc3-c3c-4ccc4ccccc34)cc2)cc1]

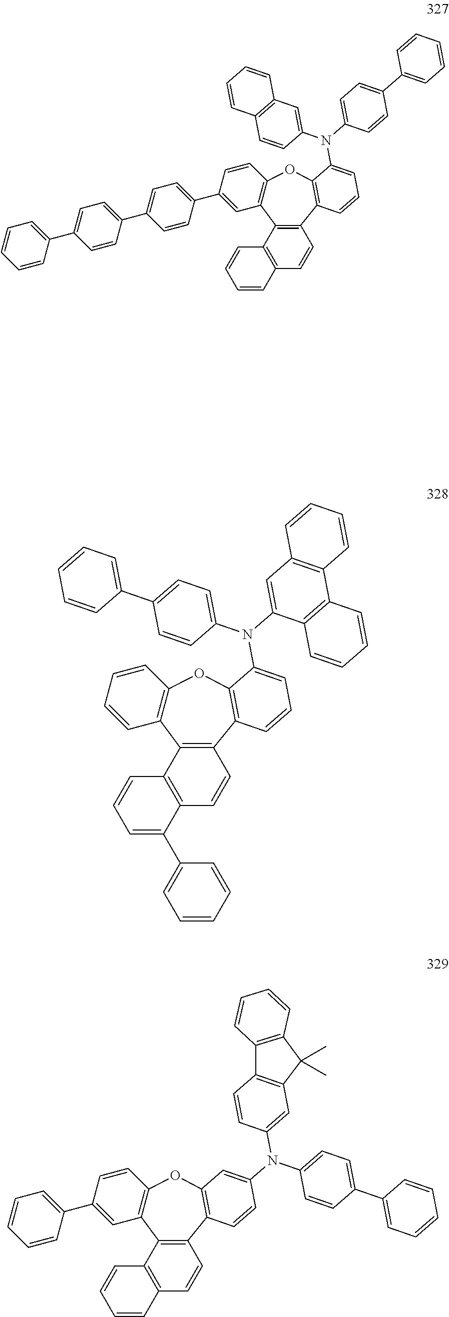 [SMILES: c1ccc(-c2ccc(-c3ccc(-c4ccc5c(c4)-c4c(ccc6ccccc46)-c4cccc(N(c6ccc(-c7ccccc7)cc6)c6ccc7ccccc7c6)c4O5)cc3)cc2)cc1 ; c1ccc(-c2ccc(N(c3cccc4c3Oc3ccccc3-c3c-4ccc4c(-c5ccccc5)cccc34)c3cc4ccccc4c4ccccc34)cc2)cc1 ; CC1(C)c2ccccc2-c2ccc(N(c3ccc(-c4ccccc4)cc3)c3ccc4c(c3)Oc3ccc(-c5ccccc5)cc3-c3c-4ccc4ccccc34)cc21]

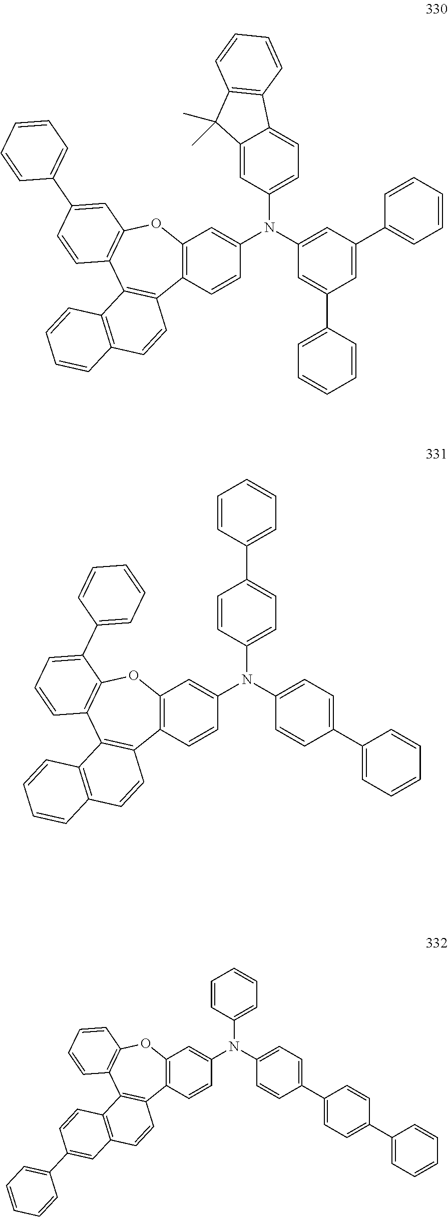 [SMILES: CC1(C)c2ccccc2-c2ccc(N(c3cc(-c4ccccc4)cc(-c4ccccc4)c3)c3ccc4c(c3)Oc3cc(-c5ccccc5)ccc3-c3c-4ccc4ccccc34)cc21 ; c1ccc(-c2ccc(N(c3ccc(-c4ccccc4)cc3)c3ccc4c(c3)Oc3c(-c5ccccc5)cccc3-c3c-4ccc4ccccc34)cc2)cc1 ; c1ccc(-c2ccc(-c3ccc(N(c4ccccc4)c4ccc5c(c4)Oc4ccccc4-c4c-5ccc5cc(-c6ccccc6)ccc45)cc3)cc2)cc1]

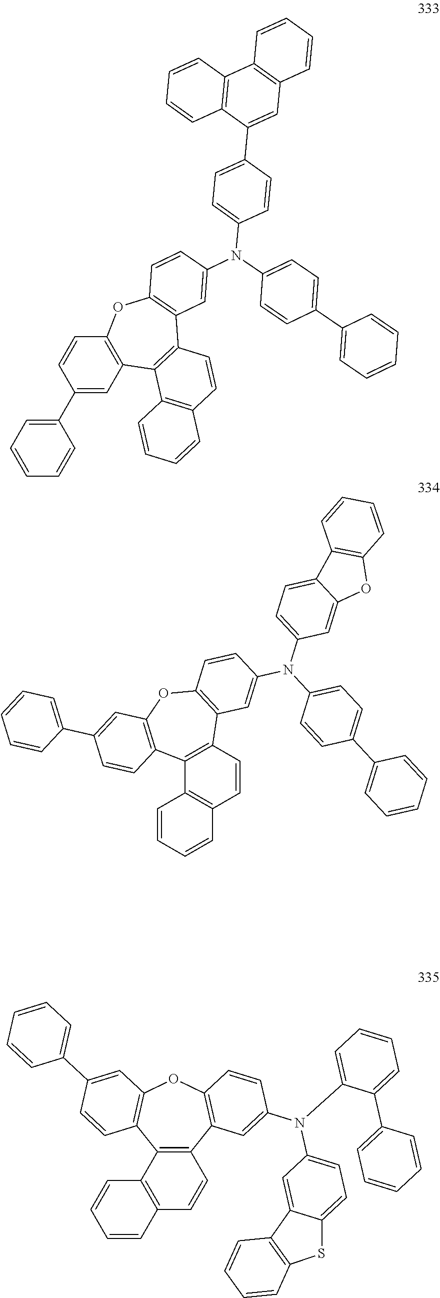 [SMILES: c1ccc(-c2ccc(N(c3ccc(-c4cc5ccccc5c5ccccc45)cc3)c3ccc4c(c3)-c3ccc5ccccc5c3-c3cc(-c5ccccc5)ccc3O4)cc2)cc1 ; c1ccc(-c2ccc(N(c3ccc4c(c3)-c3ccc5ccccc5c3-c3ccc(-c5ccccc5)cc3O4)c3ccc4c(c3)oc3ccccc34)cc2)cc1 ; c1ccc(-c2ccc3c(c2)Oc2ccc(N(c4ccc5sc6ccccc6c5c4)c4ccccc4-c4ccccc4)cc2-c2ccc4ccccc4c2-3)cc1]

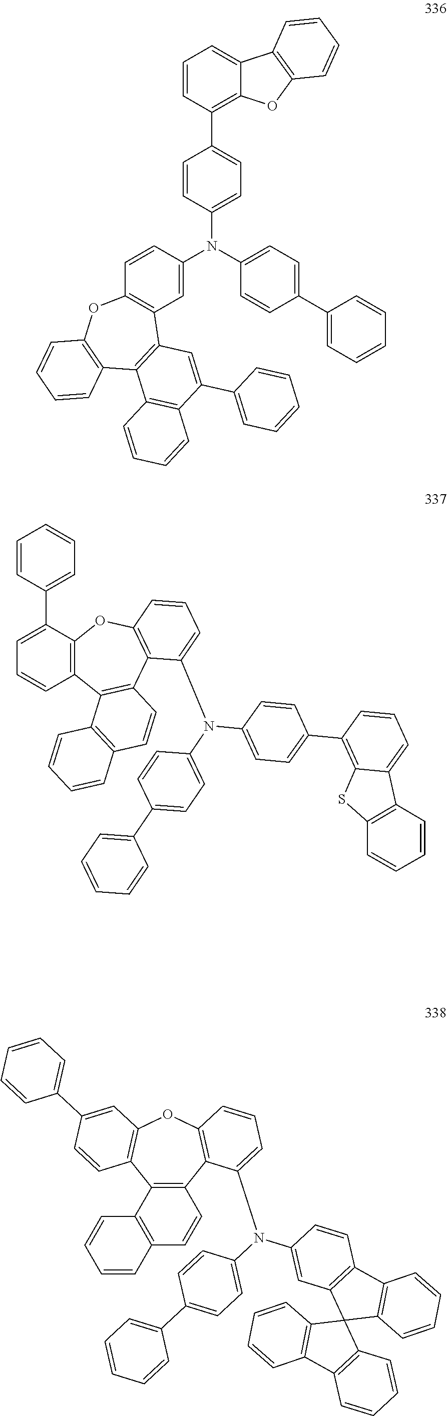 [SMILES: c1ccc(-c2ccc(N(c3ccc(-c4cccc5c4oc4ccccc45)cc3)c3ccc4c(c3)-c3cc(-c5ccccc5)c5ccccc5c3-c3ccccc3O4)cc2)cc1 ; c1ccc(-c2ccc(N(c3ccc(-c4cccc5c4sc4ccccc45)cc3)c3cccc4c3-c3ccc5ccccc5c3-c3cccc(-c5ccccc5)c3O4)cc2)cc1 ; c1ccc(-c2ccc(N(c3ccc4c(c3)C3(c5ccccc5-c5ccccc53)c3ccccc3-4)c3cccc4c3-c3ccc5ccccc5c3-c3ccc(-c5ccccc5)cc3O4)cc2)cc1]

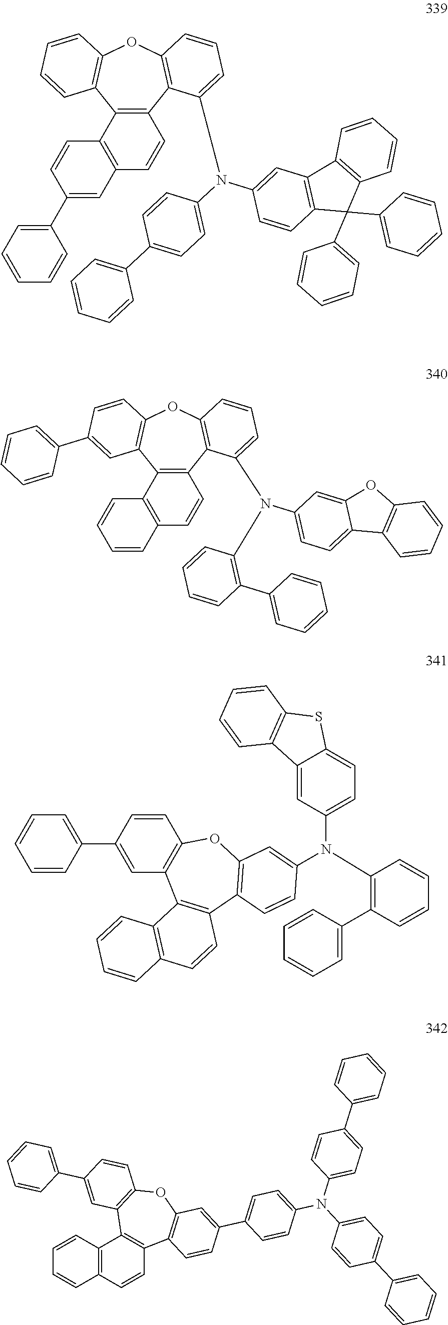 [SMILES: c1ccc(-c2ccc(N(c3ccc4c(c3)-c3ccccc3C4(c3ccccc3)c3ccccc3)c3cccc4c3-c3ccc5cc(-c6ccccc6)ccc5c3-c3ccccc3O4)cc2)cc1 ; c1ccc(-c2ccc3c(c2)-c2c(ccc4ccccc24)-c2c(cccc2N(c2ccc4c(c2)oc2ccccc24)c2ccccc2-c2ccccc2)O3)cc1 ; c1ccc(-c2ccc3c(c2)-c2c(ccc4ccccc24)-c2ccc(N(c4ccc5sc6ccccc6c5c4)c4ccccc4-c4ccccc4)cc2O3)cc1 ; c1ccc(-c2ccc(N(c3ccc(-c4ccccc4)cc3)c3ccc(-c4ccc5c(c4)Oc4ccc(-c6ccccc6)cc4-c4c-5ccc5ccccc45)cc3)cc2)cc1]

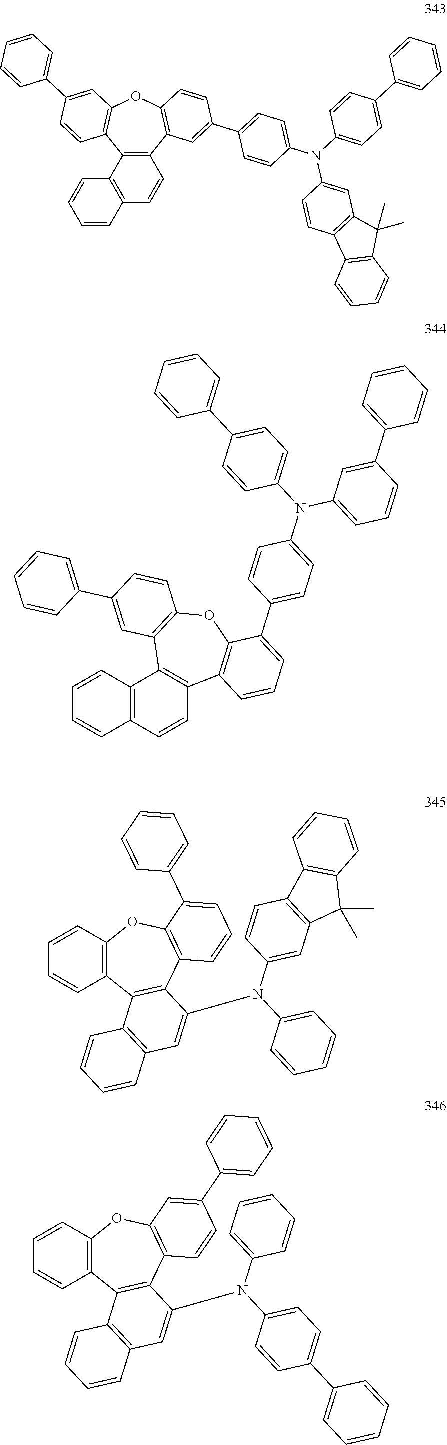 [SMILES: CC1(C)c2ccccc2-c2ccc(N(c3ccc(-c4ccccc4)cc3)c3ccc(-c4ccc5c(c4)-c4ccc6ccccc6c4-c4ccc(-c6ccccc6)cc4O5)cc3)cc21 ; c1ccc(-c2ccc(N(c3ccc(-c4cccc5c4Oc4ccc(-c6ccccc6)cc4-c4c-5ccc5ccccc45)cc3)c3cccc(-c4ccccc4)c3)cc2)cc1 ; CC1(C)c2ccccc2-c2ccc(N(c3ccccc3)c3cc4ccccc4c4c3-c3cccc(-c5ccccc5)c3Oc3ccccc3-4)cc21 ; c1ccc(-c2ccc(N(c3ccccc3)c3cc4ccccc4c4c3-c3ccc(-c5ccccc5)cc3Oc3ccccc3-4)cc2)cc1]

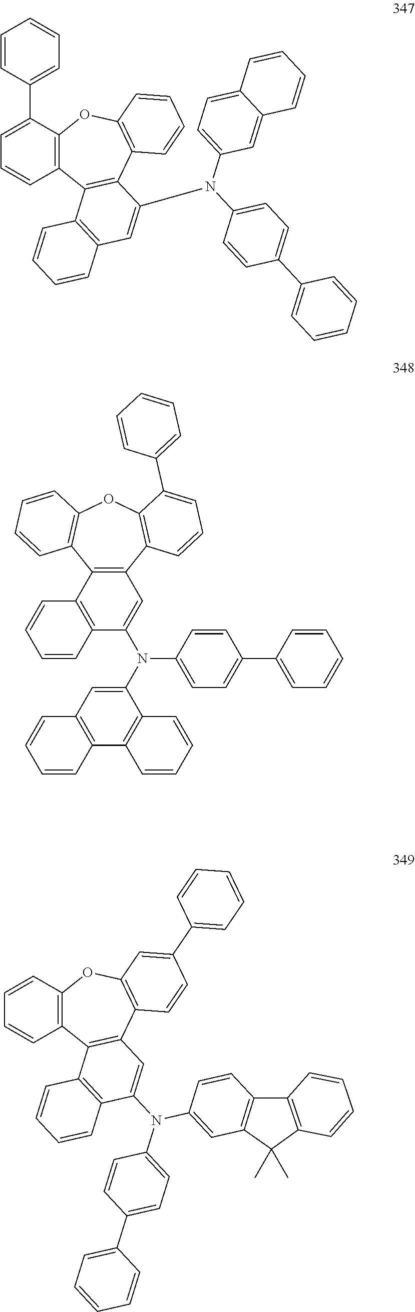 [SMILES: c1ccc(-c2ccc(N(c3ccc4ccccc4c3)c3cc4ccccc4c4c3-c3ccccc3Oc3c(-c5ccccc5)cccc3-4)cc2)cc1 ; c1ccc(-c2ccc(N(c3cc4c(c5ccccc35)-c3ccccc3Oc3c(-c5ccccc5)cccc3-4)c3cc4ccccc4c4ccccc34)cc2)cc1 ; CC1(C)c2ccccc2-c2ccc(N(c3ccc(-c4ccccc4)cc3)c3cc4c(c5ccccc35)-c3ccccc3Oc3cc(-c5ccccc5)ccc3-4)cc21]

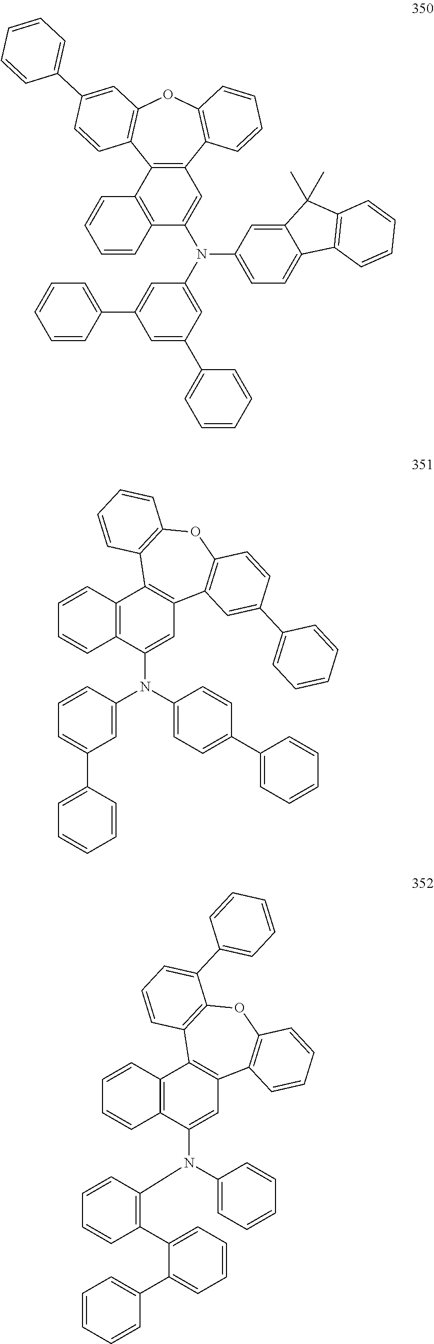 [SMILES: CC1(C)c2ccccc2-c2ccc(N(c3cc(-c4ccccc4)cc(-c4ccccc4)c3)c3cc4c(c5ccccc35)-c3ccc(-c5ccccc5)cc3Oc3ccccc3-4)cc21 ; c1ccc(-c2ccc(N(c3cccc(-c4ccccc4)c3)c3cc4c(c5ccccc35)-c3ccccc3Oc3ccc(-c5ccccc5)cc3-4)cc2)cc1 ; c1ccc(-c2ccccc2-c2ccccc2N(c2ccccc2)c2cc3c(c4ccccc24)-c2cccc(-c4ccccc4)c2Oc2ccccc2-3)cc1]

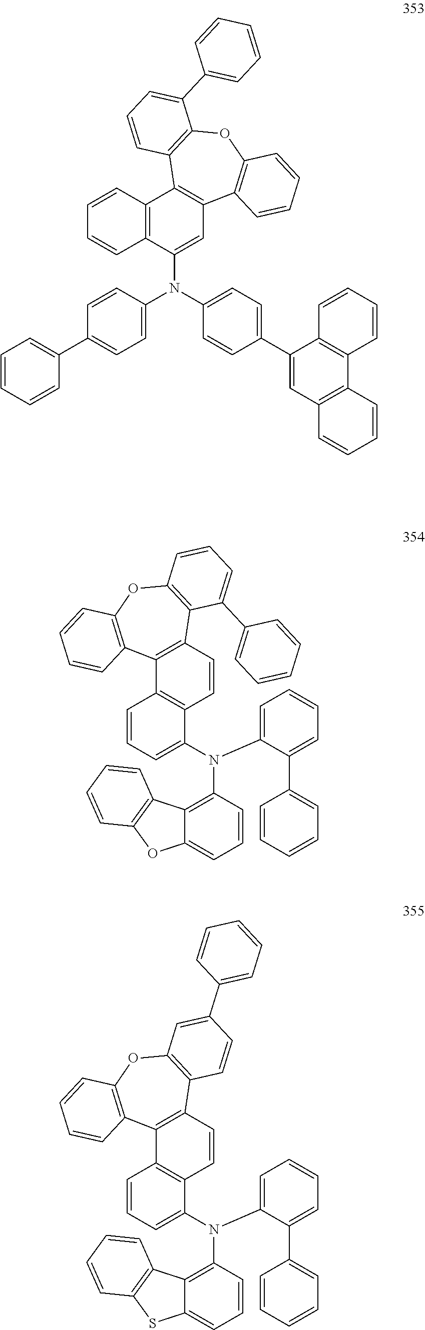 [SMILES: c1ccc(-c2ccc(N(c3ccc(-c4cc5ccccc5c5ccccc45)cc3)c3cc4c(c5ccccc35)-c3cccc(-c5ccccc5)c3Oc3ccccc3-4)cc2)cc1 ; c1ccc(-c2ccccc2N(c2cccc3c4c(ccc23)-c2c(cccc2-c2ccccc2)Oc2ccccc2-4)c2cccc3oc4ccccc4c23)cc1 ; c1ccc(-c2ccc3c(c2)Oc2ccccc2-c2c-3ccc3c(N(c4ccccc4-c4ccccc4)c4cccc5sc6ccccc6c45)cccc23)cc1]

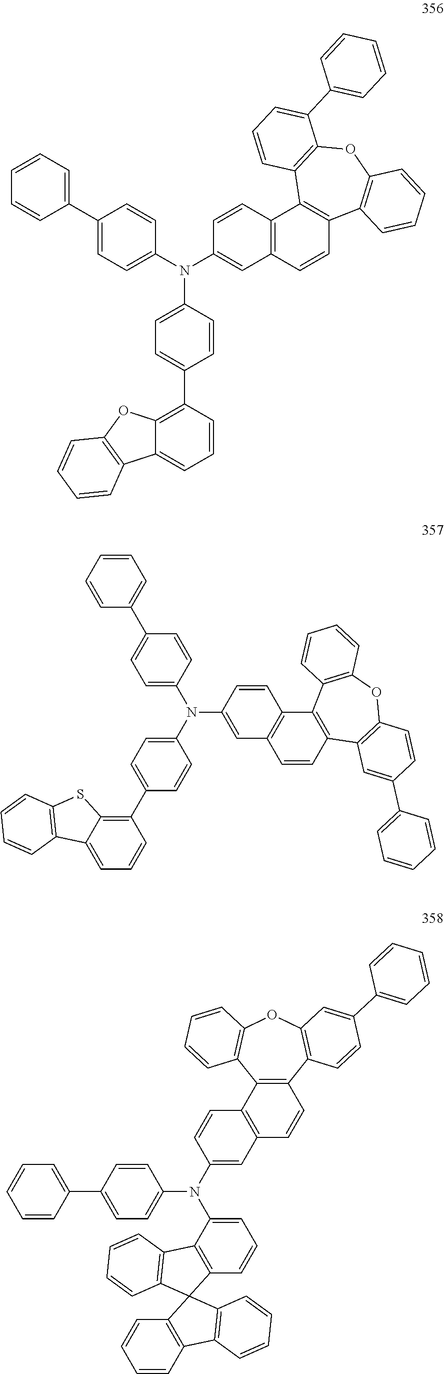 [SMILES: c1ccc(-c2ccc(N(c3ccc(-c4cccc5c4oc4ccccc45)cc3)c3ccc4c5c(ccc4c3)-c3ccccc3Oc3c(-c4ccccc4)cccc3-5)cc2)cc1 ; c1ccc(-c2ccc(N(c3ccc(-c4cccc5c4sc4ccccc45)cc3)c3ccc4c5c(ccc4c3)-c3cc(-c4ccccc4)ccc3Oc3ccccc3-5)cc2)cc1 ; c1ccc(-c2ccc(N(c3ccc4c5c(ccc4c3)-c3ccc(-c4ccccc4)cc3Oc3ccccc3-5)c3cccc4c3-c3ccccc3C43c4ccccc4-c4ccccc43)cc2)cc1]

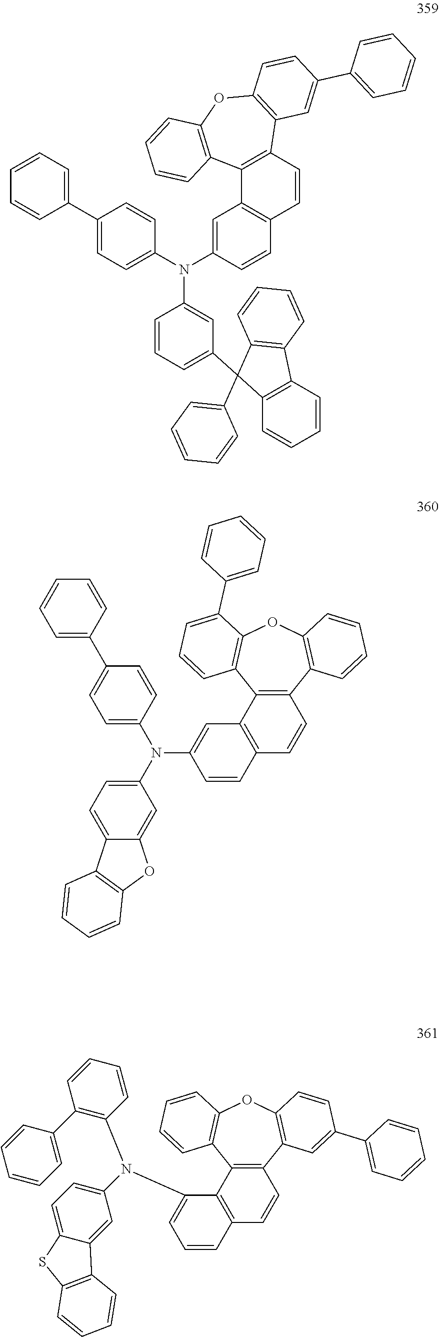 [SMILES: c1ccc(-c2ccc(N(c3cccc(C4(c5ccccc5)c5ccccc5-c5ccccc54)c3)c3ccc4ccc5c(c4c3)-c3ccccc3Oc3ccc(-c4ccccc4)cc3-5)cc2)cc1 ; c1ccc(-c2ccc(N(c3ccc4c(c3)oc3ccccc34)c3ccc4ccc5c(c4c3)-c3cccc(-c4ccccc4)c3Oc3ccccc3-5)cc2)cc1 ; c1ccc(-c2ccc3c(c2)-c2ccc4cccc(N(c5ccc6sc7ccccc7c6c5)c5ccccc5-c5ccccc5)c4c2-c2ccccc2O3)cc1]

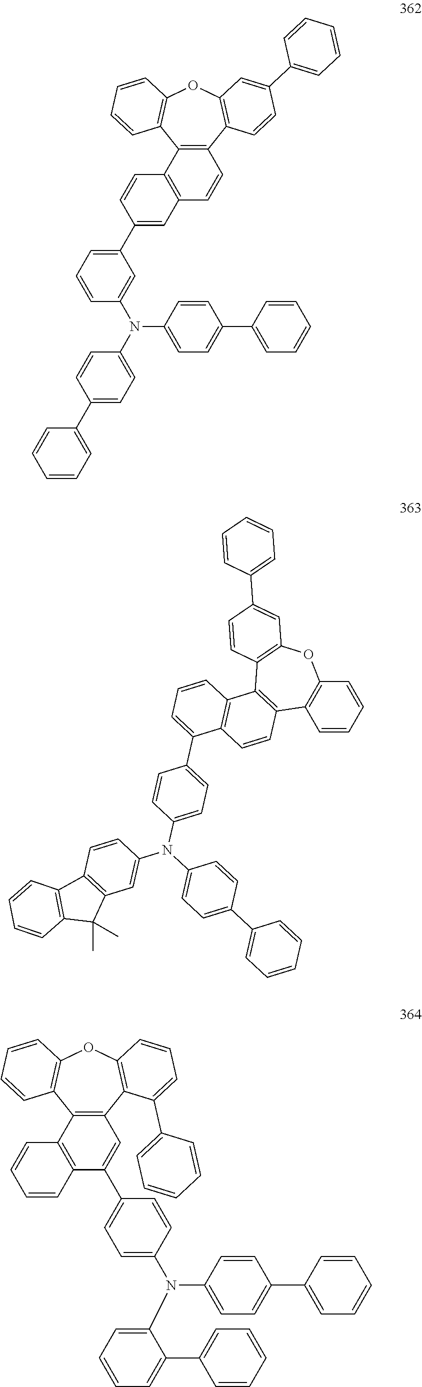 [SMILES: c1ccc(-c2ccc(N(c3ccc(-c4ccccc4)cc3)c3cccc(-c4ccc5c6c(ccc5c4)-c4ccc(-c5ccccc5)cc4Oc4ccccc4-6)c3)cc2)cc1 ; CC1(C)c2ccccc2-c2ccc(N(c3ccc(-c4ccccc4)cc3)c3ccc(-c4cccc5c6c(ccc45)-c4ccccc4Oc4cc(-c5ccccc5)ccc4-6)cc3)cc21 ; c1ccc(-c2ccc(N(c3ccc(-c4cc5c(c6ccccc46)-c4ccccc4Oc4cccc(-c6ccccc6)c4-5)cc3)c3ccccc3-c3ccccc3)cc2)cc1]

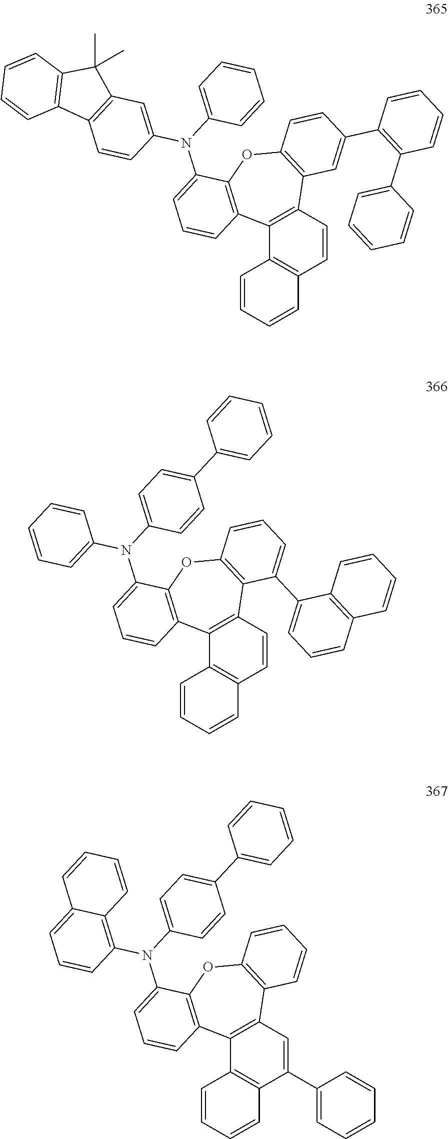 [SMILES: CC1(C)c2ccccc2-c2ccc(N(c3ccccc3)c3cccc4c3Oc3ccc(-c5ccccc5-c5ccccc5)cc3-c3ccc5ccccc5c3-4)cc21 ; c1ccc(-c2ccc(N(c3ccccc3)c3cccc4c3Oc3cccc(-c5cccc6ccccc56)c3-c3ccc5ccccc5c3-4)cc2)cc1 ; c1ccc(-c2ccc(N(c3cccc4c3Oc3ccccc3-c3cc(-c5ccccc5)c5ccccc5c3-4)c3cccc4ccccc34)cc2)cc1]

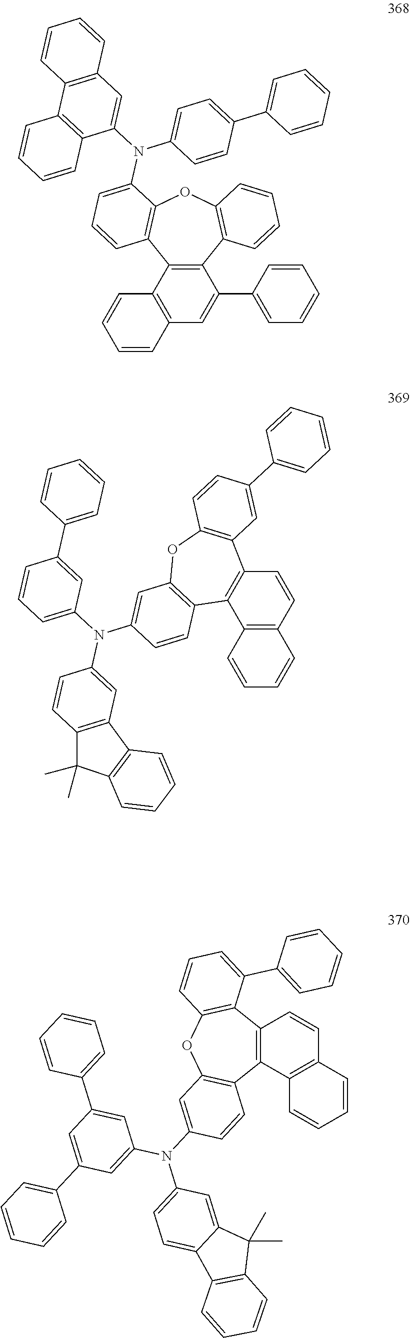 [SMILES: c1ccc(-c2ccc(N(c3cccc4c3Oc3ccccc3-c3c(-c5ccccc5)cc5ccccc5c3-4)c3cc4ccccc4c4ccccc34)cc2)cc1 ; CC1(C)c2ccccc2-c2cc(N(c3cccc(-c4ccccc4)c3)c3ccc4c(c3)Oc3ccc(-c5ccccc5)cc3-c3ccc5ccccc5c3-4)ccc21 ; CC1(C)c2ccccc2-c2ccc(N(c3cc(-c4ccccc4)cc(-c4ccccc4)c3)c3ccc4c(c3)Oc3cccc(-c5ccccc5)c3-c3ccc5ccccc5c3-4)cc21]

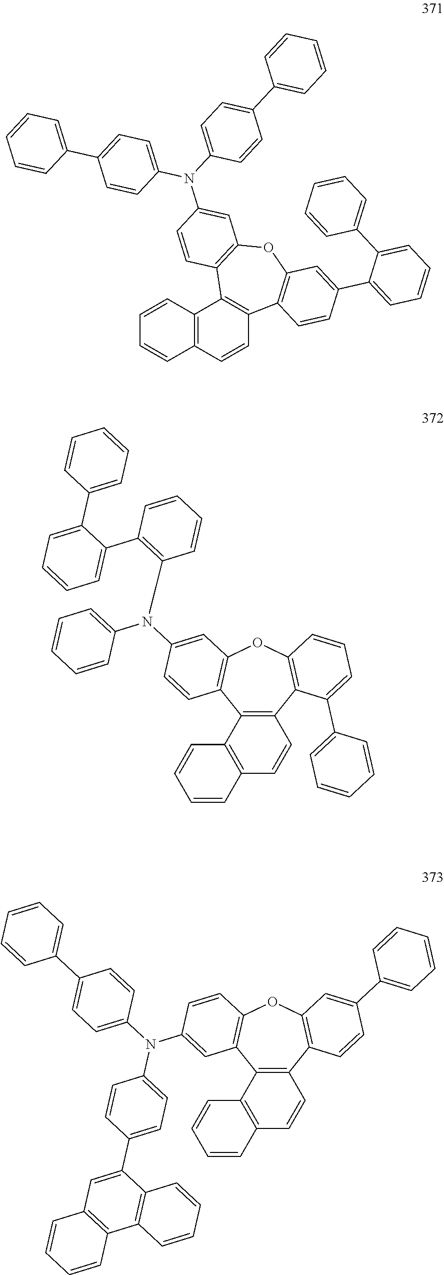 [SMILES: c1ccc(-c2ccc(N(c3ccc(-c4ccccc4)cc3)c3ccc4c(c3)Oc3cc(-c5ccccc5-c5ccccc5)ccc3-c3ccc5ccccc5c3-4)cc2)cc1 ; c1ccc(-c2ccccc2-c2ccccc2N(c2ccccc2)c2ccc3c(c2)Oc2cccc(-c4ccccc4)c2-c2ccc4ccccc4c2-3)cc1 ; c1ccc(-c2ccc(N(c3ccc(-c4cc5ccccc5c5ccccc45)cc3)c3ccc4c(c3)-c3c(ccc5ccccc35)-c3ccc(-c5ccccc5)cc3O4)cc2)cc1]

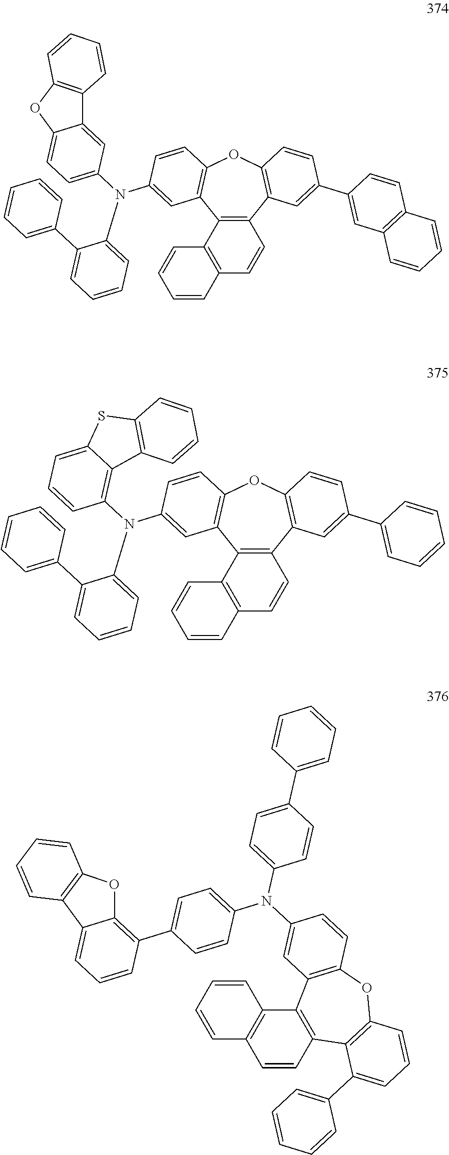 [SMILES: c1ccc(-c2ccccc2N(c2ccc3c(c2)-c2c(ccc4ccccc24)-c2cc(-c4ccc5ccccc5c4)ccc2O3)c2ccc3oc4ccccc4c3c2)cc1 ; c1ccc(-c2ccc3c(c2)-c2ccc4ccccc4c2-c2cc(N(c4ccccc4-c4ccccc4)c4cccc5sc6ccccc6c45)ccc2O3)cc1 ; c1ccc(-c2ccc(N(c3ccc(-c4cccc5c4oc4ccccc45)cc3)c3ccc4c(c3)-c3c(ccc5ccccc35)-c3c(cccc3-c3ccccc3)O4)cc2)cc1]

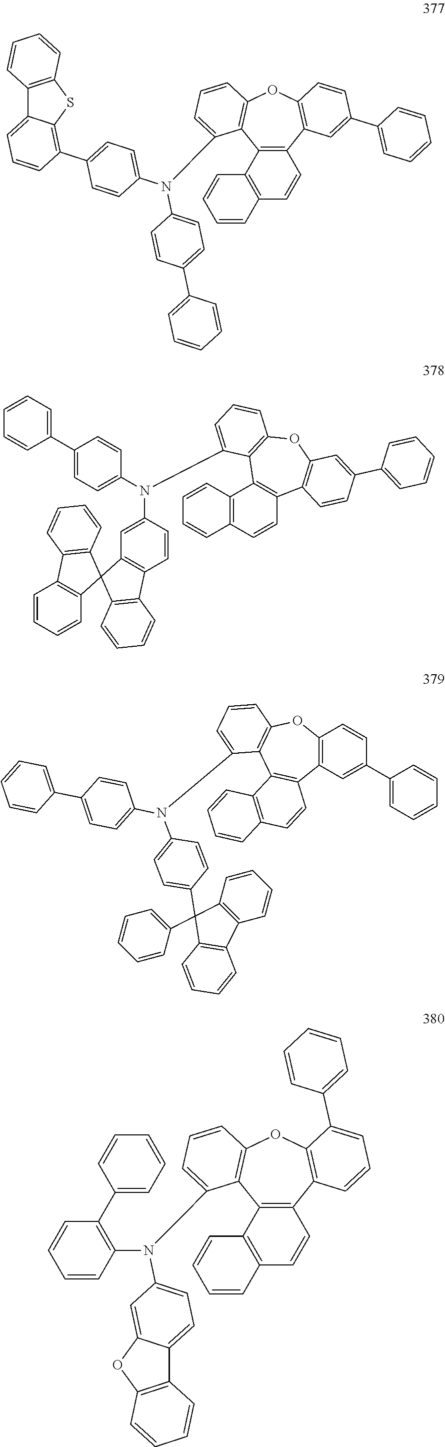 [SMILES: c1ccc(-c2ccc(N(c3ccc(-c4cccc5c4sc4ccccc45)cc3)c3cccc4c3-c3c(ccc5ccccc35)-c3cc(-c5ccccc5)ccc3O4)cc2)cc1 ; c1ccc(-c2ccc(N(c3ccc4c(c3)C3(c5ccccc5-c5ccccc53)c3ccccc3-4)c3cccc4c3-c3c(ccc5ccccc35)-c3ccc(-c5ccccc5)cc3O4)cc2)cc1 ; c1ccc(-c2ccc(N(c3ccc(C4(c5ccccc5)c5ccccc5-c5ccccc54)cc3)c3cccc4c3-c3c(ccc5ccccc35)-c3cc(-c5ccccc5)ccc3O4)cc2)cc1 ; c1ccc(-c2ccccc2N(c2ccc3c(c2)oc2ccccc23)c2cccc3c2-c2c(ccc4ccccc24)-c2cccc(-c4ccccc4)c2O3)cc1]

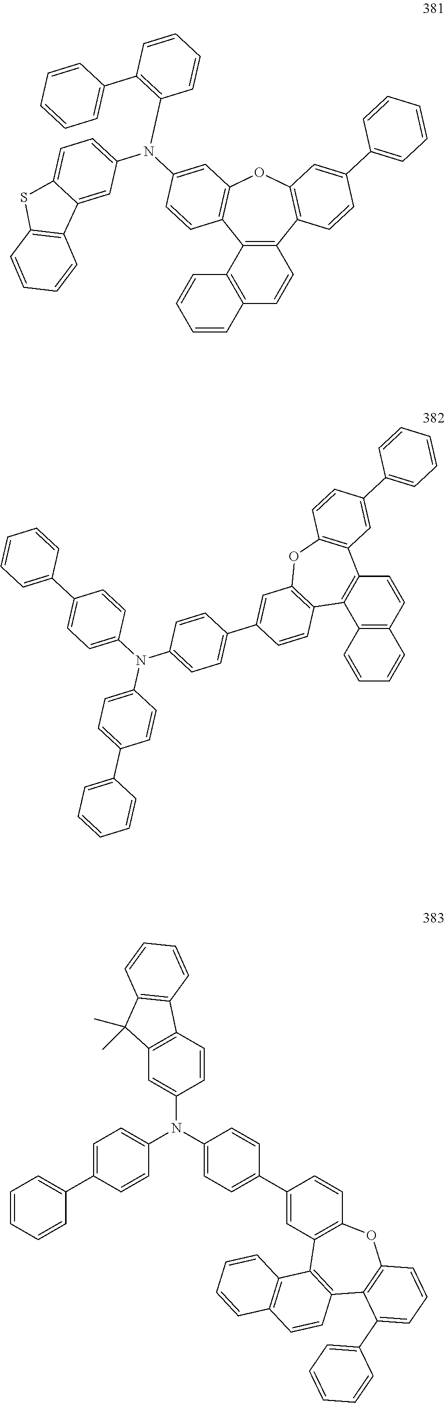 [SMILES: c1ccc(-c2ccc3c(c2)Oc2cc(N(c4ccc5sc6ccccc6c5c4)c4ccccc4-c4ccccc4)ccc2-c2c-3ccc3ccccc23)cc1 ; c1ccc(-c2ccc(N(c3ccc(-c4ccccc4)cc3)c3ccc(-c4ccc5c(c4)Oc4ccc(-c6ccccc6)cc4-c4ccc6ccccc6c4-5)cc3)cc2)cc1 ; CC1(C)c2ccccc2-c2ccc(N(c3ccc(-c4ccccc4)cc3)c3ccc(-c4ccc5c(c4)-c4c(ccc6ccccc46)-c4c(cccc4-c4ccccc4)O5)cc3)cc21]

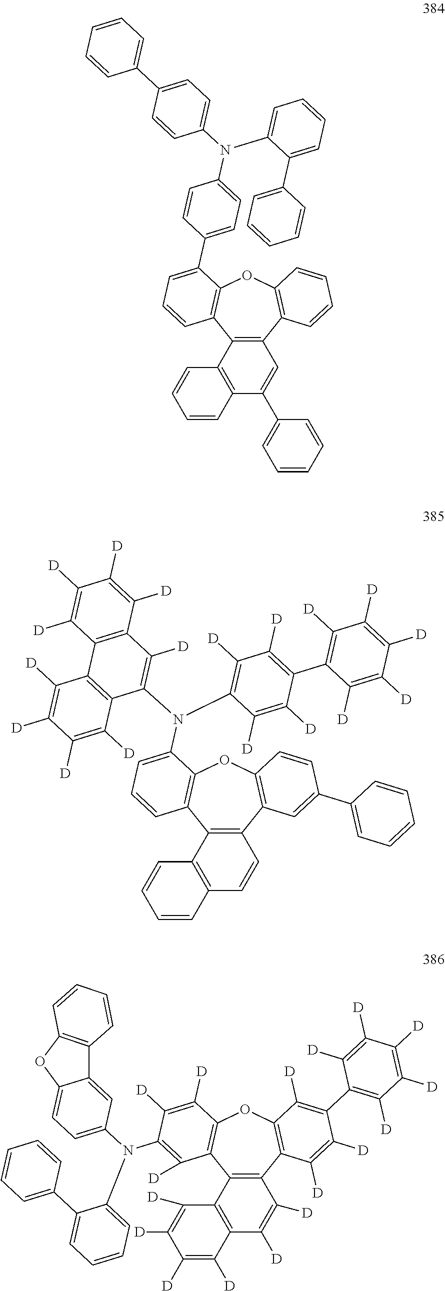 [SMILES: c1ccc(-c2ccc(N(c3ccc(-c4cccc5c4Oc4ccccc4-c4cc(-c6ccccc6)c6ccccc6c4-5)cc3)c3ccccc3-c3ccccc3)cc2)cc1 ; [2H]c1c([2H])c([2H])c(-c2c([2H])c([2H])c(N(c3cccc4c3Oc3ccc(-c5ccccc5)cc3-c3ccc5ccccc5c3-4)c3c([2H])c4c([2H])c([2H])c([2H])c([2H])c4c4c([2H])c([2H])c([2H])c([2H])c34)c([2H])c2[2H])c([2H])c1[2H] ; [2H]c1c([2H])c([2H])c(-c2c([2H])c([2H])c3c(c2[2H])Oc2c([2H])c([2H])c(N(c4ccc5oc6ccccc6c5c4)c4ccccc4-c4ccccc4)c([2H])c2-c2c-3c([2H])c([2H])c3c([2H])c([2H])c([2H])c([2H])c23)c([2H])c1[2H]]

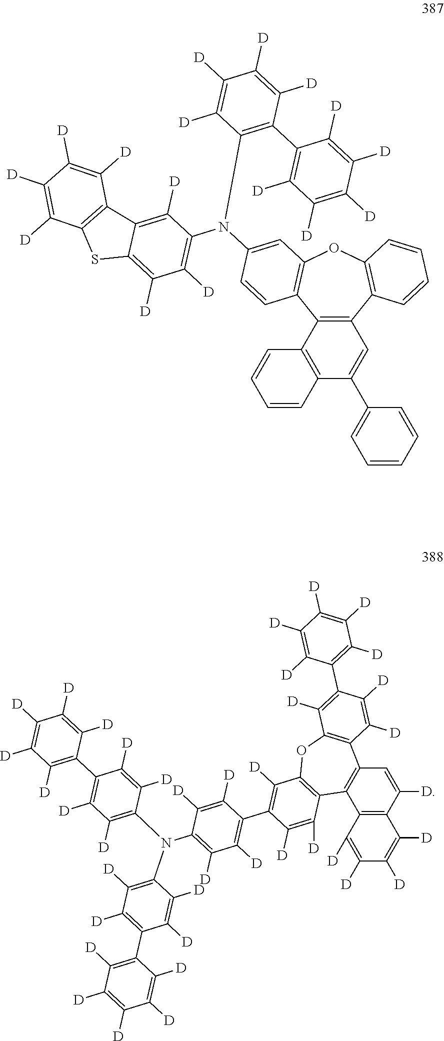

10. An organic light emitting device comprising:
a first electrode;
a second electrode provided opposite to the first electrode; and
one or more organic material layers provided between the first electrode and the second electrode,
wherein one or more layers of the organic material layers include the heterocyclic compound of claim 1.

11. The organic light emitting device of claim 10, wherein the organic material layer includes a hole transport layer, and the hole transport layer includes the heterocyclic compound.

12. The organic light emitting device of claim 10, wherein the organic material layer includes an electron blocking layer, and the electron blocking layer includes the heterocyclic compound.

13. The organic light emitting device of claim 10, further comprising one, two or more layers selected from the group consisting of a light emitting layer, a hole injection layer, a hole blocking layer, an electron injection layer, an electron transport layer and a hole blocking layer.
